(19) United States
(12) Patent Application Publication
(10) Pub. No.: US 2024/0254512 A1
(43) Pub. Date: Aug. 1, 2024

(54) ANELLOVECTORS AND METHODS OF USE

(71) Applicant: FLAGSHIP PIONEERING INNOVATIONS V, INC., Cambridge, MA (US)

(72) Inventors: Nathan Lawrence Yozwiak, Newton, MA (US); Simeon Ungerleider Springer, Lexington, MA (US); Cesar A. Arze, Boston, MA (US); Kristian Graugaard Andersen, Cambridge, MA (US)

(21) Appl. No.: 18/559,921

(22) PCT Filed: May 11, 2022
(86) PCT No.: PCT/US22/28805

§ 371 Date: Nov. 9, 2023

Related U.S. Application Data
(60) Provisional application No. 63/187,877, filed on May 12, 2021.

(51) Int. Cl. C12N 15/86; C07K 14/005

(52) U.S. Cl. CPC C12N 15/86; C07K 14/005; C12N 2750/00022; C12N 2750/00043; C12N 2750/00051

(57) ABSTRACT

This invention relates generally to anellovectors and compositions and uses thereof.

### SEQUENCE LISTING

The instant application contains a Sequence Listing which has been submitted electronically in ASCII format and is hereby incorporated by reference in its entirety. Said ASCII copy, created on May 10, 2022, is named V2057-7019WO_SL.txt and is 16,689,317 bytes in size.

### BACKGROUND

There is an ongoing need to develop suitable vectors to deliver therapeutic genetic material to patients.

### SUMMARY

The present disclosure provides an anellovector, e.g., a synthetic anellovector, that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). In some embodiments, an anellovector (e.g., particle, e.g., a viral particle, e.g., an Anellovirus particle) comprises a genetic element (e.g., a genetic element comprising a therapeutic DNA sequence) encapsulated in a proteinaceous exterior (e.g., a proteinaceous exterior comprising an Anellovirus capsid protein, e.g., an Anellovirus ORF1 protein or a polypeptide encoded by an Anellovirus ORF1 nucleic acid, e.g., as described herein), which is capable of introducing the genetic element into a cell (e.g., a mammalian cell, e.g., a human cell). In some embodiments, the anellovector is a particle comprising a proteinaceous exterior comprising a polypeptide encoded by an Anellovirus ORF1 nucleic acid (e.g., an ORF1 nucleic acid of Alphatorquevirus. Betatorquevirus. or Gammatorquevirus, e.g., an ORF1 of Alphatorquevirus clade 1, Alphatorquevirus clade 2, Alphatorquevirus clade 3, Alphatorquevirus clade 4, Alphatorquevirus clade 5, Alphatorquevirus clade 6, or Alphatorquevirus clade 7, e.g., as described herein). The genetic element of an anellovector of the present disclosure is typically a circular and/or single-stranded DNA molecule (e.g., circular and single stranded), and generally includes a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior. In some instances, the genetic element is circular or linear. In some instances, the genetic element comprises or encodes an effector (e.g., a nucleic acid effector, such as a non-coding RNA, or a polypeptide effector, e.g., a protein), e.g., which can be expressed in the cell. In some embodiments, the effector is a therapeutic agent or a therapeutic effector, e.g., as described herein. In some instances, the effector is an endogenous effector or an exogenous effector, e.g., to a wild-type Anellovirus or a target cell. In some embodiments, the effector is exogenous to a wild-type Anellovirus or a target cell. In some embodiments, the anellovector can deliver an effector into a cell by contacting the cell and introducing a genetic element encoding the effector into the cell, such that the effector is made or expressed by the cell. In certain instances, the effector is an endogenous effector (e.g., endogenous to the target cell but, e.g., provided in increased amounts by the anellovector). In other instances, the effector is an exogenous effector. The effector can, in some instances, modulate a function of the cell or modulate an activity or level of a target molecule in the cell. For example, the effector can decrease levels of a target protein in the cell (e.g., as described in Examples 3 and 4). In another example, the anellovector can deliver and express an effector, e.g., an exogenous protein, in vivo (e.g., as described in Examples 19 and 28). Anellovectors can be used, for example, to deliver genetic material to a target cell, tissue or subject; to deliver an effector to a target cell, tissue or subject; or for treatment of diseases and disorders, e.g., by delivering an effector that can operate as a therapeutic agent to a desired cell, tissue, or subject.

The invention further provides synthetic anellovectors. A synthetic anellovector has at least one structural difference compared to a wild-type virus (e.g., a wild-type Anellovirus, e.g., a described herein), e.g., a deletion, insertion, substitution, modification (e.g., enzymatic modification), relative to the wild-type virus. Generally, synthetic anellovectors include an exogenous genetic element enclosed within a proteinaceous exterior, which can be used for delivering the genetic element, or an effector (e.g., an exogenous effector or an endogenous effector) encoded therein (e.g., a polypeptide or nucleic acid effector), into eukaryotic (e.g., human) cells. In some embodiments, the anellovector does not cause a detectable and/or an unwanted immune or inflammarory response, e.g., does not cause more than a 1%, 5%, 10%, 15% increase in a molecular marker(s) of inflammation, e.g., TNF-alpha, IL-6, IL-12, IFN, as well as B-cell response e.g. reactive or neutralizing antibodies, e.g., the anellovector may be substantially non-immunogenic to the target cell, tissue or subject.

In an aspect, the invention features an anellovector comprising: (i) a genetic element comprising a promoter element and a sequence encoding an effector (e.g., an endogenous or exogenous effector), and a protein binding sequence (e.g., an exterior protein binding sequence, e.g., a packaging signal); and (ii) a proteinaceous exterior; wherein the genetic element is enclosed within the proteinaceous exterior (e.g., a capsid); and wherein the anellovector is capable of delivering the genetic element into a eukaryotic (e.g., mammalian, e.g., human) cell. In some embodiments, the genetic element is a single-stranded and/or circular DNA. Alternatively or in combination, the genetic element has one, two, three, or all of the following properties: is circular, is single-stranded, it integrates into the genome of a cell at a frequency of less than about 0.0001%, 0.001%, 0.005%, 0.01%, 0.05%, 0.1%, 0.5%, 1%, 1.5%, or 2% of the genetic element that enters the cell, and/or it integrates into the genome of a target cell at less than 1, 2, 3, 4, 5, 6, 7, 8, 9, 10, 15, 20, 25, or 30 copies per genome. In some embodiments, integration frequency is determined as described in Wang et al. (2004, Gene Therapy 11: 711-721, incorporated herein by reference in its entirety). In some embodiments, the genetic element is enclosed within the proteinaccous exterior. In some embodiments, the anellovector is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), or TTMDV sequence, e.g., a wild-type Anellovirus sequence as listed in any of Tables A1-A1417 or N1-N1417). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Anellovirus sequence as described herein, e.g., as listed in any of Tables A1-A1417 or N1-N1417). In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell.

In an aspect, the invention features an infectious (to a human cell) particle comprising an Anellovirus capsid (e.g., a capsid comprising an Anellovirus ORF, e.g., ORF1, polypeptide) encapsulating a genetic element comprising a protein binding sequence that binds to the capsid and a heterologous (to the Anellovirus) sequence encoding a therapeutic effector. In some embodiments, the particle is capable of delivering the genetic element into a mammalian, e.g., human, cell. In some embodiments, the genetic element has less than about 6% (e.g., less than 6%, 5.5%, 5%, 4.5%, 4%, 3.5%, 3%, 2.5%, 2%, 1.5%, or less) identity to a wild type Anellovirus. In some embodiments, the genetic element has no more than 1.5%, 2%, 2.5%, 3%, 3.5%, 4%, 4.5%, 5%, 5.5% or 6% identity to a wild type Anellovirus. In some embodiments, the genetic element has at least about 2% to at least about 5.5% (e.g., 2 to 5%, 3% to 5%, 4% to 5%) identity to a wild type Anellovirus. In some embodiments, the genetic element has greater than about 2000, 3000, 4000, 4500, or 5000 nucleotides of non-viral sequence (e.g., non Anellovirus genome sequence). In some embodiments, the genetic element has greater than about 2000 to 5000, 2500 to 4500, 3000 to 4500, 2500 to 4500, 3500, or 4000, 4500 (e.g., between about 3000 to 4500) nucleotides of non-viral sequence (e.g., non Anellovirus genome sequence). In some embodiments, the genetic element is a single-stranded, circular DNA. Alternatively or in combination, the genetic element has one, two or 3 of the following properties: is circular, is single stranded, it integrates into the genome of a cell at a frequency of less than about 0.001%, 0.005%, 0.01%, 0.05%, 0.1%, 0.5%, 1%, 1.5%, or 2% of the genetic element that enters the cell, it integrates into the genome of a target cell at less than 1, 2, 3, 4, 5, 6, 7, 8, 9, 10, 15, 20, 25, or 30 copies per genome or integrates at a frequency of less than about 0.0001%, 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety).

Also described herein are viral vectors and viral particles based on Anelloviruses, which can be used to deliver an agent (e.g., an exogenous effector or an endogenous effector, e.g., a therapeutic effector) to a cell (e.g., a cell in a subject to be treated therapeutically). In some embodiments, Anelloviruses can be used as effective delivery vehicles for introducing an agent, such as an effector described herein, to a target cell, e.g., a target cell in a subject to be treated therapeutically or prophylactically.

In an aspect, the invention features a polypeptide (e.g., a synthetic polypeptide, e.g., an ORF1 molecule) comprising (e.g., in series):
(i) a first region comprising an arginine-rich region, e.g., amino acid sequence having at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof),
(ii) a second region comprising a jelly-roll domain, e.g., an amino acid sequence having at least 30% (e.g., at least about 30, 35, 40, 50, 60, 70, 80, 90, 95, 96, 97, 98, 99, or 100%) sequence identity to a jelly-roll region sequence described herein or a sequence comprising at least 6 beta strands,
(iii) a third region comprising an amino acid sequence having at least 30% (e.g., at least about 30, 35, 40, 50, 60, 70, 80, 90, 95, 96, 97, 98, 99, or 100%) sequence identity to an N22 domain sequence described herein,
(iv) a fourth region comprising an amino acid sequence having at least 70% (e.g., at least about 70, 80, 90, 95, 96, 97, 98, 99, or 100%) sequence identity to an Anellovirus ORF1 C-terminal domain (CTD) sequence described herein, and
(v) optionally wherein the polypeptide has an amino acid sequence having less than 100%, 99%, 98%, 95%, 90%, 85%, 80% sequence identity to a wild type Anellovirus ORF1 protein described herein.

In some embodiments, the polypeptide comprises at least about 70, 80, 90, 95, 96, 97, 98, 99, or 100% sequence identity to an Anellovirus ORF1 molecule as described herein (e.g., as listed in any of Tables A1-A1417). In some embodiments, the polypeptide comprises at least about 70, 80, 90, 95, 96, 97, 98, 99, or 100% sequence identity to a subsequence (e.g., an arginine (Arg)-rich domain, a jelly-roll domain, a hypervariable region (HVR), an N22 domain, or a C-terminal domain (CTD)) of an Anellovirus ORF1 molecule as described herein (e.g., as listed in any of Tables A1-A1417). In one embodiment, the amino acid sequences of the (i), (ii), (iii), and (iv) region have at least 90% sequence identity to their respective references and wherein the polypeptide has an amino acid sequence having less than 100%, 99%, 98%, 95%, 90%, 85%, 80% sequence identity to a wild type Anellovirus ORF1 protein described herein.

In an aspect, the invention features a complex comprising a polypeptide as described herein (e.g., an Anellovirus ORF1 molecule as described herein) and a genetic element comprising a promoter element and a nucleic acid sequence (e.g., a DNA sequence) encoding an effector (e.g., an exogenous effector or an endogenous effector), and a protein binding sequence.

The present disclosure further provides nucleic acid molecules (e.g., a nucleic acid molecule that includes a genetic element as described herein, or a nucleic acid molecule that includes a sequence encoding a proteinaceous exterior protein as described herein). A nucleic acid molecule of the invention may include one or both of (a) a genetic element as described herein, and (b) a nucleic acid sequence encoding a proteinaceous exterior protein as described herein.

In an aspect, the invention features an isolated nucleic acid molecule comprising a genetic element comprising a promoter element operably linked to a sequence encoding an effector, e.g., a payload, and an exterior protein binding sequence. In some embodiments, the exterior protein binding sequence includes a sequence at least 75% (at least 80%, 85%, 90%, 95%, 97%, 100%) identical to a 5′UTR sequence of an Anellovirus, as disclosed herein. In some embodiments, the genetic element is a single-stranded DNA, is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome or integrates at a frequency of less than about 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety). In embodiments, the effector does not originate from TTV and is not an SV40-miR-S1. In embodiments, the nucleic acid molecule does not comprise the polynucleotide sequence of TTMV-LY2. In embodiments, the promoter element is capable of directing expression of the effector in a eukaryotic (e.g., mammalian, e.g., human) cell.

In some embodiments, the nucleic acid molecule is circular. In some embodiments, the nucleic acid molecule is linear. In some embodiments, a nucleic acid molecule described herein comprises one or more modified nucleotides (e.g., a base modification, sugar modification, or backbone modification).

In some embodiments, the nucleic acid molecule comprises a sequence encoding an ORF1 molecule (e.g., an Anellovirus ORF1 protein, e.g., as described herein). In some embodiments, the nucleic acid molecule comprises a sequence encoding an ORF2 molecule (e.g., an Anellovirus ORF2 protein, e.g., as described herein). In some embodiments, the nucleic acid molecule comprises a sequence encoding an ORF3 molecule (e.g., an Anellovirus ORF3 protein, e.g., as described herein). In an aspect, the invention features a genetic element comprising one, two, or three of: (i) a promoter element and a sequence encoding an effector, e.g., an exogenous or endogenous effector; (ii) at least 72 contiguous nucleotides (e.g., at least 72, 73, 74, 75, 76, 77, 78, 79, 80, 90, 100, or 150 nucleotides) having at least 75% (e.g., at least 75, 76, 77, 78, 79, 80, 90, 91, 92, 93, 94, 95, 96, 97, 98, 99, or 100%) sequence identity to a wild-type Anellovirus sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus sequence; and (iii) a protein binding sequence, e.g., an exterior protein binding sequence, and wherein the nucleic acid construct is a single-stranded DNA; and wherein the nucleic acid construct is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome In some embodiments, a genetic element encoding an effector (e.g., an exogenous or endogenous effector, e.g., as described herein) is codon optimized. In some embodiments, the genetic element is circular. In some embodiments, the genetic element is linear. In some embodiments, the genetic element comprises an anellovector, e.g., as described herein. In some embodiments, a genetic element described herein comprises one or more modified nucleotides (e.g., a base modification, sugar modification, or backbone modification). In some embodiments, the genetic element comprises a sequence encoding an ORF1 molecule (e.g., an Anellovirus ORF1 protein, e.g., as described herein). In some embodiments, the genetic element comprises a sequence encoding an ORF2 molecule (e.g., an Anellovirus ORF2 protein, e.g., as described herein). In some embodiments, the genetic element comprises a sequence encoding an ORF3 molecule (e.g., an Anellovirus ORF3 protein, e.g., as described herein).

In an aspect, the invention features a host cell or helper cell comprising: (a) a nucleic acid comprising a sequence encoding one or more of an ORF1 molecule, an ORF2 molecule, or an ORF3 molecule (e.g. a sequence encoding an Anellovirus ORF1 polypeptide described herein), wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (ii) a protein binding sequence that binds the polypeptide of (a), wherein optionally the genetic element does not encode an ORF1 polypeptide (e.g., an ORF1 protein). For example, the host cell or helper cell comprises (a) and (b) either in cis (both part of the same nucleic acid molecule) or in trans (each part of a different nucleic acid molecule). In embodiments, the genetic element of (b) is circular, single-stranded DNA. In some embodiments, the host cell is a manufacturing cell line. In some embodiments, the host cell or helper cell is adherent or in suspension, or both. In some embodiments, the host cell or helper cell is grown in a microcarrier. In some embodiments, the host cell or helper cell is compatible with cGMP manufacturing practices. In some embodiments, the host cell or helper cell is grown in a medium suitable for promoting cell growth. In certain embodiments, once the host cell or helper cell has grown sufficiently (e.g., to an appropriate cell density), the medium may be exchanged with a medium suitable for production of anellovectors by the host cell or helper cell.

In an aspect, the invention features a pharmaceutical composition comprising an anellovector (e.g., a synthetic anellovector) as described herein. In embodiments, the pharmaceutical composition further comprises a pharmaceutically acceptable carrier or excipient. In embodiments, the pharmaceutical composition comprises a unit dose comprising about 10⁵-10¹⁴ genome equivalents of the anellovector per kilogram of a target subject. In some embodiments, the pharmaceutical composition comprising the preparation will be stable over an acceptable period of time and temperature, and/or be compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of anellovector genomes or genomic equivalents (e.g., as defined by number of genomes per volume).

In an aspect, the invention features a method of treating a disease or disorder in a subject, the method comprising administering to the subject an anellovector, e.g., a synthetic anellovector, e.g., as described herein.

In an aspect, the invention features a method of delivering an effector or payload (e.g., an endogenous or exogenous effector) to a cell, tissue or subject, the method comprising administering to the subject an anellovector, e.g., a synthetic anellovector, e.g., as described herein, wherein the anellovector comprises a nucleic acid sequence encoding the effector. In embodiments, the payload is a nucleic acid. In embodiments, the payload is a polypeptide.

In an aspect, the invention features a method of delivering an anellovector to a cell, comprising contacting the anellovector, e.g., a synthetic anellovector, e.g., as described herein, with a cell, e.g., a eukaryotic cell, e.g., a mammalian cell, e.g., in vivo or ex vivo.

In an aspect, the invention features a method of making an anellovector, e.g., a synthetic anellovector. The method includes:
a) providing a host cell comprising:
(i) a first nucleic acid molecule comprising the nucleic acid sequence of a genetic element of an anellovector, e.g., a synthetic anellovector, as described herein, and
(ii) the first nucleic acid or a second nucleic acid molecule encoding one or more of an amino acid sequence chosen from ORF1, ORF2, ORF2/2, ORF2/3, ORF1/1, or ORF1/2, e.g., as listed in any of Tables A1-A1417, or an amino acid sequence having at least 70% (e.g., at least 70%, 75%, 80%, 85%, 90%, 95%, 96%, 97%, 98%, 99%, or 100%) sequence identity thereto; and
b) incubating the host cell under conditions suitable to make the anellovector.

In some embodiments, the method further includes, prior to step (a), introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is a helper (e.g., a helper plasmid or the genome of a helper virus).

In another aspect, the invention features a method of manufacturing an anellovector composition, comprising:
a) providing a host cell comprising, e.g., expressing one or more components (e.g., all of the components) of an anellovector, e.g., a synthetic anellovector, e.g., as described herein. For example, the host cell comprises (a) a nucleic acid comprising a sequence encoding an Anellovirus ORF1 polypeptide described herein, wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (i) a protein binding sequence (e.g, packaging sequence) that binds the polypeptide of (a), wherein the host cell or helper cell comprises (a) and (b) either in cis or in trans. In embodiments, the genetic element of (b) is circular, single-stranded DNA. In some embodiments, the host cell is a manufacturing cell line:
b) culturing the host cell under conditions suitable for producing a preparation of anellovectors from the host cell, wherein the anellovectors of the preparation comprise a proteinaceous exterior (e.g., comprising an ORF1 molecule) encapsulating the genetic element (e.g., as described herein), thereby making a preparation of anellovectors; and
optionally, c) formulating the preparation of anellovectors, e.g., as a pharmaceutical composition suitable for administration to a subject.

In some embodiments, the components of the anellovector are introduced into the host cell at the time of production (e.g., by transient transfection). In some embodiments, the host cell stably expresses the components of the anellovector (e.g., wherein one or more nucleic acids encoding the components of the anellovector are introduced into the host cell, or a progenitor thereof, e.g., by stable transfection).

In some embodiments, the method further comprises one or more purification steps (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

In an aspect, the invention features a method of manufacturing an anellovector composition, comprising: a) providing a plurality of anellovectors described herein, or a preparation of anellovectors described herein; and b) formulating the anellovectors or preparation thereof, e.g., as a pharmaceutical composition suitable for administration to a subject.

In an aspect, the invention features a method of making a host cell, e.g., a first host cell or a producer cell (e.g., as shown in FIG. 12), e.g., a population of first host cells, comprising an anellovector, the method comprising introducing a genetic element, e.g., as described herein, to a host cell and culturing the host cell under conditions suitable for production of the anellovector. In some embodiments, the method further comprises introducing a helper, e.g., a helper virus, to the host cell. In some embodiments, the introducing comprises transfection (e.g., chemical transfection) or electroporation of the host cell with the anellovector.

In an aspect, the invention features a method of making an anellovector, comprising providing a host cell, e.g., a first host cell or producer cell (e.g., as shown in FIG. 12), comprising an anellovector, e.g., as described herein, and purifying the anellovector from the host cell. In some embodiments, the method further comprises, prior to the providing step, contacting the host cell with an anellovector, e.g., as described herein, and incubating the host cell under conditions suitable for production of the anellovector. In some embodiments, the host cell is the first host cell or producer cell described in the above method of making a host cell. In some embodiments, purifying the anellovector from the host cell comprises lysing the host cell.

In some embodiments, the method further comprises a second step of contacting the anellovector produced by the first host cell or producer cell with a second host cell, e.g., a permissive cell (e.g., as shown in FIG. 12), e.g., a population of second host cells. In some embodiments, the method further comprises incubating the second host cell inder conditions suitable for production of the anellovector. In some embodiments, the method further comprises purifying an anellovector from the second host cell, e.g., thereby producing an anellovector seed population. In some embodiments, at least about 2-100-fold more of the anellovector is produced from the population of second host cells than from the population of first host cells. In some embodiments, purifying the anellovector from the second host cell comprises lysing the second host cell. In some embodiments, the method further comprises a second step of contacting the anellovector produced by the second host cell with a third host cell, e.g., permissive cells (e.g., as shown in FIG. 12), e.g., a population of third host cells. In some embodiments, the method further comprises incubating the third host cell inder conditions suitable for production of the anellovector. In some embodiments, the method further comprises purifying an anellovector from the third host cell, e.g., thereby producing an anellovector stock population. In some embodiments, purifying the anellovector from the third host cell comprises lysing the third host cell. In some embodiments, at least about 2-100-fold more of the anellovector is produced from the population of third host cells than from the population of second host cells.

In some embodiments, the host cell is grown in a medium suitable for promoting cell growth. In certain embodiments, once the host cell has grown sufficiently (e.g., to an appropriate cell density), the medium may be exchanged with a medium suitable for production of anellovectors by the host cell. In some embodiments, anellovectors produced by a host cell separated from the host cell (e.g., by lysing the host cell) prior to contact with a second host cell. In some embodiments, anellovectors produced by a host cell are contacted with a second host cell without an intervening purification step.

In an aspect, the invention features a method of making a pharmaceutical anellovector preparation. The method comprises (a) making an anellovector preparation as described herein, (b) evaluating the preparation (e.g., a pharmaceutical anellovector preparation, anellovector seed population or the anellovector stock population) for one or more pharmaceutical quality control parameters, e.g., identity, purity, titer, potency (e.g., in genomic equivalents per anellovector particle), and/or the nucleic acid sequence, e.g., from the genetic element comprised by the anellovector, and (c) formulating the preparation for pharmaceutical use of the evaluation meets a predetermined criterion, e.g, meets a pharmaceutical specification. In some embodiments, evaluating identity comprises evaluating (e.g., confirming) the sequence of the genetic element of the anellovector, e.g., the sequence encoding the effector. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted anellovectors (e.g., an anellovector other than the desired anellovector, e.g., a synthetic anellovector as described herein), free viral capsid protein, adventitious agents, and aggregates. In some embodiments, evalating titer comprises evaluating the ratio of functional versus non-functional (e.g., infectious vs non-infectious) anellovectors in the preparation (e.g., as evaluated by HPLC). In some embodiments, evaluating potency comprises evaluating the level of anellovector function (e.g., expression and/or function of an effector encoded therein or genomic equivalents) detectable in the preparation.

In some embodiments, the formulated preparation is substantially free of pathogens, host cell contaminants or impurities; has a predetermined level of non-infectious particles or a predetermined ratio of particles; infectious units (e.g., <300:1, <200:1, <100:1, or <50:1). In some embodiments, multiple anellovectors can be produced in a single batch. In some embodiments, the levels of the anellovectors produced in the batch can be evaluated (e.g., individually or together).

In an aspect, the invention features a host cell comprising:
(i) a first nucleic acid molecule comprising the nucleic acid sequence of a genetic element of an anellovector as described herein, and
(ii) optionally, a second nucleic acid molecule encoding one or more of an amino acid sequence chosen from ORF1, ORF2, ORF2/2, ORF2/3, ORF1/1, or ORF1/2 as listed in any of Tables A1-A1417, or an amino acid sequence having at least about 70% (e.g., at least about 70, 80, 90, 95, 96, 97, 98, 99, or 100%) sequence identity thereto.

In an aspect, the invention features a reaction mixture comprising an anellovector described herein and a helper virus, wherein the helper virus comprises a polynucleotide, e.g., a polynucleotide encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, an anellovector (e.g., a synthetic anellovector) is isolated, e.g., isolated from a host cell and/or isolated from other constituents in a solution (e.g., a supernatant). In some embodiments, an anellovector (e.g., a synthetic anellovector) is purified, e.g., from a solution (e.g., a supernatant). In some embodiments, an anellovector is enriched in a solution relative to other constituents in the solution.

In some embodiments of any of the aforesaid anellovectors, compositions or methods, providing an anellovector comprises separating (e.g., harvesting) an anellovector from a composition comprising an anellovector-producing cell, e.g., as described herein. In other embodiments, providing an anellovector comprises obtaining an anellovector or a preparation thereof, e.g., from a third party.

In some embodiments of any of the aforesaid anellovectors, anellovectors, compositions or methods, the genetic element comprises an anellovector genome, e.g., as identified according to the method described in Example 9. In embodiments, the anellovector genome is an anellovector genome capable of self-replication and/or self-amplification. In some embodiments, the anellovector genome is not capable of self-replication and/or self-amplification. In some embodiments, the anellovector genome is capable of replicating and/or being amplified in trans. e.g., in the presence of a helper, e.g., a helper virus.

Additional features of any of the aforesaid anellovectors, anellovectors, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### Enumerated Embodiments

1. An anellovector comprising:
(i) a proteinaceous exterior comprising an Anellovirus ORF1 protein as listed in any of Tables A1-A1417, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.

2. An anellovector comprising:
(i) a proteinaceous exterior comprising an Anellovirus ORF1 protein as listed in any of Tables A1-A1417, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

3. An anellovector comprising:
(i) a proteinaceous exterior comprising a polypeptide encoded by an Anellovirus ORF1 nucleic acid sequence as listed in any of Tables N1-N1417, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.

4. An anellovector comprising:
(i) a proteinaceous exterior comprising a polypeptide encoded by an Anellovirus ORF1 nucleic acid sequence as listed in any of Tables N1-N1417, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

5. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in any of Tables N1-N1417, or a nucleic acid sequence having at least 70%, 75%, 80%, 85%, 90%, 95%, 96%, 97%, 98%, 99%, or 100% sequence identity thereto, and (b) a promoter element operably linked to a nucleic acid sequence (e.g., a DNA sequence) encoding an exogenous effector.

6. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in any of Tables N1-N1417, or a nucleic acid sequence having at least 70%, 75%, 80%, 85%, 90%, 95%, 96%, 97%, 98%, 99%, or 100% sequence identity thereto, and (b) a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

7. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an exogenous effector, and wherein the genetic element has at least about 70%, 75%, 80%, 85%, 90%, 95%, 96%, 97%, 98%, 99%, or 100% sequence identity to an Anellovirus genome sequence as listed in any of Tables N1-N1417.

8. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector), and wherein the genetic element has at least about 70%, 75%, 80%, 85%, 90%, 95%, 96%, 97%, 98%, 99%, or 100% sequence identity to an Anellovirus genome sequence as listed in any of Tables N1-N1417;
wherein the proteinaceous exterior and/or the genetic element comprises at least one difference (e.g., a mutation, chemical modification, or epigenetic alteration) relative to a wild-type Anellovirus ORF 1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

9. The anellovector of any of the preceding embodiments, wherein the at least one difference relative to a wild-type Anellovirus ORF1 protein and/or wild-type Anellovirus genome comprises encoding an exogenous effector.

10. The anellovector of any of the preceding embodiments, wherein the proteinaceous exterior comprises the amino acid sequence YNPX²DXGX²N (SEQ ID NO: 5202), wherein Xⁿ is a contiguous sequence of any n amino acids.

11. An isolated ORF1 molecule comprising the amino acid sequence of an ORF1 as listed in any of Tables A1-A1417, or an amino acid sequence having at least about 70%, 75%, 80%, 85%, 90%, 95%, 96%, 97%, 98%, 99%, or 100% sequence identity thereto:
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

12. An isolated ORF1 molecule comprising the amino acid sequence of the jelly-roll domain of an ORF1 as listed in any of Tables A1-A1417, or an amino acid sequence having at least about 70%, 75%, 80%, 85%, 90%, 95%, 96%, 97%, 98%, 99%, or 100% sequence identity thereto:
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

13. The ORF1 molecule of any one of embodiments 11-12, wherein the ORF1 molecule comprises the amino acid sequence YNPX²DXGX²N (SEQ ID NO: 5202), wherein Xⁿ is a contiguous sequence of any n amino acids.

14. The ORF1 molecule of embodiment 13, wherein the amino acid sequence YNPX²DXGX²N (SEQ ID NO: 5202) is comprised in an N22 domain of the ORF1 molecule.

15. The ORF1 molecule of any one of embodiments 11-14, wherein the ORF1 molecule comprises one or more (e.g., 1, 2, 3, 4, or all 5) of the following Anellovirus ORF1 subdomains: an arginine-rich region, a jelly-roll region, a hypervariable region, an N22 domain, a C-terminal domain (CTD) (e.g., as described herein), e.g., of an Anellovirus ORF1 protein as listed in any of Tables A1-A1417 (or a sequence having at least about 70%, 75%, 80%, 85%, 90%, 95%, 96%, 97%, 98%, 99%, or 100% sequence identity thereto).

16. An isolated ORF2 molecule comprising the amino acid sequence of an ORF2 as listed in any of Tables A1-A1417, or an amino acid sequence having at least about 70%, 75%, 80%, 85%, 90%, 95%, 96%, 97%, 98%, 99%, or 100% sequence identity thereto;
wherein the ORF2 molecule comprises at least one difference (e.g., a mutation, chemical modification, or epigenetic alteration) relative to a wild-type ORF2 protein (e.g., as described herein), e.g., an insertion, substitution, chemical or enzymatic modification, and/or deletion, e.g., a deletion of a domain.

17. The ORF2 molecule of embodiment 16, wherein the ORF2 molecule comprises the amino acid sequence [W/F]X⁷HX³CX¹CX⁵H (SEQ ID NO: 5203), wherein Xⁿ is a contiguous sequence of any n amino acids.

18. An isolated nucleic acid molecule (e.g., a genetic element construct or a genetic element) comprising the nucleic acid sequence of a 5′ UTR conserved domain as listed in any of Tables N1-N1417, or a nucleic acid sequence having at least 70%, 75%, 80%, 85%, 90%, 95%, 96%, 97%, 98%, or 99% sequence identity thereto.

19. An isolated nucleic acid molecule (e.g., a genetic element construct or a construct for providing an ORF1 molecule in trans. e.g., as described herein) comprising the nucleic acid sequence of an ORF1 gene as listed in any of Tables N1-N1417, or a nucleic acid sequence having at least 70%, 75%, 80%, 85%, 90%, 95%, 96%, 97%, 98%, or 99% sequence identity thereto.

20. An isolated nucleic acid molecule (e.g., a genetic element construct or a construct for providing an ORF2 molecule in trans. e.g., as described herein) comprising the nucleic acid sequence of an ORF2 gene as listed in any of Tables N1-N1417, or a nucleic acid sequence having at least 70%, 75%, 80%, 85%, 90%, 95%, 96%, 97%, 98%, or 99% sequence identity thereto.

21. An isolated nucleic acid molecule (e.g., a genetic element construct, a genetic element, or a construct for providing an ORF1 or ORF2 molecule in trans. e.g., as described herein) comprising an Anellovirus genome sequence as listed in any of Tables N1-N1417, or a nucleic acid sequence having at least 50%, 60%, 70%, 75%, 80%, 85%, 90%, 95%, 96%, 97%, 98%, or 99% sequence identity thereto.

22. The isolated nucleic acid molecule of any of embodiments 18-21, wherein the isolated nucleic acid molecule comprises at least one difference (e.g., a mutation, chemical modification, or epigenetic alteration) relative to a wild-type Anellovirus genome sequence (e.g., as described herein) 23. The isolated nucleic acid molecule of embodiment 22, wherein the at least one difference comprises a deletion (e.g., lacks one or more of: a 5′ UTR conserved domain, an ORF1 gene, an ORF2 gene, a GC-rich region, an ORF3 gene, or a functional fragment thereof).

24. The isolated nucleic acid molecule of any of embodiments 18-23, wherein the isolated nucleic acid molecule is substantially unable to be enclosed in an Anellovirus capsid (e.g., a proteinaceous exterior of an anellovector as described herein).

25. The isolated nucleic acid molecule of embodiment 18 or 21, wherein the isolated nucleic acid molecule encodes an effector (e.g., an exogenous effector or an endogenous effector).

26. A genetic element comprising:
(a) a 5′ UTR conserved domain as listed in any of Tables N1-N1417, or a nucleic acid sequence having at least 70%, 75%, 80%, 85%, 90%, 95%, 96%, 97%, 98%, 99%, or 100% sequence identity thereto, and
(b) a promoter element operably linked to a nucleic acid sequence (e.g., a DNA sequence) encoding an exogenous effector.

27. A pharmaceutical composition comprising the anellovector, ORF1 molecule, ORF2 molecule, or nucleic acid molecule of any of the preceding embodiments, and a pharmaceutically acceptable carrier and/or excipient.

28. The pharmaceutical composition of embodiment 27, wherein the pharmaceutical composition has one or more of the following characteristics:
a) the pharmaceutical composition meets a pharmaceutical or good manufacturing practices (GMP) standard;
b) the pharmaceutical composition was made according to good manufacturing practices (GMP);
c) the pharmaceutical composition has a pathogen level below a predetermined reference value, e.g., is substantially free of pathogens;
d) the pharmaceutical composition has a contaminant level below a predetermined reference value, e.g., is substantially free of contaminants;
e) the pharmaceutical composition has a predetermined level of non-infectious particles or a predetermined ratio of particles:infectious units (e.g., <300:1, <200:1, <100:1, or <50:1), or
f) the pharmaceutical composition has low immunogenicity or is substantially non-immunogenic, e.g., as described herein.

29. The pharmaceutical composition of any one of embodiments 27-28, wherein the pharmaceutical composition has a contaminant level below a predetermined reference value, e.g., is substantially free of contaminants.

30. The pharmaceutical composition of embodiment 29, wherein the contaminant is selected from the group consisting of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted anellovectors (e.g., an anellovector other than the desired anellovector, e.g., a synthetic anellovector as described herein), free viral capsid protein, adventitious agents, and aggregates.

31. The pharmaceutical composition of embodiment 29, wherein the contaminant is host cell DNA and the threshold amount is about 10 ng of host cell DNA per dose of the pharmaceutical composition.

32. The pharmaceutical composition of any one of embodiments 27-31, wherein the pharmaceutical composition comprises less than 10% (e.g., less than about 10%, 5%, 4%, 3%, 2%, 1%, 0.5%, or 0.1%) contaminant by weight.

33. An isolated cell, e.g., a host cell, comprising:
(a) a nucleic acid molecule encoding an ORF1 polypeptide and/or an ORF2 polypeptide of any of the preceding embodiments, wherein the nucleic acid is a plasmid, is a viral nucleic acid, or is integrated into a cell chromosome, and
(b) a genetic element construct comprising a promoter element and a nucleic acid sequence (e.g., a DNA sequence) encoding an effector (e.g., an exogenous effector or an endogenous effector), and a protein binding sequence, wherein optionally the genetic element does not encode an ORF1 polypeptide (e.g., an ORF1 protein).

34. An isolated cell, e.g., a host cell, comprising:
(i) a first nucleic acid molecule comprising the nucleic acid sequence of a genetic element of an anellovector of any of the preceding embodiments (optionally wherein the genetic element does not encode an ORF1 molecule), and
(ii) a second nucleic acid molecule, encoding an amino acid sequence of an ORF1 or ORF2 as listed in any of Tables A1-A1417, or an amino acid sequence having at least 70% (e.g., at least 70%, 75%, 80%, 85%, 90%, 95%, 96%, 97%, 98%, 99%, or 100%) sequence identity thereto.

35. A method of manufacturing an anellovector composition, the method comprising:
(a) providing a cell, e.g., a host cell as described herein;
(b) introducing a genetic element construct encoding the genetic element of an anellovector of any of the preceding embodiments into the cell;
(c) incubating the cell under conditions that allow the cell to produce anellovector, and
(d) formulating the anellovectors, e.g., as a pharmaceutical composition suitable for administration to a subject, thereby making the anellovector composition.

36. A method of manufacturing an anellovector composition, the method comprising:
(a) providing a cell, e.g., a host cell as described herein;
(b) introducing a nucleic acid molecule encoding an ORF1 or ORF2 polypeptide as listed in any of Tables A1-A1417 (or an amino acid sequence having at least about 70%, 75%, 80%, 85%, 90%, 95%, 96%, 97%, 98%, or 99% sequence identity thereto) into the cell;
(c) introducing a genetic element construct into the cell (e.g., before, after, or simultaneously with (b)),
(d) incubating the cell under conditions that allow the cell to produce anellovector; and
(e) formulating the anellovectors, e.g., as a pharmaceutical composition suitable for administration to a subject, thereby making the anellovector composition.

37. A method of manufacturing an anellovector composition, the method comprising:
(a) providing a cell, e.g., a host cell as described herein;
(b) introducing a nucleic acid molecule encoding an ORF1 or ORF2 polypeptide into the cell;
(c) introducing a genetic element construct into the cell as listed in any of Tables N1-N1417 (or a nucleic acid sequence having at least about 70%, 75%, 80%, 85%, 90%, 95%, 96%, 97%, 98%, or 99% sequence identity thereto) (e.g., before, after, or simultaneously with (b)),
(d) incubating the cell under conditions that allow the cell to produce anellovector; and
(e) formulating the anellovectors, e.g., as a pharmaceutical composition suitable for administration to a subject,
   thereby making the anellovector composition.

38. A method of making an anellovector, e.g., a synthetic anellovector, comprising:
(a) providing a host cell comprising:
   (i) a nucleic acid molecule, e.g., a first nucleic acid molecule, comprising the nucleic acid sequence of a Anellovirus genome as listed in any of Tables N1-N1417 (or a nucleic acid sequence having at least 70%, 75%, 80%, 85%, 90%, 95%, 96%, 97%, 98%, or 99% sequence identity thereto), and
   (ii) a nucleic acid molecule, e.g., a second nucleic acid molecule, encoding one or more of an amino acid sequence chosen from ORF1, ORF2, ORF2/2, ORF2/3, ORF1/1, or ORF1/2, e.g., as listed in any of Tables A1-A1417, or an amino acid sequence having at least 70% 75%, 80%, 85%, 90%, 95%, 96%, 97%, 98%, or 99% sequence identity thereto; and
(b) culturing the host cell under conditions suitable to make the anellovector.

39. The method of embodiment 38, further comprising, prior to step (a), introducing the first nucleic acid molecule and/or the second nucleic acid molecule into the host cell.

40. The method of embodiment 38 or 39, wherein the second nucleic acid molecule is introduced into the host cell prior to, concurrently with, or after the first nucleic acid molecule.

41. The method of any of embodiments 35-40, further comprising separating the anellovectors from the cell.

42. A method of manufacturing an ORF1 molecule, the method comprising:
(a) providing a host cell (e.g., a host cell described herein) comprising a nucleic acid encoding the ORF1 polypeptide of any of the preceding embodiments, and
(b) maintaining the host cell under conditions that allow the cell to produce the polypeptide;
thereby manufacturing the ORF1 molecule.

43. A method of delivering an effector (e.g., an exogenous effector or an endogenous effector, e.g., overexpressing an endogenous effector) to a subject, the method comprising administering to the subject an anellovector or pharmaceutical composition of any of the preceding embodiments.

44. A method of delivering an effector (e.g., an exogenous effector or an endogenous effector, e.g., overexpressing an endogenous effector) to a target cell, the method comprising contacting the target cell with an anellovector of any of the preceding embodiments.

45. A method of delivering an effector (e.g., an exogenous effector or an endogenous effector, e.g., overexpressing an endogenous effector) to a target cell ex vivo (e.g., a target cell isolated from a subject, e.g., a patient), the method comprising contacting the target cell with an anellovector of any of the preceding embodiments.

46. A method of modulating, e.g., enhancing or inhibiting, a biological function (e.g., as described herein) in a subject, the method comprising administering the anellovector or the pharmaceutical composition of any of the preceding embodiments to the subject.

47. A method of treating a disease or disorder in a subject in need thereof, the method comprising administering to the subject an anellovector or pharmaceutical composition of any of the preceding embodiments.

48. Use of the anellovector of any one of embodiments 1-10 or pharmaceutical composition of any one of embodiments 27-32 for treating a disease or disorder (e.g., as described herein) in a subject.

49. The anellovector of any one of embodiments 1-10 or pharmaceutical composition of any one of embodiments 27-32 for use in treating a disease or disorder (e.g., as described herein) in a subject.

50. Use of the anellovector of any one of embodiments 1-10 or pharmaceutical composition of any one of embodiments 27-32 in the manufacture of a medicament for treating a disease or disorder (e.g., as described herein) in a subject.

51. The anellovector, ORF1 molecule, ORF2 molecule, nucleic acid molecule, or method of any of the preceding embodiments, wherein the genetic element is single-stranded.

52. The anellovector, ORF1 molecule, ORF2 molecule, nucleic acid molecule, or method of any of the preceding embodiments, wherein the genetic element is circular.

53. The anellovector, ORF1 molecule, ORF2 molecule, nucleic acid molecule, or method of any of the preceding embodiments, wherein the genetic element comprises DNA.

54. The anellovector, ORF1 molecule, ORF2 molecule, nucleic acid molecule, or method of any of the preceding embodiments, wherein the genetic element is double-stranded.

55. The anellovector, ORF1 molecule, ORF2 molecule, nucleic acid molecule, or method of any of embodiments A1-L1 or L3-L4, wherein the genetic element is linear.

56. The anellovector, ORF1 molecule, ORF2 molecule, nucleic acid molecule, or method of any of the preceding embodiments, wherein the genetic element comprises RNA.

57. The anellovector, ORF1 molecule, ORF2 molecule, nucleic acid molecule, or method of any of the preceding embodiments, wherein the genetic element comprises a nucleic acid sequence encoding an Anellovirus ORF1 molecule (e.g., an ORF1 protein as listed in any of Tables A1-A1417 or an amino acid sequence having at least 70%, 75%, 80%, 85%, 90%, 95%, 96%, 97%, 98%, or 99% sequence identity thereto).

58. The anellovector, ORF1 molecule, ORF2 molecule, nucleic acid molecule, or method of any of the preceding embodiments, wherein the genetic element does not comprise a nucleic acid sequence encoding an Anellovirus ORF1 molecule (e.g., an ORF1 protein as listed in any of Tables A1-A1417 or an amino acid sequence having at least 70%, 75%, 80%, 85%, 90%, 95%, 96%, 97%, 98%, or 99% sequence identity thereto).

59. The anellovector, ORF1 molecule, ORF2 molecule, nucleic acid molecule, or method of any of the preceding embodiments, wherein the genetic element comprises a nucleic acid sequence encoding an Anellovirus ORF2 molecule (e.g., an ORF2 protein as listed in any of Tables A1-A1417 or an amino acid sequence having at least 70%, 75%, 80%, 85%, 90%, 95%, 96%, 97%, 98%, or 99% sequence identity thereto).

60. The anellovector, ORF1 molecule, ORF2 molecule, nucleic acid molecule, or method of any of the preceding embodiments, wherein the genetic element does not comprise a nucleic acid sequence encoding an Anellovirus ORF2 molecule (e.g., an ORF2 protein as listed in any of Tables A1-A1417 or an amino acid sequence having at least 70%, 75%, 80%, 85%, 90%, 95%, 96%, 97%, 98%, or 99% sequence identity thereto).

61. The anellovector, ORF1 molecule, ORF2 molecule, nucleic acid molecule, or method of any of the preceding embodiments, wherein the genetic element comprises at least 20, 25, 30, 31, 32, 33, 34, 35, 36, 37, 38, 39, or 40 consecutive nucleotides having a GC content of at least 70%, 75%, 80%, 85%, 90%, 95%, or 99%.

62. The anellovector, ORF1 molecule, ORF2 molecule, nucleic acid molecule, or method of any of the preceding embodiments, wherein the proteinaceous exterior comprises the amino acid sequence YNPX²DXGX²N (SEQ ID NO: 5202), wherein Xⁿ is a contiguous sequence of any n amino acids.

63. The anellovector, ORF1 molecule, ORF2 molecule, nucleic acid molecule, or method of embodiment 62, wherein the amino acid sequence YNPX²DXGX²N (SEQ ID NO: 5202) is comprised in an N22 domain.

64. The anellovector, ORF1 molecule, ORF2 molecule, nucleic acid molecule, or method of any of the preceding embodiments, wherein the ORF1 molecule comprises an arginine-rich region (e.g., having at least 70%, 80%, 85%, 90%, 95%, 96%, 97%, 98%, 99%, or 100% identity to an arginine-rich region sequence of an ORF1 protein listed in any of Tables A1-A1417).

65. The anellovector, ORF1 molecule, ORF2 molecule, nucleic acid molecule, or method of any of the preceding embodiments, wherein the proteinaceous exterior comprises an amino acid sequence of at least 15, 20, 25, 26, 27, 28, 29, 30, 31, 32, 33, 34, 35, 36, 37, 38, 39, 40, 45, or 50 consecutive nucleotides comprising at least 40% (e.g., at least 40%, 41%, 42%, 43%, 44%, 45%, 46%, 47%, 48%, 49%, 50%, 55%, 60%, 65%, 66%, 67%, 68%, 69%, 70%, 75%, 80%, 85%, 90%, or 95%) arginine residues.

66. The anellovector, ORF1 molecule, ORF2 molecule, nucleic acid molecule, or method of embodiment 64 or 65, wherein the arginine-rich region is located at the N-terminal or C-terminal end of the ORF1 molecule.

67. The anellovector, ORF1 molecule, ORF2 molecule, nucleic acid molecule, or method of any of the preceding embodiments, wherein the ORF1 molecule comprises a jelly-roll domain having at least 70%, 80%, 85%, 90%, 95%, 96%, 97%, 98%, 99%, or 100% identity to a jelly-roll domain sequence of an ORF1 protein listed in any of Tables A1-A1417.

68. The anellovector, ORF1 molecule, ORF2 molecule, nucleic acid molecule, or method of any of the preceding embodiments, wherein the ORF1 molecule comprises an N22 domain having at least 70%, 80%, 85%, 90%, 95%, 96%, 97%, 98%, 99%, or 100% identity to an N22 domain sequence of an ORF1 protein listed in any of Tables A1-A1417.

69. The anellovector, ORF1 molecule, ORF2 molecule, nucleic acid molecule, or method of any of the preceding embodiments, wherein the ORF1 molecule comprises a C-terminal domain (CTD) having at least 70%, 80%, 85%, 90%, 95%, 96%, 97%, 98%, 99%, or 100% identity to a CTD domain sequence of an ORF1 protein listed in any of Tables A1-A1417.

70. The anellovector, ORF1 molecule, ORF2 molecule, nucleic acid molecule, or method of any of the preceding embodiments, wherein the genetic element comprises one or more of: a TATA box, an initiator element, a cap site, a transcriptional start site, an ORF1/1-encoding sequence, an ORF1/2-encoding sequence, an ORF2/2-encoding sequence, an ORF2/3-encoding sequence, an ORF2/3t-encoding sequence, a three open-reading frame region, a poly(A) signal, and/or a GC-rich region from an Anellovirus described herein (e.g., as listed in any of Tables N1-N1417), or a sequence having at least 70%, 75%, 80%, 85%, 90%, 95%, 96%, 97%, 98%, 99%, or 100% sequence identity thereto.

71. The anellovector, ORF1 molecule, ORF2 molecule, nucleic acid molecule, or method of any of the preceding embodiments, wherein the genetic element comprises at least 75% (e.g., at least 75, 76, 77, 78, 79, 80, 85, 90, 91, 92, 93, 94, 95, 96, 97, 98, 99, or 100%) sequence identity to a 5′ UTR conserved domain sequence as listed in any of Tables N1-N1417.

72. The anellovector, ORF1 molecule, ORF2 molecule, nucleic acid molecule, or method of any of the preceding embodiments, wherein the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

73. The anellovector, ORF1 molecule, ORF2 molecule, nucleic acid molecule, or method of any of the preceding embodiments, wherein the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, comprises one or more desired carbohydrates (e.g., glycosylations), is pH and temperature stable, is detergent resistant, and is non-immunogenic or non-pathogenic in a host.

74. The anellovector, ORF1 molecule, ORF2 molecule, nucleic acid molecule, or method of any of the preceding embodiments, wherein the promoter comprises an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc).

75. The anellovector, ORF1 molecule, ORF2 molecule, nucleic acid molecule, or method of any of the preceding embodiments, wherein the effector encodes a therapeutic agent, e.g., a therapeutic peptide or polypeptide or a therapeutic nucleic acid.

76. The anellovector, ORF1 molecule, ORF2 molecule, nucleic acid molecule, or method of any of the preceding embodiments, wherein the effector is an exogenous effector.

77. The anellovector, ORF1 molecule, ORF2 molecule, nucleic acid molecule, or method of any of the preceding embodiments, wherein the effector is an endogenous effector (e.g., wherein the anellovector overexpresses the endogenous effector in a target cell).

78. The anellovector, ORF1 molecule, ORF2 molecule, nucleic acid molecule, or method of any of the preceding embodiments, wherein the effector comprises a regulatory nucleic acid, e.g., an miRNA, siRNA, mRNA, lncRNA, RNA, DNA, an antisense RNA, gRNA; a fluorescent tag or marker, an antigen, a peptide, a synthetic or analog peptide from a naturally-bioactive peptide, an agonist or antagonist peptide, an anti-microbial peptide, a pore-forming peptide, a bicyclic peptide, a targeting or cytotoxic peptide, a degradation or self-destruction peptide, a small molecule, an immune effector (e.g., influences susceptibility to an immune response/signal), a death protein (e.g., an inducer of apoptosis or necrosis), a non-lytic inhibitor of a tumor (e.g., an inhibitor of an oncoprotein), an epigenetic modifying agent, an epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand, an antibody, a receptor, or a CRISPR system or component.

79. The anellovector, ORF1 molecule, ORF2 molecule, nucleic acid molecule, or method of any of the preceding embodiments, wherein the effector comprises a miRNA, e.g., wherein the miRNA decreases expression of a target gene.

80. The anellovector, ORF1 molecule, ORF2 molecule, nucleic acid molecule, or method of any of the preceding embodiments, wherein the effector modulates expression or activity of a gene or protein, e.g., increases or decreases expression or activity of the gene or protein.

81. The anellovector, ORF1 molecule, ORF2 molecule, nucleic acid molecule, or method of any of the preceding embodiments, wherein the anellovector is capable of replicating autonomously 82. The anellovector, ORF1 molecule, ORF2 molecule, nucleic acid molecule, or method of any of the preceding embodiments, wherein the anellovector is replication-deficient (e.g., incapable of replicating autonomously).

83. The anellovector, ORF1 molecule, ORF2 molecule, nucleic acid molecule, or method of any of the preceding embodiments, wherein the genetic element integrates into the genome of a eukaryotic cell at a frequency of less than about 0.001%, 0.005%, 0.01%, 0.05%, 0.1%, 0.5%, 1%, 1.5%, or 2% of the genetic element that enters the cell.

84. The anellovector, ORF1 molecule, ORF2 molecule, nucleic acid molecule, or method of any of the preceding embodiments, wherein the anellovector is substantially non-pathogenic, e.g., does not induce a detectable deleterious symptom in a subject (e.g., elevated cell death or toxicity, e.g., relative to a subject not exposed to the anellovector).

85. The anellovector, ORF1 molecule, ORF2 molecule, nucleic acid molecule, or method of any of the preceding embodiments, wherein the anellovector is substantially non-immnuogenic, e.g., does not induce a detectable and/or unwanted immune response.

86. The anellovector, ORF1 molecule, ORF2 molecule, nucleic acid molecule, or method of any of the preceding embodiments, wherein a population of at least 1000 of the anellovectors is capable of delivering at least about 100 copies (e.g., at least 1, 2, 3, 4, 5, 10, 20, 30, 40, 50, 100, 200, 300, 400, 500, 600, 700, 800, 900, or 1000 copies) of the genetic element into one or more eukaryotic cells (e.g., mammalian cells, e.g., human cells).

87. The anellovector, ORF1 molecule, ORF2 molecule, nucleic acid molecule, or method of any of the preceding embodiments, wherein a population of the anellovectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering the genetic element into at least 10%, 20%, 30%, 40%, 50%, 60%, 70%, 80%, 90%, 95%, 99%, or more of a population of eukaryotic cells (e.g., mammalian cells, e.g., human cells).

88. The anellovector, ORF1 molecule, ORF2 molecule, nucleic acid molecule, or method of any of the preceding embodiments, wherein a population of the anellovectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering at least 1, 2, 3, 4, 5, 6, 7, 8, 9, 10, 20, 50, 100, 200, 500, 1000, 2000, 5000, 8,000, 1×10⁴, 1×10⁵, 1×10⁶, 1×10⁷ or greater copies of the genetic element per cell to a population of eukaryotic cells (e.g., mammalian cells, e.g., human cells).

89. The anellovector, ORF1 molecule, ORF2 molecule, nucleic acid molecule, or method of any of the preceding embodiments, wherein a population of the anellovectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering 1-3, 1-4, 1-5, 1-6, 1-7, 1-8, 1-9, 1-10, 5-10, 10-20, 20-50, 50-100, 100-1000, 1000-10⁴, 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of the genetic element per cell to a population of eukaryotic cells (e.g., mammalian cells, e.g., human cells).

90. The anellovector, ORF1 molecule, ORF2 molecule, nucleic acid molecule, or method of any of the preceding embodiments, wherein the target cells into which the genetic element is delivered each receive at least 10, 50, 100, 500, 1000, 10,000, 50,000, 100,000, or more copies of the genetic element.

91. The anellovector, ORF1 molecule, ORF2 molecule, nucleic acid molecule, or method of any of the preceding embodiments, wherein the anellovector is resistant to degradation by a detergent (e.g., a mild detergent, e.g., a biliary salt, e.g., sodium deoxycholate) relative to a viral particle comprising an external lipid bilayer, e.g., a retrovirus.

92. The anellovector, ORF1 molecule, ORF2 molecule, nucleic acid molecule, or method of any of the preceding embodiments, wherein the genetic element enclosed by the proteinaceous exterior is resistant to degradation by a nuclease enzyme (e.g., a DNase).

93. The anellovector, ORF1 molecule, ORF2 molecule, nucleic acid molecule, or method of any of the preceding embodiments, wherein the anellovector is capable of infecting mammalian cells, e.g., human cells, e.g., in vitro, in vivo, or ex vivo.

94. The anellovector, ORF1 molecule, ORF2 molecule, nucleic acid molecule, or method of any of the preceding embodiments, wherein the anellovector selectively delivers the effector to, or is present at higher levels in (e.g., preferentially accumulates in), a desired cell type, tissue, or organ (e.g., bone marrow, blood, heart, GI, skin, photoreceptors in the retina, epithelial linings, or pancreas).

95. The anellovector, ORF1 molecule, ORF2 molecule, nucleic acid molecule, or method of any of the preceding embodiments, wherein genetic element or genetic element construct is capable of replicating (e.g., by rolling circle replication), e.g., capable of generating at least 1, 2, 3, 4, 5, 6, 7, 8, 9, 10, 20, 30, 40, 50, 60, 70, 80, 90, 102, 2×10², 5×10², 10³, 2×10³, 5×10³, or 10⁴ genomic equivalents of the genetic element per cell, e.g., as measured by a quantitative PCR assay.

96. The anellovector, ORF1 molecule, ORF2 molecule, nucleic acid molecule, or method of any of the preceding embodiments, wherein the proteinaceous exterior is provided in cis relative to the genetic element.

97. The anellovector, ORF1 molecule, ORF2 molecule, nucleic acid molecule, or method of any of the preceding embodiments, wherein the proteinaceous exterior is provided in trans relative to the genetic element.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings.

FIG. 1A is an illustration showing percent sequence similarity of amino acid regions of capsid protein sequences.

FIG. 1B is an illustration showing percent sequence similarity of capsid protein sequences.

FIG. 2 is an illustration showing one embodiment of an anellovector.

FIG. 3 depicts a schematic of a kanamycin vector encoding the LY1 strain of TTMiniV (“Anellovector 1”).

FIG. 4 depicts a schematic of a kanamycin vector encoding the LY2 strain of TTMiniV (“Anellovector 2”).

FIG. 5 depicts transfection efficiency of synthetic anellovectors in 293T and A549 cells.

FIGS. 6A and 6B depict quantitative PCR results that illustrate successful infection of 293T cells by synthetic anellovectors.

FIGS. 7A and 7B depict quantitative PCR results that illustrate successful infection of A549 cells by synthetic anellovectors.

FIGS. 8A and 8B depict quantitative PCR results that illustrate successful infection of Raji cells by synthetic anellovectors.

FIGS. 9A and 9B depict quantitative PCR results that illustrate successful infection of Jurkat cells by synthetic anellovectors.

FIGS. 10A and 10B depict quantitative PCR results that illustrate successful infection of Chang cells by synthetic anellovectors.

FIGS. 11A-11B are a series of graphs showing luciferase expression from cells transfected or infected with TTMV-LY21574-1371, 11432-2210,2610::nLuc. Luminescence was observed in infected cells, indicating successful replication and packaging.

FIG. 11C is a diagram depicting the phylogenetic tree of Alphatorquevirus (Torque Teno Virus; TTV), with clades highlighted. At least 100 Anellovirus strains are represented. Exemplary sequences from several clades is provided herein.

FIG. 12 is a schematic showing an exemplary workflow for production of anellovectors (e.g., replication-competent or replication-deficient anellovectors as described herein).

FIG. 13 is a graph showing primer specificity for primer sets designed for quantification of TTV and TTMV genomic equivalents. Quantitative PCR based on SYBR green chemistry shows one distinct peak for each of the amplification products using TTMV or TTV specific primer sets, as indicated, on plasmids encoding the respective genomes.

FIG. 14 is a series of graphs showing PCR efficiencies in the quantification of TTV genome equivalents by qPCR. Increasing concentrations of primers and a fixed concentration of hydrolysis probe (250 nM) were used with two different commercial qPCR master mixes. Efficiencies of 90-110% resulted in minimal error propagation during quantification.

FIG. 15 is a graph showing an exemplary amplification plot for linear amplification of TTMV (Target 1) or TTV (Target 2) over a 7 log 10 of genome equivalent concentrations. Genome equivalents were quantified over 7 10-fold dilutions with high PCR efficiencies and linearity (R² TTMV: 0.996; R² TTV: 0.997).

FIGS. 16A-16B are a series of graphs showing quantification of TTMV genome equivalents in an anellovector stock. (A) Amplification plot of two stocks, each diluted 1:10 and run in duplicate. (B) The same two samples as shown in panel A, here shown in the context of the linear range. Shown are the upper and lower limits in the two representative samples. PCR Efficiency: 99.58%, R²: 0988.

FIG. 17 is a graph showing fold change in miR-625 expression in HEK293T cells transfected with the indicated plasmid.

FIG. 18 is a diagram showing pairwise identity for alignments of representative sequences from each Alphatorquevirus clade. DNA sequences for TTV-CT30F, TTV-P13-1, TTV-tth8, TTV-HD20a, TTV-16, TTV-TJN02, and TTV-HD16d were aligned. Pairwise percent identity across a 50-bp sliding window is shown along the length of the alignment. Brackets above indicate non-coding and coding regions with pairwise identities are indicated. Brackets below indicate regions of high or low sequence conservation.

FIG. 19 is a diagram showing pairwise identity for amino acid alignments for putative proteins across the seven Alphatorquevirus clades. Amino acid sequences for putative proteins from TTV-CT30F, TTV-P13-1, TTV-tth8, TTV-HD20a, TTV-16, TTV-TJN02, and TTV-HD16d were aligned. Pairwise percent identity across a 15-aa sliding window is shown along the length of each alignment. Pairwise identity for both open reading frame DNA sequence and protein amino acid sequence is indicated. (*) Putative ORF2t/3 amino acid sequences were aligned for TTV-CT30F, TTV-tth8, TTV-16, and TTV-TJN02.

FIG. 20 is a diagram showing that a domain within the 5′ UTR is highly conserved across the seven Alphatorquevirus clades (SEQ ID NOS 5205, 5114, 5121, 5118, 5123-5124, 5117 and 5126, respectively, in order of appearance). The 71-bp 5′UTR conserved domain sequences for each representative Alphatorquevirus were aligned. The sequence has 95.2% pairwise identity between the seven clades.

FIG. 21 is a diagram showing an alignment of the GC-rich domains from the seven Alphatorquevirus clades. Each Anellovirus has a region downstream of the ORFs with greater than 70% GC content. Shown is an alignment of the GC-rich regions from TTV-CT30F, TTV-P13-1, TTV-tth8, TTV-HD20a, TTV-16, TTV-TJN02, and TTV-HD16d. The regions vary in length, but where they do align they have 75.4% pairwise identity.

FIG. 22 is a diagram showing infection of Raji B cells with anellovectors encoding a miRNA targeting n-myc interacting protein (NMI). Shown is quantification of genome equivalents of anellovectors detected after infection of Raji B cells (arrow) or control cells with NMI miRNA-encoding anellovectors.

FIG. 23 is a diagram showing infection of Raji B cells with anellovectors encoding a miRNA targeting n-myc interacting protein (NMI). The Western blot shows that anellovectors encoding the miRNA against NMI reduced NMI protein expression in Raji B cells, whereas Raji B cells infected with anellovectors lacking the miRNA showed comparable NMI protein expression to controls.

FIG. 24 is a series of graphs showing quantification of anellovector particles generated in host cells after infection with an anellovector comprising an endogenous miRNA-encoding sequence and a corresponding anellovector in which the endogenous miRNA-encoding sequence was deleted.

FIGS. 25A-25C are a series of diagrams showing intracellular localization of ORFs from TTMV-LY2 fused to nano-luciferase. (A) In Vero cells, ORF2 (top row) appeared to localize to the cytoplasm while ORF1/1 (bottom row) appeared to localize to the nucleus. (B) In HEK293 cells, ORF2 (top row) appeared to localize to the cytoplasm while ORF1/1 (bottom row) appeared to localize to the nucleus. (C) Localization patterns for ORF1/2 and ORF2/2 in cells.

FIG. 26 is a series of diagrams showing sequential deletion controls in the 3′ non-coding region (NCR) of TTV-tth8. The top row shows the structure of the wild-type TTV-tth8 Anellovirus. The second row shows TTV-tth8 with a deletion of 36 nucleotides in the GC-rich region of the 3′ NCR (Δ36nt (GC)). The third row shows TTV-tth8 with the 36 nucleotide deletion and an additional deletion of the miRNA sequence, resulting in a total deletion of 78 nucleotides (Δ36nt (GC) ΔmiR). The fourth row shows TTV-tth8 with a deletion of 171 nucleotides from the 3′ NCR, which includes both the 36 nucleotide deletion region and the miRNA sequence (43′ NCR).

FIGS. 27A-27D are a series of diagrams showing that sequential deletions in the 3′ NCR of TTV-tth8 have significant effects on Anellovirus ORF transcript levels. Shown are expression of ORF1 and ORF2 at day 2 (A), ORF1/1 and ORF2/2 at day 2 (B), ORF1/2 and ORF2/3 at day 2 (C), and ORF2t3 at day 2 (D).

FIGS. 28A-28B are a series of diagrams showing constructs used to produce anellovectors expressing nano-luciferase (A) and a series of anellovector/plasmid combinations used to transfect cells (B).

FIGS. 29A-29C are a series of diagrams showing nano-luciferase expression in mice injected with anellovectors. (A) Nano-luciferase expression in mice at days 0-9 after injection. (B) Nano-luciferase expression in mice injected with various anellovector/plasmid construct combinations, as indicated. (C) Quantification of nano-luciferase luminescence detected in mice after injection. Group A received a TTMV-LY2 vector±nano-luciferase. Group B received a nano-luciferase protein and TTMV-LY2 ORFs.

FIGS. 29D-1 to 29D-2 are a schematic of the genomic organization of representative anellos from seven different Alphatorquevirus clades. Sequences for TTV-CT30F, TTV-P13-1, TTV-tth8, TTV-HD20a, TTV-16, TTV-TJN02, and TTV-HD16d were aligned, with key regions annotated. Putative open reading frames (ORFs) are represented in light gray, TATA boxes are represented in dark gray, and key putative regulatory regions are represented in medium gray, including the initiator element, the 5′UTR conserved domain, and the GC-rich region (e.g., as indicated).

FIG. 30 is a schematic showing an exemplary workflow for determining the endogenous target of Anellovirus pre-miRNAs.

FIGS. 31A-31B are a series of diagrams showing that a tandem Anellovirus plasmid can increase anellovirus or anellovector production. (A) Plasmid map for an exemplary tandem Anellovirus plasmid. (B) Transfection of HEK293T cells with a tandem Anellovirus plasmid resulted in production of four times the number of viral genomes compared to single-copy harboring plasmids.

FIG. 31C is a gel electrophoresis image showing circularization of TTMV-LY2 plasmids pVL46-063 and pVL46-240.

FIG. 31D is a chromatogram showing copy numbers for linear and circular TTMV-LY2 constructs, as determined by size exclusion chromatography (SEC).

FIG. 32 is a diagram showing an alignment of 36-nucleotide GC-rich regions from nine Anellovirus genome sequences, and a consensus sequence based thereon (SEQ ID NOS 5206, 5172-5176 and 5176-5179, respectively, in order of appearance).

FIG. 33 is a series of diagrams showing ORF1 structures from Anellovirus strains LY2 and CBD203. Putative domains are labeled: arginine-rich region (arg-rich), core region comprising a jelly-roll domain, hypervariable region (HVR), N22 region, and C-terminal domain (CTD), as indicated.

FIG. 34 is a diagram showing an ORF1 structure from Betatorquevirus strain CBS203. Residues showing high similarity among a set of 110 betatorqueviruses are indicated. Indicated are residues of 60-79.9% similarity, residues of 80-99.9% similarity, and residues of 100% similarity among all strains evaluated.

FIG. 35 is a diagram showing the consensus sequence (SEQ ID NO: 5207) from alignment of 258 sequences of Alphatorqueviruses with residues with high similarity scores highlighted dark gray (100%), medium gray (80-99.9%), light gray (60-80%). Putative domains are indicated in boxes. Percent identity is also indicated by the box graph below the consensus sequence, with medium-gray boxes indicating 100% identity, light gray boxes indicating 30-99% identity, and dark gray boxes indicating below 30% identity.

FIG. 36 is a schematic showing the domains of an Anellovirus ORF1 molecule and the hypervariable region to be replaced with a hypervariable domain from a different Anellovirus.

FIG. 37 is a schematic showing the domains of ORF1 and the hypervariable region that will be replaced with a protein or peptide of interest (POI) from a non-anellovirus source.

FIG. 38 is a series of diagrams showing the design of an exemplary anellovector genetic element based on an Anellovirus genome. The protein-coding region was deleted from the anellovirus genome (left), leaving the anelloviral non-coding region (NCR), including the viral promoter, 5′UTR conserved domain (5CD), and GC-rich region. Payload DNA was inserted into the non-coding region at the protein-coding locus (right). The resulting anellovector harbored the payload DNA (including open reading frames, genes, non-coding RNAs, etc.) and the essential anellovirus cis replication and packaging elements, but lacked the essential protein elements for replication and packaging.

FIG. 39 is a bar graph showing that anellovectors comprising a genetic element encoding an exogenous human immunoadhesin successfully transduced the human lung-derived cell line EKVX.

FIG. 40 is a graph showing that anellovectors based on tth8 or LY2, engineered to contain a sequence encoding human erythropoietin (hEpo), could deliver a functional transgene to mammalian cells.

FIGS. 41A and 41B are a series of graphs showing that engineered anellovectors administered to mice were detectable seven days after intravenous injection.

FIG. 42 is a graph showing that hGH mRNA was detected in the cellular fraction of whole blood seven days after intravenous administration of an engineered anellovector encoding hGH.

FIGS. 43A-43D are a series of diagrams illustrating a highly conserved motif in Anellovirus ORF2. FIG. 43 discloses SEQ ID NO: 5203.

FIGS. 44A and 44B are a series of diagrams showing evidence of full-length ORF1 mRNA expression in human tissues.

FIG. 45 is a graph showing the ability of an in vitro circularized (IVC) TTV-tth8 genome (IVC TTV-tth8) compared to a TTV-tth8 genome in a plasmid to yield TTV-tth8 genome copies at the expected density in HEK293T cells.

FIG. 46 is a series of graphs showing the ability of an in vitro circularized (IVC) LY2 genome (WT LY2 IVC) and a wild-type LY2 genome in plasmid (WT LY2 Plasmid) to yield LY2 genome copies at the expected density in Jurkat cells.

FIG. 47 is a diagram showing an alignment of secondary structure of the jelly roll domain of Anellovirus ORF1 proteins from Alphatorquevirus, Betatorquevirus, and Gammatorquevirus (SEQ ID NOs: 5235-5260, respectively, in order of appearance). These secondary structural elements are highly conserved.

FIG. 48 is a diagram showing the conserved sequence and secondary structure of the ORF1 motif located in the N22 domain (SEQ ID NOS 5208-5234, respectively, in order of appearance). The conserved YNPXXDXGXXN (SEQ ID NO: 5202) motif of human TTV ORF1 has a conserved secondary structure. In particular, the tyrosine in the motif breaks a beta strand, and a second beta strand starts on the terminal asparagine of the motif.

### DETAILED DESCRIPTION OF CERTAIN EMBODIMENTS

### Definitions

The present invention will be described with respect to particular embodiments and with reference to certain figures, but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

As used herein, the term “anellovector” refers to a vehicle comprising a genetic element, e.g., an episome, e.g., circular DNA, enclosed in a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior, e.g., prior to entry into a host cell. In some embodiments, the anellovector is purified, e.g., it is separated from its original source and/or substantially free (>50%, >60%, >70%, >80%, >90%) of other components.

As used herein, the term “anellovector” refers to a vector that comprises sufficient nucleic acid sequence derived from or highly similar to (e.g., at least 85%, 90%, 95%, 96%, 97%, 98%, 99%, or 100% identical to) an Anellovirus genome sequence or a contiguous portion thereof to allow packaging into a proteinaceous exterior (e.g., a capsid), and further comprises a heterologous sequence. In some embodiments, the anellovector is a viral vector or a naked nucleic acid. In some embodiments, the anellovector comprises at least about 50, 60, 70, 71, 72, 73, 74, 75, 80, 90, 100, 150, 200, 300, 400, 500, 600, 700, 800, 900, 1000, 1100, 1200, 1300, 1400, 1500, 1600, 1700, 1800, 1900, 2000, 2500, 3000, or 3500 consecutive nucleotides of a native Anellovirus sequence or a sequence highly similar (e.g., at least 85%, 90%, 95%, 96%, 97%, 98%, 99%, or 100% identical) thereto. In some embodiments, the anellovector further comprises one or more of an Anellovirus ORF1, ORF2, or ORF3. In some embodiments, the heterologous sequence comprises a multiple cloning site, comprises a heterologous promoter, comprises a coding region for a therapeutic protein, or encodes a therapeutic nucleic acid. In some embodiments, the capsid is a wild-type Anellovirus capsid. In embodiments, an anellovector comprises a genetic element described herein, e.g., comprises a genetic element comprising a promoter, a sequence encoding a therapeutic effector, and a capsid binding sequence.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

As used herein, a nucleic acid “encoding” refers to a nucleic acid sequence encoding an amino acid sequence or a functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid sequence, generally in an anellovector. It is understood that the genetic element can be produced as naked DNA and optionally further assembled into a proteinaceous exterior. It is also understood that an anellovector can insert its genetic element into a cell, resulting in the genetic element being present in the cell and the proteinaceous exterior not necessarily entering the cell.

As used herein, the term “ORF1 molecule” refers to a polypeptide having an activity and/or a structural feature of an Anellovirus ORF1 protein (e.g., an Anellovirus ORF1 protein as described herein, e.g., as listed in any of Tables A1-A1417), or a functional fragment thereof. An ORF1 molecule may, in some instances, comprise one or more of (e.g., 1, 2, 3 or 4 of): a first region comprising at least 60% basic residues (e.g., at least 60% arginine residues), a second region comprising at least about six beta strands (e.g., at least 4, 5, 6, 7, 8, 9, 10, 11, or 12 beta strands), a third region comprising a structure or an activity of an Anellovirus N22 domain (e.g., as described herein, e.g., an N22 domain from an Anellovirus ORF1 protein as described herein), and/or a fourth region comprising a structure or an activity of an Anellovirus C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid (e.g., as listed in any of Tables N1-N1417). An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein), e.g., an ORF1 protein having the amino acid sequence as listed in any of Tables A1-A1417, or as encoded by the ORF1 gene as listed in any of Tables Tables N1-N1417.

As used herein, the term “ORF2 molecule” refers to a polypeptide having an activity and/or a structural feature of an Anellovirus ORF2 protein (e.g., an Anellovirus ORF2 protein as described herein, e.g., as listed in any of Tables A1-A1417), or a functional fragment thereof. An “Anellovirus ORF2 protein,” as used herein, refers to an ORF2 protein encoded by an Anellovirus genome (e.g., a wild-type Anellovirus genome, e.g., as described herein), e.g., an ORF2 protein having the amino acid sequence as listed in any of Tables A1-A1417, or as encoded by the ORF2 gene as listed in any of Tables N1-N1417.

As used herein, the term “proteinaceous exterior” refers to an exterior component that is predominantly (e.g., >50%, >60%, >70%, >80%, >90%) protein.

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “replication protein” refers to a protein, e.g., a viral protein, that is utilized during infection, viral genome replication/expression, viral protein synthesis, and/or assembly of the viral components.

As used herein, a “substantially non-pathogenic” organism, particle, or component, refers to an organism, particle (e.g., a virus or an anellovector, e.g., as described herein), or component thereof that does not cause or induce a detectable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce a detectable disease or pathogenic condition, e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization.

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In some embodiments, the substantially non-immunogenic organism, particle, or component does not produce a detectable immune response. In some embodiments, the substantially non-immunogenic anellovector does not produce a detectable immune response against a protein comprising an amino acid sequence or encoded by a nucleic acid sequence shown in any of Tables N1-N1417. In some embodiments, an immune response (e.g., an undesired or untargeted immune response) is detected by assaying antibody presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subseqence comprising an exogenous sequence or a sequence heterologous to the remainder of the larger sequence, e.g., a corresponding subsequence from a different Anellovirus).

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder); and supportive treatment (treatment employed to supplement another therapy).

As used herein, the term “virome” refers to viruses in a particular environment, e.g., a part of a body, e.g., in an organism, e.g. in a cell, e.g. in a tissue.

This invention relates generally to anellovectors, e.g., synthetic anellovectors, and uses thereof. The present disclosure provides anellovectors, compositions comprising anellovectors, and methods of making or using anellovectors. Anellovectors are generally useful as delivery vehicles, e.g., for delivering a therapeutic agent to a eukaryotic cell. Generally, an anellovector will include a genetic element comprising a nucleic acid sequence (e.g., encoding an effector, e.g., an exogenous effector or an endogenous effector) enclosed within a proteinaceous exterior. An anellovector may include one or more deletions of sequences (e.g., regions or domains as described herein) relative to an Anellovirus sequence (e.g., as described herein). Anellovectors can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

### TABLE OF CONTENTS

I. Anellovectors
   A. Anelloviruses
   B. ORF1 molecules
   C. ORF2 molecules
   D. Genetic elements
   E. Protein binding sequences
   F. 5′ UTR Regions
   G. GC-rich regions
   H. Effectors
   I. Proteinaceous exterior
II. Compositions and Methods for Making Anellovectors
   A. Components and Assembly of Anellovectors
      i. ORF1 molecules for assembly of anellovectors
      ii. ORF2 molecules for assembly of anellovectors
      iii. Production of protein components
   B. Genetic Element Constructs
      i. Plasmids
      ii. Circular nucleic acid constructs
      iii. In vitro circularization
      iv. Tandem constructs
      v. Cis/trans constructs
      vi. Expression cassettes
      vii. Design and production of a genetic element construct
   C. Effectors
   D. Host Cells
      i. Introduction of genetic elements into host cells
      ii. Methods for providing protein(s) in cis or trans
      iii. Exemplary cell types
   E. Culture Conditions
   F. Harvest
   G. In vitro assembly methods
   H. Enrichment and Purification
III. Vectors
IV. Compositions
V. Host cells
VI. Methods of use
VII. Methods of production
VIII. Administration/Delivery

### I. Anellovectors

In some aspects, the invention described herein comprises compositions and methods of using and making an anellovector, anellovector preparations, and therapeutic compositions. In some embodiments, the anellovector has a sequence, structure, and/or function that is based on an Anellovirus (e.g., an Anellovirus as described herein, e.g., an Anellovirus comprising a nucleic acid or polypeptide comprising a sequence as shown in any of Tables A1-A1417 or N1-N1417), or fragments or portions thereof, or other substantially non-pathogenic virus, e.g., a symbiotic virus, commensal virus, native virus. In some embodiments, an Anellovirus-based anellovector comprises at least one element exogenous to that Anellovirus, e.g., an exogenous effector or a nucleic acid sequence encoding an exogenous effector disposed within a genetic element of the anellovector. In some embodiments, an Anellovirus-based anellovector comprises at least one element heterologous to another element from that Anellovirus, e.g., an effector-encoding nucleic acid sequence that is heterologous to another linked nucleic acid sequence, such as a promoter element. In some embodiments, an anellovector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An anellovector may be a delivery vehicle (e.g., a substantially non-pathogenic delivery vehicle) for a payload into a host, e.g., a human. In some embodiments, the anellovector is capable of replicating in a eukaryotic cell, e.g., a mammalian cell, e.g., a human cell. In some embodiments, the anellovector is substantially non-pathogenic and/or substantially non-integrating in the mammalian (e.g., human) cell. In some embodiments, the anellovector is substantially non-immunogenic in a mammal, e.g., a human. In some embodiments, the anellovector is replication-deficient. In some embodiments, the anellovector is replication-competent.

In some embodiments the anellovector comprises a curon, or a component thereof (e.g., a genetic element, e.g., comprising a sequence encoding an effector, and/or a proteinaceous exterior), e.g., as described in PCT Application No. PCT/US2018/037379, which is incorporated herein by reference in its entirety.

In an aspect, the invention includes an anellovector comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the anellovector is capable of delivering the genetic element into a eukaryotic cell.

In some embodiments of the anellovector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the anellovectors administered to a subject will integrate into the genome of one or more host cells in the subject. In some embodiments, the genetic elements of a population of anellovectors, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes an anellovector comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence as listed in any of Tables N1-N1417); and (ii) a proteinaceous exterior; wherein the genetic element is enclosed within the proteinaceous exterior; and wherein the anellovector is capable of delivering the genetic element into a eukaryotic cell.

In one aspect, the invention includes an anellovector comprising:
a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and
b) a proteinaceous exterior that is associated with, e.g., envelops or encloses, the genetic element.

In some embodiments, the anellovector includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anellovector modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In some embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In some embodiments, the promoter element comprises a TATA box. In some embodiments, the promoter element is endogenous to a wild-type Anellovirus, e.g., as described herein.

In some embodiments, the genetic element comprises one or more of the following characteristics: single-stranded, circular, negative strand, and/or DNA. In some embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

The anellovectors, compositions comprising anellovectors, methods using such anellovectors, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaccious exteriors, for example a capsid disclosed in Arch Virol (2007) 152: 1961-1975, to produce anellovectors which can then be used to deliver an effector to cells (e.g., animal cells, e.g., human cells or non-human animal cells such as pig or mouse cells). In embodiments, the effector can silence expression of a factor such as an interferon. The examples further describe how anellovectors can be made by inserting effectors into sequences derived, e.g., from an Anellovirus. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specifc Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an anellovector, or the genetic element comprised in the anellovector, is introduced into a cell (e.g., a human cell). In some embodiments, the effector (e.g., an RNA, e.g., an miRNA), e.g., encoded by the genetic element of an anellovector, is expressed in a cell (e.g., a human cell), e.g., once the anellovector or the genetic element has been introduced into the cell. In some embodiments, introduction of the anellovector, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In some embodiments, introduction of the anellovector, or genetic element comprised therein, decreases level of interferon produced by the cell. In some embodiments, introduction of the anellovector, or genetic element comprised therein, into a cell modulates (e.g., increases or decreases) a function of the cell. In some embodiments, introduction of the anellovector, or genetic element comprised therein, into a cell modulates (e.g., increases or decreases) the viability of the cell. In some embodiments, introduction of the anellovector, or genetic element comprised therein, into a cell decreases viability of a cell (e.g., a cancer cell).

In some embodiments, an anellovector (e.g., a synthetic anellovector) described herein induces an antibody prevalence of less than 70% (e.g., less than about 60%, 50%, 40%, 30%, 20%, or 10% antibody prevalence). In some embodiments, antibody prevalence is determined according to methods known in the art. In some embodiments, antibody prevalence is determined by detecting antibodies against an Anellovirus (e.g., as described herein), or an anellovector based thereon, in a biological sample, e.g., according to the anti-TTV antibody detection method described in Tsuda et al. (1999; J. Virol. Methods 77: 199-206; incorporated herein by reference) and/or the method for determining anti-TTV lgG seroprevalence described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies. e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, and/or ORF2t/3, e.g., as described herein). In some embodiments, the machinery or components not encoded by the genetic element may be provided in trans (e.g., using a helper, e.g., a helper virus or helper plasmid, or encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaccous exterior comprises a capsid or a portion thereof, e.g., comprising a polypeptide encoded by an ORF1 nucleic acid, e.g., as described herein). In some embodiments, a packaging deficient genetic element is packaged into a proteinaccous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein). In some embodiments, the packaging defective genetic element cannot be packaged into a proteinaceous exterior even in the presence of factors (e.g., ORF1, ORF1/1, ORF1/2, ORF2, ORF2/2. ORF2/3, or ORF2t/3) that would permit packaging of the genetic element of a wild-type Anellovirus (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein), even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

In some embodiments, a packaging competent genetic element can be packaged into a proteinaccous exterior (e.g., wherein the proteinaceous exterior comprises a capsid or a portion thereof, e.g., comprising a polypeptide encoded by an ORF1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein) in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

### Anelloviruses

In some embodiments, an anellovector, e.g., as described herein, comprises sequences or expression products derived from an Anellovirus. In some embodiments, an anellovector includes one or more sequences or expression products that are exogenous relative to the Anellovirus. In some embodiments, an anellovector includes one or more sequences or expression products that are endogenous relative to the Anellovirus. In some embodiments, an anellovector includes one or more sequences or expression products that are heterologous relative to one or more other sequences or expression products in the anellovector. Anelloviruses generally have single-stranded circular DNA genomes with negative polarity. Anelloviruses have not generally been linked to any human disease. However, attempts to link Anellovirus infection with human disease are confounded by the high incidence of asymptomatic Anellovirus viremia in control cohort population(s), the remarkable genomic diversity within the anellovirus viral family, the historical inability to propagate the agent in vitro, and the lack of animal model(s) of Anellovirus disease (Yzebe et al., Panminerva Med. (2002) 44:167-177; Biagini, P., Vet. Microbiol. (2004) 98:95-101).

Anelloviruses are generally transmitted by oronasal or fecal-oral infection, mother-to-infant and/or in utero transmission (Gerner et al., Ped. Infect. Dis. J. (2000) 19:1074-1077). Infected persons can, in some instances, be characterized by a prolonged (months to years) Anellovirus viremia. Humans may be co-infected with more than one genogroup or strain (Saback, et al., Scad. J. Infect. Dis. (2001) 33:121-125). There is a suggestion that these genogroups can recombine within infected humans (Rey et al., Infect. (2003) 31:226-233). The double stranded isoform (replicative) intermediates have been found in several tissues, such as liver, peripheral blood mononuclear cells and bone marrow (Kikuchi et al., J. Med. Virol. (2000) 61:165-170; Okamoto et al., Biochem. Biophys. Res. Commun. (2002) 270:657-662; Rodriguez-Inigo et al., Am. J. Pathol. (2000) 156:1227-1234).

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the anellovector comprises a nucleic acid sequence selected from a sequence as shown in any of Tables N1-N1417, or a sequence having at least 70%, 75%, 80%, 85%, 90%, 95%, 96%, 97%, 98%, 99%, or 100% sequence identity thereto. In embodiments, the anellovector comprises a polypeptide comprising a sequence as shown in any of Tables A1-A1417, or a sequence having at least 70%, 75%, 80%, 85%, 90%, 95%, 96%, 97%, 98%, 99%, or 100% sequence identity thereto.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviruses described herein (e.g., an Anellovirus sequence as annotated, or as encoded by a sequence listed, in any of Tables N1-N1417. In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein (e.g., an Anellovirus sequence as annotated, or as encoded by a sequence listed, in any of Tables N1-N1417). In some embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 or ORF2 protein (e.g., an ORF1 or ORF2 amino acid sequence as shown in any of Tables A1-A1417, or an ORF1 or ORF2 amino acid sequence encoded by a nucleic acid sequence as shown in any of Tables N1-N1417). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (e.g., an ORF1 amino acid sequence as shown in any of Tables A1-A1417, or an ORF1 amino acid sequence encoded by a nucleic acid sequence as shown in any of Tables N1-N1417).

### Nucleic Acid Sequences

In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of any of Tables N1-N1417. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of any of Tables N1-N1417. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of any of Tables N1-N1417.

### Amino Acid Sequences Encoded by Nucleic Acid Sequences

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of any of Tables A1-A1417. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of any of Tables A1-A1417.

### Proteins Comprising Amino Acid Sequences

In embodiments, the anellovector described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of any of Tables A1-A1417. In embodiments, the anellovector described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of any of Tables A1-A1417. In some embodiments, an ORF1 molecule (e.g., comprised in the anellovector) comprises a polypeptide encoded by the Anellovirus ORF1 nucleic acid sequence of nucleotides 287-2038 of the nucleic acid sequence of any of Tables N1-N1417. In some embodiments, the ORF1 molecule (e.g., comprised in the anellovector) comprises an Anellovirus ORF1 protein of any of Tables A1-A1417 or a splice variant or post-translationally processed (e.g., proteolytically processed) variant thereof. In some embodiments, an ORF2 molecule (e.g., comprised in the anellovector) comprises a polypeptide encoded by the Anellovirus ORF2 nucleic acid sequence of nucleotides 105-431 of the nucleic acid sequence of any of Tables N1-N1417. In some embodiments, the ORF1 molecule (e.g., comprised in the anellovector) comprises an Anellovirus ORF1 protein of any of Tables A1-A1417 or a splice variant or post-translationally processed (e.g., proteolytically processed) variant thereof.

### Polypeptides Comprising Amino Acid Sequences

In some embodiments, the polypeptide described herein comprises an amino acid sequence having at least about 70%, 75%, 80%, 85%, 90%, 95%, 96%, 97%, 98%, 99%, or 100% sequence identity to an Anellovirus ORF1 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anellovirus ORF1 amino acid sequence of any of Tables A1-A1417.

In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anellovirus ORF1 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anellovirus ORF1 nucleic acid as listed in any of Tables A1-A1417.

In some embodiments, the polypeptide comprises an amino acid sequence (e.g., an ORF1, ORF1/1, ORF1/2, ORF2, ORF2/2, ORF2/3, or ORF2t/3 sequence) as shown in any of Tables A1-A1417, or a sequence having at least 70%, 75%, 80%, 85%, 90%, 95%, 96%, 97%, 98%, 99%, or 100% sequence identity thereto.

Lengthy table referenced here
US20240254512A1-20240801-T00001
Please refer to the end of the specification for access instructions.

Lengthy table referenced here
US20240254512A1-20240801-T00002
Please refer to the end of the specification for access instructions.

Lengthy table referenced here
US20240254512A1-20240801-T00003
Please refer to the end of the specification for access instructions.

Lengthy table referenced here
US20240254512A1-20240801-T00004
Please refer to the end of the specification for access instructions.

Lengthy table referenced here
US20240254512A1-20240801-T00005
Please refer to the end of the specification for access instructions.

Lengthy table referenced here
US20240254512A1-20240801-T00006
Please refer to the end of the specification for access instructions.

Lengthy table referenced here
US20240254512A1-20240801-T00007
Please refer to the end of the specification for access instructions.

Lengthy table referenced here
US20240254512A1-20240801-T00008
Please refer to the end of the specification for access instructions.

Lengthy table referenced here
US20240254512A1-20240801-T00009
Please refer to the end of the specification for access instructions.

Lengthy table referenced here
US20240254512A1-20240801-T00010
Please refer to the end of the specification for access instructions.

Lengthy table referenced here
US20240254512A1-20240801-T00011
Please refer to the end of the specification for access instructions.

Lengthy table referenced here
US20240254512A1-20240801-T00012
Please refer to the end of the specification for access instructions.

Lengthy table referenced here
US20240254512A1-20240801-T00013
Please refer to the end of the specification for access instructions.

Lengthy table referenced here
US20240254512A1-20240801-T00014
Please refer to the end of the specification for access instructions.

Lengthy table referenced here
US20240254512A1-20240801-T00015
Please refer to the end of the specification for access instructions.

Lengthy table referenced here
US20240254512A1-20240801-T00016
Please refer to the end of the specification for access instructions.

Lengthy table referenced here
US20240254512A1-20240801-T00017
Please refer to the end of the specification for access instructions.

Lengthy table referenced here
US20240254512A1-20240801-T00018
Please refer to the end of the specification for access instructions.

Lengthy table referenced here
US20240254512A1-20240801-T00019
Please refer to the end of the specification for access instructions.

Lengthy table referenced here
US20240254512A1-20240801-T00020
Please refer to the end of the specification for access instructions.

Lengthy table referenced here
US20240254512A1-20240801-T00021
Please refer to the end of the specification for access instructions.

Lengthy table referenced here
US20240254512A1-20240801-T00022
Please refer to the end of the specification for access instructions.

Lengthy table referenced here
US20240254512A1-20240801-T00023
Please refer to the end of the specification for access instructions.

Lengthy table referenced here
US20240254512A1-20240801-T00024
Please refer to the end of the specification for access instructions.

Lengthy table referenced here
US20240254512A1-20240801-T00025
Please refer to the end of the specification for access instructions.

Lengthy table referenced here
US20240254512A1-20240801-T00026
Please refer to the end of the specification for access instructions.

Lengthy table referenced here
US20240254512A1-20240801-T00027
Please refer to the end of the specification for access instructions.

Lengthy table referenced here
US20240254512A1-20240801-T00028
Please refer to the end of the specification for access instructions.

Lengthy table referenced here
US20240254512A1-20240801-T00029
Please refer to the end of the specification for access instructions.

Lengthy table referenced here
US20240254512A1-20240801-T00030
Please refer to the end of the specification for access instructions.

Lengthy table referenced here
US20240254512A1-20240801-T00031
Please refer to the end of the specification for access instructions.

Lengthy table referenced here
US20240254512A1-20240801-T00032
Please refer to the end of the specification for access instructions.

Lengthy table referenced here
US20240254512A1-20240801-T00033
Please refer to the end of the specification for access instructions.

Lengthy table referenced here
US20240254512A1-20240801-T00034
Please refer to the end of the specification for access instructions.

Lengthy table referenced here
US20240254512A1-20240801-T00035
Please refer to the end of the specification for access instructions.

Lengthy table referenced here
US20240254512A1-20240801-T00036
Please refer to the end of the specification for access instructions.

Lengthy table referenced here
US20240254512A1-20240801-T00037
Please refer to the end of the specification for access instructions.

Lengthy table referenced here
US20240254512A1-20240801-T00038
Please refer to the end of the specification for access instructions.

Lengthy table referenced here
US20240254512A1-20240801-T00039
Please refer to the end of the specification for access instructions.

Lengthy table referenced here
US20240254512A1-20240801-T00040
Please refer to the end of the specification for access instructions.

Lengthy table referenced here
US20240254512A1-20240801-T00041
Please refer to the end of the specification for access instructions.

Lengthy table referenced here
US20240254512A1-20240801-T00042
Please refer to the end of the specification for access instructions.

Lengthy table referenced here
US20240254512A1-20240801-T00043
Please refer to the end of the specification for access instructions.

Lengthy table referenced here
US20240254512A1-20240801-T00044
Please refer to the end of the specification for access instructions.

Lengthy table referenced here
US20240254512A1-20240801-T00045
Please refer to the end of the specification for access instructions.

Lengthy table referenced here
US20240254512A1-20240801-T00046
Please refer to the end of the specification for access instructions.

Lengthy table referenced here
US20240254512A1-20240801-T00047
Please refer to the end of the specification for access instructions.

Lengthy table referenced here
US20240254512A1-20240801-T00048
Please refer to the end of the specification for access instructions.

Lengthy table referenced here
US20240254512A1-20240801-T00049
Please refer to the end of the specification for access instructions.

Lengthy table referenced here
US20240254512A1-20240801-T00050
Please refer to the end of the specification for access instructions.

Lengthy table referenced here
US20240254512A1-20240801-T00051
Please refer to the end of the specification for access instructions.

Lengthy table referenced here
US20240254512A1-20240801-T00052
Please refer to the end of the specification for access instructions.

Lengthy table referenced here
US20240254512A1-20240801-T00053
Please refer to the end of the specification for access instructions.

Lengthy table referenced here
US20240254512A1-20240801-T00054
Please refer to the end of the specification for access instructions.

Lengthy table referenced here
US20240254512A1-20240801-T00055
Please refer to the end of the specification for access instructions.

Lengthy table referenced here
US20240254512A1-20240801-T00056
Please refer to the end of the specification for access instructions.

Lengthy table referenced here
US20240254512A1-20240801-T00057
Please refer to the end of the specification for access instructions.

Lengthy table referenced here
US20240254512A1-20240801-T00058
Please refer to the end of the specification for access instructions.

Lengthy table referenced here
US20240254512A1-20240801-T00059
Please refer to the end of the specification for access instructions.

Lengthy table referenced here
US20240254512A1-20240801-T00060
Please refer to the end of the specification for access instructions.

Lengthy table referenced here
US20240254512A1-20240801-T00061
Please refer to the end of the specification for access instructions.

Lengthy table referenced here
US20240254512A1-20240801-T00062
Please refer to the end of the specification for access instructions.

Lengthy table referenced here
US20240254512A1-20240801-T00063
Please refer to the end of the specification for access instructions.

Lengthy table referenced here
US20240254512A1-20240801-T00064
Please refer to the end of the specification for access instructions.

Lengthy table referenced here
US20240254512A1-20240801-T00065
Please refer to the end of the specification for access instructions.

Lengthy table referenced here
US20240254512A1-20240801-T00066
Please refer to the end of the specification for access instructions.

Lengthy table referenced here
US20240254512A1-20240801-T00067
Please refer to the end of the specification for access instructions.

Lengthy table referenced here
US20240254512A1-20240801-T00068
Please refer to the end of the specification for access instructions.

Lengthy table referenced here
US20240254512A1-20240801-T00069
Please refer to the end of the specification for access instructions.

Lengthy table referenced here
US20240254512A1-20240801-T00070
Please refer to the end of the specification for access instructions.

Lengthy table referenced here
US20240254512A1-20240801-T00071
Please refer to the end of the specification for access instructions.

Lengthy table referenced here
US20240254512A1-20240801-T00072
Please refer to the end of the specification for access instructions.

Lengthy table referenced here
US20240254512A1-20240801-T00073
Please refer to the end of the specification for access instructions.

Lengthy table referenced here
US20240254512A1-20240801-T00074
Please refer to the end of the specification for access instructions.

Lengthy table referenced here
US20240254512A1-20240801-T00075
Please refer to the end of the specification for access instructions.

Lengthy table referenced here
US20240254512A1-20240801-T00076
Please refer to the end of the specification for access instructions.

Lengthy table referenced here
US20240254512A1-20240801-T00077
Please refer to the end of the specification for access instructions.

Lengthy table referenced here
US20240254512A1-20240801-T00078
Please refer to the end of the specification for access instructions.

Lengthy table referenced here
US20240254512A1-20240801-T00079
Please refer to the end of the specification for access instructions.

Lengthy table referenced here
US20240254512A1-20240801-T00080
Please refer to the end of the specification for access instructions.

Lengthy table referenced here
US20240254512A1-20240801-T00081
Please refer to the end of the specification for access instructions.

Lengthy table referenced here
US20240254512A1-20240801-T00082
Please refer to the end of the specification for access instructions.

Lengthy table referenced here
US20240254512A1-20240801-T00083
Please refer to the end of the specification for access instructions.

Lengthy table referenced here
US20240254512A1-20240801-T00084
Please refer to the end of the specification for access instructions.

Lengthy table referenced here
US20240254512A1-20240801-T00085
Please refer to the end of the specification for access instructions.

Lengthy table referenced here
US20240254512A1-20240801-T00086
Please refer to the end of the specification for access instructions.

Lengthy table referenced here
US20240254512A1-20240801-T00087
Please refer to the end of the specification for access instructions.

Lengthy table referenced here
US20240254512A1-20240801-T00088
Please refer to the end of the specification for access instructions.

Lengthy table referenced here
US20240254512A1-20240801-T00089
Please refer to the end of the specification for access instructions.

Lengthy table referenced here
US20240254512A1-20240801-T00090
Please refer to the end of the specification for access instructions.

Lengthy table referenced here
US20240254512A1-20240801-T00091
Please refer to the end of the specification for access instructions.

Lengthy table referenced here
US20240254512A1-20240801-T00092
Please refer to the end of the specification for access instructions.

Lengthy table referenced here
US20240254512A1-20240801-T00093
Please refer to the end of the specification for access instructions.

Lengthy table referenced here
US20240254512A1-20240801-T00094
Please refer to the end of the specification for access instructions.

Lengthy table referenced here
US20240254512A1-20240801-T00095
Please refer to the end of the specification for access instructions.

Lengthy table referenced here
US20240254512A1-20240801-T00096
Please refer to the end of the specification for access instructions.

Lengthy table referenced here
US20240254512A1-20240801-T00097
Please refer to the end of the specification for access instructions.

Lengthy table referenced here
US20240254512A1-20240801-T00098
Please refer to the end of the specification for access instructions.

Lengthy table referenced here
US20240254512A1-20240801-T00099
Please refer to the end of the specification for access instructions.

Lengthy table referenced here
US20240254512A1-20240801-T00100
Please refer to the end of the specification for access instructions.

Lengthy table referenced here
US20240254512A1-20240801-T00101
Please refer to the end of the specification for access instructions.

Lengthy table referenced here
US20240254512A1-20240801-T00102
Please refer to the end of the specification for access instructions.

Lengthy table referenced here
US20240254512A1-20240801-T00103
Please refer to the end of the specification for access instructions.

Lengthy table referenced here
US20240254512A1-20240801-T00104
Please refer to the end of the specification for access instructions.

Lengthy table referenced here
US20240254512A1-20240801-T00105
Please refer to the end of the specification for access instructions.

Lengthy table referenced here
US20240254512A1-20240801-T00106
Please refer to the end of the specification for access instructions.

Lengthy table referenced here
US20240254512A1-20240801-T00107
Please refer to the end of the specification for access instructions.

Lengthy table referenced here
US20240254512A1-20240801-T00108
Please refer to the end of the specification for access instructions.

Lengthy table referenced here
US20240254512A1-20240801-T00109
Please refer to the end of the specification for access instructions.

Lengthy table referenced here
US20240254512A1-20240801-T00110
Please refer to the end of the specification for access instructions.

Lengthy table referenced here
US20240254512A1-20240801-T00111
Please refer to the end of the specification for access instructions.

Lengthy table referenced here
US20240254512A1-20240801-T00112
Please refer to the end of the specification for access instructions.

Lengthy table referenced here
US20240254512A1-20240801-T00113
Please refer to the end of the specification for access instructions.

Lengthy table referenced here
US20240254512A1-20240801-T00114
Please refer to the end of the specification for access instructions.

Lengthy table referenced here
US20240254512A1-20240801-T00115
Please refer to the end of the specification for access instructions.

Lengthy table referenced here
US20240254512A1-20240801-T00116
Please refer to the end of the specification for access instructions.

Lengthy table referenced here
US20240254512A1-20240801-T00117
Please refer to the end of the specification for access instructions.

Lengthy table referenced here
US20240254512A1-20240801-T00118
Please refer to the end of the specification for access instructions.

Lengthy table referenced here
US20240254512A1-20240801-T00119
Please refer to the end of the specification for access instructions.

Lengthy table referenced here
US20240254512A1-20240801-T00120
Please refer to the end of the specification for access instructions.

Lengthy table referenced here
US20240254512A1-20240801-T00121
Please refer to the end of the specification for access instructions.

Lengthy table referenced here
US20240254512A1-20240801-T00122
Please refer to the end of the specification for access instructions.

Lengthy table referenced here
US20240254512A1-20240801-T00123
Please refer to the end of the specification for access instructions.

Lengthy table referenced here
US20240254512A1-20240801-T00124
Please refer to the end of the specification for access instructions.

Lengthy table referenced here
US20240254512A1-20240801-T00125
Please refer to the end of the specification for access instructions.

Lengthy table referenced here
US20240254512A1-20240801-T00126
Please refer to the end of the specification for access instructions.

Lengthy table referenced here
US20240254512A1-20240801-T00127
Please refer to the end of the specification for access instructions.

Lengthy table referenced here
US20240254512A1-20240801-T00128
Please refer to the end of the specification for access instructions.

Lengthy table referenced here
US20240254512A1-20240801-T00129
Please refer to the end of the specification for access instructions.

Lengthy table referenced here
US20240254512A1-20240801-T00130
Please refer to the end of the specification for access instructions.

Lengthy table referenced here
US20240254512A1-20240801-T00131
Please refer to the end of the specification for access instructions.

Lengthy table referenced here
US20240254512A1-20240801-T00132
Please refer to the end of the specification for access instructions.

Lengthy table referenced here
US20240254512A1-20240801-T00133
Please refer to the end of the specification for access instructions.

Lengthy table referenced here
US20240254512A1-20240801-T00134
Please refer to the end of the specification for access instructions.

Lengthy table referenced here
US20240254512A1-20240801-T00135
Please refer to the end of the specification for access instructions.

Lengthy table referenced here
US20240254512A1-20240801-T00136
Please refer to the end of the specification for access instructions.

Lengthy table referenced here
US20240254512A1-20240801-T00137
Please refer to the end of the specification for access instructions.

Lengthy table referenced here
US20240254512A1-20240801-T00138
Please refer to the end of the specification for access instructions.

Lengthy table referenced here
US20240254512A1-20240801-T00139
Please refer to the end of the specification for access instructions.

Lengthy table referenced here
US20240254512A1-20240801-T00140
Please refer to the end of the specification for access instructions.

Lengthy table referenced here
US20240254512A1-20240801-T00141
Please refer to the end of the specification for access instructions.

Lengthy table referenced here
US20240254512A1-20240801-T00142
Please refer to the end of the specification for access instructions.

Lengthy table referenced here
US20240254512A1-20240801-T00143
Please refer to the end of the specification for access instructions.

Lengthy table referenced here
US20240254512A1-20240801-T00144
Please refer to the end of the specification for access instructions.

Lengthy table referenced here
US20240254512A1-20240801-T00145
Please refer to the end of the specification for access instructions.

Lengthy table referenced here
US20240254512A1-20240801-T00146
Please refer to the end of the specification for access instructions.

Lengthy table referenced here
US20240254512A1-20240801-T00147
Please refer to the end of the specification for access instructions.

Lengthy table referenced here
US20240254512A1-20240801-T00148
Please refer to the end of the specification for access instructions.

Lengthy table referenced here
US20240254512A1-20240801-T00149
Please refer to the end of the specification for access instructions.

Lengthy table referenced here
US20240254512A1-20240801-T00150
Please refer to the end of the specification for access instructions.

Lengthy table referenced here
US20240254512A1-20240801-T00151
Please refer to the end of the specification for access instructions.

Lengthy table referenced here
US20240254512A1-20240801-T00152
Please refer to the end of the specification for access instructions.

Lengthy table referenced here
US20240254512A1-20240801-T00153
Please refer to the end of the specification for access instructions.

Lengthy table referenced here
US20240254512A1-20240801-T00154
Please refer to the end of the specification for access instructions.

Lengthy table referenced here
US20240254512A1-20240801-T00155
Please refer to the end of the specification for access instructions.

Lengthy table referenced here
US20240254512A1-20240801-T00156
Please refer to the end of the specification for access instructions.

Lengthy table referenced here
US20240254512A1-20240801-T00157
Please refer to the end of the specification for access instructions.

Lengthy table referenced here
US20240254512A1-20240801-T00158
Please refer to the end of the specification for access instructions.

Lengthy table referenced here
US20240254512A1-20240801-T00159
Please refer to the end of the specification for access instructions.

Lengthy table referenced here
US20240254512A1-20240801-T00160
Please refer to the end of the specification for access instructions.

Lengthy table referenced here
US20240254512A1-20240801-T00161
Please refer to the end of the specification for access instructions.

Lengthy table referenced here
US20240254512A1-20240801-T00162
Please refer to the end of the specification for access instructions.

Lengthy table referenced here
US20240254512A1-20240801-T00163
Please refer to the end of the specification for access instructions.

Lengthy table referenced here
US20240254512A1-20240801-T00164
Please refer to the end of the specification for access instructions.

Lengthy table referenced here
US20240254512A1-20240801-T00165
Please refer to the end of the specification for access instructions.

Lengthy table referenced here
US20240254512A1-20240801-T00166
Please refer to the end of the specification for access instructions.

Lengthy table referenced here
US20240254512A1-20240801-T00167
Please refer to the end of the specification for access instructions.

Lengthy table referenced here
US20240254512A1-20240801-T00168
Please refer to the end of the specification for access instructions.

Lengthy table referenced here
US20240254512A1-20240801-T00169
Please refer to the end of the specification for access instructions.

Lengthy table referenced here
US20240254512A1-20240801-T00170
Please refer to the end of the specification for access instructions.

Lengthy table referenced here
US20240254512A1-20240801-T00171
Please refer to the end of the specification for access instructions.

Lengthy table referenced here
US20240254512A1-20240801-T00172
Please refer to the end of the specification for access instructions.

Lengthy table referenced here
US20240254512A1-20240801-T00173
Please refer to the end of the specification for access instructions.

Lengthy table referenced here
US20240254512A1-20240801-T00174
Please refer to the end of the specification for access instructions.

Lengthy table referenced here
US20240254512A1-20240801-T00175
Please refer to the end of the specification for access instructions.

Lengthy table referenced here
US20240254512A1-20240801-T00176
Please refer to the end of the specification for access instructions.

Lengthy table referenced here
US20240254512A1-20240801-T00177
Please refer to the end of the specification for access instructions.

Lengthy table referenced here
US20240254512A1-20240801-T00178
Please refer to the end of the specification for access instructions.

Lengthy table referenced here
US20240254512A1-20240801-T00179
Please refer to the end of the specification for access instructions.

Lengthy table referenced here
US20240254512A1-20240801-T00180
Please refer to the end of the specification for access instructions.

Lengthy table referenced here
US20240254512A1-20240801-T00181
Please refer to the end of the specification for access instructions.

Lengthy table referenced here
US20240254512A1-20240801-T00182
Please refer to the end of the specification for access instructions.

Lengthy table referenced here
US20240254512A1-20240801-T00183
Please refer to the end of the specification for access instructions.

Lengthy table referenced here
US20240254512A1-20240801-T00184
Please refer to the end of the specification for access instructions.

Lengthy table referenced here
US20240254512A1-20240801-T00185
Please refer to the end of the specification for access instructions.

Lengthy table referenced here
US20240254512A1-20240801-T00186
Please refer to the end of the specification for access instructions.

Lengthy table referenced here
US20240254512A1-20240801-T00187
Please refer to the end of the specification for access instructions.

Lengthy table referenced here
US20240254512A1-20240801-T00188
Please refer to the end of the specification for access instructions.

Lengthy table referenced here
US20240254512A1-20240801-T00189
Please refer to the end of the specification for access instructions.

Lengthy table referenced here
US20240254512A1-20240801-T00190
Please refer to the end of the specification for access instructions.

Lengthy table referenced here
US20240254512A1-20240801-T00191
Please refer to the end of the specification for access instructions.

Lengthy table referenced here
US20240254512A1-20240801-T00192
Please refer to the end of the specification for access instructions.

Lengthy table referenced here
US20240254512A1-20240801-T00193
Please refer to the end of the specification for access instructions.

Lengthy table referenced here
US20240254512A1-20240801-T00194
Please refer to the end of the specification for access instructions.

Lengthy table referenced here
US20240254512A1-20240801-T00195
Please refer to the end of the specification for access instructions.

Lengthy table referenced here
US20240254512A1-20240801-T00196
Please refer to the end of the specification for access instructions.

Lengthy table referenced here
US20240254512A1-20240801-T00197
Please refer to the end of the specification for access instructions.

Lengthy table referenced here
US20240254512A1-20240801-T00198
Please refer to the end of the specification for access instructions.

Lengthy table referenced here
US20240254512A1-20240801-T00199
Please refer to the end of the specification for access instructions.

Lengthy table referenced here
US20240254512A1-20240801-T00200
Please refer to the end of the specification for access instructions.

Lengthy table referenced here
US20240254512A1-20240801-T00201
Please refer to the end of the specification for access instructions.

Lengthy table referenced here
US20240254512A1-20240801-T00202
Please refer to the end of the specification for access instructions.

Lengthy table referenced here
US20240254512A1-20240801-T00203
Please refer to the end of the specification for access instructions.

Lengthy table referenced here
US20240254512A1-20240801-T00204
Please refer to the end of the specification for access instructions.

Lengthy table referenced here
US20240254512A1-20240801-T00205
Please refer to the end of the specification for access instructions.

Lengthy table referenced here
US20240254512A1-20240801-T00206
Please refer to the end of the specification for access instructions.

Lengthy table referenced here
US20240254512A1-20240801-T00207
Please refer to the end of the specification for access instructions.

Lengthy table referenced here
US20240254512A1-20240801-T00208
Please refer to the end of the specification for access instructions.

Lengthy table referenced here
US20240254512A1-20240801-T00209
Please refer to the end of the specification for access instructions.

Lengthy table referenced here
US20240254512A1-20240801-T00210
Please refer to the end of the specification for access instructions.

Lengthy table referenced here
US20240254512A1-20240801-T00211
Please refer to the end of the specification for access instructions.

Lengthy table referenced here
US20240254512A1-20240801-T00212
Please refer to the end of the specification for access instructions.

Lengthy table referenced here
US20240254512A1-20240801-T00213
Please refer to the end of the specification for access instructions.

Lengthy table referenced here
US20240254512A1-20240801-T00214
Please refer to the end of the specification for access instructions.

Lengthy table referenced here
US20240254512A1-20240801-T00215
Please refer to the end of the specification for access instructions.

Lengthy table referenced here
US20240254512A1-20240801-T00216
Please refer to the end of the specification for access instructions.

Lengthy table referenced here
US20240254512A1-20240801-T00217
Please refer to the end of the specification for access instructions.

Lengthy table referenced here
US20240254512A1-20240801-T00218
Please refer to the end of the specification for access instructions.

Lengthy table referenced here
US20240254512A1-20240801-T00219
Please refer to the end of the specification for access instructions.

Lengthy table referenced here
US20240254512A1-20240801-T00220
Please refer to the end of the specification for access instructions.

Lengthy table referenced here
US20240254512A1-20240801-T00221
Please refer to the end of the specification for access instructions.

Lengthy table referenced here
US20240254512A1-20240801-T00222
Please refer to the end of the specification for access instructions.

Lengthy table referenced here
US20240254512A1-20240801-T00223
Please refer to the end of the specification for access instructions.

Lengthy table referenced here
US20240254512A1-20240801-T00224
Please refer to the end of the specification for access instructions.

Lengthy table referenced here
US20240254512A1-20240801-T00225
Please refer to the end of the specification for access instructions.

Lengthy table referenced here
US20240254512A1-20240801-T00226
Please refer to the end of the specification for access instructions.

Lengthy table referenced here
US20240254512A1-20240801-T00227
Please refer to the end of the specification for access instructions.

Lengthy table referenced here
US20240254512A1-20240801-T00228
Please refer to the end of the specification for access instructions.

Lengthy table referenced here
US20240254512A1-20240801-T00229
Please refer to the end of the specification for access instructions.

Lengthy table referenced here
US20240254512A1-20240801-T00230
Please refer to the end of the specification for access instructions.

Lengthy table referenced here
US20240254512A1-20240801-T00231
Please refer to the end of the specification for access instructions.

Lengthy table referenced here
US20240254512A1-20240801-T00232
Please refer to the end of the specification for access instructions.

Lengthy table referenced here
US20240254512A1-20240801-T00233
Please refer to the end of the specification for access instructions.

Lengthy table referenced here
US20240254512A1-20240801-T00234
Please refer to the end of the specification for access instructions.

Lengthy table referenced here
US20240254512A1-20240801-T00235
Please refer to the end of the specification for access instructions.

Lengthy table referenced here
US20240254512A1-20240801-T00236
Please refer to the end of the specification for access instructions.

Lengthy table referenced here
US20240254512A1-20240801-T00237
Please refer to the end of the specification for access instructions.

Lengthy table referenced here
US20240254512A1-20240801-T00238
Please refer to the end of the specification for access instructions.

Lengthy table referenced here
US20240254512A1-20240801-T00239
Please refer to the end of the specification for access instructions.

Lengthy table referenced here
US20240254512A1-20240801-T00240
Please refer to the end of the specification for access instructions.

Lengthy table referenced here
US20240254512A1-20240801-T00241
Please refer to the end of the specification for access instructions.

Lengthy table referenced here
US20240254512A1-20240801-T00242
Please refer to the end of the specification for access instructions.

Lengthy table referenced here
US20240254512A1-20240801-T00243
Please refer to the end of the specification for access instructions.

Lengthy table referenced here
US20240254512A1-20240801-T00244
Please refer to the end of the specification for access instructions.

Lengthy table referenced here
US20240254512A1-20240801-T00245
Please refer to the end of the specification for access instructions.

Lengthy table referenced here
US20240254512A1-20240801-T00246
Please refer to the end of the specification for access instructions.

Lengthy table referenced here
US20240254512A1-20240801-T00247
Please refer to the end of the specification for access instructions.

Lengthy table referenced here
US20240254512A1-20240801-T00248
Please refer to the end of the specification for access instructions.

Lengthy table referenced here
US20240254512A1-20240801-T00249
Please refer to the end of the specification for access instructions.

Lengthy table referenced here
US20240254512A1-20240801-T00250
Please refer to the end of the specification for access instructions.

Lengthy table referenced here
US20240254512A1-20240801-T00251
Please refer to the end of the specification for access instructions.

Lengthy table referenced here
US20240254512A1-20240801-T00252
Please refer to the end of the specification for access instructions.

Lengthy table referenced here
US20240254512A1-20240801-T00253
Please refer to the end of the specification for access instructions.

Lengthy table referenced here
US20240254512A1-20240801-T00254
Please refer to the end of the specification for access instructions.

Lengthy table referenced here
US20240254512A1-20240801-T00255
Please refer to the end of the specification for access instructions.

Lengthy table referenced here
US20240254512A1-20240801-T00256
Please refer to the end of the specification for access instructions.

Lengthy table referenced here
US20240254512A1-20240801-T00257
Please refer to the end of the specification for access instructions.

Lengthy table referenced here
US20240254512A1-20240801-T00258
Please refer to the end of the specification for access instructions.

Lengthy table referenced here
US20240254512A1-20240801-T00259
Please refer to the end of the specification for access instructions.

Lengthy table referenced here
US20240254512A1-20240801-T00260
Please refer to the end of the specification for access instructions.

Lengthy table referenced here
US20240254512A1-20240801-T00261
Please refer to the end of the specification for access instructions.

Lengthy table referenced here
US20240254512A1-20240801-T00262
Please refer to the end of the specification for access instructions.

Lengthy table referenced here
US20240254512A1-20240801-T00263
Please refer to the end of the specification for access instructions.

Lengthy table referenced here
US20240254512A1-20240801-T00264
Please refer to the end of the specification for access instructions.

Lengthy table referenced here
US20240254512A1-20240801-T00265
Please refer to the end of the specification for access instructions.

Lengthy table referenced here
US20240254512A1-20240801-T00266
Please refer to the end of the specification for access instructions.

Lengthy table referenced here
US20240254512A1-20240801-T00267
Please refer to the end of the specification for access instructions.

Lengthy table referenced here
US20240254512A1-20240801-T00268
Please refer to the end of the specification for access instructions.

Lengthy table referenced here
US20240254512A1-20240801-T00269
Please refer to the end of the specification for access instructions.

Lengthy table referenced here
US20240254512A1-20240801-T00270
Please refer to the end of the specification for access instructions.

Lengthy table referenced here
US20240254512A1-20240801-T00271
Please refer to the end of the specification for access instructions.

Lengthy table referenced here
US20240254512A1-20240801-T00272
Please refer to the end of the specification for access instructions.

Lengthy table referenced here
US20240254512A1-20240801-T00273
Please refer to the end of the specification for access instructions.

Lengthy table referenced here
US20240254512A1-20240801-T00274
Please refer to the end of the specification for access instructions.

Lengthy table referenced here
US20240254512A1-20240801-T00275
Please refer to the end of the specification for access instructions.

Lengthy table referenced here
US20240254512A1-20240801-T00276
Please refer to the end of the specification for access instructions.

Lengthy table referenced here
US20240254512A1-20240801-T00277
Please refer to the end of the specification for access instructions.

Lengthy table referenced here
US20240254512A1-20240801-T00278
Please refer to the end of the specification for access instructions.

Lengthy table referenced here
US20240254512A1-20240801-T00279
Please refer to the end of the specification for access instructions.

Lengthy table referenced here
US20240254512A1-20240801-T00280
Please refer to the end of the specification for access instructions.

Lengthy table referenced here
US20240254512A1-20240801-T00281
Please refer to the end of the specification for access instructions.

Lengthy table referenced here
US20240254512A1-20240801-T00282
Please refer to the end of the specification for access instructions.

Lengthy table referenced here
US20240254512A1-20240801-T00283
Please refer to the end of the specification for access instructions.

Lengthy table referenced here
US20240254512A1-20240801-T00284
Please refer to the end of the specification for access instructions.

Lengthy table referenced here
US20240254512A1-20240801-T00285
Please refer to the end of the specification for access instructions.

Lengthy table referenced here
US20240254512A1-20240801-T00286
Please refer to the end of the specification for access instructions.

Lengthy table referenced here
US20240254512A1-20240801-T00287
Please refer to the end of the specification for access instructions.

Lengthy table referenced here
US20240254512A1-20240801-T00288
Please refer to the end of the specification for access instructions.

Lengthy table referenced here
US20240254512A1-20240801-T00289
Please refer to the end of the specification for access instructions.

Lengthy table referenced here
US20240254512A1-20240801-T00290
Please refer to the end of the specification for access instructions.

Lengthy table referenced here
US20240254512A1-20240801-T00291
Please refer to the end of the specification for access instructions.

Lengthy table referenced here
US20240254512A1-20240801-T00292
Please refer to the end of the specification for access instructions.

Lengthy table referenced here
US20240254512A1-20240801-T00293
Please refer to the end of the specification for access instructions.

Lengthy table referenced here
US20240254512A1-20240801-T00294
Please refer to the end of the specification for access instructions.

Lengthy table referenced here
US20240254512A1-20240801-T00295
Please refer to the end of the specification for access instructions.

Lengthy table referenced here
US20240254512A1-20240801-T00296
Please refer to the end of the specification for access instructions.

Lengthy table referenced here
US20240254512A1-20240801-T00297
Please refer to the end of the specification for access instructions.

Lengthy table referenced here
US20240254512A1-20240801-T00298
Please refer to the end of the specification for access instructions.

Lengthy table referenced here
US20240254512A1-20240801-T00299
Please refer to the end of the specification for access instructions.

Lengthy table referenced here
US20240254512A1-20240801-T00300
Please refer to the end of the specification for access instructions.

Lengthy table referenced here
US20240254512A1-20240801-T00301
Please refer to the end of the specification for access instructions.

Lengthy table referenced here
US20240254512A1-20240801-T00302
Please refer to the end of the specification for access instructions.

Lengthy table referenced here
US20240254512A1-20240801-T00303
Please refer to the end of the specification for access instructions.

Lengthy table referenced here
US20240254512A1-20240801-T00304
Please refer to the end of the specification for access instructions.

Lengthy table referenced here
US20240254512A1-20240801-T00305
Please refer to the end of the specification for access instructions.

Lengthy table referenced here
US20240254512A1-20240801-T00306
Please refer to the end of the specification for access instructions.

Lengthy table referenced here
US20240254512A1-20240801-T00307
Please refer to the end of the specification for access instructions.

Lengthy table referenced here
US20240254512A1-20240801-T00308
Please refer to the end of the specification for access instructions.

Lengthy table referenced here
US20240254512A1-20240801-T00309
Please refer to the end of the specification for access instructions.

Lengthy table referenced here
US20240254512A1-20240801-T00310
Please refer to the end of the specification for access instructions.

Lengthy table referenced here
US20240254512A1-20240801-T00311
Please refer to the end of the specification for access instructions.

Lengthy table referenced here
US20240254512A1-20240801-T00312
Please refer to the end of the specification for access instructions.

Lengthy table referenced here
US20240254512A1-20240801-T00313
Please refer to the end of the specification for access instructions.

Lengthy table referenced here
US20240254512A1-20240801-T00314
Please refer to the end of the specification for access instructions.

Lengthy table referenced here
US20240254512A1-20240801-T00315
Please refer to the end of the specification for access instructions.

Lengthy table referenced here
US20240254512A1-20240801-T00316
Please refer to the end of the specification for access instructions.

Lengthy table referenced here
US20240254512A1-20240801-T00317
Please refer to the end of the specification for access instructions.

Lengthy table referenced here
US20240254512A1-20240801-T00318
Please refer to the end of the specification for access instructions.

Lengthy table referenced here
US20240254512A1-20240801-T00319
Please refer to the end of the specification for access instructions.

Lengthy table referenced here
US20240254512A1-20240801-T00320
Please refer to the end of the specification for access instructions.

Lengthy table referenced here
US20240254512A1-20240801-T00321
Please refer to the end of the specification for access instructions.

Lengthy table referenced here
US20240254512A1-20240801-T00322
Please refer to the end of the specification for access instructions.

Lengthy table referenced here
US20240254512A1-20240801-T00323
Please refer to the end of the specification for access instructions.

Lengthy table referenced here
US20240254512A1-20240801-T00324
Please refer to the end of the specification for access instructions.

Lengthy table referenced here
US20240254512A1-20240801-T00325
Please refer to the end of the specification for access instructions.

Lengthy table referenced here
US20240254512A1-20240801-T00326
Please refer to the end of the specification for access instructions.

Lengthy table referenced here
US20240254512A1-20240801-T00327
Please refer to the end of the specification for access instructions.

Lengthy table referenced here
US20240254512A1-20240801-T00328
Please refer to the end of the specification for access instructions.

Lengthy table referenced here
US20240254512A1-20240801-T00329
Please refer to the end of the specification for access instructions.

Lengthy table referenced here
US20240254512A1-20240801-T00330
Please refer to the end of the specification for access instructions.

Lengthy table referenced here
US20240254512A1-20240801-T00331
Please refer to the end of the specification for access instructions.

Lengthy table referenced here
US20240254512A1-20240801-T00332
Please refer to the end of the specification for access instructions.

Lengthy table referenced here
US20240254512A1-20240801-T00333
Please refer to the end of the specification for access instructions.

Lengthy table referenced here
US20240254512A1-20240801-T00334
Please refer to the end of the specification for access instructions.

Lengthy table referenced here
US20240254512A1-20240801-T00335
Please refer to the end of the specification for access instructions.

Lengthy table referenced here
US20240254512A1-20240801-T00336
Please refer to the end of the specification for access instructions.

Lengthy table referenced here
US20240254512A1-20240801-T00337
Please refer to the end of the specification for access instructions.

Lengthy table referenced here
US20240254512A1-20240801-T00338
Please refer to the end of the specification for access instructions.

Lengthy table referenced here
US20240254512A1-20240801-T00339
Please refer to the end of the specification for access instructions.

Lengthy table referenced here
US20240254512A1-20240801-T00340
Please refer to the end of the specification for access instructions.

Lengthy table referenced here
US20240254512A1-20240801-T00341
Please refer to the end of the specification for access instructions.

Lengthy table referenced here
US20240254512A1-20240801-T00342
Please refer to the end of the specification for access instructions.

Lengthy table referenced here
US20240254512A1-20240801-T00343
Please refer to the end of the specification for access instructions.

Lengthy table referenced here
US20240254512A1-20240801-T00344
Please refer to the end of the specification for access instructions.

Lengthy table referenced here
US20240254512A1-20240801-T00345
Please refer to the end of the specification for access instructions.

Lengthy table referenced here
US20240254512A1-20240801-T00346
Please refer to the end of the specification for access instructions.

Lengthy table referenced here
US20240254512A1-20240801-T00347
Please refer to the end of the specification for access instructions.

Lengthy table referenced here
US20240254512A1-20240801-T00348
Please refer to the end of the specification for access instructions.

Lengthy table referenced here
US20240254512A1-20240801-T00349
Please refer to the end of the specification for access instructions.

Lengthy table referenced here
US20240254512A1-20240801-T00350
Please refer to the end of the specification for access instructions.

Lengthy table referenced here
US20240254512A1-20240801-T00351
Please refer to the end of the specification for access instructions.

Lengthy table referenced here
US20240254512A1-20240801-T00352
Please refer to the end of the specification for access instructions.

Lengthy table referenced here
US20240254512A1-20240801-T00353
Please refer to the end of the specification for access instructions.

Lengthy table referenced here
US20240254512A1-20240801-T00354
Please refer to the end of the specification for access instructions.

Lengthy table referenced here
US20240254512A1-20240801-T00355
Please refer to the end of the specification for access instructions.

Lengthy table referenced here
US20240254512A1-20240801-T00356
Please refer to the end of the specification for access instructions.

Lengthy table referenced here
US20240254512A1-20240801-T00357
Please refer to the end of the specification for access instructions.

Lengthy table referenced here
US20240254512A1-20240801-T00358
Please refer to the end of the specification for access instructions.

Lengthy table referenced here
US20240254512A1-20240801-T00359
Please refer to the end of the specification for access instructions.

Lengthy table referenced here
US20240254512A1-20240801-T00360
Please refer to the end of the specification for access instructions.

Lengthy table referenced here
US20240254512A1-20240801-T00361
Please refer to the end of the specification for access instructions.

Lengthy table referenced here
US20240254512A1-20240801-T00362
Please refer to the end of the specification for access instructions.

Lengthy table referenced here
US20240254512A1-20240801-T00363
Please refer to the end of the specification for access instructions.

Lengthy table referenced here
US20240254512A1-20240801-T00364
Please refer to the end of the specification for access instructions.

Lengthy table referenced here
US20240254512A1-20240801-T00365
Please refer to the end of the specification for access instructions.

Lengthy table referenced here
US20240254512A1-20240801-T00366
Please refer to the end of the specification for access instructions.

Lengthy table referenced here
US20240254512A1-20240801-T00367
Please refer to the end of the specification for access instructions.

Lengthy table referenced here
US20240254512A1-20240801-T00368
Please refer to the end of the specification for access instructions.

Lengthy table referenced here
US20240254512A1-20240801-T00369
Please refer to the end of the specification for access instructions.

Lengthy table referenced here
US20240254512A1-20240801-T00370
Please refer to the end of the specification for access instructions.

Lengthy table referenced here
US20240254512A1-20240801-T00371
Please refer to the end of the specification for access instructions.

Lengthy table referenced here
US20240254512A1-20240801-T00372
Please refer to the end of the specification for access instructions.

Lengthy table referenced here
US20240254512A1-20240801-T00373
Please refer to the end of the specification for access instructions.

Lengthy table referenced here
US20240254512A1-20240801-T00374
Please refer to the end of the specification for access instructions.

Lengthy table referenced here
US20240254512A1-20240801-T00375
Please refer to the end of the specification for access instructions.

Lengthy table referenced here
US20240254512A1-20240801-T00376
Please refer to the end of the specification for access instructions.

Lengthy table referenced here
US20240254512A1-20240801-T00377
Please refer to the end of the specification for access instructions.

Lengthy table referenced here
US20240254512A1-20240801-T00378
Please refer to the end of the specification for access instructions.

Lengthy table referenced here
US20240254512A1-20240801-T00379
Please refer to the end of the specification for access instructions.

Lengthy table referenced here
US20240254512A1-20240801-T00380
Please refer to the end of the specification for access instructions.

Lengthy table referenced here
US20240254512A1-20240801-T00381
Please refer to the end of the specification for access instructions.

Lengthy table referenced here
US20240254512A1-20240801-T00382
Please refer to the end of the specification for access instructions.

Lengthy table referenced here
US20240254512A1-20240801-T00383
Please refer to the end of the specification for access instructions.

Lengthy table referenced here
US20240254512A1-20240801-T00384
Please refer to the end of the specification for access instructions.

Lengthy table referenced here
US20240254512A1-20240801-T00385
Please refer to the end of the specification for access instructions.

Lengthy table referenced here
US20240254512A1-20240801-T00386
Please refer to the end of the specification for access instructions.

Lengthy table referenced here
US20240254512A1-20240801-T00387
Please refer to the end of the specification for access instructions.

Lengthy table referenced here
US20240254512A1-20240801-T00388
Please refer to the end of the specification for access instructions.

Lengthy table referenced here
US20240254512A1-20240801-T00389
Please refer to the end of the specification for access instructions.

Lengthy table referenced here
US20240254512A1-20240801-T00390
Please refer to the end of the specification for access instructions.

Lengthy table referenced here
US20240254512A1-20240801-T00391
Please refer to the end of the specification for access instructions.

Lengthy table referenced here
US20240254512A1-20240801-T00392
Please refer to the end of the specification for access instructions.

Lengthy table referenced here
US20240254512A1-20240801-T00393
Please refer to the end of the specification for access instructions.

Lengthy table referenced here
US20240254512A1-20240801-T00394
Please refer to the end of the specification for access instructions.

Lengthy table referenced here
US20240254512A1-20240801-T00395
Please refer to the end of the specification for access instructions.

Lengthy table referenced here
US20240254512A1-20240801-T00396
Please refer to the end of the specification for access instructions.

Lengthy table referenced here
US20240254512A1-20240801-T00397
Please refer to the end of the specification for access instructions.

Lengthy table referenced here
US20240254512A1-20240801-T00398
Please refer to the end of the specification for access instructions.

Lengthy table referenced here
US20240254512A1-20240801-T00399
Please refer to the end of the specification for access instructions.

Lengthy table referenced here
US20240254512A1-20240801-T00400
Please refer to the end of the specification for access instructions.

Lengthy table referenced here
US20240254512A1-20240801-T00401
Please refer to the end of the specification for access instructions.

Lengthy table referenced here
US20240254512A1-20240801-T00402
Please refer to the end of the specification for access instructions.

Lengthy table referenced here
US20240254512A1-20240801-T00403
Please refer to the end of the specification for access instructions.

Lengthy table referenced here
US20240254512A1-20240801-T00404
Please refer to the end of the specification for access instructions.

Lengthy table referenced here
US20240254512A1-20240801-T00405
Please refer to the end of the specification for access instructions.

Lengthy table referenced here
US20240254512A1-20240801-T00406
Please refer to the end of the specification for access instructions.

Lengthy table referenced here
US20240254512A1-20240801-T00407
Please refer to the end of the specification for access instructions.

Lengthy table referenced here
US20240254512A1-20240801-T00408
Please refer to the end of the specification for access instructions.

Lengthy table referenced here
US20240254512A1-20240801-T00409
Please refer to the end of the specification for access instructions.

Lengthy table referenced here
US20240254512A1-20240801-T00410
Please refer to the end of the specification for access instructions.

Lengthy table referenced here
US20240254512A1-20240801-T00411
Please refer to the end of the specification for access instructions.

Lengthy table referenced here
US20240254512A1-20240801-T00412
Please refer to the end of the specification for access instructions.

Lengthy table referenced here
US20240254512A1-20240801-T00413
Please refer to the end of the specification for access instructions.

Lengthy table referenced here
US20240254512A1-20240801-T00414
Please refer to the end of the specification for access instructions.

Lengthy table referenced here
US20240254512A1-20240801-T00415
Please refer to the end of the specification for access instructions.

Lengthy table referenced here
US20240254512A1-20240801-T00416
Please refer to the end of the specification for access instructions.

Lengthy table referenced here
US20240254512A1-20240801-T00417
Please refer to the end of the specification for access instructions.

Lengthy table referenced here
US20240254512A1-20240801-T00418
Please refer to the end of the specification for access instructions.

Lengthy table referenced here
US20240254512A1-20240801-T00419
Please refer to the end of the specification for access instructions.

Lengthy table referenced here
US20240254512A1-20240801-T00420
Please refer to the end of the specification for access instructions.

Lengthy table referenced here
US20240254512A1-20240801-T00421
Please refer to the end of the specification for access instructions.

Lengthy table referenced here
US20240254512A1-20240801-T00422
Please refer to the end of the specification for access instructions.

Lengthy table referenced here
US20240254512A1-20240801-T00423
Please refer to the end of the specification for access instructions.

Lengthy table referenced here
US20240254512A1-20240801-T00424
Please refer to the end of the specification for access instructions.

Lengthy table referenced here
US20240254512A1-20240801-T00425
Please refer to the end of the specification for access instructions.

Lengthy table referenced here
US20240254512A1-20240801-T00426
Please refer to the end of the specification for access instructions.

Lengthy table referenced here
US20240254512A1-20240801-T00427
Please refer to the end of the specification for access instructions.

Lengthy table referenced here
US20240254512A1-20240801-T00428
Please refer to the end of the specification for access instructions.

Lengthy table referenced here
US20240254512A1-20240801-T00429
Please refer to the end of the specification for access instructions.

Lengthy table referenced here
US20240254512A1-20240801-T00430
Please refer to the end of the specification for access instructions.

Lengthy table referenced here
US20240254512A1-20240801-T00431
Please refer to the end of the specification for access instructions.

Lengthy table referenced here
US20240254512A1-20240801-T00432
Please refer to the end of the specification for access instructions.

Lengthy table referenced here
US20240254512A1-20240801-T00433
Please refer to the end of the specification for access instructions.

Lengthy table referenced here
US20240254512A1-20240801-T00434
Please refer to the end of the specification for access instructions.

Lengthy table referenced here
US20240254512A1-20240801-T00435
Please refer to the end of the specification for access instructions.

Lengthy table referenced here
US20240254512A1-20240801-T00436
Please refer to the end of the specification for access instructions.

Lengthy table referenced here
US20240254512A1-20240801-T00437
Please refer to the end of the specification for access instructions.

Lengthy table referenced here
US20240254512A1-20240801-T00438
Please refer to the end of the specification for access instructions.

Lengthy table referenced here
US20240254512A1-20240801-T00439
Please refer to the end of the specification for access instructions.

Lengthy table referenced here
US20240254512A1-20240801-T00440
Please refer to the end of the specification for access instructions.

Lengthy table referenced here
US20240254512A1-20240801-T00441
Please refer to the end of the specification for access instructions.

Lengthy table referenced here
US20240254512A1-20240801-T00442
Please refer to the end of the specification for access instructions.

Lengthy table referenced here
US20240254512A1-20240801-T00443
Please refer to the end of the specification for access instructions.

Lengthy table referenced here
US20240254512A1-20240801-T00444
Please refer to the end of the specification for access instructions.

Lengthy table referenced here
US20240254512A1-20240801-T00445
Please refer to the end of the specification for access instructions.

Lengthy table referenced here
US20240254512A1-20240801-T00446
Please refer to the end of the specification for access instructions.

Lengthy table referenced here
US20240254512A1-20240801-T00447
Please refer to the end of the specification for access instructions.

Lengthy table referenced here
US20240254512A1-20240801-T00448
Please refer to the end of the specification for access instructions.

Lengthy table referenced here
US20240254512A1-20240801-T00449
Please refer to the end of the specification for access instructions.

Lengthy table referenced here
US20240254512A1-20240801-T00450
Please refer to the end of the specification for access instructions.

Lengthy table referenced here
US20240254512A1-20240801-T00451
Please refer to the end of the specification for access instructions.

Lengthy table referenced here
US20240254512A1-20240801-T00452
Please refer to the end of the specification for access instructions.

Lengthy table referenced here
US20240254512A1-20240801-T00453
Please refer to the end of the specification for access instructions.

Lengthy table referenced here
US20240254512A1-20240801-T00454
Please refer to the end of the specification for access instructions.

Lengthy table referenced here
US20240254512A1-20240801-T00455
Please refer to the end of the specification for access instructions.

Lengthy table referenced here
US20240254512A1-20240801-T00456
Please refer to the end of the specification for access instructions.

Lengthy table referenced here
US20240254512A1-20240801-T00457
Please refer to the end of the specification for access instructions.

Lengthy table referenced here
US20240254512A1-20240801-T00458
Please refer to the end of the specification for access instructions.

Lengthy table referenced here
US20240254512A1-20240801-T00459
Please refer to the end of the specification for access instructions.

Lengthy table referenced here
US20240254512A1-20240801-T00460
Please refer to the end of the specification for access instructions.

Lengthy table referenced here
US20240254512A1-20240801-T00461
Please refer to the end of the specification for access instructions.

Lengthy table referenced here
US20240254512A1-20240801-T00462
Please refer to the end of the specification for access instructions.

Lengthy table referenced here
US20240254512A1-20240801-T00463
Please refer to the end of the specification for access instructions.

Lengthy table referenced here
US20240254512A1-20240801-T00464
Please refer to the end of the specification for access instructions.

Lengthy table referenced here
US20240254512A1-20240801-T00465
Please refer to the end of the specification for access instructions.

Lengthy table referenced here
US20240254512A1-20240801-T00466
Please refer to the end of the specification for access instructions.

Lengthy table referenced here
US20240254512A1-20240801-T00467
Please refer to the end of the specification for access instructions.

Lengthy table referenced here
US20240254512A1-20240801-T00468
Please refer to the end of the specification for access instructions.

Lengthy table referenced here
US20240254512A1-20240801-T00469
Please refer to the end of the specification for access instructions.

Lengthy table referenced here
US20240254512A1-20240801-T00470
Please refer to the end of the specification for access instructions.

Lengthy table referenced here
US20240254512A1-20240801-T00471
Please refer to the end of the specification for access instructions.

Lengthy table referenced here
US20240254512A1-20240801-T00472
Please refer to the end of the specification for access instructions.

Lengthy table referenced here
US20240254512A1-20240801-T00473
Please refer to the end of the specification for access instructions.

Lengthy table referenced here
US20240254512A1-20240801-T00474
Please refer to the end of the specification for access instructions.

Lengthy table referenced here
US20240254512A1-20240801-T00475
Please refer to the end of the specification for access instructions.

Lengthy table referenced here
US20240254512A1-20240801-T00476
Please refer to the end of the specification for access instructions.

Lengthy table referenced here
US20240254512A1-20240801-T00477
Please refer to the end of the specification for access instructions.

Lengthy table referenced here
US20240254512A1-20240801-T00478
Please refer to the end of the specification for access instructions.

Lengthy table referenced here
US20240254512A1-20240801-T00479
Please refer to the end of the specification for access instructions.

Lengthy table referenced here
US20240254512A1-20240801-T00480
Please refer to the end of the specification for access instructions.

Lengthy table referenced here
US20240254512A1-20240801-T00481
Please refer to the end of the specification for access instructions.

Lengthy table referenced here
US20240254512A1-20240801-T00482
Please refer to the end of the specification for access instructions.

Lengthy table referenced here
US20240254512A1-20240801-T00483
Please refer to the end of the specification for access instructions.

Lengthy table referenced here
US20240254512A1-20240801-T00484
Please refer to the end of the specification for access instructions.

Lengthy table referenced here
US20240254512A1-20240801-T00485
Please refer to the end of the specification for access instructions.

Lengthy table referenced here
US20240254512A1-20240801-T00486
Please refer to the end of the specification for access instructions.

Lengthy table referenced here
US20240254512A1-20240801-T00487
Please refer to the end of the specification for access instructions.

Lengthy table referenced here
US20240254512A1-20240801-T00488
Please refer to the end of the specification for access instructions.

Lengthy table referenced here
US20240254512A1-20240801-T00489
Please refer to the end of the specification for access instructions.

Lengthy table referenced here
US20240254512A1-20240801-T00490
Please refer to the end of the specification for access instructions.

Lengthy table referenced here
US20240254512A1-20240801-T00491
Please refer to the end of the specification for access instructions.

Lengthy table referenced here
US20240254512A1-20240801-T00492
Please refer to the end of the specification for access instructions.

Lengthy table referenced here
US20240254512A1-20240801-T00493
Please refer to the end of the specification for access instructions.

Lengthy table referenced here
US20240254512A1-20240801-T00494
Please refer to the end of the specification for access instructions.

Lengthy table referenced here
US20240254512A1-20240801-T00495
Please refer to the end of the specification for access instructions.

Lengthy table referenced here
US20240254512A1-20240801-T00496
Please refer to the end of the specification for access instructions.

Lengthy table referenced here
US20240254512A1-20240801-T00497
Please refer to the end of the specification for access instructions.

Lengthy table referenced here
US20240254512A1-20240801-T00498
Please refer to the end of the specification for access instructions.

Lengthy table referenced here
US20240254512A1-20240801-T00499
Please refer to the end of the specification for access instructions.

Lengthy table referenced here
US20240254512A1-20240801-T00500
Please refer to the end of the specification for access instructions.

Lengthy table referenced here
US20240254512A1-20240801-T00501
Please refer to the end of the specification for access instructions.

Lengthy table referenced here
US20240254512A1-20240801-T00502
Please refer to the end of the specification for access instructions.

Lengthy table referenced here
US20240254512A1-20240801-T00503
Please refer to the end of the specification for access instructions.

Lengthy table referenced here
US20240254512A1-20240801-T00504
Please refer to the end of the specification for access instructions.

Lengthy table referenced here
US20240254512A1-20240801-T00505
Please refer to the end of the specification for access instructions.

Lengthy table referenced here
US20240254512A1-20240801-T00506
Please refer to the end of the specification for access instructions.

Lengthy table referenced here
US20240254512A1-20240801-T00507
Please refer to the end of the specification for access instructions.

Lengthy table referenced here
US20240254512A1-20240801-T00508
Please refer to the end of the specification for access instructions.

Lengthy table referenced here
US20240254512A1-20240801-T00509
Please refer to the end of the specification for access instructions.

Lengthy table referenced here
US20240254512A1-20240801-T00510
Please refer to the end of the specification for access instructions.

Lengthy table referenced here
US20240254512A1-20240801-T00511
Please refer to the end of the specification for access instructions.

Lengthy table referenced here
US20240254512A1-20240801-T00512
Please refer to the end of the specification for access instructions.

Lengthy table referenced here
US20240254512A1-20240801-T00513
Please refer to the end of the specification for access instructions.

Lengthy table referenced here
US20240254512A1-20240801-T00514
Please refer to the end of the specification for access instructions.

Lengthy table referenced here
US20240254512A1-20240801-T00515
Please refer to the end of the specification for access instructions.

Lengthy table referenced here
US20240254512A1-20240801-T00516
Please refer to the end of the specification for access instructions.

Lengthy table referenced here
US20240254512A1-20240801-T00517
Please refer to the end of the specification for access instructions.

Lengthy table referenced here
US20240254512A1-20240801-T00518
Please refer to the end of the specification for access instructions.

Lengthy table referenced here
US20240254512A1-20240801-T00519
Please refer to the end of the specification for access instructions.

Lengthy table referenced here
US20240254512A1-20240801-T00520
Please refer to the end of the specification for access instructions.

Lengthy table referenced here
US20240254512A1-20240801-T00521
Please refer to the end of the specification for access instructions.

Lengthy table referenced here
US20240254512A1-20240801-T00522
Please refer to the end of the specification for access instructions.

Lengthy table referenced here
US20240254512A1-20240801-T00523
Please refer to the end of the specification for access instructions.

Lengthy table referenced here
US20240254512A1-20240801-T00524
Please refer to the end of the specification for access instructions.

Lengthy table referenced here
US20240254512A1-20240801-T00525
Please refer to the end of the specification for access instructions.

Lengthy table referenced here
US20240254512A1-20240801-T00526
Please refer to the end of the specification for access instructions.

Lengthy table referenced here
US20240254512A1-20240801-T00527
Please refer to the end of the specification for access instructions.

Lengthy table referenced here
US20240254512A1-20240801-T00528
Please refer to the end of the specification for access instructions.

Lengthy table referenced here
US20240254512A1-20240801-T00529
Please refer to the end of the specification for access instructions.

Lengthy table referenced here
US20240254512A1-20240801-T00530
Please refer to the end of the specification for access instructions.

Lengthy table referenced here
US20240254512A1-20240801-T00531
Please refer to the end of the specification for access instructions.

Lengthy table referenced here
US20240254512A1-20240801-T00532
Please refer to the end of the specification for access instructions.

Lengthy table referenced here
US20240254512A1-20240801-T00533
Please refer to the end of the specification for access instructions.

Lengthy table referenced here
US20240254512A1-20240801-T00534
Please refer to the end of the specification for access instructions.

Lengthy table referenced here
US20240254512A1-20240801-T00535
Please refer to the end of the specification for access instructions.

Lengthy table referenced here
US20240254512A1-20240801-T00536
Please refer to the end of the specification for access instructions.

Lengthy table referenced here
US20240254512A1-20240801-T00537
Please refer to the end of the specification for access instructions.

Lengthy table referenced here
US20240254512A1-20240801-T00538
Please refer to the end of the specification for access instructions.

Lengthy table referenced here
US20240254512A1-20240801-T00539
Please refer to the end of the specification for access instructions.

Lengthy table referenced here
US20240254512A1-20240801-T00540
Please refer to the end of the specification for access instructions.

Lengthy table referenced here
US20240254512A1-20240801-T00541
Please refer to the end of the specification for access instructions.

Lengthy table referenced here
US20240254512A1-20240801-T00542
Please refer to the end of the specification for access instructions.

Lengthy table referenced here
US20240254512A1-20240801-T00543
Please refer to the end of the specification for access instructions.

Lengthy table referenced here
US20240254512A1-20240801-T00544
Please refer to the end of the specification for access instructions.

Lengthy table referenced here
US20240254512A1-20240801-T00545
Please refer to the end of the specification for access instructions.

Lengthy table referenced here
US20240254512A1-20240801-T00546
Please refer to the end of the specification for access instructions.

Lengthy table referenced here
US20240254512A1-20240801-T00547
Please refer to the end of the specification for access instructions.

Lengthy table referenced here
US20240254512A1-20240801-T00548
Please refer to the end of the specification for access instructions.

Lengthy table referenced here
US20240254512A1-20240801-T00549
Please refer to the end of the specification for access instructions.

Lengthy table referenced here
US20240254512A1-20240801-T00550
Please refer to the end of the specification for access instructions.

Lengthy table referenced here
US20240254512A1-20240801-T00551
Please refer to the end of the specification for access instructions.

Lengthy table referenced here
US20240254512A1-20240801-T00552
Please refer to the end of the specification for access instructions.

Lengthy table referenced here
US20240254512A1-20240801-T00553
Please refer to the end of the specification for access instructions.

Lengthy table referenced here
US20240254512A1-20240801-T00554
Please refer to the end of the specification for access instructions.

Lengthy table referenced here
US20240254512A1-20240801-T00555
Please refer to the end of the specification for access instructions.

Lengthy table referenced here
US20240254512A1-20240801-T00556
Please refer to the end of the specification for access instructions.

Lengthy table referenced here
US20240254512A1-20240801-T00557
Please refer to the end of the specification for access instructions.

Lengthy table referenced here
US20240254512A1-20240801-T00558
Please refer to the end of the specification for access instructions.

Lengthy table referenced here
US20240254512A1-20240801-T00559
Please refer to the end of the specification for access instructions.

Lengthy table referenced here
US20240254512A1-20240801-T00560
Please refer to the end of the specification for access instructions.

Lengthy table referenced here
US20240254512A1-20240801-T00561
Please refer to the end of the specification for access instructions.

Lengthy table referenced here
US20240254512A1-20240801-T00562
Please refer to the end of the specification for access instructions.

Lengthy table referenced here
US20240254512A1-20240801-T00563
Please refer to the end of the specification for access instructions.

Lengthy table referenced here
US20240254512A1-20240801-T00564
Please refer to the end of the specification for access instructions.

Lengthy table referenced here
US20240254512A1-20240801-T00565
Please refer to the end of the specification for access instructions.

Lengthy table referenced here
US20240254512A1-20240801-T00566
Please refer to the end of the specification for access instructions.

Lengthy table referenced here
US20240254512A1-20240801-T00567
Please refer to the end of the specification for access instructions.

Lengthy table referenced here
US20240254512A1-20240801-T00568
Please refer to the end of the specification for access instructions.

Lengthy table referenced here
US20240254512A1-20240801-T00569
Please refer to the end of the specification for access instructions.

Lengthy table referenced here
US20240254512A1-20240801-T00570
Please refer to the end of the specification for access instructions.

Lengthy table referenced here
US20240254512A1-20240801-T00571
Please refer to the end of the specification for access instructions.

Lengthy table referenced here
US20240254512A1-20240801-T00572
Please refer to the end of the specification for access instructions.

Lengthy table referenced here
US20240254512A1-20240801-T00573
Please refer to the end of the specification for access instructions.

Lengthy table referenced here
US20240254512A1-20240801-T00574
Please refer to the end of the specification for access instructions.

Lengthy table referenced here
US20240254512A1-20240801-T00575
Please refer to the end of the specification for access instructions.

Lengthy table referenced here
US20240254512A1-20240801-T00576
Please refer to the end of the specification for access instructions.

Lengthy table referenced here
US20240254512A1-20240801-T00577
Please refer to the end of the specification for access instructions.

Lengthy table referenced here
US20240254512A1-20240801-T00578
Please refer to the end of the specification for access instructions.

Lengthy table referenced here
US20240254512A1-20240801-T00579
Please refer to the end of the specification for access instructions.

Lengthy table referenced here
US20240254512A1-20240801-T00580
Please refer to the end of the specification for access instructions.

Lengthy table referenced here
US20240254512A1-20240801-T00581
Please refer to the end of the specification for access instructions.

Lengthy table referenced here
US20240254512A1-20240801-T00582
Please refer to the end of the specification for access instructions.

Lengthy table referenced here
US20240254512A1-20240801-T00583
Please refer to the end of the specification for access instructions.

Lengthy table referenced here
US20240254512A1-20240801-T00584
Please refer to the end of the specification for access instructions.

Lengthy table referenced here
US20240254512A1-20240801-T00585
Please refer to the end of the specification for access instructions.

Lengthy table referenced here
US20240254512A1-20240801-T00586
Please refer to the end of the specification for access instructions.

Lengthy table referenced here
US20240254512A1-20240801-T00587
Please refer to the end of the specification for access instructions.

Lengthy table referenced here
US20240254512A1-20240801-T00588
Please refer to the end of the specification for access instructions.

Lengthy table referenced here
US20240254512A1-20240801-T00589
Please refer to the end of the specification for access instructions.

Lengthy table referenced here
US20240254512A1-20240801-T00590
Please refer to the end of the specification for access instructions.

Lengthy table referenced here
US20240254512A1-20240801-T00591
Please refer to the end of the specification for access instructions.

Lengthy table referenced here
US20240254512A1-20240801-T00592
Please refer to the end of the specification for access instructions.

Lengthy table referenced here
US20240254512A1-20240801-T00593
Please refer to the end of the specification for access instructions.

Lengthy table referenced here
US20240254512A1-20240801-T00594
Please refer to the end of the specification for access instructions.

Lengthy table referenced here
US20240254512A1-20240801-T00595
Please refer to the end of the specification for access instructions.

Lengthy table referenced here
US20240254512A1-20240801-T00596
Please refer to the end of the specification for access instructions.

Lengthy table referenced here
US20240254512A1-20240801-T00597
Please refer to the end of the specification for access instructions.

Lengthy table referenced here
US20240254512A1-20240801-T00598
Please refer to the end of the specification for access instructions.

Lengthy table referenced here
US20240254512A1-20240801-T00599
Please refer to the end of the specification for access instructions.

Lengthy table referenced here
US20240254512A1-20240801-T00600
Please refer to the end of the specification for access instructions.

Lengthy table referenced here
US20240254512A1-20240801-T00601
Please refer to the end of the specification for access instructions.

Lengthy table referenced here
US20240254512A1-20240801-T00602
Please refer to the end of the specification for access instructions.

Lengthy table referenced here
US20240254512A1-20240801-T00603
Please refer to the end of the specification for access instructions.

Lengthy table referenced here
US20240254512A1-20240801-T00604
Please refer to the end of the specification for access instructions.

Lengthy table referenced here
US20240254512A1-20240801-T00605
Please refer to the end of the specification for access instructions.

Lengthy table referenced here
US20240254512A1-20240801-T00606
Please refer to the end of the specification for access instructions.

Lengthy table referenced here
US20240254512A1-20240801-T00607
Please refer to the end of the specification for access instructions.

Lengthy table referenced here
US20240254512A1-20240801-T00608
Please refer to the end of the specification for access instructions.

Lengthy table referenced here
US20240254512A1-20240801-T00609
Please refer to the end of the specification for access instructions.

Lengthy table referenced here
US20240254512A1-20240801-T00610
Please refer to the end of the specification for access instructions.

Lengthy table referenced here
US20240254512A1-20240801-T00611
Please refer to the end of the specification for access instructions.

Lengthy table referenced here
US20240254512A1-20240801-T00612
Please refer to the end of the specification for access instructions.

Lengthy table referenced here
US20240254512A1-20240801-T00613
Please refer to the end of the specification for access instructions.

Lengthy table referenced here
US20240254512A1-20240801-T00614
Please refer to the end of the specification for access instructions.

Lengthy table referenced here
US20240254512A1-20240801-T00615
Please refer to the end of the specification for access instructions.

Lengthy table referenced here
US20240254512A1-20240801-T00616
Please refer to the end of the specification for access instructions.

Lengthy table referenced here
US20240254512A1-20240801-T00617
Please refer to the end of the specification for access instructions.

Lengthy table referenced here
US20240254512A1-20240801-T00618
Please refer to the end of the specification for access instructions.

Lengthy table referenced here
US20240254512A1-20240801-T00619
Please refer to the end of the specification for access instructions.

Lengthy table referenced here
US20240254512A1-20240801-T00620
Please refer to the end of the specification for access instructions.

Lengthy table referenced here
US20240254512A1-20240801-T00621
Please refer to the end of the specification for access instructions.

Lengthy table referenced here
US20240254512A1-20240801-T00622
Please refer to the end of the specification for access instructions.

Lengthy table referenced here
US20240254512A1-20240801-T00623
Please refer to the end of the specification for access instructions.

Lengthy table referenced here
US20240254512A1-20240801-T00624
Please refer to the end of the specification for access instructions.

Lengthy table referenced here
US20240254512A1-20240801-T00625
Please refer to the end of the specification for access instructions.

Lengthy table referenced here
US20240254512A1-20240801-T00626
Please refer to the end of the specification for access instructions.

Lengthy table referenced here
US20240254512A1-20240801-T00627
Please refer to the end of the specification for access instructions.

Lengthy table referenced here
US20240254512A1-20240801-T00628
Please refer to the end of the specification for access instructions.

Lengthy table referenced here
US20240254512A1-20240801-T00629
Please refer to the end of the specification for access instructions.

Lengthy table referenced here
US20240254512A1-20240801-T00630
Please refer to the end of the specification for access instructions.

Lengthy table referenced here
US20240254512A1-20240801-T00631
Please refer to the end of the specification for access instructions.

Lengthy table referenced here
US20240254512A1-20240801-T00632
Please refer to the end of the specification for access instructions.

Lengthy table referenced here
US20240254512A1-20240801-T00633
Please refer to the end of the specification for access instructions.

Lengthy table referenced here
US20240254512A1-20240801-T00634
Please refer to the end of the specification for access instructions.

Lengthy table referenced here
US20240254512A1-20240801-T00635
Please refer to the end of the specification for access instructions.

Lengthy table referenced here
US20240254512A1-20240801-T00636
Please refer to the end of the specification for access instructions.

Lengthy table referenced here
US20240254512A1-20240801-T00637
Please refer to the end of the specification for access instructions.

Lengthy table referenced here
US20240254512A1-20240801-T00638
Please refer to the end of the specification for access instructions.

Lengthy table referenced here
US20240254512A1-20240801-T00639
Please refer to the end of the specification for access instructions.

Lengthy table referenced here
US20240254512A1-20240801-T00640
Please refer to the end of the specification for access instructions.

Lengthy table referenced here
US20240254512A1-20240801-T00641
Please refer to the end of the specification for access instructions.

Lengthy table referenced here
US20240254512A1-20240801-T00642
Please refer to the end of the specification for access instructions.

Lengthy table referenced here
US20240254512A1-20240801-T00643
Please refer to the end of the specification for access instructions.

Lengthy table referenced here
US20240254512A1-20240801-T00644
Please refer to the end of the specification for access instructions.

Lengthy table referenced here
US20240254512A1-20240801-T00645
Please refer to the end of the specification for access instructions.

Lengthy table referenced here
US20240254512A1-20240801-T00646
Please refer to the end of the specification for access instructions.

Lengthy table referenced here
US20240254512A1-20240801-T00647
Please refer to the end of the specification for access instructions.

Lengthy table referenced here
US20240254512A1-20240801-T00648
Please refer to the end of the specification for access instructions.

Lengthy table referenced here
US20240254512A1-20240801-T00649
Please refer to the end of the specification for access instructions.

Lengthy table referenced here
US20240254512A1-20240801-T00650
Please refer to the end of the specification for access instructions.

Lengthy table referenced here
US20240254512A1-20240801-T00651
Please refer to the end of the specification for access instructions.

Lengthy table referenced here
US20240254512A1-20240801-T00652
Please refer to the end of the specification for access instructions.

Lengthy table referenced here
US20240254512A1-20240801-T00653
Please refer to the end of the specification for access instructions.

Lengthy table referenced here
US20240254512A1-20240801-T00654
Please refer to the end of the specification for access instructions.

Lengthy table referenced here
US20240254512A1-20240801-T00655
Please refer to the end of the specification for access instructions.

Lengthy table referenced here
US20240254512A1-20240801-T00656
Please refer to the end of the specification for access instructions.

Lengthy table referenced here
US20240254512A1-20240801-T00657
Please refer to the end of the specification for access instructions.

Lengthy table referenced here
US20240254512A1-20240801-T00658
Please refer to the end of the specification for access instructions.

Lengthy table referenced here
US20240254512A1-20240801-T00659
Please refer to the end of the specification for access instructions.

Lengthy table referenced here
US20240254512A1-20240801-T00660
Please refer to the end of the specification for access instructions.

Lengthy table referenced here
US20240254512A1-20240801-T00661
Please refer to the end of the specification for access instructions.

Lengthy table referenced here
US20240254512A1-20240801-T00662
Please refer to the end of the specification for access instructions.

Lengthy table referenced here
US20240254512A1-20240801-T00663
Please refer to the end of the specification for access instructions.

Lengthy table referenced here
US20240254512A1-20240801-T00664
Please refer to the end of the specification for access instructions.

Lengthy table referenced here
US20240254512A1-20240801-T00665
Please refer to the end of the specification for access instructions.

Lengthy table referenced here
US20240254512A1-20240801-T00666
Please refer to the end of the specification for access instructions.

Lengthy table referenced here
US20240254512A1-20240801-T00667
Please refer to the end of the specification for access instructions.

Lengthy table referenced here
US20240254512A1-20240801-T00668
Please refer to the end of the specification for access instructions.

Lengthy table referenced here
US20240254512A1-20240801-T00669
Please refer to the end of the specification for access instructions.

Lengthy table referenced here
US20240254512A1-20240801-T00670
Please refer to the end of the specification for access instructions.

Lengthy table referenced here
US20240254512A1-20240801-T00671
Please refer to the end of the specification for access instructions.

Lengthy table referenced here
US20240254512A1-20240801-T00672
Please refer to the end of the specification for access instructions.

Lengthy table referenced here
US20240254512A1-20240801-T00673
Please refer to the end of the specification for access instructions.

Lengthy table referenced here
US20240254512A1-20240801-T00674
Please refer to the end of the specification for access instructions.

Lengthy table referenced here
US20240254512A1-20240801-T00675
Please refer to the end of the specification for access instructions.

Lengthy table referenced here
US20240254512A1-20240801-T00676
Please refer to the end of the specification for access instructions.

Lengthy table referenced here
US20240254512A1-20240801-T00677
Please refer to the end of the specification for access instructions.

Lengthy table referenced here
US20240254512A1-20240801-T00678
Please refer to the end of the specification for access instructions.

Lengthy table referenced here
US20240254512A1-20240801-T00679
Please refer to the end of the specification for access instructions.

Lengthy table referenced here
US20240254512A1-20240801-T00680
Please refer to the end of the specification for access instructions.

Lengthy table referenced here
US20240254512A1-20240801-T00681
Please refer to the end of the specification for access instructions.

Lengthy table referenced here
US20240254512A1-20240801-T00682
Please refer to the end of the specification for access instructions.

Lengthy table referenced here
US20240254512A1-20240801-T00683
Please refer to the end of the specification for access instructions.

Lengthy table referenced here
US20240254512A1-20240801-T00684
Please refer to the end of the specification for access instructions.

Lengthy table referenced here
US20240254512A1-20240801-T00685
Please refer to the end of the specification for access instructions.

Lengthy table referenced here
US20240254512A1-20240801-T00686
Please refer to the end of the specification for access instructions.

Lengthy table referenced here
US20240254512A1-20240801-T00687
Please refer to the end of the specification for access instructions.

Lengthy table referenced here
US20240254512A1-20240801-T00688
Please refer to the end of the specification for access instructions.

Lengthy table referenced here
US20240254512A1-20240801-T00689
Please refer to the end of the specification for access instructions.

Lengthy table referenced here
US20240254512A1-20240801-T00690
Please refer to the end of the specification for access instructions.

Lengthy table referenced here
US20240254512A1-20240801-T00691
Please refer to the end of the specification for access instructions.

Lengthy table referenced here
US20240254512A1-20240801-T00692
Please refer to the end of the specification for access instructions.

Lengthy table referenced here
US20240254512A1-20240801-T00693
Please refer to the end of the specification for access instructions.

Lengthy table referenced here
US20240254512A1-20240801-T00694
Please refer to the end of the specification for access instructions.

Lengthy table referenced here
US20240254512A1-20240801-T00695
Please refer to the end of the specification for access instructions.

Lengthy table referenced here
US20240254512A1-20240801-T00696
Please refer to the end of the specification for access instructions.

Lengthy table referenced here
US20240254512A1-20240801-T00697
Please refer to the end of the specification for access instructions.

Lengthy table referenced here
US20240254512A1-20240801-T00698
Please refer to the end of the specification for access instructions.

Lengthy table referenced here
US20240254512A1-20240801-T00699
Please refer to the end of the specification for access instructions.

Lengthy table referenced here
US20240254512A1-20240801-T00700
Please refer to the end of the specification for access instructions.

Lengthy table referenced here
US20240254512A1-20240801-T00701
Please refer to the end of the specification for access instructions.

Lengthy table referenced here
US20240254512A1-20240801-T00702
Please refer to the end of the specification for access instructions.

Lengthy table referenced here
US20240254512A1-20240801-T00703
Please refer to the end of the specification for access instructions.

Lengthy table referenced here
US20240254512A1-20240801-T00704
Please refer to the end of the specification for access instructions.

Lengthy table referenced here
US20240254512A1-20240801-T00705
Please refer to the end of the specification for access instructions.

Lengthy table referenced here
US20240254512A1-20240801-T00706
Please refer to the end of the specification for access instructions.

Lengthy table referenced here
US20240254512A1-20240801-T00707
Please refer to the end of the specification for access instructions.

Lengthy table referenced here
US20240254512A1-20240801-T00708
Please refer to the end of the specification for access instructions.

Lengthy table referenced here
US20240254512A1-20240801-T00709
Please refer to the end of the specification for access instructions.

Lengthy table referenced here
US20240254512A1-20240801-T00710
Please refer to the end of the specification for access instructions.

Lengthy table referenced here
US20240254512A1-20240801-T00711
Please refer to the end of the specification for access instructions.

Lengthy table referenced here
US20240254512A1-20240801-T00712
Please refer to the end of the specification for access instructions.

Lengthy table referenced here
US20240254512A1-20240801-T00713
Please refer to the end of the specification for access instructions.

In some embodiments, an anellovector as described herein is a chimeric anellovector. In some embodiments, a chimeric anellovector further comprises one or more elements, polypeptides, or nucleic acids from a virus other than an Anellovirus.

In some embodiments, the chimeric anellovector comprises a plurality of polypeptides (e.g., Anellovirus ORF1, ORF1/1, ORF1/2, ORF2, ORF2/2, ORF2/3, and/or ORF2t/3) comprising sequences from a plurality of different Anelloviruses (e.g., as described herein).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from an Anellovirus (e.g., as described herein) and at least one portion from a different virus (e.g., as described herein).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from one Anellovirus (e.g., as described herein) and at least one portion from a different Anellovirus (e.g., as described herein). In some embodiments, the anellovector comprises a chimeric ORF1 molecule comprising at least one portion of an ORF1 molecule from one Anellovirus (e.g., as described herein), or an ORF1 molecule having at least 75%, 80%, 85%, 90%, 95%, 96%, 97%, 98%, or 99% amino acid sequence identity thereto, and at least one portion of an ORF1 molecule from a different Anellovirus (e.g., as described herein), or an ORF1 molecule having at least 75%, 80%, 85%, 90%, 95%, 96%, 97%, 98%, or 99% amino acid sequence identity thereto. In some embodiments, the chimeric ORF1 molecule comprises an ORF1 jelly-roll domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 arginine-rich region from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 hypervariable domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 N22 domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 C-terminal domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto.

In some embodiments, the anellovector comprises a chimeric ORF1/1 molecule comprising at least one portion of an ORF1/1 molecule from one Anellovirus (e.g., as described herein), or an ORF1/1 molecule having at least 75%, 80%, 85%, 90%, 95%, 96%, 97%, 98%, or 99% amino acid sequence identity thereto, and at least one portion of an ORF1/1 molecule from a different Anellovirus (e.g., as described herein), or an ORF1/1 molecule having at least 75%, 80%, 85%, 90%, 95%, 96%, 97%, 98%, or 99% amino acid sequence identity thereto. In some embodiments, the anellovector comprises a chimeric ORF1/2 molecule comprising at least one portion of an ORF1/2 molecule from one Anellovirus (e.g., as described herein), or an ORF1/2 molecule having at least 75%, 80%, 85%, 90%, 95%, 96%, 97%, 98%, or 99% amino acid sequence identity thereto, and at least one portion of an ORF1/2 molecule from a different Anellovirus (e.g., as described herein), or an ORF1/2 molecule having at least 75%, 80%, 85%, 90%, 95%, 96%, 97%, 98%, or 99% amino acid sequence identity thereto. In some embodiments, the anellovector comprises a chimeric ORF2 molecule comprising at least one portion of an ORF2 molecule from one Anellovirus (e.g., as described herein), or an ORF2 molecule having at least 75%, 80%, 85%, 90%, 95%, 96%, 97%, 98%, or 99% amino acid sequence identity thereto, and at least one portion of an ORF2 molecule from a different Anellovirus (e.g., as described herein), or an ORF2 molecule having at least 75%, 80%, 85%, 90%, 95%, 96%, 97%, 98%, or 99% amino acid sequence identity thereto. In some embodiments, the anellovector comprises a chimeric ORF2/2 molecule comprising at least one portion of an ORF2/2 molecule from one Anellovirus (e.g., as described herein), or an ORF2/2 molecule having at least 75%, 80%, 85%, 90%, 95%, 96%, 97%, 98%, or 99% amino acid sequence identity thereto, and at least one portion of an ORF2/2 molecule from a different Anellovirus (e.g., as described herein), or an ORF2/2 molecule having at least 75%, 80%, 85%, 90%, 95%, 96%, 97%, 98%, or 99% amino acid sequence identity thereto. In some embodiments, the anellovector comprises a chimeric ORF2/3 molecule comprising at least one portion of an ORF2/3 molecule from one Anellovirus (e.g., as described herein), or an ORF2/3 molecule having at least 75%, 80%, 85%, 90%, 95%, 96%, 97%, 98%, or 99% amino acid sequence identity thereto, and at least one portion of an ORF2/3 molecule from a different Anellovirus (e.g., as described herein), or an ORF2/3 molecule having at least 75%, 80%, 85%, 90%, 95%, 96%, 97%, 98%, or 99% amino acid sequence identity thereto. In some embodiments, the anellovector comprises a chimeric ORF2T/3 molecule comprising at least one portion of an ORF2T/3 molecule from one Anellovirus (e.g., as described herein), or an ORF2T/3 molecule having at least 75%, 80%, 85%, 90%, 95%, 96%, 97%, 98%, or 99% amino acid sequence identity thereto, and at least one portion of an ORF2T/3 molecule from a different Anellovirus (e.g., as described herein), or an ORF2T/3 molecule having at least 75%, 80%, 85%, 90%, 95%, 96%, 97%, 98%, or 99% amino acid sequence identity thereto.

In some embodiments, an anellovector comprises a nucleic acid comprising a sequence listed in PCT Application No. PCT/US2018/037379, incorporated herein by reference in its entirety. In some embodiments, an anellovector comprises a polypeptide comprising a sequence listed in PCT Application No. PCT/US2018/037379, incorporated herein by reference in its entirety.

In some embodiments, an anellovector comprises an Anellovirus genome, e.g., as identified according to the method described in Example 9. In some embodiments, an anellovector comprises an Anellovirus sequence, or a portion thereof, as described in Example 13.

In some embodiments, an anellovector comprises a genetic element comprising a consensus Anellovirus motif, e.g., as shown in Table 19. In some embodiments, an anellovector comprises a genetic element comprising a consensus Anellovirus ORF1 motif, e.g., as shown in Table 19. In some embodiments, an anellovector comprises a genetic element comprising a consensus Anellovirus ORF1/1 motif, e.g., as shown in Table 19. In some embodiments, an anellovector comprises a genetic element comprising a consensus Anellovirus ORF1/2 motif, e.g., as shown in Table 19. In some embodiments, an anellovector comprises a genetic element comprising a consensus Anellovirus ORF2/2 motif, e.g., as shown in Table 19. In some embodiments, an anellovector comprises a genetic element comprising a consensus Anellovirus ORF2/3 motif, e.g., as shown in Table 19. In some embodiments, an anellovector comprises a genetic element comprising a consensus Anellovirus ORF2t/3 motif, e.g., as shown in Table 19. In some embodiments, X, as shown in Table 19, indicates any amino acid. In some embodiments, Z, as shown in Table 19, indicates glutamic acid or glutamine. In some embodiments, B, as shown in Table 19, indicates aspartic acid or asparagine. In some embodiments, J, as shown in Table 19, indicates leucine or isoleucine.

TABLE 19
Consensus motifs in open reading frames (ORFs) of Anelloviruses

| Consensus | Open | | | |
|---|---|---|---|---|
| Threshold | Reading Frame | Position | Motif | SEQ ID NO: |
| 50 | ORF1 | 79 | LIJRQWQPXXIRRCXIXGYXPLIXC | 5075 |
| 50 | ORF1 | 111 | NYXXHXD | 5076 |
| 50 | ORF1 | 135 | FSLXXLYDZ | 5077 |
| 50 | ORF1 | 149 | NXWTXSNXDLDLCRYXGC | 5078 |
| 50 | ORF1 | 194 | TXPSXHPGXMXLXKHK | 5079 |
| 50 | ORF1 | 212 | IPSLXTRPXG | 5080 |
| 50 | ORF1 | 228 | RIXPPXLFXDKWYFQXDL | 5081 |
| 50 | ORF1 | 250 | LLXIXATA | 5082 |
| 50 | ORF1 | 260 | LXXPFXSPXTD | 5083 |
| 50 | ORF1 | 448 | YNPXXDKGXGNXIW | 5084 |
| 50 | ORF1 | 519 | CPYTZPXL | 5085 |
| 50 | ORF1 | 542 | XFGXGXMP | 5086 |
| 50 | ORF1 | 569 | HQXEVXEX | 5087 |
| 50 | ORF1 | 600 | KYXFXFXWGGNP | 5088 |
| 50 | ORF1 | 653 | HSWDXRRG | 5089 |
| 50 | ORF1 | 666 | AIKRXQQ | 5090 |
| 50 | ORF1 | 750 | XQZQXXLR | 5091 |
| 50 | ORF1/1 | 73 | PRXJQXXDP | 5092 |
| 50 | ORF1/1 | 91 | HSWDXRRG | 5093 |
| 50 | ORF1/1 | 105 | AIKRXQQ | 5094 |
| 50 | ORF1/1 | 187 | QZQXXLR | 5095 |
| 50 | ORF1/2 | 97 | KXKRRRR | 5096 |
| 50 | ORF2/2 | 158 | PIXSLXXYKXXTR | 5097 |
| 50 | ORF2/2 | 189 | LAXQLLKECXKN | 5098 |
| 50 | ORF2/3 | 39 | HLNXLA | 5099 |
| 50 | ORF2/3 | 272 | DRPPR | 5100 |
| 50 | ORF2/3 | 281 | DXPFYPWXP | 5101 |
| 50 | ORF2/3 | 300 | VXFKLXF | 5102 |
| 50 | ORF2t/3 | 4 | WXPPVHBVXGIERXW | 5103 |
| 50 | ORF2t/3 | 37 | AKRKLX | 5104 |
| 50 | ORF2t/3 | 140 | PSSXDWXXEY | 5105 |
| 50 | ORF2t/3 | 156 | DRPPR | 5106 |
| 50 | ORF2t/3 | 167 | PFYPW | 5107 |
| 50 | ORF2t/3 | 183 | NVXFKLXF | 5108 |
| 50 | ORF1 | 84 | JXXXXWQPXXXXXCXIXGXXXJWQP | 5109 |
| 50 | ORF1 | 149 | NXWXXXNXXXXLXRY | 5110 |
| 50 | ORF1 | 448 | YNPXXDXG | 5111 |

### ORF1 Molecules

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein, e.g., as listed in any of Tables A1-A1417), or a functional fragment thereof. In some embodiments, the ORF1 molecule comprises a truncation relative to an Anellovirus ORF1 protein (e.g., an Anellovirus ORF1 protein as described herein, e.g., as listed in any of Tables A1-A1417). In some 10 embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 70%, 75%, 80%, 85%, 90%, 95%, 96%, 97%, 98%, 99%, or 100% sequence identity to an Anellovirus ORF1 protein sequence as shown in any of Tables A1-A1417. In some embodiments, an ORF1 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

In some embodiments, an ORF1 molecule as described herein comprises an amino acid sequence (e.g., an ORF1 sequence, or an arginine-rich region, jelly-roll domain, HVR, N22, or C-terminal domain sequence) as listed in any of Tables A2, A4, A6, A8, A10, A12, C1-C5, 2, 4, 6, 8, 10, 12, 14, 16, 18, 20-37, or D1-D10 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may encapsidate a nucleic acid molecule (e.g., a genetic element as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer (e.g., comprising a plurality of distinct ORF1 molecules). It is also contemplated that an ORF1 molecule may have replicase activity.

An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands).

### Arginine-Rich Region

An arginine rich region has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

### Jelly Roll Domain

A jelly-roll domain or region comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) β-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first β-sheet in antiparallel orientation to a second β-sheet. In certain embodiments, the first β-sheet comprises about four (e.g., 3, 4, 5, or 6) β-strands. In certain embodiments, the second β-sheet comprises about four (e.g., 3, 4, 5, or 6) β-strands. In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an ORF1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

An ORF1 molecule may also include a third region comprising the structure or activity of an Anellovirus N22 domain (e.g., as described herein, e.g., an N22 domain from an Anellovirus ORF1 protein as described herein), and/or a fourth region comprising the structure or activity of an Anellovirus C-terminal domain (CTD) (e.g., as described herein, e.g., a CTD from an Anellovirus ORF1 protein as described herein). In some embodiments, the ORF1 molecule comprises, in N-terminal to C-terminal order, the first, second, third, and fourth regions.

The ORF1 molecule may, in some embodiments, further comprise a hypervariable region (HVR), e.g., an HVR from an Anellovirus ORF1 protein, e.g., as described herein. In some embodiments, the HVR is positioned between the second region and the third region. In some embodiments, the HVR comprises comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 arginine-rich region (e.g., an arginine-rich region from an Anellovirus ORF1 protein, e.g., as described herein). In some embodiments, the first region comprises a nuclear localization sigal.

In some embodiments, the second region comprises a jelly-roll domain, e.g., the structure or activity of a viral ORF1 jelly-roll domain (e.g., a jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, one or more of the first region, second region, third region, fourth region, and/or HVR may be replaced by a heterologous amino acid sequence (e.g., the corresponding region from a heterologous ORF1 molecule). In some embodiments, the heterologous amino acid sequence has a desired functionality, e.g., as described herein.

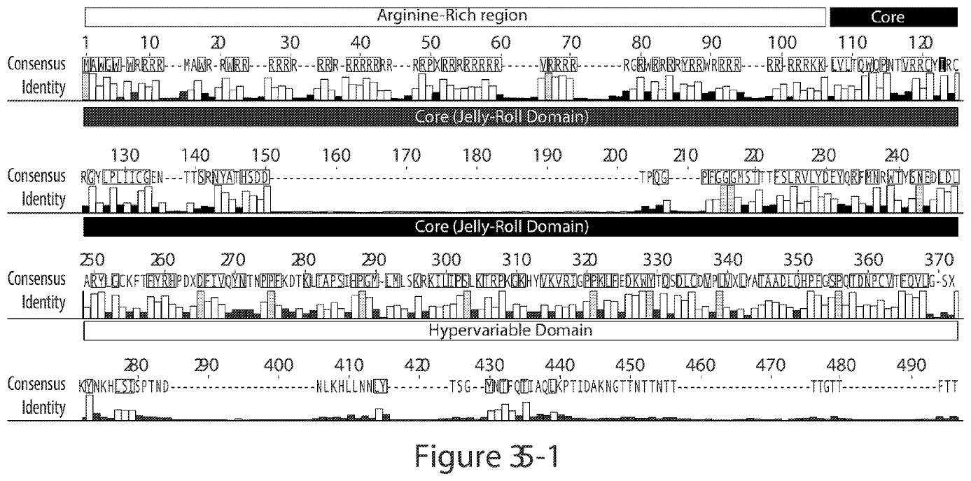

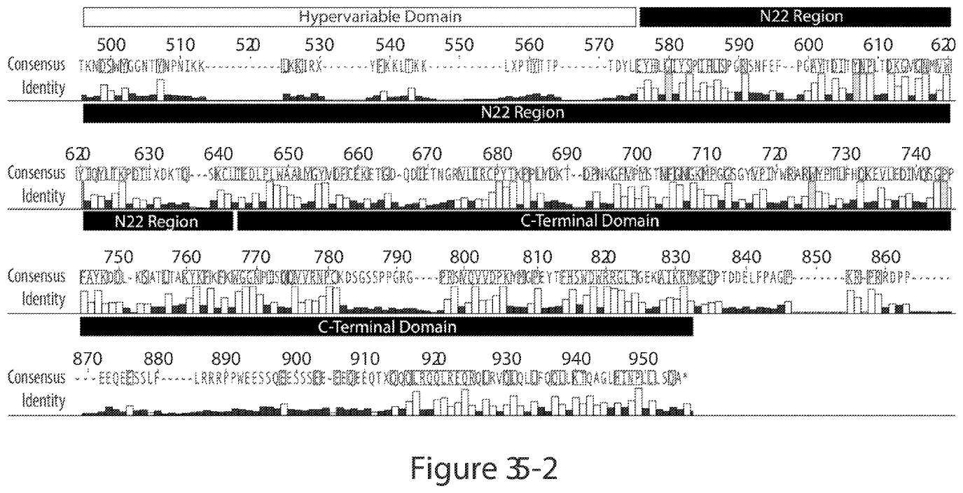

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids) (e.g., as shown in FIG. 34). In some embodiments, the conserved motifs may show 60, 70, 80, 85, 90, 95, or 100% sequence identity to an ORF1 protein of one or more wild-type Anellovirus clades (e.g., Alphatorquevirus, clade 1; Alphatorquevirus, clade 2; Alphatorquevirus, clade 3; Alphatorquevirus, clade 4; Alphatorquevirus, clade 5; Alphatorquevirus, clade 6; Alphatorquevirus, clade 7; Betatorquevirus; and/or Gammatorquevirus). In some embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids. In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade. In some embodiments, the conserved motifs comprise one or more amino acid sequences as listed in Table 19.

In some embodiments, an ORF1 molecule comprises at least one difference (e.g., a mutation, chemical modification, or epigenetic alteration) relative to a wild-type ORF1 protein, e.g., as described herein (e.g., as shown in any of Tables A1-A1417).

### Conserved ORF1 Motif in N22 Domain

In some embodiments, a polypeptide (e.g., an ORF1 molecule) described herein comprises the amino acid sequence YNPX²DXGX²N (SEQ ID NO: 5202), wherein Xⁿ is a contiguous sequence of any n amino acids. For example, X² indicates a contiguous sequence of any two amino acids. In some embodiments, the YNPX²DXGX²N (SEQ ID NO: 5202) is comprised within the N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 5202), wherein Xⁿ is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 5202) motif, e.g., in an N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 5202) motif. In some embodiments, the YNPX²DXGX²N (SEQ ID NO: 5202) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 5202) motif.

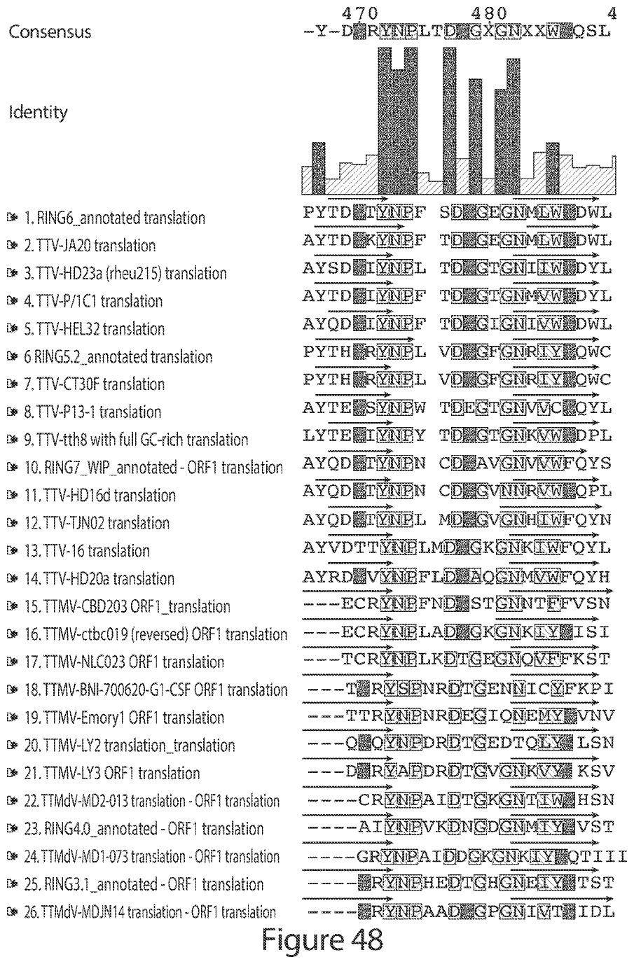

Exemplary YNPX²DXGX²N (SEQ ID NO: 5202) motif-flanking secondary structures are described in Example 47 and FIG. 48. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48, flanking a YNPX²DXGX²N (SEQ ID NO: 5202) motif (e.g., as described herein).

### Conserved Secondary Structural Motif in ORF1 Jelly-Roll Domain

In some embodiments, a polypeptide (e.g., an ORF1 molecule) described herein comprises one or more secondary structural elements comprised by an Anellovirus ORF1 protein (e.g., as described herein). In some embodiments, an ORF1 molecule comprises one or more secondary structural elements comprised by the jelly-roll domain of an Anellovius ORF1 protein (e.g., as described herein). Generally, an ORF1 jelly-roll domain comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and a ninth beta strand. In some embodiments, an ORF1 molecule comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and/or a ninth beta strand.

In some embodiments, a pair of the conserved secondary structural elements (i.e., the beta strands and/or alpha helices) are separated by an interstitial amino acid sequence, e.g., comprising a random coil sequence, a beta strand, or an alpha helix, or a combination thereof. Interstitial amino acid sequences between the conserved secondary structural elements may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, an ORF1 molecule may further comprise one or more additional beta strands and/or alpha helices (e.g., in the jelly-roll domain). In some embodiments, consecutive beta strands or consecutive alpha helices may be combined. In some embodiments, the first beta strand and the second beta strand are comprised in a larger beta strand. In some embodiments, the third beta strand and the fourth beta strand are comprised in a larger beta strand. In some embodiments, the fourth beta strand and the fifth beta strand are comprised in a larger beta strand. In some embodiments, the sixth beta strand and the seventh beta strand are comprised in a larger beta strand. In some embodiments, the seventh beta strand and the eighth beta strand are comprised in a larger beta strand. In some embodiments, the eighth beta strand and the ninth beta strand are comprised in a larger beta strand.

In some embodiments, the first beta strand is about 5-7 (e.g., 3, 4, 5, 6, 7, 8, 9, or 10) amino acids in length. In some embodiments, the second beta strand is about 15-16 (e.g., 13, 14, 15, 16, 17, 18, or 19) amino acids in length. In some embodiments, the first alpha helix is about 15-17 (e.g., 13, 14, 15, 16, 17, 18, 19, or 20) amino acids in length. In some embodiments, the third beta strand is about 3-4 (e.g., 1, 2, 3, 4, 5, or 6) amino acids in length. In some embodiments, the fourth beta strand is about 10-11 (e.g., 8, 9, 10, 11, 12, or 13) amino acids in length. In some embodiments, the fifth beta strand is about 6-7 (e.g., 4, 5, 6, 7, 8, 9, or 10) amino acids in length. In some embodiments, the second alpha helix is about 8-14 (e.g., 5, 6, 7, 8, 9, 10, 11, 12, 13, 14, 15, 16, or 17) amino acids in length. In some embodiments, the second alpha helix may be broken up into two smaller alpha helices (e.g., separated by a random coil sequence). In some embodiments, each of the two smaller alpha helices are about 4-6 (e.g., 2, 3, 4, 5, 6, 7, or 8) amino acids in length. In some embodiments, the sixth beta strand is about 4-5 (e.g., 2, 3, 4, 5, 6, or 7) amino acids in length. In some embodiments, the seventh beta strand is about 5-6 (e.g., 3, 4, 5, 6, 7, 8, or 9) amino acids in length. In some embodiments, the eighth beta strand is about 7-9 (e.g., 5, 6, 7, 8, 9, 10, 11, 12, or 13) amino acids in length. In some embodiments, the ninth beta strand is about 5-7 (e.g., 3, 4, 5, 6, 7, 8, 9, or 10) amino acids in length.

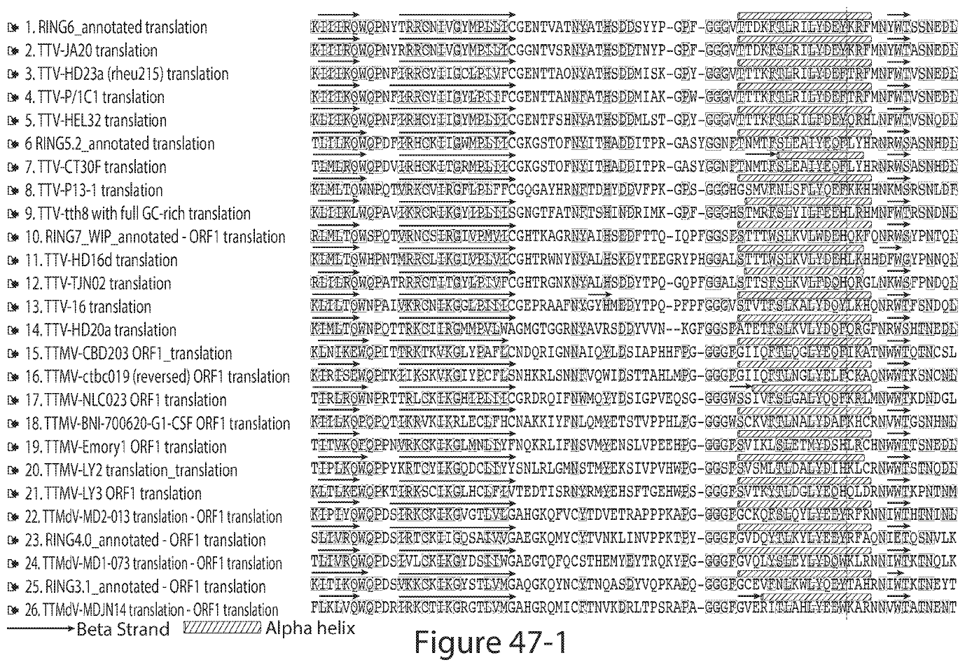

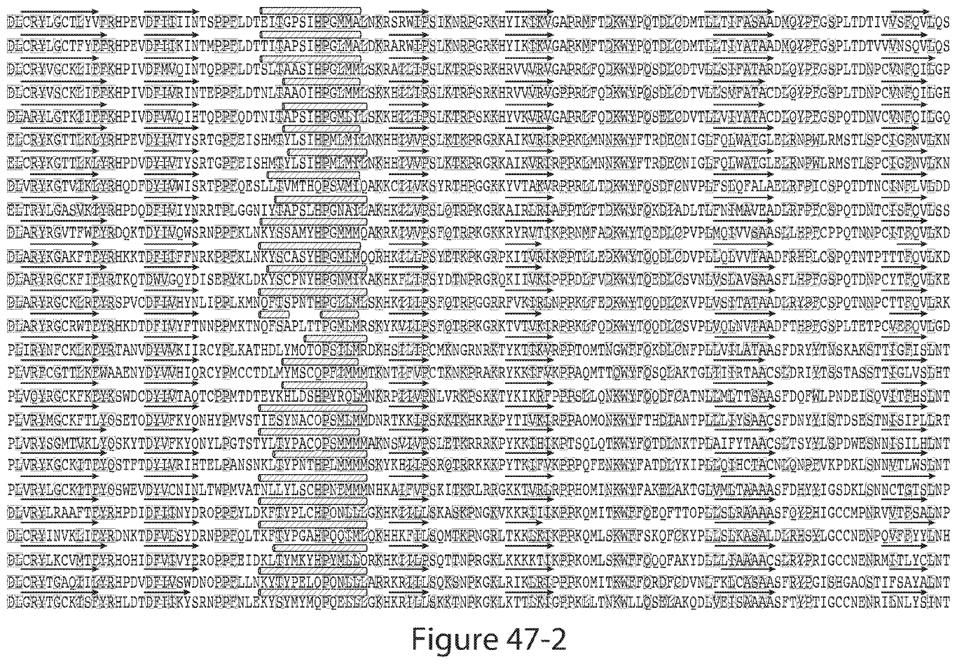

Exemplary jelly-roll domain secondary structures are described in Example 47 and FIG. 47. In some embodiments, an ORF1 molecule comprises a region comprising one or more (e.g., 1, 2, 3, 4, 5, 6, 7, 8, 9, 10, or all) of the secondary structural elements (e.g., beta strands and/or alpha helices) of any of the jelly-roll domain secondary structures shown in FIG. 47.

### Exemplary ORF1 Sequences

In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described herein). In some embodiments, an anellovector described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein. In some embodiments, an anellovector described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed herein), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses. In some embodiments, the ORF1 molecule comprises one or more of an Arg-rich domain, a jelly-roll domain, an N22 domain, and a CTD from one Anellovirus, and an HVR from another. In some embodiments, the ORF1 molecule comprises one or more of a jelly-roll domain, an HVR, an N22 domain, and a CTD from one Anellovirus, and an Arg-rich domain from another. In some embodiments, the ORF1 molecule comprises one or more of an Arg-rich domain, an HVR, an N22 domain, and a CTD from one Anellovirus, and a jelly-roll domain from another. In some embodiments, the ORF1 molecule comprises one or more of an Arg-rich domain, a jelly-roll domain, an HVR, and a CTD from one Anellovirus, and an N22 domain from another. In some embodiments, the ORF1 molecule comprises one or more of an Arg-rich domain, a jelly-roll domain, an HVR, and an N22 domain from one Anellovirus, and a CTD from another.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) as described in PCT Publication No. WO2020/123816 (incorporated herein by reference in entirety).

### Consensus ORF1 Domain Sequences

In some embodiments, an ORF1 molecule, e.g., as described herein, comprises one or more of a jelly-roll domain, N22 domain, and/or C-terminal domain (CTD). In some embodiments, the jelly-roll domain comprises an amino acid sequence having a jelly-roll domain consensus sequence as described herein (e.g., as listed in any of Tables 37A-37C). In some embodiments, the N22 domain comprises an amino acid sequence having a N22 domain consensus sequence as described herein (e.g., as listed in any of Tables 37A-37C). In some embodiments, the CTD domain comprises an amino acid sequence having a CTD domain consensus sequence as described herein (e.g., as listed in any of Tables 37A-37C). In some embodiments, the amino acids listed in any of Tables 37A-37C in the format “(Xa-b)” comprise a contiguous series of amino acids, in which the series comprises at least a, and at most b, amino acids. In certain embodiments, all of the amino acids in the series are identical. In other embodiments, the series comprises at least two (e.g., at least 2, 3, 4, 5, 6, 7, 8, 9, 10, 11, 12, 13, 14, 15, 16, 17, 18, 19, 20, or 21) different amino acids.

TABLE 37A
Alphatorquevius ORF1 domain consensus sequences

| Domain | Sequence | SEQ ID NO: |
|---|---|---|
| Jelly-Roll | LVLTQWQPNTVRRCYIRGYLPLIICGEN(X₀₋₃)TTSRNYA | 5180 |
| | THSDDTIQKGPFGGGMSTTTFSLRVLYDEYQRFMNRW | |
| | TYSNEDLDLARYLGCKFTFYRHPDXDFIVQYNTNPPFK | |
| | DTKLTAPSIHP(X₁₋₅)GMLMLSKRKILIPSLKTRPKGKHY | |
| | VKVRIGPPKLFEDKWYTQSDLCDVPLVXLYATAADLQ | |
| | HPFGSPQTDNPCVTFQVLGSXYNKHLSISP; | |
| | wherein X = any amino acid. | |
| N22 | SNFEFPGAYTDITYNPLTDKGVGNMVWIQYLTKPDTIX | 5181 |
| | DKTQS(X₀₋₃)KCLIEDLPLWAALYGYVDFCEKETGDSAII | |
| | XNXGRVLIRCPYTKPPLYDKT(X₀₋₄)NKGFVPYSTNFGN | |
| | GKMPGGSGYVPIYWRARWYPTLFHQKEVLEDIVQSGP | |
| | FAYKDEKPSTQLVMKYCFNFN; | |
| | wherein X = any amino acid. | |
| CTD | WGGNPISQQVVRNPCKDSG(X₀₋₃)SGXGRQPRSVQVVD | 5182 |
| | PKYMGPEYTFHSWDWRRGLFGEKAIKRMSEQPTDDEI | |
| | FTGGXPKRPRRDPPTXQXPEE(X₁₋₄)QKESSSFR(X₂₋₁₄)PW | |
| | ESSSQEXESESQEEEE(X₀₋₃₀)EQTVQQQLRQQLREQRRL | |
| | RVQLQLLFQQLLKT(X₀₋₄)QAGLHINPLLLSQA(X₀₋₄₀)*; | |
| | wherein X = any amino acid. | |

TABLE 37B
Betatorquevius ORF1 domain consensus sequences

| Domain | Sequence | SEQ ID NO: |
|---|---|---|
| Jelly-Roll | LKQWQPSTIRKCKIKGYLPLFQCGKGRISNNYTQYKESI | 5183 |
| | VPHHEPGGGGWSIQQFTLGALYEEHLKLRNWWTKSN | |
| | DGLPLVRYLGCTIKLYRSEDTDYIVTYQRCYPMTATKL | |
| | TYLSTQPSRMLMNKHKIIVPSKXT(X₁₋₄)NKKKKPYKKIF | |
| | IKPPSQMQNKWYFQQDIANTPLLQLTXTACSLDRMYL | |
| | SSDSISNNITFTSLNTNFFQNPNFQ; | |
| | wherein X = any amino acid. | |
| N22 | (X₄₋₁₀)TPLYFECRYNPFKDKGTGNKVYLVSNN(X₁₋₈)TG | 5184 |
| | WDPPTDPDLIIEGFPLWLLLWGWLDWQKKLGKIQNID | |
| | TDYILVIQSXYYIPP(X₁₋₃)KLPYYVPLDXD(X₀₋₂)FLHGRS | |
| | PY(X₃₋₁₆)PSDKQHWHPKVRFQXETINNIALTGPGTPKLP | |
| | NQKSIQAHMKYKFYFK; | |
| | wherein X = any amino acid. | |
| CTD | WGGCPAPMETITDPCKQPKYPIPNNLLQTTSLQXPTTPI | 5185 |
| | ETYLYKFDERRGLLTKKAAKRIKKDXTTETTLFTDTGX | |
| | XTSTTLPTXXQTETTQEEXTSEEE(X₀₋₅)ETLLQQLQQLR | |
| | RKQKQLRXRILQLLQLLXLL(X₀₋₂₆)*; | |
| | wherein X = any amino acid. | |

TABLE 37C
Gammatorquevius ORF1 domain consensus sequences

| Domain | Sequence | SEQ ID NO: |
|---|---|---|
| Jelly-Roll | TIPLKQWQPESIRKCKIKGYGTLVLGAEGRQFYCYTNE | 5186 |
| | KDEYTPPKAPGGGGFGVELFSLEYLYEQWKARNNIWT | |
| | KSNXYKDLCRYTGCKITFYRHPTTDFIVXYSRQPPFEID | |
| | KXTYMXXHPQXLLLRKHKKIILSKATNPKGKLKKKIKI | |
| | KPPKQMLNKWFFQKQFAXYGLVQLQAAACBLRYPRL | |
| | GCCNENRLITLYYLN; | |
| | wherein X = any amino acid. | |
| N22 | LPIVVARYNPAXDTGKGNKXWLXSTLNGSXWAPPTTD | 5187 |
| | KDLIIEGLPLWLALYGYWSYJKKVKKDKGILQSHMFV | |
| | VKSPAIQPLXTATTQXTFYPXIDNSFIQGKXPYDEPJTX | |
| | NQKKLWYPTLEHQQETINAIVESGPYVPKLDNQKNST | |
| | WELXYXYTFYFK; | |
| | wherein X = any amino acid. | |
| CTD | WGGPQIPDQPVEDPKXQGTYPVPDTXQQTIQIXNPLKQ | 5188 |
| | KPETMFHDWDYRRGIITSTALKRMQENLETDSSFXSDS | |
| | EETP(X₀₋₂)KKKKRLTXELPXPQEETEEIQSCLLSLCEEST | |
| | CQEE(X₁₋₆)ENLQQLIHQQQQQQQQLKHNILKLLSDLKZ | |
| | KQRLLQLQTGILE(X₁₋₁₀)* | |
| | wherein X = any amino acid. | |

In some embodiments, the jelly-roll domain comprises a jelly-roll domain amino acid sequence as listed in any of Tables 37A-37C, or an amino acid sequence having at least 70%, 75%, 80%, 8%, 90%, 95%, 96%, 97%, 98%, 99%, or 100% sequence identity thereto. In some embodiments, the N22 domain comprises a N22 domain amino acid sequence as listed in any of Tables 37A-37C, or an amino acid sequence having at least 70%, 75%, 80%, 8%, 90%, 95%, 96%, 97%, 98%, 99%, or 100% sequence identity thereto. In some embodiments, the CTD domain comprises a CTD domain amino acid sequence as listed in any of Tables 37A-37C, or an amino acid sequence having at least 70%, 75%, 80%, 8%, 90%, 95%, 96%, 97%, 98%, 99%, or 100% sequence identity thereto.

### Identification of ORF1 Protein Sequences

In some embodiments, an Anellovirus ORF1 protein sequence, or a nucleic acid sequence encoding an ORF1 protein, can be identified from the genome of an Anellovirus (e.g., a putative Anellovirus genome identified, for example, by nucleic acid sequencing techniques, e.g., deep sequencing techniques). In some embodiments, an ORF1 protein sequence is identified by one or more (e.g., 1, 2, or all 3) of the following selection criteria:

(i) Length Selection: Protein sequences (e.g., putative Anellovirus ORF1 sequences passing the criteria described in (ii) or (iii) below) may be size-selected for those greater than about 600 amino acid residues to identify putative Anellovirus ORF1 proteins. In some embodiments, an Anellovirus ORF1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an Anellovirus ORF1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.

(ii) Presence of ORF1 motif: Protein sequences (e.g., putative Anellovirus ORF1 sequences passing the criteria described in (i) above or (iii) below) may be filtered to identify those that contain the conserved ORF1 motif in the N22 domain described above. In some embodiments, a putative Anellovirus ORF1 sequence comprises the sequence YNPXXDXGXXN (SEQ ID NO: 5202). In some embodiments, a putative Anellovirus ORF1 sequence comprises the sequence Y[NCS]PXXDX[GASKR|XX[NTSVAK] (SEQ ID NO: 5204).

(iii) Presence of arginine-rich region: Protein sequences (e.g., putative Anellovirus ORF1 sequences passing the criteria described in (i) and/or (ii) above) may be filtered for those that include an arginine-rich region (e.g., as described herein). In some embodiments, a putative Anellovirus ORF1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues. In some embodiments, a putative Anellovirus ORF1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the arginine-rich region is positioned at least about 30, 40, 50, 60, 70, or 80 amino acids downstream of the start codon of the putative Anellovirus ORF1 protein. In some embodiments, the arginine-rich region is positioned at least about 50 amino acids downstream of the start codon of the putative Anellovirus ORF1 protein.

In some embodiments, an ORF1 protein is identified in an Anellovirus genome sequence as described in Example 36 PCT Publication No. WO2020/123816 (incorporated herein by reference in its entirety).

### ORF2 Molecules

In some embodiments, the anellovector comprises an ORF2 molecule and/or a nucleic acid encoding an ORF2 molecule. Generally, an ORF2 molecule comprises a polypeptide having the structural features and/or activity of an Anellovirus ORF2 protein (e.g., an Anellovirus ORF2 protein as described herein, e.g., as listed in any of Tables A1-A1417), or a functional fragment thereof. In some embodiments, an ORF2 molecule comprises an amino acid sequence having at least 70%, 75%, 80%, 85%, 90%, 95%, 96%, 97%, 98%, 99%, or 100% sequence identity to an Anellovirus ORF2 protein sequence as shown in any of Tables A1-A1417.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 molecule has protein phosphatase activity. In some embodiments, an ORF2 molecule comprises at least one difference (e.g., a mutation, chemical modification, or epigenetic alteration) relative to a wild-type ORF2 protein, e.g., as described herein (e.g., as shown in any of Tables A1-A1417).

### Conserved ORF2 Motif

In some embodiments, a polypeptide (e.g., an ORF2 molecule) described herein comprises the amino acid sequence [W/F]X⁷HX³CX¹CX⁵H (SEQ ID NO: 5203), wherein Xⁿ is a contiguous sequence of any n amino acids. In embodiments, X⁷ indicates a contiguous sequence of any seven amino acids. In some embodiments, X³ indicates a contiguous sequence of any three amino acids. In some embodiments, X¹ indicates any single amino acid. In some embodiments, X⁵ indicates a contiguous sequence of any five amino acids. In some embodiments, the [W/F] can be either tryptophan or phenylalanine. In some embodiments, the [W/F]X⁷HX³CX¹CXSH (SEQ ID NO: 5203) is comprised within the N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 5203), wherein Xⁿ is a contiguous sequence of any n amino acids.

### Genetic Elements

In some embodiments, the anellovector comprises a genetic element. In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus nucleic acid sequence, e.g., has less than 70%, 60%, 50%, 40%, 30%, 20%, 10%, 5% sequence identity to an Anellovirus nucleic acid sequence, e.g., as described herein. In some embodiments, outside of the packaging or capsid-binding sequence, the genetic element has less than 500, 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus nucleic acid sequence. In certain embodiments, the genetic element is a circular, single stranded DNA that comprises a promoter sequence, a sequence encoding a therapeutic effector, and a capsid binding protein.

In some embodiments, the genetic element has at least about 70%, 75%, 80%, 8%, 90%, 95%, 96%, 97%, 98%, 99%, or 100% sequence identity to an Anellovirus nucleic acid sequence, e.g., as described herein (e.g., as described in any of Tables N1-N1417), or a fragment thereof, or encodes an amino acid sequence having at least about 70%, 75%, 80%, 8%, 90%, 95%, 96%, 97%, 98%, 99%, or 100% sequence identity to an Anellovirus amino acid sequence (e.g., as described in any of Tables A1-A1417), or a fragment thereof. In some embodiments, the genetic element comprises a sequence encoding an effector (e.g., an endogenous effector or an exogenous effector, e.g., a payload), e.g., a polypeptide effector (e.g., a protein) or nucleic acid effector (e.g., a non-coding RNA, e.g., a miRNA, siRNA, mRNA, lncRNA, RNA, DNA, an antisense RNA, gRNA).

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences. In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences described herein, an Anellovirus amino acid sequence, e.g., as listed in any of Tables A1-A1417.

In some embodiments, the genetic element was produced from a double-stranded circular DNA (e.g., produced by in vitro circularization). In some embodiments, the genetic element was produced by rolling circle replication from the double-stranded circular DNA. In some embodiments, the rolling circle replication occurs in a cell (e.g., a host cell, e.g., a mammalian cell, e.g., a human cell, e.g., a HEK293T cell, an A549 cell, or a Jurkat cell). In some embodiments, the genetic element can be amplified exponentially by rolling circle replication in the cell. In some embodiments, the genetic element can be amplified linearly by rolling circle replication in the cell. In some embodiments, the double-stranded circular DNA or genetic element is capable of yielding at least 2, 4, 8, 16, 32, 64, 128, 256, 518, 1024 or more times the original quantity by rolling circle replication in the cell. In some embodiments, the double-stranded circular DNA was introduced into the cell, e.g., as described herein.

In some embodiments, the double-stranded circular DNA and/or the genetic element does not comprise one or more bacterial plasmid elements (e.g., a bacterial origin of replication or a selectable marker, e.g., a bacterial resistance gene). In some embodiments, the double-stranded circular DNA and/or the genetic element does not comprise a bacterial plasmid backbone.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native Anellovirus sequence, e.g., as described herein).

In some embodiments, a genetic element as described herein comprises a sequence (e.g., a TATA box, cap site, transcriptional start site, 5′ UTR, open reading frame (ORF), poly(A) signal, or GC-rich region sequence) as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, a genetic element comprises a sequence encoding an effector (e.g., an exogenous effector). In some embodiments, the effector-encoding sequence is inserted into an Anellovirus genome sequence (e.g., as described herein). In some embodiments, the effector-encoding sequence replaces a contiguous sequence (e.g., of at least 5, 10, 15, 20, 25, 30, 40, 50, 60, 70, 80, 90, 100, 200, 300, 400, 500, 600, 700, 800, 900, 1000, or more nucleotides) from the Anellovirus genome sequence. In some embodiments, the effector-encoding sequence replaces a TATA box, cap site, transcriptional start site, 5′ UTR, open reading frame (ORF), poly(A) signal, or GC-rich region sequence, or a portion thereof (e.g., a portion consisting of at least 5, 10, 15, 20, 25, 30, 40, 50, 60, 70, 80, 90, 100, 200, 300, 400, 500, 600, 700, 800, 900, 1000, or more nucleotides) e.g., as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, the sequence of a first nucleic acid element comprised in a genetic element (e.g., a TATA box, cap site, transcriptional start site, 5′ UTR, open reading frame (ORF), poly(A) signal, or GC-rich region) overlaps with the sequence of a second nucleic acid element (e.g., a TATA box, cap site, transcriptional start site, 5′ UTR, open reading frame (ORF), poly(A) signal, or GC-rich region), e.g., by at least 5, 10, 15, 20, 25, 30, 40, 50, 60, 70, 80, 90, 100, 150, 200, 250, 300, 400, or 500 nucleotides. In some embodiments, the sequence of a first nucleic acid element comprised in a genetic element (e.g., a TATA box, cap site, transcriptional start site, 5′ UTR, open reading frame (ORF), poly(A) signal, or GC-rich region) does not overlap with the sequence of a second nucleic acid element (e.g., a TATA box, cap site, transcriptional start site, 5′ UTR, open reading frame (ORF), poly(A) signal, or GC-rich region).

### Protein Binding Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77(24), 13036-13041.

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence as described in Example 8. In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence (e.g., as shown in any of Tables N1-N1417).

In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of any of Tables N1-N1417.

### 5′ UTR Regions

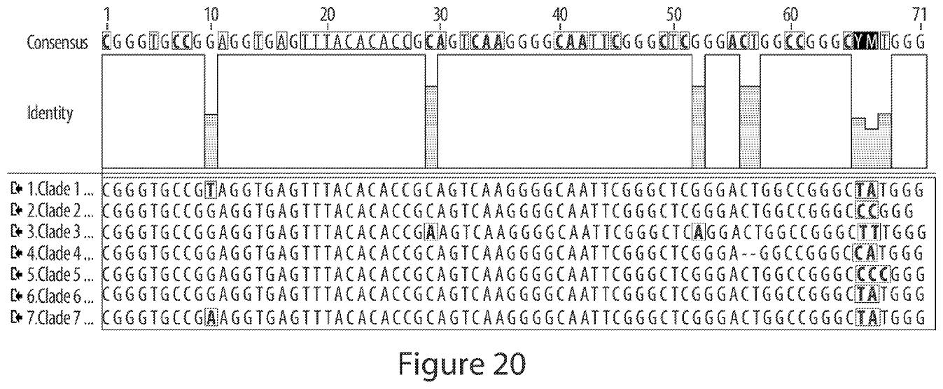

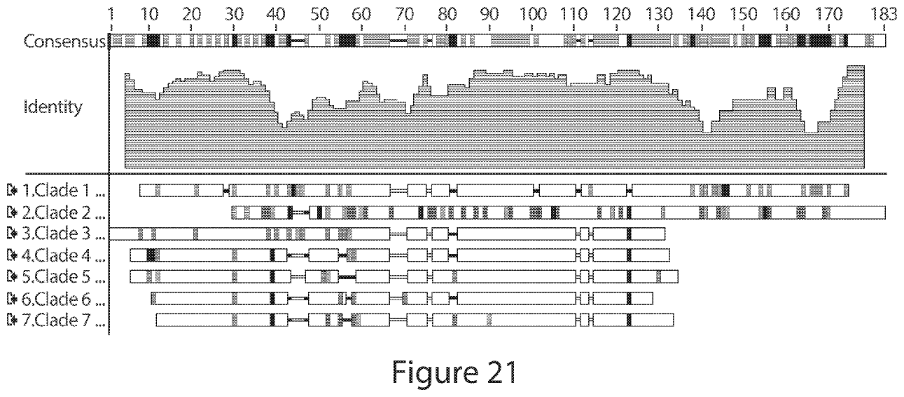

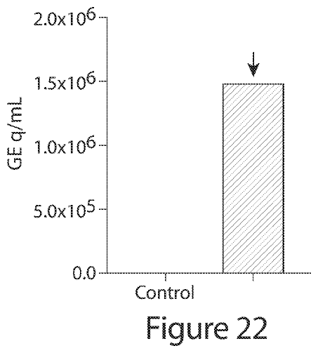

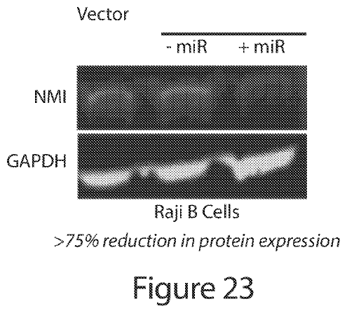

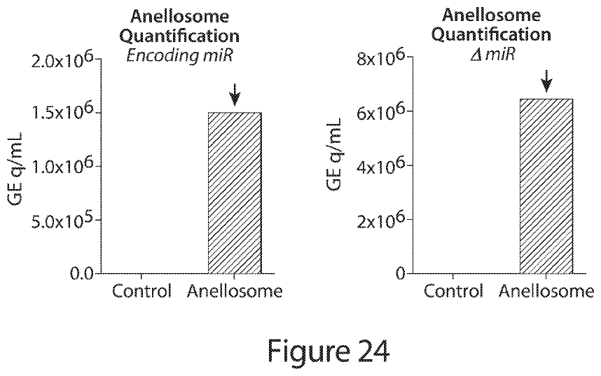

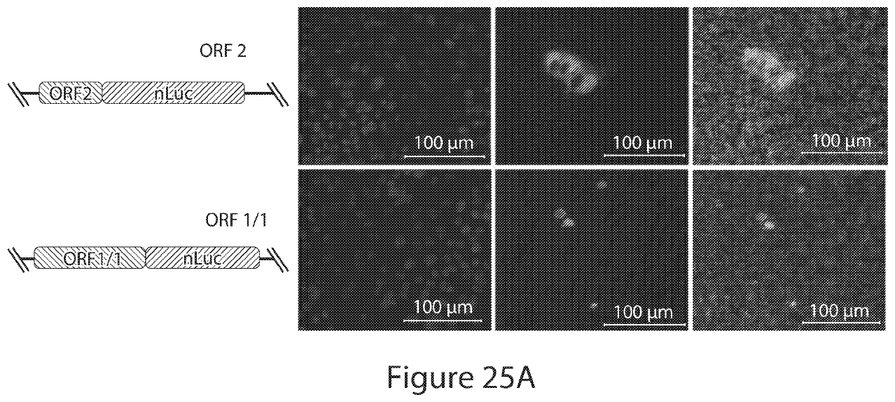

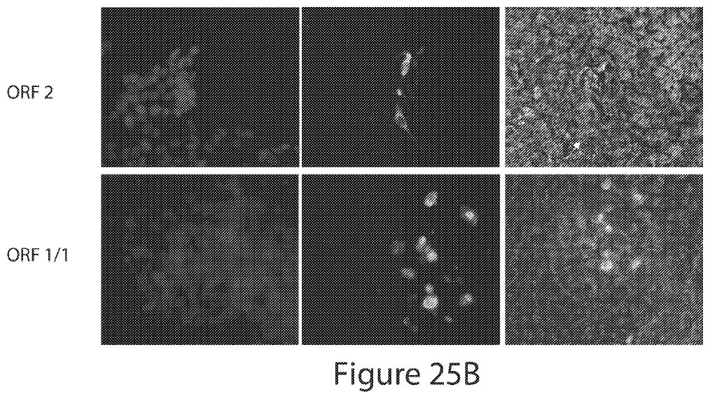

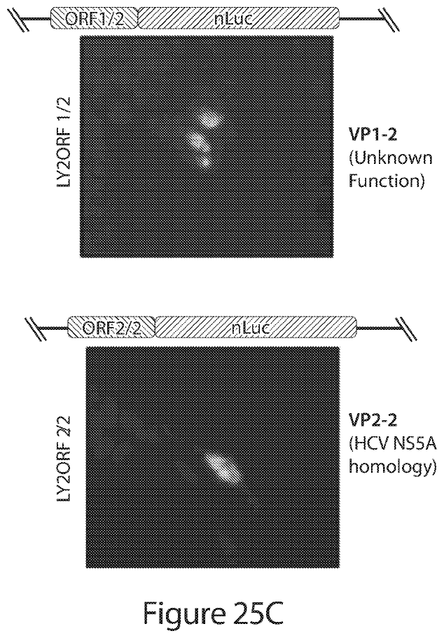

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 38 and/or FIG. 20. In some embodiments, the genetic element (e.g., protein-binding sequence of the genetic element) comprises a nucleic acid sequence of the Consensus 5′ UTR sequence shown in Table 38, wherein X₁, X₂, X₃, X₄, and X₅ are each independently any nucleotide, e.g., wherein X₁=G or T, X₂=C or A, X₃=G or A, X₄=T or C, and X₅=A, C, or T). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJN02 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 38.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 1 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 2 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 3 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 4 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 5 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 6 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 7 5′ UTR sequence shown in Table 38.

In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A1 (e.g., nucleotides 165-235 of the nucleic acid sequence of Table A1). In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A3 (e.g., nucleotides 175-245 of the nucleic acid sequence of Table A3). In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A5 (e.g., nucleotides 138-208 of the nucleic acid sequence of Table A5). In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A7 (e.g., nucleotides 174-244 of the nucleic acid sequence of Table A7). In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A9 (e.g., nucleotides 100-171 of the nucleic acid sequence of Table A9). In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A11 (e.g., nucleotides 294-364 of the nucleic acid sequence of Table A11).

In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of any of Tables N1-N1417.

TABLE 38
Exemplary 5′ UTR sequences from Anelloviruses

| Source | Sequence | SEQ ID NO: |
|---|---|---|
| Consensus | CGGGTGCCGX₁AGGTGAGTTTACACACCGX₂AGT | 5112 |
| | CAAGGGGCAATTCGGGCTCX₃GGACTGGCCGGG | |
| | CX₄X₅TGGG | |
| | X₁ = G or T | |
| | X₂ = C or A | |
| | X₃ = Gor A | |
| | X₄ = T or C | |
| | X₅ = A, C, or T | |
| Exemplary TTV Sequence | CGGGTGCCGGAGGTGAGTTTACACACCGCAGTC | 5113 |
| | AAGGGGCAATTCGGGCTCGGGACTGGCCGGGCT | |
| | WTGGG | |
| TTV-CT30F | CGGGTGCCGTAGGTGAGTTTACACACCGCAGTC | 5114 |
| | AAGGGGCAATTCGGGCTCGGGACTGGCCGGGCT | |
| | ATGGG | |
| TTV-HD23a | CGGGTGCCGGAGGTGAGTTTACACACCGCAGTC | 5115 |
| | AAGGGGCAATTCGGGCTCGGGACTGGCCGGGCC | |
| | CTGGG | |
| TTV-JA20 | CGGGTGCCGGAGGTGAGTTTACACACCGCAGTC | 5116 |
| | AAGGGGCAATTCGGGCTCGGGACTGGCCGGGCT | |
| | TTGGG | |
| TTV-TJN02 | CGGGTGCCGGAGGTGAGTTTACACACCGCAGTC | 5117 |
| | AAGGGGCAATTCGGGCTCGGGACTGGCCGGGCT | |
| | ATGGG | |
| TTV-tth8 | CGGGTGCCGGAGGTGAGTTTACACACCGAAGTC | 5118 |
| | AAGGGGCAATTCGGGCTCAGGACTGGCCGGGCT | |
| | TTGGG | |
| Alphatorquevirus | CGGGTGCCGGAGGTGAGTTTACACACCGCAGTC | 5119 |
| Consensus 5′ UTR | AAGGGGCAATTCGGGCTCGGGACTGGCCGGGC | |
| | X₁X₂TGGG; wherein X₁ comprises T or C, and wherein | |
| | X₂ comprises A, C, or T. | |
| Alphatorquevirus | CGGGTGCCGTAGGTGAGTTTACACACCGCAGTC | 5120 |
| Clade 1 5′ UTR (e.g., | AAGGGGCAATTCGGGCTCGGGACTGGCCGGGCT | |
| TTV-CT30F) | ATGGG | |
| Alphatorquevirus | CGGGTGCCGGAGGTGAGTTTACACACCGCAGTC | 5121 |
| Clade 2 5′ UTR (e.g., | AAGGGGCAATTCGGGCTCGGGACTGGCCGGGCC | |
| TTV-P13-1) | CGGG | |
| Alphatorquevirus | CGGGTGCCGGAGGTGAGTTTACACACCGAAGTC | 5122 |
| Clade 3 5′ UTR (e.g., | AAGGGGCAATTCGGGCTCAGGACTGGCCGGGCT | |
| TTV-tth8) | TTGGG | |
| Alphatorquevirus | CGGGTGCCGGAGGTGAGTTTACACACCGCAGTC | 5123 |
| Clade 4 5′ UTR (e.g., | AAGGGGCAATTCGGGCTCGGGAGGCCGGGCCAT | |
| TTV-HD20a) | GGG | |
| Alphatorquevirus | CGGGTGCCGGAGGTGAGTTTACACACCGCAGTC | 5124 |
| Clade 5 5′ UTR (e.g., | AAGGGGCAATTCGGGCTCGGGACTGGCCGGGCC | |
| TTV-16) | CCGGG | |
| Alphatorquevirus | CGGGTGCCGGAGGTGAGTTTACACACCGCAGTC | 5125 |
| Clade 6 5′ UTR (e.g., | AAGGGGCAATTCGGGCTCGGGACTGGCCGGGCT | |
| TTV-TJN02) | ATGGG | |
| Alphatorquevirus | CGGGTGCCGAAGGTGAGTTTACACACCGCAGTC | 5126 |
| Clade 7 5′ UTR (e.g., | AAGGGGCAATTCGGGCTCGGGACTGGCCGGGCT | |
| TTV-HD16d) | ATGGG | |

### Identification of 5′ UTR Sequences

In some embodiments, an Anellovirus 5′ UTR sequence can be identified within the genome of an Anellovirus (e.g., a putative Anellovirus genome identified, for example, by nucleic acid sequencing techniques, e.g., deep sequencing techniques). In some embodiments, an Anellovirus 5′ UTR sequence is identified by one or both of the following steps:

(i) Identification of circularization junction point: In some embodiments, a 5′ UTR will be positioned near a circularization junction point of a full-length, circularized Anellovirus genome. A circularization junction point can be identified, for example, by identifying overlapping regions of the sequence. In some embodiments, an overlapping region of the sequence can be trimmed from the sequence to produce a full-length Anellovirus genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anellovirus genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anellovirus, e.g., as described herein).

(ii) Identification of 5′ UTR sequence: Once a putative Anellovirus genome sequence has been obtained, the sequence (or portions thereof, e.g., having a length between about 40-50, 50-60, 60-70, 70-80, 80-90, or 90-100 nucleotides) can be compared to one or more Anellovirus 5′ UTR sequences (e.g., as described herein) to identify sequences having substantial homology thereto. In some embodiments, a putative Anellovirus 5′ UTR region has at least 50%, 60%, 70%, 75%, 80%, 85%, 90%, 95%, 96%, 97%, 98%, 99%, or 100% sequence identity to an Anellovirus 5′ UTR sequence as described herein.

### GC-Rich Regions

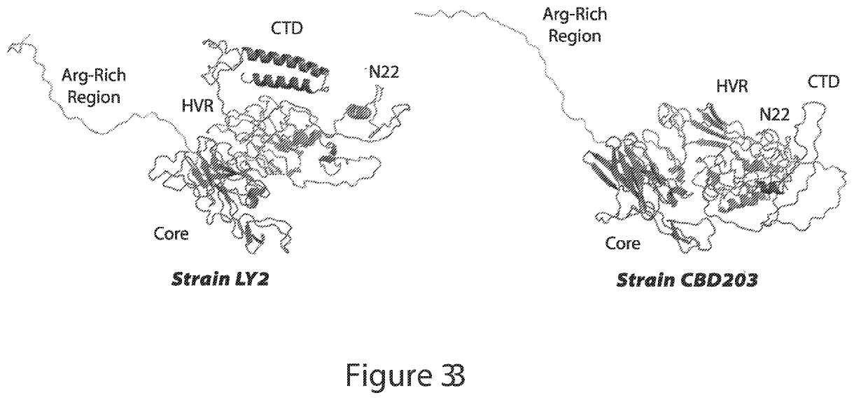

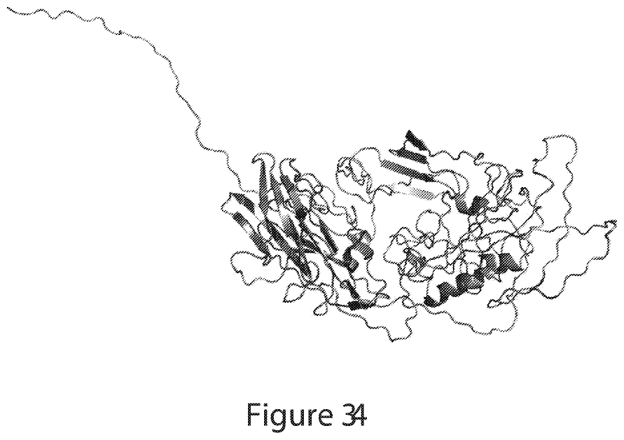

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in any of Table 39 and/or FIGS. 20 and 32. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a GC-rich sequence shown in Table 39.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GHI 36-nucleotide region, TTV Clade 3 sle 1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region). In some embodiments, the genetic element (e.g., protein-binding sequence of the genetic element) comprises a nucleic acid sequence comprising at least 10, 15, 20, 25, 30, 31, 32, 33, 34, 35, or 36 consecutive nucleotides of a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GHI 36-nucleotide region, TTV Clade 3 sle 1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region).

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39. In some embodiments, the genetic element (e.g., protein-binding sequence of the genetic element) comprises a nucleic acid sequence comprising at least 10, 15, 20, 25, 30, 35, 40, 45, 50, 60, 70, 80, 90, 100, 104, 105, 108, 110, 111, 115, 120, 122, 130, 140, 145, 150, 155, or 156 consecutive nucleotides of an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39.

In some embodiments, the 36-nucleotide GC-rich sequence is selected from:

(i)
(SEQ ID NO: 5167)
CGCGCTGCGCGCGCCGCCCAGTAGGGGGAGCCATGC,
(ii)
(SEQ ID NO: 5171)
GCGCTX₁CGCGCGCGCGCCGGGGGGCTGCGCCCCCCC,

wherein X₁ is selected from T, G, or A:

(iii)
(SEQ ID NO: 5172)
GCGCTTCGCGCGCCGCCCACTAGGGGGCGTTGCGCG;
(iv)
(SEQ ID NO: 5173)
GCGCTGCGCGCGCCGCCCAGTAGGGGGCGCAATGCG;
(v)
(SEQ ID NO: 5174)
GCGCTGCGCGCGCGGCCCCCGGGGGAGGCATTGCCT;
(vi)
(SEQ ID NO: 5175)
GCGCTGCGCGCGCGCGCCGGGGGGGCGCCAGCGCCC;
(vii)
(SEQ ID NO: 5176)
GCGCTTCGCGCGCGCGCCGGGGGGCTCCGCCCCCCC;
(viii)
(SEQ ID NO: 5177)
GCGCTTCGCGCGCGCGCCGGGGGGCTGCGCCCCCCC;
(ix)
(SEQ ID NO: 5178)
GCGCTACGCGCGCGCGCCGGGGGGCTGCGCCCCCCC;
or
(x)
(SEQ ID NO: 5179)
GCGCTACGCGCGCGCGCCGGGGGGCTCTGCCCCCCC.

In some embodiments, the genetic element (e.g., protein-binding sequence of the genetic element) comprises the nucleic acid sequence CGCGCTGCGCGCGCCGCCCAGTAGGGGGAGCCATGC (SEQ ID NO: 5167).

In some embodiments, the genetic element (e.g., protein-binding sequence of the genetic element) comprises a nucleic acid sequence of the Consensus GC-rich sequence shown in Table 39, wherein X₁, X₄, X₅, X₆, X₇, X₁₂, X₁₃, X₁₄, X₁₅, X₂₀, X₂₁, X₂₂, X₂₆, X₂₉, X₃₀, and X₃₃ are each independently any nucleotide and wherein X₂, X₃, X₈, X₉, X₁₀, X₁₁, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 39 (e.g., the full sequence, Fragment 1, Fragment 2, Fragment 3, or any combination thereof, e.g., Fragments 1-3 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT3OF GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 39 (e.g., the full sequence, Fragment 1, Fragment 2, Fragment 3, Fragment 4, Fragment 5, Fragment 6, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 39 (e.g., the full sequence, Fragment 1, Fragment 2, or any combination thereof, e.g., Fragments 1 and 2 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJN02 GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 39 (e.g., the full sequence, Fragment 1, Fragment 2, Fragment 3, Fragment 4, Fragment 5, Fragment 6, Fragment 7, Fragment 8, Fragment 9, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 7 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 8 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 9 shown in Table 39.

TABLE 39
Exemplary GC-rich sequences from Anelloviruses

| Source | | Sequence | SEQ ID NO: |
|---|---|---|---|
| Consensus | | CGGCGGX₁GGX₂GX₃X₄X₅CGCGCTX₆CGCGC | 5127 |
| | | GCX₇X₈X₉X₁₀CX₁₁X₁₂X₁₃X₁₄GGGGX₁₅X₁₆X₁₇X₁₈ | |
| | | X₁₉X₂₀X₂₁GCX₂₂X₂₃X₂₄X₂₅CCCCCCCX₂₆CGCGC | |
| | | ATX₂₇X₂₈GCX₂₉CGGGX₃₀CCCCCCCCCX₃₁X₃₂X₃₃ | |
| | | GGGGGGCTCCGX₃₄CCCCCCGGCCCCCC | |
| | | X₁ = G or C | |
| | | X₂ = G, C, or absent | |
| | | X₃ = C or absent | |
| | | X₄ = G or C | |
| | | X₅ = G or C | |
| | | X₆ = T, G, or A | |
| | | X₇ = G or C | |
| | | X₈ = G or absent | |
| | | X₉ = C or absent | |
| | | X₁₀ = C or absent | |
| | | X₁₁ = G, A, or absent | |
| | | X₁₂ = G or C | |
| | | X₁₃ = C or T | |
| | | X₁₄ = G or A | |
| | | X₁₅ = G or A | |
| | | X₁₆ = A, G, T, or absent | |
| | | X₁₇ = G, C, or absent | |
| | | X₁₈ = G, C, or absent | |
| | | X₁₉ = C, A, or absent | |
| | | X₂₀ = C or A | |
| | | X₂₁ = T or A | |
| | | X₂₂ = G or C | |
| | | X₂₃ = G, T, or absent | |
| | | X₂₄ = C or absent | |
| | | X₂₅ = G, C, or absent | |
| | | X₂₆ = G or C | |
| | | X₂₇ = G or absent | |
| | | X₂₈ = C or absent | |
| | | X₂₉ = G or A | |
| | | X₃₀ = G or T | |
| | | X₃₁ = C, T, or absent | |
| | | X₃₂ = G, C, A, or absent | |
| | | X₃₃ = G or C | |
| | | X₃₄ = C or absent | |
| Exemplary TTV | Full sequence | GCCGCCGCGGCGGCGGSGGNGNSGCGCGCT | 5128 |
| | | DCGCGCGCSNNNCRCCRGGGGGNNNNCWG | |
| | | CSNCNCCCCCCCCCGCGCATGCGCGGGKCC | |
| Sequence | | CCCCCCCNNCGGGGGGCTCCGCCCCCCGGC | |
| | | CCCCCCCCGTGCTAAACCCACCGCGCATGC | |
| | | GCGACCACGCCCCCGCCGCC | |
| | Fragment 1 | GCCGCCGCGGCGGCGGSGGNGNSGCGCGCT | 5129 |
| | | DCGCGCGCSNNNCRCCRGGGGGNNNNCWG | |
| | | CSNCNCCCCCCCCCGCGCAT | |
| | Fragment 2 | GCGCGGGKCCCCCCCCCNNCGGGGGGCTC | 5130 |
| | | CG | |
| | Fragment 3 | CCCCCCGGCCCCCCCCCGTGCTAAACCCAC | 5131 |
| | | CGCGCATGCGCGACCACGCCCCCGCCGCC | |
| TTV-CT30F | Full sequence | GCGGCGG-GGGGGCG-GCCGCG- | 5132 |
| | | TTCGCGCGCCGCCCACCAGGGGGTG-- | |
| | | CTGCG-CGCCCCCCCCCGCGCAT | |
| | | GCGCGGGGCCCCCCCCC-- | |
| | | GGGGGGGCTCCGCCCCCCCGGCCCCCCCCC | |
| | | GTGCTAAACCCACCGCGCATGCGCGACCAC | |
| | | GCCCCCGCCGCC | |
| | Fragment 1 | GCGGCGG | 5133 |
| | Fragment 2 | GGGGGCG | 5134 |
| | Fragment 3 | GCCGCG | 5135 |
| | Fragment 4 | TTCGCGCGCCGCCCACCAGGGGGTG | 5136 |
| | Fragment 5 | CTGCG | 5137 |
| | Fragment 6 | CGCCCCCCCCCGCGCAT | 5138 |
| | Fragment 7 | GCGCGGGGCCCCCCCCC | 5139 |
| | Fragment 8 | GGGGGGGCTCCGCCCCCCCGGCCCCCCCCC | 5140 |
| | | GTGCTAAACCCACCGCGCATGCGCGACCAC | |
| | | GCCCCCGCCGCC | |
| TTV-HD23a | Full sequence | CGGCGGCGGCGGCG- | 5141 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGCTCC | |
| | | G CCCCCCGGCCCCCCCCC | |
| | Fragment 1 | CGGCGGCGGCGGCG | 5142 |
| | Fragment 2 | CGCGCGCTGCGCGCGCG | 5143 |
| | Fragment 3 | CGCCGGGGGGGCGCCAGCG | 5144 |
| | Fragment 4 | CCCCCCCCCCCGCGCAT | 5145 |
| | Fragment 5 | GCACGGGTCCCCCCCCCCACGGGGGGCTCC | 5146 |
| | | G | |
| | Fragment 6 | CCCCCCGGCCCCCCCCC | 5147 |
| TTV-JA20 | Full sequence | CCGTCGGCGGGGGGGCCGCGCGCTGCGCG | 5148 |
| | | CGCGGCCC- | |
| | | CCGGGGGAGGCACAGCCTCCCCCCCCCGCG | |
| | | CGCATGCGCGCGGGTCCCCCCCCCTCCGGG | |
| | | GGGCTCCGCCCCCCGGCCCCCCCC | |
| | Fragment 1 | CCGTCGGCGGGGGGGCCGCGCGCTGCGCG | 5149 |
| | | CGCGGCCC | |
| | Fragment 2 | CCGGGGGAGGCACAGCCTCCCCCCCCCGCG | 5150 |
| | | CGCATGCGCGCGGGTCCCCCCCCCTCCGGG | |
| | | GGGCTCCGCCCCCCGGCCCCCCCC | |
| TTV-TJN02 | Full sequence | CGGCGGCGGCG-CGCGCGCTACGCGCGCG-- | 5151 |
| | | -CGCCGGGGGG----CTGCCGC- | |
| | | CCCCCCCCCGCGCAT | |
| | | GCGCGGGGCCCCCCCCC- | |
| | | GCGGGGGGCTCCG CCCCCCGGCCCCCC | |
| | Fragment 1 | CGGCGGCGGCG | 5152 |
| | Fragment 2 | CGCGCGCTACGCGCGCG | 5153 |
| | Fragment 3 | CGCCGGGGGG | 5154 |
| | Fragment 4 | CTGCCGC | 5155 |
| | Fragment 5 | CCCCCCCCCGCGCAT | 5156 |
| | Fragment 6 | GCGCGGGGCCCCCCCCC | 5157 |
| | Fragment 7 | GCGGGGGGCTCCG | 5158 |
| | Fragment 8 | CCCCCCGGCCCCCC | 5159 |
| TTV-tth8 | Full sequence | GCCGCCGCGGCGGCGGGGG- | 5160 |
| | | GCGGCGCGCTGCGCGCGCCGCCCAGTAGG | |
| | | GGGAGCCATGCG---CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 5161 |
| | Fragment 2 | GCGGCGCGCTGCGCGCGCCGCCCAGTAGG | 5162 |
| | | GGGAGCCATGCG | |
| | Fragment 3 | CCCCCCCCCGCGCAT | 5163 |
| | Fragment 4 | GCGCGGGGCCCCCCCCC | 5164 |
| | Fragment 5 | GCGGGGGGCTCCG | 5165 |
| | Fragment 6 | CCCCCCGGCCCCCCCCG | 5166 |
| | Fragment 7 | CGCGCTGCGCGCGCCGCCCAGTAGGGGGA | 5167 |
| | | GCCATGC | |
| | Fragment 8 | CCGCCATCTTAAGTAGTTGAGGCGGACGGT | 5168 |
| | | GGCGTGAGTTCAAAGGTCACCATCAGCCAC | |
| | | ACCTACTCAAAATGGTGG | |
| | Fragment 9 | CTTAAGTAGTTGAGGCGGACGGTGGCGTGA | 5169 |
| | | GTTCAAAGGTCACCATCAGCCACACCTACT | |
| | | CAAAATGGTGGACAATTTCTTCCGGGTCAA | |
| | | AGGTTACAGCCGCCATGTTAAAACACGTGA | |
| | | CGTATGACGTCACGGCCGCCATTTTGTGAC | |
| | | ACAAGATGGCCGACTTCCTTCC | |
| Additional GC-rich | 36-nucleotide | CGCGCTGCGCGCGCCGCCCAGTAGGGGGA | 5170 |
| Sequences (as shown | consensus GC- | GCCATGC | |
| in FIG. 32) | rich region | | |
| | sequence 1 | | |
| | 36-nucleotide | GCGCTX₁CGCGCGCGCGCCGGGGGGCTGCG | 5171 |
| | region | CCCCCCC, wherein X₁ is selected from | |
| | consensus | T, G, or A | |
| | sequence 2 | | |
| | TTV Clade 1 | GCGCTTCGCGCGCCGCCCACTAGGGGGCGT | 5172 |
| | 36-nucleotide | TGCGCG | |
| | region | | |
| | TTV Clade 3 | GCGCTGCGCGCGCCGCCCAGTAGGGGGCG | 5173 |
| | 36-nucleotide | CAATGCG | |
| | region | | |
| | TTV Clade 3 | GCGCTGCGCGCGCGGCCCCCGGGGGAGGC | 5174 |
| | isolate GH1 36- | ATTGCCT | |
| | nucleotide | | |
| | region | | |
| | TTV Clade 3 | GCGCTGCGCGCGCGCGCCGGGGGGGCGCC | 5175 |
| | sle 1932 36- | AGCGCCC | |
| | nucleotide | | |
| | region | | |
| | TTV Clade 4 | GCGCTTCGCGCGCGCGCCGGGGGGCTCCGC | 5176 |
| | ctdc002 36- | CCCCCC | |
| | nucleotide | | |
| | region | | |
| | TTV Clade 5 | GCGCTTCGCGCGCGCGCCGGGGGGCTGCGC | 5177 |
| | 36-nucleotide | CCCCCC | |
| | region | | |
| | TTV Clade 6 | GCGCTACGCGCGCGCGCCGGGGGGCTGCG | 5178 |
| | 36-nucleotide | CCCCCCC | |
| | region | | |
| | TTV Clade 7 | GCGCTACGCGCGCGCGCCGGGGGGCTCTGC | 5179 |
| | 36-nucleotide | CCCCCC | |
| | region | | |
| Additional | TTV-CT30F | GCGGCGGGGGGGCGGCCGCGTTCGCGCGC | 5195 |
| Alphatorquevirus | | CGCCCACCAGGGGGTGCTGCGCGCCCCCCC | |
| GC-rich region | | CCGCGCATGCGCGGGGCCCCCCCCCGGGG | |
| sequences | | GGGCTCCGCCCCCCCGGCCCCCCCCCGTGC | |
| | | TAAACCCACCGCGCATGCGCGACCACGCCC | |
| | | CCGCCGCC | |
| | TTV-P13-1 | CCGAGCGTTAGCGAGGAGTGCGACCCTACC | 5196 |
| | | CCCTGGGCCCACTTCTTCGGAGCCGCGCGC | |
| | | TACGCCTTCGGCTGCGCGCGGCACCTCAGA | |
| | | CCCCCGCTCGTGCTGACACGCTTGCGCGTG | |
| | | TCAGACCACTTCGGGCTCGCGGGGGTCGGG | |
| | TTV-tth8 | GCCGCCGCGGCGGCGGGGGGCGGCGCGCT | 5197 |
| | | GCGCGCGCCGCCCAGTAGGGGGAGCCATG | |
| | | CGCCCCCCCCCGCGCATGCGCGGGGCCCCC | |
| | | CCCCGCGGGGGGCTCCGCCCCCCGGCCCCC | |
| | | CCCG | |
| | TTV-HD20a | CGGCCCAGCGGCGGCGCGCGCGCTTCGCGC | 5198 |
| | | GCGCGCCGGGGGGCTCCGCCCCCCCCCGCG | |
| | | CATGCGCGGGGCCCCCCCCCGCGGGGGGCT | |
| | | CCGCCCCCCGGTCCCCCCCCG | |
| | TTV-16 | CGGCCGTGCGGCGGCGCGCGCGCTTCGCGC | 5199 |
| | | GCGCGCCGGGGGCTGCCGCCCCCCCCCGCG | |
| | | CATGCGCGCGGGGCCCCCCCCCGCGGGGG | |
| | | GCTCCGCCCCCCGGCCCCCCCCCCCG | |
| | TTV-TJN02 | CGGCGGCGGCGCGCGCGCTACGCGCGCGC | 5200 |
| | | GCCGGGGGGCTGCCGCCCCCCCCCCGCGCA | |
| | | TGCGCGGGGCCCCCCCCCGCGGGGGGCTCC | |
| | | GCCCCCCGGCCCCCC | |
| | TTV-HD16d | GGCGGCGGCGCGCGCGCTACGCGCGCGCG | 5201 |
| | | CCGGGGAGCTCTGCCCCCCCCCGCGCATGC | |
| | | GCGCGGGTCCCCCCCCCGCGGGGGGCTCCG | |
| | | CCCCCCGGTCCCCCCCCCG | |

In some embodiments, the genetic element comprises a nucleic acid sequence having at least about 70%, 75%, 80%, 85%, 90%, 95%, 96%, 97%, 98%, 99%, or 100% sequence identity to the Anellovirus GC-rich nucleotide sequence of any of Tables N1-N1417.

### Effector

In some embodiments, the genetic element may include one or more sequences that encode a functional effector, e.g., an endogenous effector or an exogenous effector, e.g., a therapeutic polypeptide or nucleic acid, e.g., cytotoxic or cytolytic RNA or protein. In some embodiments, the functional nucleic acid is a non-coding RNA. In some embodiments, the functional nucleic acid is a coding RNA. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector is part of the genetic element, e.g., it can be inserted at an insert site as described in Example 10, 12, or 22. In some embodiments, the sequence encoding an effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the sequence encoding an effector is inserted into the genetic element at about nucleotide 3588 of a TTV-tth8 plasmid, e.g., as described herein or at about nucleotide 2843 of a TTMV-LY2 plasmid, e.g., as described herein. In some embodiments, the sequence encoding an effector is inserted into the genetic element at or within nucleotides 336-3015 of a TTV-tth8 plasmid, e.g., as described herein, or at or within nucleotides 242-2812 of a TTV-LY2 plasmid, e.g., as described herein. In some embodiments, the sequence encoding an effector replaces part or all of an open reading frame (e.g., an ORF as described herein, e.g., an ORF1, ORF1/1, ORF1/2, ORF2, ORF2/2, ORF2/3, and/or ORF2t/3 as shown in any of Tables A1-A1417 or N1-N1417).

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the effector is a nucleic acid or protein payload, e.g., as described in Example 11.

### Regulatory Nucleic Acid

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In some embodiments, the regulatory nucleic acid encodes an miRNA.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

In some embodiments, the regulatory nucleic acid is at least one miRNA, e.g., 2, 3, 4, 5, 6, or more. In some embodiments, the genetic element comprises a sequence that encodes an miRNA at least about 75%, 80%, 85%, 90% 95%, 96%, 97%, 98%, 99% or 100% nucleotide sequence identity to any one of the nucleotide sequences or a sequence that is complementary to a sequence described herein.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the anellovector may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

### Therapeutic Effectors (e.g., Peptides or Polypeptides)

In some embodiments, the genetic element comprises a therapeutic expression sequence, e.g., a sequence that encodes a therapeutic peptide or polypeptide, e.g., an intracellular peptide or intracellular polypeptide, a secreted polypeptide, or a protein replacement therapeutic. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range there between.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

### Exemplary Intracellular Polypeptide Effectors

In some embodiments, the effector comprises a cytosolic polypeptide or cytosolic peptide. In some embodiments, the effector comprises cytosolic peptide is a DPP-4 inhibitor, an activator of GLP-1 signaling, or an inhibitor of neutrophil elastase. In some embodiments, the effector increases the level or activity of a growth factor or receptor thereof (e.g., an FGF receptor, e.g., FGFR3). In some embodiments, the effector comprises an inhibitor of n-myc interacting protein activity (e.g., an n-myc interacting protein inhibitor); an inhibitor of EGFR activity (e.g., an EGFR inhibitor); an inhibitor of IDH1 and/or IDH2 activity (e.g., an IDH1 inhibitor and/or an IDH2 inhibitor); an inhibitor of LRP5 and/or DKK2 activity (e.g., an LRP5 and/or DKK2 inhibitor); an inhibitor of KRAS activity; an activator of HTT activity; or inhibitor of DPP-4 activity (e.g., a DPP-4 inhibitor).

In some embodiments, the effector comprises a regulatory intracellular polyeptpide. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

### Exemplary Secreted Polypeptide Effectors

Exemplary secreted therapeutics are described herein, e.g., in the tables below.

TABLE 50
Exemplary cytokines and cytokine receptors

| Cytokine | Cytokine receptor(s) | Entrez Gene ID | UniProt ID |
|---|---|---|---|
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| e | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and IL-22R1/ | 50604 | Q9NYY1 |
| | IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and IL- | 11009 | Q13007 |
| | 22R1/IL-20R2 | | |
| IL-25 | IL-17RA and IL-17RB | 64806 | Q9H293 |
| IL-26 | IL-10R2 chain and IL-20R1 | 55801 | Q9NPH9 |
| | chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e.g., p35, EBI3, or | IL-12Rβ2/gp130; IL- | 10148 | Q14213 |
| a heterodimer thereof) | 12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

In some embodiments, an effector described herein comprises a cytokine of Table 50, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 50 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 50 or a functional variant or fragment thereof) and a second, heterologous region. In some embodiments, the first region is a first cytokine polypeptide of Table 50. In some embodiments, the second region is a second cytokine polypeptide of Table 50, wherein the first and second cytokine polypeptides form a cytokine heterodimer with each other in a wild-type cell. In some embodiments, the polypeptide of Table 50 or functional variant thereof comprises a signal sequence, e.g., a signal sequence that is endogenous to the effector, or a heterologous signal sequence. In some embodiments, an anellovector encoding a cytokine of Table 50, or a functional variant thereof, is used for the treatment of a disease or disorder described herein.

In some embodiments, an effector described herein comprises an antibody molecule (e.g., an scFv) that binds a cytokine of Table 50. In some embodiments, an effector described herein comprises an antibody molecule (e.g., an scFv) that binds a cytokine receptor of Table 50. In some embodiments, the antibody molecule comprises a signal sequence.

Exemplary cytokines and cytokine receptors are described, e.g., in Akdis et al., “Interleukins (from IL-1 to IL-38), interferons, transforming growth factor β, and TNF-α; Receptors, functions, and roles in diseases” October 2016 Volume 138, Issue 4, Pages 984-1010, which is herein incorporated by reference in its entirety, including Table I therein.

TABLE 51
Exemplary polypeptide hormones and receptors

| Hormone | Receptor | Entrez Gene ID | UniProt ID |
|---|---|---|---|
| Natriuretic Peptide, e.g., Atrial | NPRA, NPRB, NPRC | 4878 | P01160 |
| Natriuretic Peptide (ANP) | | | |
| Brain Natriuretic Peptide (BNP) | NPRA, NPRB | 4879 | P16860 |
| C-type natriuretic peptide | NPRB | 4880 | P23582 |
| (CNP) | | | |
| Growth hormone (GH) | GHR | 2690 | P10912 |
| Human growth hormone (hGH) | hGH receptor (human | 2690 | P10912 |
| | GHR) | | |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (LH) | LHR | 3973 | P22888 |
| Antidiuretic hormone (ADH) | Vasopressin receptors, e.g., | 554 | P30518 |
| | V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |

In some embodiments, an effector described herein comprises a hormone of Table 51, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 51 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 51 or functional variant thereof comprises a signal sequence, e.g., a signal sequence that is endogenous to the effector, or a heterologous signal sequence. In some embodiments, an anellovector encoding a hormone of Table 51, or a functional variant thereof, is used for the treatment of a disease or disorder described herein.

In some embodiments, an effector described herein comprises an antibody molecule (e.g., an scFv) that binds a hormone of Table 51. In some embodiments, an effector described herein comprises an antibody molecule (e.g., an scFv) that binds a hormone receptor of Table 51. In some embodiments, the antibody molecule comprises a signal sequence.

TABLE 52
Exemplary growth factors

| | | |
|---|---|---|
| Growth Factor | Entrez Gene ID | UniProt ID |

PDGF family

| PDGF (e.g., PDGF-1, | PDGF receptor, e.g., | 5156 | P16234 |
|---|---|---|---|
| PDGF-2, or a | PDGFRα, PDGFRβ | | |
| heterodimer thereof) | | | |
| CSF-1 | CSF1R | 1435 | P09603 |
| SCF | CD117 | 3815 | P10721 |
| VEGF family | | | |
| VEGF (e.g., isoforms | VEGFR-1, VEGFR- | 2321 | P17948 |
| VEGF 121, VEGF 165, | 2 | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR-3 | | |
| PIGF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, 2249, | P05230, P09038, |
| FGF-4, FGF-5, FGF-6, | FGFR3, and FGFR4 | 2250, 2251, 2252, 2253, | P11487, P08620, |
| FGF-7, FGF-8, FGF-9 | | 2254 | P12034, P10767, |
| | | | P21781, P55075, P31371 |
| Insulin family | | | |
| Insulin | IR | 3630 | P01308 |
| IGF-I | IGF-I receptor, IGF- | 3479 | P05019 |
| | II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |

In some embodiments, an effector described herein comprises a growth factor of Table 52, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 52 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 52 or functional variant thereof comprises a signal sequence, e.g., a signal sequence that is endogenous to the effector, or a heterologous signal sequence. In some embodiments, an anellovector encoding a growth factor of Table 52, or a functional variant thereof, is used for the treatment of a disease or disorder described herein.

In some embodiments, an effector described herein comprises an antibody molecule (e.g., an scFv) that binds a growth factor of Table 52. In some embodiments, an effector described herein comprises an antibody molecule (e.g., an scFv) that binds a growth factor receptor of Table 52. In some embodiments, the antibody molecule comprises a signal sequence.

Exemplary growth factors and growth factor receptors are described, e.g., in Bafico et al., “Classification of Growth Factors and Their Receptors” Holland-Frei Cancer Medicine. 6th edition, which is herein incorporated by reference in its entirety.

TABLE 53
Clotting-associated factors

| Effector | Indication | Entrez Gene ID | UniProt ID |
|---|---|---|---|
| Factor I | Afibrinogenomia | 2243, 2266, | P02671, P02679, |
| (fibrinogen) | | 2244 | P02675 |
| Factor II | Factor II Deficiency | 2147 | P00734 |
| Factor IX | Hemophilia B | 2158 | P00740 |
| Factor V | Owren′s disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| vWF | von Willebrand disease | 7450 | P04275 |

In some embodiments, an effector described herein comprises a polypeptide of Table 53, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 53 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, the polypeptide of Table 53 or functional variant thereof comprises a signal sequence, e.g., a signal sequence that is endogenous to the effector, or a heterologous signal sequence. In some embodiments, an anellovector encoding a polypeptide of Table 53, or a functional variant thereof is used for the treatment of a disease or disorder of Table 53.

### Exemplary Protein Replacement Therapeutics

Exemplary protein replacement therapeutics are described herein, e.g., in the tables below.

TABLE 54
Exemplary enzymatic effectors and corresponding indications

| Effector | deficiency | Entrez Gene ID | UniProt ID |
|---|---|---|---|
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo′s syndrome) | | |
| acetyltransferase | Type III-C | | |
| ADAMTS13 | Thrombotic | 11093 | Q76LX8 |
| | Thrombocytopenia Purpura | | |
| adenine | Adenine | 353 | P07741 |
| phosphoribosyl- | phosphoribosyltransferase | | |
| transferase | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe′s disease) | | |
| Arginase | Familial hyperarginemia | 383, 384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber′s disease | 125981, 340485, | Q8TDN7, |
| | (lipogranulomatosis) | 55331 | Q5QJU3, Q9NUN7 |
| Cystathionine B | Homocystinuria | 875 | P35520 |
| synthase | | | |
| Dolichol-P-mannose | Congenital disorders of N- | 8813, 54344 | O60762, Q9P2X0 |
| synthase | glycosylation CDG Ie | | |
| Dolicho-P- | Congenital disorders of N- | 84920 | Q5BKT4 |
| Glc:Man9GlcNAc2-PP- | glycosylation CDG Ic | | |
| dolichol | | | |
| glucosyltransferase | | | |
| Dolicho-P- | Congenital disorders of N- | 10195 | Q92685 |
| Man:Man5GlcNAc2- | glycosylation CDG Id | | |
| PP-dolichol | | | |
| mannosyltransferase | | | |
| Dolichyl-P-glucose:Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichyl-P- | Congenital disorders of N- | 79087 | Q9BV10 |
| mannose:Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren′s disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio′s syndrome) | | |
| | Type IV-A | | |
| Galactosylceramide β- | Krabbe′s disease | 2581 | P54803 |
| galactosidase | | | |
| Ganglioside β- | GM1 gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher′s disease | 2629 | P04062 |
| glucosidase | | | |
| Heparan-S-sulfate | Mucopolysaccharidosis MPS | 6448 | P51688 |
| sulfamidase | III (Sanfilippo′s syndrome) | | |
| | Type III-A | | |
| homogentisate oxidase | Alkaptonuria | 3081 | Q93099 |
| Hyaluronidase | Mucopolysaccharidosis MPS | 3373, 8692, 8372, | Q12794, Q12891, |
| | IX (hyaluronidase deficiency) | 23553 | O43820, Q2M3T9 |
| Iduronate sulfate | Mucopolysaccharidosis MPS | 3423 | P22304 |
| sulfatase | II (Hunter′s syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman′s disease | 3988 | P38571 |
| Lysosomal pepstatin- | Ceroid lipofuscinosis Late | 1200 | 014773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyltransf | | | |
| erase | | | |
| Metalloproteinase-2 | Winchester syndrome | 4313 | P08253 |
| methylmalonyl-CoA | Methylmalonic acidemia | 4594 | P22033 |
| mutase | (vitamin b12 non-responsive) | | |
| N-Acetyl | Mucopolysaccharidosis MPS | 411 | P15848 |
| galactosamine α-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo′s syndrome) | | |
| | Type III-B | | |
| N-Acetyl- | Schindler′s disease Type I | 4668 | P17050 |
| galactosaminidase | (infantile severe form) | | |
| N-Acetyl- | Schindler′s disease Type II | 4668 | P17050 |
| galactosaminidase | (Kanzaki disease, adult-onset | | |
| | form) | | |
| N-Acetyl- | Schindler′s disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo′s syndrome) | | |
| | Type III-D | | |
| N-acetylglucosaminyl-1- | Mucolipidosis ML III | 79158 | Q3T906 |
| phosphotransferase | (pseudo-Hurler′s | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminy1-1- | Mucolipidosis ML III | 84572 | Q9UJJ9 |
| phosphotransferase, | (pseudo-Hurler′s | | |
| substrate-recognition | polydystrophy) Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs′ disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | 015305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9HOPO |
| nucleotidase | pyrimidine 5′ nucleotidase | | |
| | deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide α- | Fabry′s disease | 2717 | P06280 |
| galactosidase | | | |
| tyrosinase, e.g., OCA1 | albinism, e.g., ocular albinism | 7299 | P14679 |
| UDP-GlcNAc:dolichyl- | Congenital disorders of N- | 1798 | Q9H3H5 |
| P NAcGlc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type I (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-1-Iduronidase | Mucopolysaccharidosis MPS | 2517 | P04066 |
| | I H/S (Hurler-Scheie | | |
| | syndrome) | | |
| α-1-Iduronidase | Mucopolysaccharidosis MPS | 3425 | P35475 |
| | I-H (Hurler′s syndrome) | | |
| α-1-Iduronidase | Mucopolysaccharidosis MPS | 3425 | P35475 |
| | I-S (Scheie′s syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio′s syndrome) | | |
| | Type IV-B | | |
| β-Glucuronidase | Mucopolysaccharidosis MPS | 2720 | P16278 |
| | VII (Sly′s syndrome) | | |
| β-Hexosaminidase A | Tay-Sachs disease | 2990 | P08236 |
| β-Hexosaminidase B | Sandhoff′s disease | 3073 | P06865 |

In some embodiments, an effector described herein comprises an enzyme of Table 54, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 54 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, an anellovector encoding an enzyme of Table 54, or a functional variant thereof is used for the treatment of a disease or disorder of Table 54. In some 10 embodiments, an anellovector is used to deliver uridine diphosphate glucuronyl-transferase or a functional variant thereof to a target cell, e.g., a liver cell. In some embodiments, an anellovector is used to deliver OCA1 or a functional variant thereof to a target cell, e.g., a retinal cell.

TABLE 55
Exemplary non-enzymatic effectors and corresponding indications

| Effector | Indication | Entrez Gene ID | UniProt ID |
|---|---|---|---|
| Survival motor neuron | spinal muscular atrophy | 6606 | Q16637 |
| protein (SMN) | | | |
| Dystrophin or micro- | muscular dystrophy | 1756 | P11532 |
| dystrophin | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | Ile | | |
| Prosaposin | Prosaposin deficiency | 5660 | P07602 |
| Protective | Galactosialidosis | 5476 | P10619 |
| protein/cathepsin A | (Goldberg′s syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher′s activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, | P02671, P02675, |
| | | 2266 | P02679 |
| erythropoietin (hEPO) | | | |

In some embodiments, an effector described herein comprises an erythropoietin (EPO), e.g., a human erythropoietin (hEPO), or a functional variant thereof. In some embodiments, an anellovector encoding an erythropoietin, or a functional variant thereof is used for stimulating erythropoiesis. In some embodiments, an anellovector encoding an erythropoietin, or a functional variant thereof is used for the treatment of a disease or disorder, e.g., anemia. In some embodiments, an anellovector is used to deliver EPO or a functional variant thereof to a target cell, e.g., a red blood cell.

In some embodiments, an effector described herein comprises a polypeptide of Table 55, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 55 by reference to its UniProt ID. In some embodiments, an anellovector encoding a polypeptide of Table 55, or a functional variant thereof is used for the treatment of a disease or disorder of Table 55. In some embodiments, an anellovector is used to deliver SMN or a functional variant thereof to a target cell, e.g., a cell of the spinal cord and/or a motor neuron. In some embodiments, an anellovector is used to deliver a micro-dystrophin to a target cell, e.g., a myocyte.

Exemplary micro-dystrophins are described in Duan, “Systemic AAV Micro-dystrophin Gene Therapy for Duchenne Muscular Dystrophy.” Mol Ther. 2018 Oct. 3:26(10): 2337-2356. doi: 10.1016/j.ymthe.2018.07.011. Epub 2018 Jul. 17.

In some embodiments, an effector described herein comprises a clotting factor, e.g., a clotting factor listed in Table 54 or Table 55 herein. In some embodiments, an effector described herein comprises a protein that, when mutated, causes a lysosomal storage disorder, e.g., a protein listed in Table 54 or Table 55 herein. In some embodiments, an effector described herein comprises a transporter protein, e.g., a transporter protein listed in Table 55 herein.

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polyeptpide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

### Regeneration, Repair, and Fibrosis Factors

Therapeutic polypeptides described herein also include growth factors, e.g., as disclosed in Table 56, or functional variants thereof, e.g., a protein having at least 80%, 85%, 90%, 95%, 967%, 98%, 99% identity to a protein sequence disclosed in Table 56 by reference to its UniProt ID. Also included are antibodies or fragments thereof against such growth factors, or miRNAs that promote regeneration and repair.

TABLE 56
Exemplary regeneration, repair, and fibrosis factors

| Target | Gene accession # | Protein accession # |
|---|---|---|
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR-199-3p | MIMAT0000232 | |
| miR-590-3p | MIMAT0004801 | |
| mi-17-92 | MI0000071 | https://www.ncbi.nlm.nih.gov/pmc/ |
| | | articles/PMC2732113/figure/F1/ |
| miR-222 | MI0000299 | |
| miR-302-367 | MIR302A And | https://www.ncbi.nlm.nih.gov/pmc/ |
| | MIR367 | articles/PMC4400607/ |

### Transformation Factors

Therapeutic polypeptides described herein also include transformation factors, e.g., protein factors that transform fibroblasts into differentiated cell e.g., factors disclosed in Table 57 or functional variants thereof, .g., a protein having at least 80%, 85%, 90%, 95%, 967%, 98%, 99% identity to a protein sequence disclosed in Table 57 by reference to its UniProt ID.

TABLE 57
Exemplary transformation factors

| | | Gene | Protein |
|---|---|---|---|
| Target | Indication | accession # | accession # |
| MESP1 | Organ Repair by | Gene ID: 55897 | EAX02066 |
| | transforming fibroblasts | | |
| ETS2 | Organ Repair by | GeneID: 2114 | NP_005230 |
| | transforming fibroblasts | | |
| HAND2 | Organ Repair by | GeneID: 9464 | NP_068808 |
| | transforming fibroblasts | | |
| MYOCARDIN | Organ Repair by | GeneID: 93649 | NP_001139784 |
| | transforming fibroblasts | | |
| ESRRA | Organ Repair by | Gene ID: 2101 | AAH92470 |
| | transforming fibroblasts | | |
| miR-1 | Organ Repair by | MI0000651 | n/a |
| | transforming fibroblasts | | |
| miR-133 | Organ Repair by | MI0000450 | n/a |
| | transforming fibroblasts | | |
| TGFb | Organ Repair by | GeneID: 7040 | NP_000651.3 |
| | transforming fibroblasts | | |
| WNT | Organ Repair by | Gene ID: 7471 | NP_005421 |
| | transforming fibroblasts | | |
| JAK | Organ Repair by | Gene ID: 3716 | NP_001308784 |
| | transforming fibroblasts | | |
| NOTCH | Organ Repair by | GeneID: 4851 | XP_011517019 |
| | transforming fibroblasts | | |

Proteins that Stimulate Cellular Regeneration

Therapeutic polypeptides described herein also include proteins that stimulate cellular regeneration e.g., proteins disclosed in Table 58 or functional variants thereof, e.g., a protein having at least 80%, 85%, 90%, 95%, 967%, 98%, 99% identity to a protein sequence disclosed in Table 58 by reference to its UniProt ID.

TABLE 58
Exemplary proteins that stimulate cellular regeneration

| Target | Gene accession # | Protein accession # |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

### STING Modulator Effectors

In some embodiments, a secreted effector described herein modulates STING/cGAS signaling. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679, which is incorporated herein by reference in its entirety. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8): 1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7(283): 283ra52, each of which is incorporated herein by reference in its entirety.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7): 1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition or anellovector described herein includes a polypeptide linked to a ligand that is capable of targeting a specific location, tissue, or cell.

### Gene Editing Components

The genetic element of the anellovector may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaca. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type Vendonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the anellovector includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. In addition, one promoter element can increase an amount of products expressed for multiple DNA sequences attached in tandem. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic beta.-actin promoter and the phosphoglycerol kinase (PGK) promoter.

In another embodiment, inducible promoters may be desired. Inducible promoters are those which are regulated by exogenously supplied compounds, either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268: 1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

### Replication Proteins

In some embodiments, the genetic element of the anellovector, e.g., synthetic anellovector, may include sequences that encode one or more replication proteins. In some embodiments, the anellovector may replicate by a rolling-circle replication method, e.g., synthesis of the leading strand and the lagging strand is uncoupled. In such embodiments, the anellovector comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi: 10.1186/1743-422X-6-60.

The genetic element may comprise a sequence encoding a polymerase, e.g., RNA polymerase or a DNA polymerase.

### Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the anellovector in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

### Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

### Viral Sequence

In some embodiments, the genetic element comprises at least one viral sequence. In some embodiments, the sequence has homology or identity to one or more sequence from a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an Anellovirus. Recent changes in nomenclature have classified the three Anelloviruses able to infect human cells into Alphatorquevirus (TT), Betatorquevirus (TTM), and Gammatorquevirus (TTMD) Genera of the Anelloviridae family of viruses. To date Anelloviruses have not been linked to any human disease. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

In some embodiments, the genetic element comprises one or more sequences with homology or identity to one or more sequences from one or more non-Anelloviruses, e.g., adenovirus, herpes virus, pox virus, vaccinia virus, SV40, papilloma virus, an RNA virus such as a retrovirus, e.g., lentivirus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the anellovectors described herein include cell lines known in the art. e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine. (2) asparagine, glutamine, and histidine, (3) cysteine and serine. (4) glycine and proline. (5) isoleucine, leucine and valine, (6) methionine, leucine and isoleucine, (7) phenylalanine, methionine, leucine, and tyrosine, (8) serine and threonine, (9) tryptophan and tyrosine. (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more.

In some embodiments, the genetic element comprises a nucleotide sequence with at least about 75% nucleotide sequence identity, at least about 80%, 85%, 90% 95%, 96%, 97%, 98%, 99% or 100% nucleotide sequence identity to any one of the nucleotide sequences described herein, e.g., as listed in any of Tables N1-N1417. Since the genetic code is degenerate, a homologous nucleotide sequence can include any number of silent base changes, i.e., nucleotide substitutions that nonetheless encode the same amino acid.

### Gene Editing Component

The genetic element of the anellovector may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013): 397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaca. CRISPR systems use RNA-guided nucleases termed CRISPR-associated or “Cas” endonucleases (e.g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas9 generates only a single-strand break; a catalytically inactive Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), cSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

As used herein, a “biologically active portion of an effector domain” is a portion that maintains the function (e.g. completely, partially, or minimally) of an effector domain (e.g., a “minimal” or “core” domain). In some embodiments, the anellovector includes a gene encoding a fusion of a dCas9 with all or a portion of one or more effector domains to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Proteinaceous Exterior

In some embodiments, the anellovector, e.g., synthetic anellovector, comprises a proteinaccous exterior that encloses the genetic element. The proteinaceous exterior can comprise a substantially non-pathogenic exterior protein that fails to elicit an unwanted immune response in a mammal. The proteinaceous exterior of the anellovectors typically comprises a substantially non-pathogenic protein that may self-assemble into an icosahedral formation that makes up the proteinaceous exterior.

In some embodiments, the proteinaceous exterior protein is encoded by a sequence of the genetic element of the anellovector (e.g., is in cis with the genetic element). In other embodiments, the proteinaceous exterior protein is encoded by a nucleic acid separate from the genetic element of the anellovector (e.g., is in trans with the genetic element).

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., an Anellovirus ORF1 sequence or a capsid protein sequence as listed in any of Tables A1-A1417. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to any one of the nucleotide sequences described herein, e.g., an Anellovirus capsid sequence or a capsid protein sequence as listed in any of Tables A1-A1417. In some embodiments, the protein comprises a capsid protein or a functional fragment of a capsid protein that is encoded by a capsid nucleotide sequence or a sequence having at least about 60%, 65%, 70%, 75%, 80%, 85%, 90% 95%, 96%, 97%, 98%, 99%, or 100% nucleotide sequence identity to any one of the nucleotide sequences described herein, e.g., an Anellovirus capsid sequence or a capsid protein sequence as listed in any of Tables N1-N1417.

In some embodiments, the anellovector comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to any one of the amino acid sequences described herein, e.g., an Anellovirus capsid sequence or a capsid protein sequence in any of Tables A1-A1417. In some embodiments, the anellovector comprises a nucleotide sequence encoding a capsid protein or a functional fragment of a capsid protein or a sequence having at least about 60%, 65%, 70%, 75%, 80%, 85%, 90% 95%, 96%, 97%, 98%, 99%, or 100% sequence identity to any one of the amino acid sequences described herein, e.g., an Anellovirus capsid sequence or a capsid protein sequence in any of Tables A1-A1417.

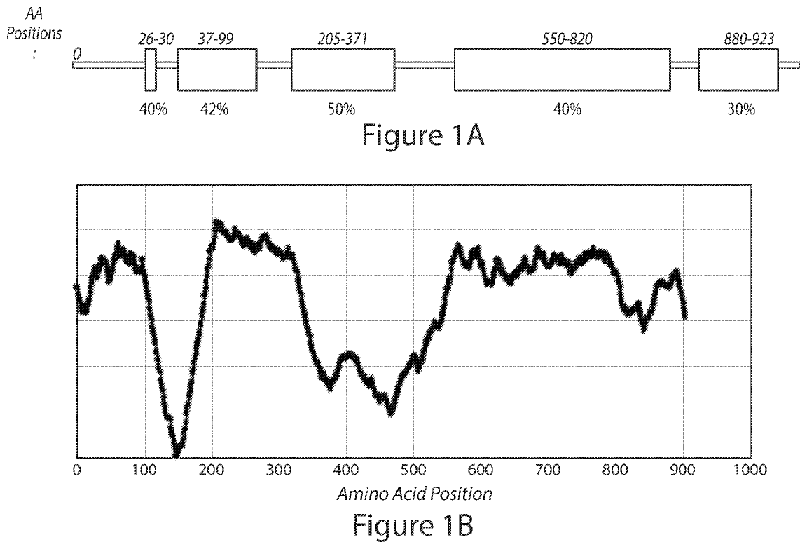

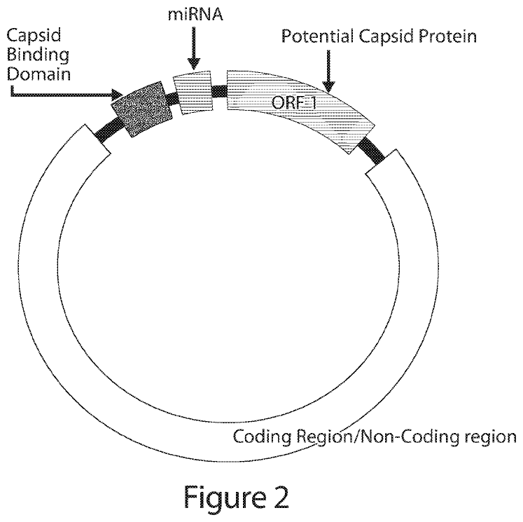

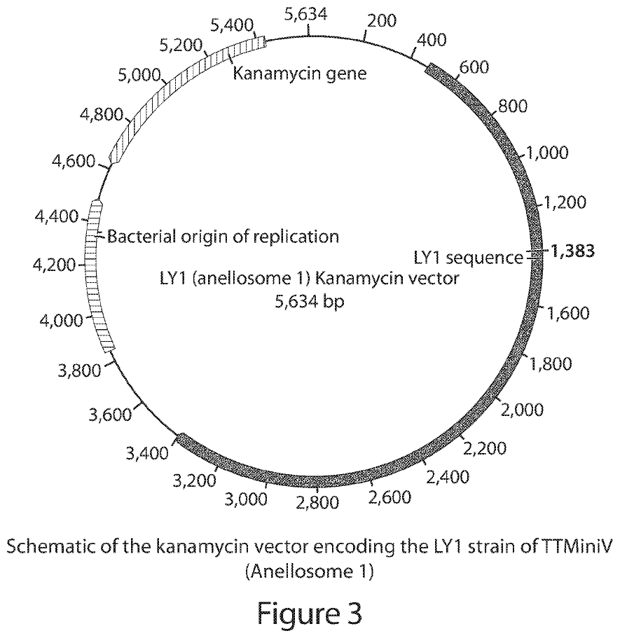

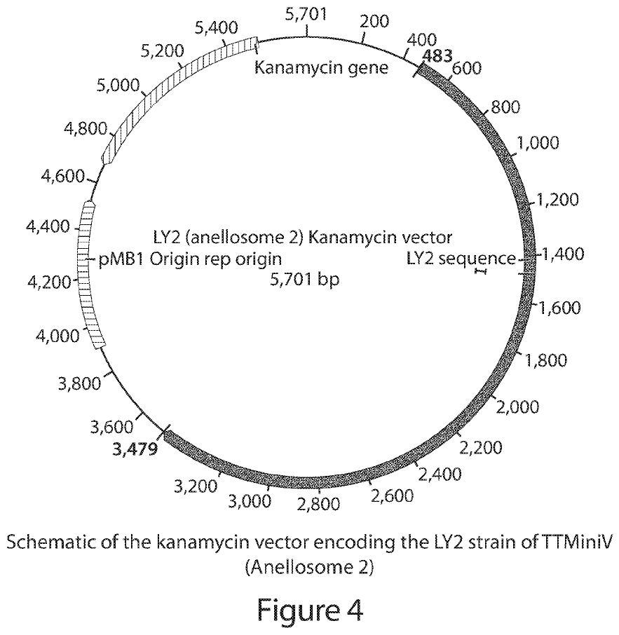

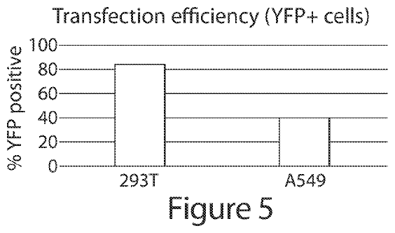

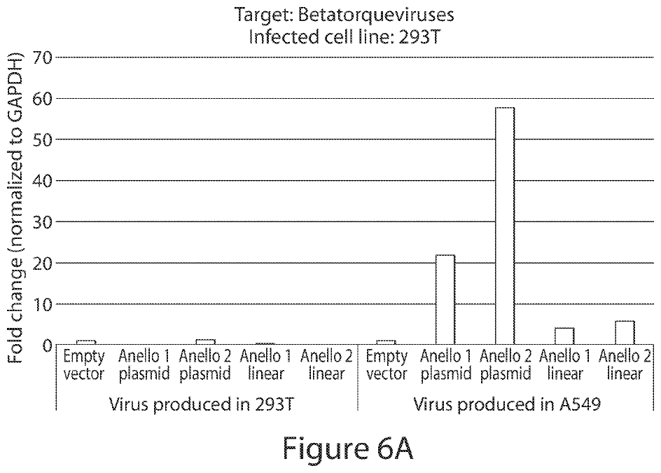

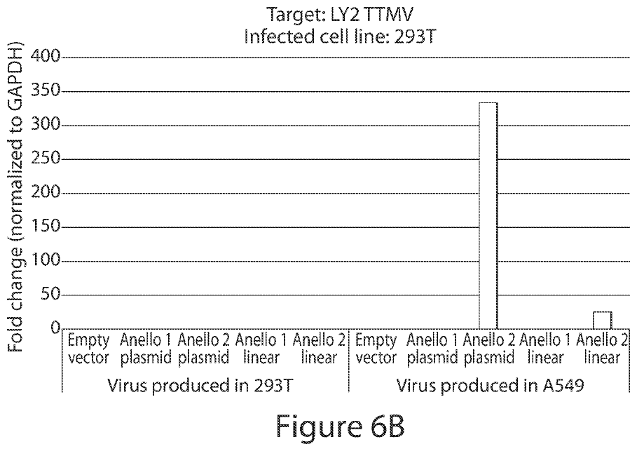

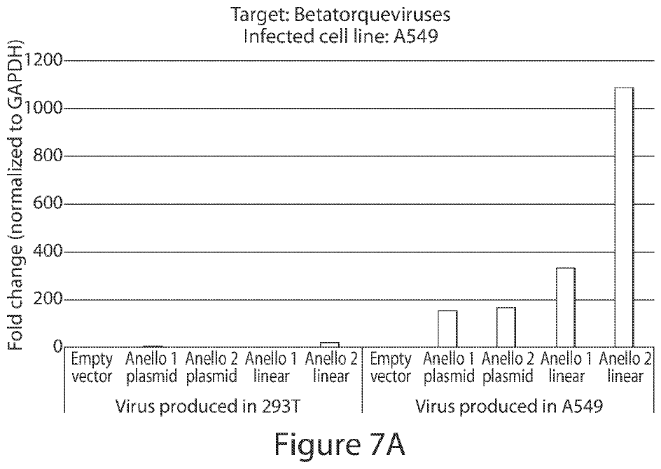

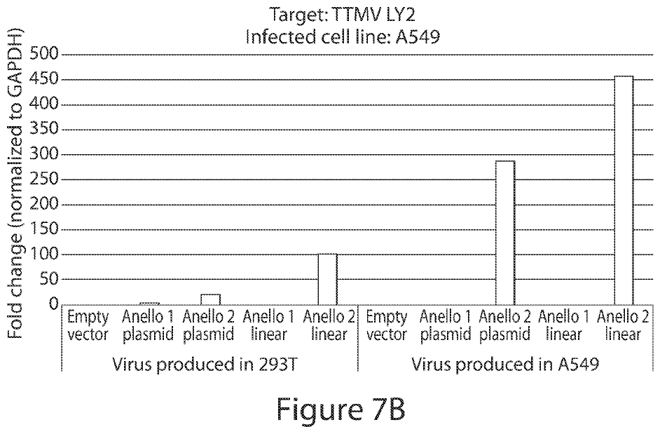

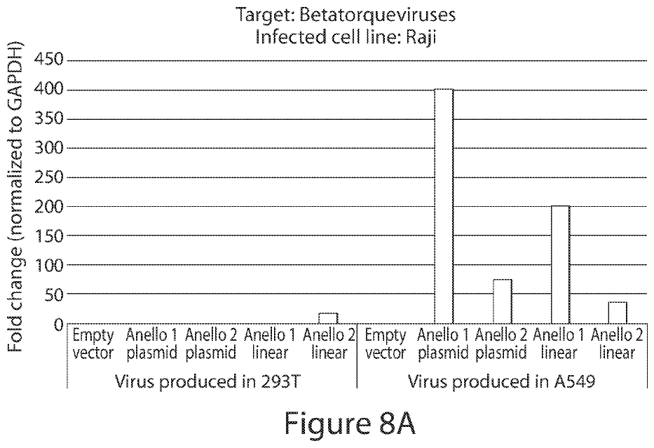

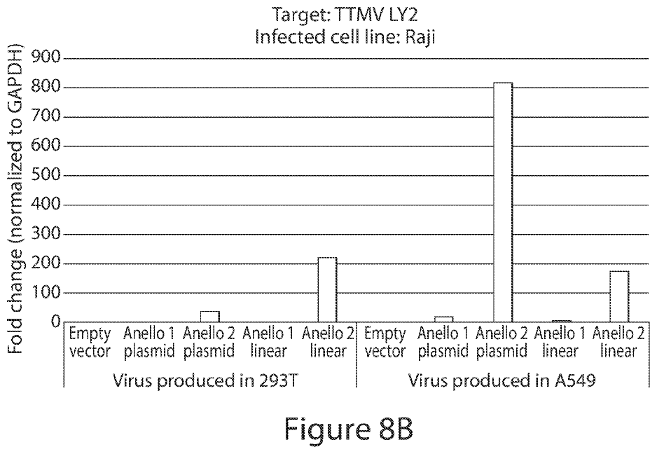

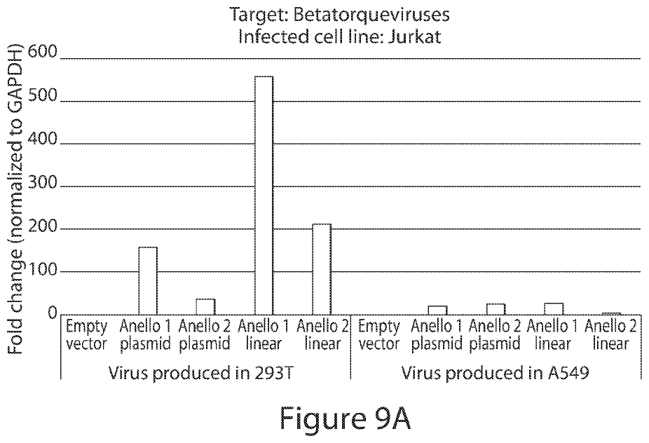

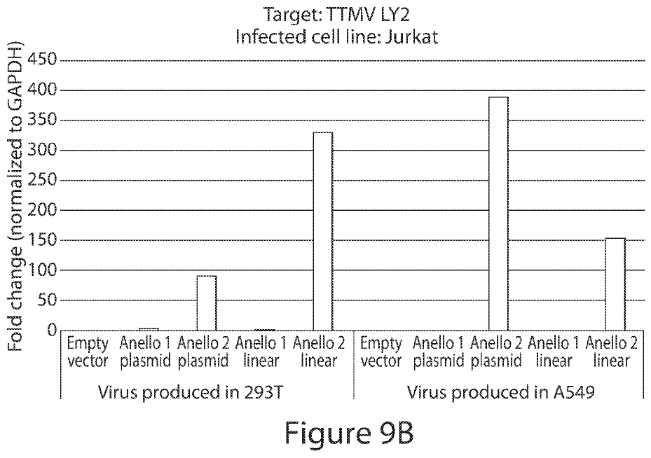

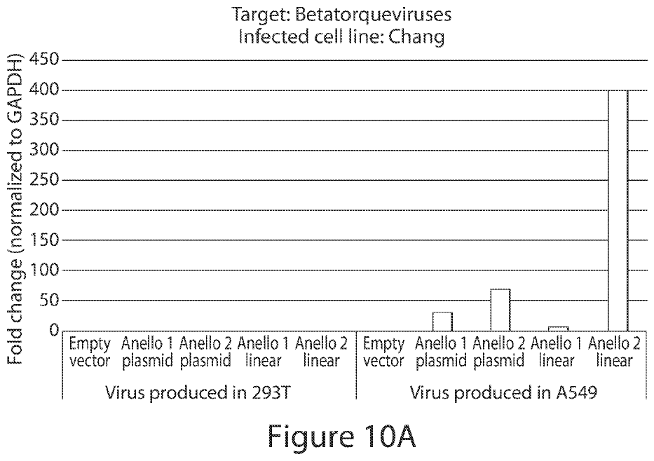

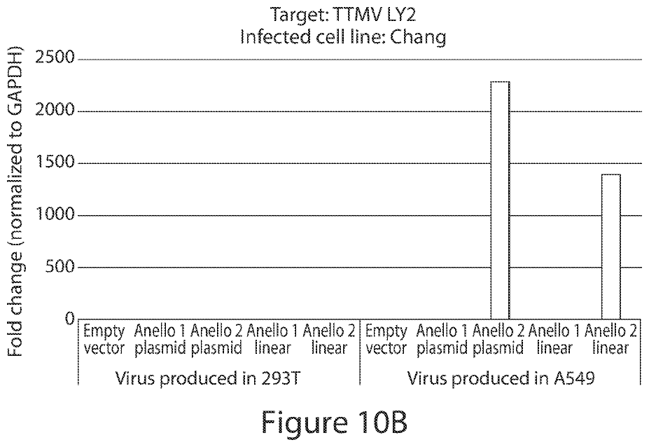

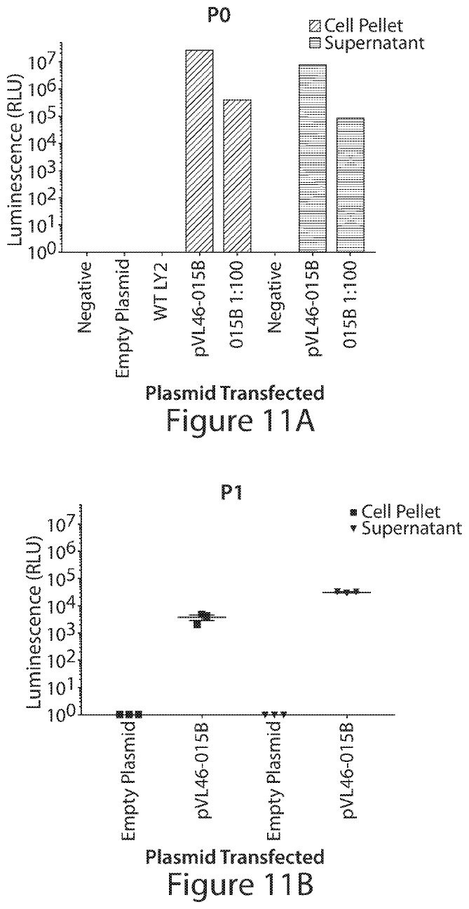

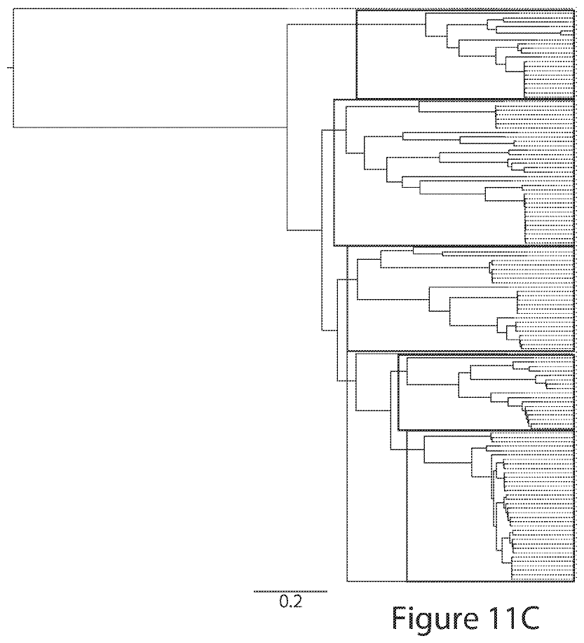

In some embodiments, the anellovector comprises a nucleotide sequence encoding an amino acid sequence having about position 1 to about position 150 (e.g., or any subset of amino acids within each range, e.g., about position 20 to about position 35, about position 25 to about position 30, about position 26 to about 30), about position 150 to about position 390 (e.g., or any subset of amino acids within each range, e.g., about position 200 to about position 380, about position 205 to about position 375, about position 205 to about 371), about 390 to about position 525, about position 525 to about position 850 (e.g., or any subset of amino acids within each range, e.g., about position 530 to about position 840, about position 545 to about position 830, about position 550 to about 820), about 850 to about position 950 (e.g., or any subset of amino acids within each range, e.g., about position 860 to about position 940, about position 870 to about position 930, about position 880 to about 923) of the amino acid sequences described herein, an Anellovirus amino acid sequence, e.g., as listed in any of Tables A1-A1417, or shown in FIG. 1, or a functional fragment thereof. In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to about position 1 to about position 150 (e.g., or any subset of amino acids within each range as described herein), about position 150 to about position 390, about position 390 to about position 525, about position 525 to about position 850, about position 850 to about position 950 of the amino acid sequences described herein, an Anellovirus amino acid sequence, e.g., as listed in any of Tables A1-A1417, or as shown in FIG. 1. In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences or ranges of amino acids described herein, an Anellovirus amino acid sequence, e.g., as listed in any of Tables A1-A1417, or shown in FIG. 1. In some embodiments, the ranges of amino acids with less sequence identity may provide one or more of the properties described herein and differences in cell/tissue/species specificity (e.g. tropism).

In some embodiments, the anellovector lacks lipids in the proteinaceous exterior. In some embodiments, the anellovector lacks a lipid bilayer, e.g., a viral envelope. In some embodiments, the interior of the anellovector is entirely covered (e.g., 100% coverage) by a proteinaceous exterior. In some embodiments, the interior of the anellovector is less than 100% covered by the proteinaceous exterior, e.g., 95%, 90%, 85%, 80%, 70%, 60%, 50% or less coverage. In some embodiments, the proteinaccous exterior comprises gaps or discontinuities, e.g., permitting permeability to water, ions, peptides, or small molecules, so long as the genetic element is retained in the anellovector.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by an Anellovirus ORF1 described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

### II. Compositions and Methods for Making Anellovectors

The present disclosure provides, in some aspects, anellovectors and methods thereof for delivering effectors. In some embodiments, the anellovectors or components thereof can be made as described below. In some embodiments, the compositions and methods described herein can be used to produce a genetic element or a genetic element construct. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule), e.g., in a host cell. In some embodiments, the anellovectors or components thereof can be made using a tandem construct, e.g., as described in U.S. Provisional Application 63/038,483, which is incorporated herein by reference in its entirety. In some embodiments, the anellovectors or components thereof can be made using a bacmid/insect cell system, e.g., as described as described in U.S. Provisional Application No. 63/038,603, which is incorporated herein by reference in its entirety.

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) the genetic element region. The Rep protein may then proceed through the genetic element region, resulting in the synthesis of the genetic element. The genetic element may then be circularized and then enclosed within a proteinaceous exterior to form an anellovector.

### Components and Assembly of Anellovectors

The compositions and methods herein can be used to produce anellovectors. As described herein, an anellovector generally comprises a genetic element (e.g., a single-stranded, circular DNA molecule, e.g., comprising a 5′ UTR region as described herein) enclosed within a proteinaceous exterior (e.g., comprising a polypeptide encoded by an Anellovirus ORF1 nucleic acid, e.g., as described herein). In some embodiments, the genetic element comprises one or more sequences encoding Anellovirus ORFs (e.g., one or more of an Anellovirus ORF1, ORF2, ORF2/2, ORF2/3, ORF1/1, or ORF1/2). As used herein, an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described in PCT/US2018/037379 or PCT/US19/65995 (each of which is incorporated by reference herein in their entirety). In embodiments, the genetic element comprises a sequence encoding an Anellovirus ORF1, or a splice variant or functional fragment thereof (e.g., a jelly-roll region, e.g., as described herein). In some embodiments, the proteinaceous exterior comprises a polypeptide encoded by an Anellovirus ORF1 nucleic acid (e.g., an Anellovirus ORF1 molecule or a splice variant or functional fragment thereof).

In some embodiments, an anellovector is assembled by enclosing a genetic element (e.g., as described herein) within a proteinaceous exterior (e.g., as described herein). In some embodiments, the genetic element is enclosed within the proteinaceous exterior in a host cell (e.g., as described herein). In some embodiments, the host cell expresses one or more polypeptides comprised in the proteinaceous exterior (e.g., a polypeptide encoded by an Anellovirus ORF1 nucleic acid, e.g., an ORF1 molecule). For example, in some embodiments, the host cell comprises a nucleic acid sequence encoding an Anellovirus ORF1 molecule, e.g., a splice variant or a functional fragment of an Anellovirus ORF1 polypeptide (e.g., a wild-type Anellovirus ORF1 protein or a polypeptide encoded by a wild-type Anellovirus ORF1 nucleic acid, e.g., as described herein). In embodiments, the nucleic acid sequence encoding the Anellovirus ORF1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 molecule is integrated into the genome of the host cell.

In some embodiments, the host cell comprises the genetic element and/or a nucleic acid construct comprising the sequence of the genetic element. In some embodiments, the nucleic acid construct is selected from a plasmid, viral nucleic acid, minicircle, bacmid, or artificial chromosome. In some embodiments, the genetic element is excised from the nucleic acid construct and, optionally, converted from a double-stranded form to a single-stranded form (e.g., by denaturation). In some embodiments, the genetic element is generated by a polymerase based on a template sequence in the nucleic acid construct. In some embodiments, the polymerase produces a single-stranded copy of the genetic element sequence, which can optionally be circularized to form a genetic element as described herein. In other embodiments, the nucleic acid construct is a double-stranded minicircle produced by circularizing the nucleic acid sequence of the genetic element in vitro. In embodiments, the in vitro-circularized (IVC) minicircle is introduced into the host cell, where it is converted to a single-stranded genetic element suitable for enclosure in a proteinaceous exterior, as described herein.

### ORF1 Molecules, e.g., for Assembly of Anellovectors

An anellovector can be made, for example, by enclosing a genetic element within a proteinaceous exterior. The proteinaceous exterior of an Anellovector generally comprises a polypeptide encoded by an Anellovirus ORF1 nucleic acid (e.g., an Anellovirus ORF1 molecule or a splice variant or functional fragment thereof, e.g., as described herein). An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands). In embodiments, the proteinaceous exterior comprises one or more (e.g., 1, 2, 3, 4, or all 5) of an Anellovirus ORF1 arginine-rich region, jelly-roll region, N22 domain, hypervariable region, and/or C-terminal domain. In some embodiments, the proteinaceous exterior comprises an Anellovirus ORF1 jelly-roll region (e.g., as described herein). In some embodiments, the proteinaceous exterior comprises an Anellovirus ORF1 arginine-rich region (e.g., as described herein). In some embodiments, the proteinaceous exterior comprises an Anellovirus ORF1 N22 domain (e.g., as described herein). In some embodiments, the proteinaceous exterior comprises an Anellovirus hypervariable region (e.g., as described herein). In some embodiments, the proteinaceous exterior comprises an Anellovirus ORF1 C-terminal domain (e.g., as described herein).

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein), or a functional fragment thereof. In some embodiments, the ORF1 molecule comprises a truncation relative to an Anellovirus ORF1 protein (e.g., an Anellovirus ORF1 protein as described herein). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may enclose a nucleic acid molecule (e.g., a genetic element as described herein, e.g., produced using a composition or construct as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer.

In some embodiments, a first plurality of anellovectors comprising an ORF1 molecule as described herein is administered to a subject. In some embodiments, a second plurality of anellovectors comprising an ORF1 molecule described herein, is subsequently administered to the subject following administration of the first plurality. In some embodiments the second plurality of anellovectors comprises an ORF1 molecule having the same amino acid sequence as the ORF1 molecule comprised by the anellovectors of the first plurality. In some embodiments the second plurality of anellovectors comprises an ORF1 molecule having at least 70%, 75%, 80%, 85%, 90%, 95%, 96%, 97%, 98%, 99%, or 100% amino acid sequence identity to the ORF1 molecule comprised by the anellovectors of the first plurality.

### ORF2 Molecules, e.g., for Assembly of Anellovectors

Producing an anellovector using the compositions or methods described herein may involve expression of an Anellovirus ORF2 molecule (e.g., as described herein), or a splice variant or functional fragment thereof. In some embodiments, the anellovector comprises an ORF2 molecule, or a splice variant or functional fragment thereof, and/or a nucleic acid encoding an ORF2 molecule, or a splice variant or functional fragment thereof. In some embodiments, the anellovector does not comprise an ORF2 molecule, or a splice variant or functional fragment thereof, and/or a nucleic acid encoding an ORF2 molecule, or a splice variant or functional fragment thereof. In some embodiments, producing the anellovector comprises expression of an ORF2 molecule, or a splice variant or functional fragment thereof, but the ORF2 molecule is not incorporated into the anellovector.

### Production of Protein Components

Protein components of an anellovector, e.g., ORF1, can be produced in a variety of ways, e.g., as described herein. In some embodiments, the protein components of an anellovector, including, e.g., the proteinaceous exterior, are produced in the same host cell that packages the genetic elements into the proteinaceous exteriors, thereby producing the anellovectors. In some embodiments, the protein components of an anellovector, including, e.g., the proteinaceous exterior, are produced in a cell that does not comprise a genetic element and/or a genetic element construct (e.g., as described herein).

### Baculovirus Expression Systems

A viral expression system, e.g., a baculovirus expression system, may be used to express proteins (e.g., for production of anellovectors), e.g., as described herein. Baculoviruses are rod-shaped viruses with a circular, supercoiled double-stranded DNA genome. Genera of baculoviruses include: Alphabaculovirus (nucleopolyhedroviruses (NPVs) isolated from Lepidoptera), Betabaculoviruses (granuloviruses (GV) isolated from Lepidoptera), Gammabaculoviruses (NPVs isolated from Hymenoptera) and Deltabaculoviruses (NPVs isolated from Diptera). While GVs typically contain only one nucleocapsid per envelope, NPVs typically contain either single (SNPV) or multiple (MNPV) nucleocapsids per envelope. The enveloped virions are further occluded in granulin matrix in GVs and polyhedrin in NPVs. Baculoviruses typically have both lytic and occluded life cycles. In some embodiments, the lytic and occluded life cycles manifest independently throughout the three phases of virus replication: early, late, and very late phase. In some embodiments, during the early phase, viral DNA replication takes place following viral entry into the host cell, early viral gene expression and shut-off of the host gene expression machinery. In some embodiments, in the late phase late genes that code for viral DNA replication are expressed, viral particles are assembled, and extracellular virus (EV) is produced by the host cell. In some embodiments, in the very late phase the polyhedrin and p10 genes are expressed, occluded viruses (OV) are produced by the host cell, and the host cell is lysed. Since baculoviruses infect insect species, they can be used as biological agents to produce exogenous proteins in baculoviruses-permissive insect cells or larvae. Different isolates of baculovirus, such as Autographa californica multiple nuclear polyhedrosis virus (AcMNPV) and Bombyx mori (silkworm) nuclear polyhedrosis virus (BmNPV) may be used in exogenous protein expression. Various baculoviral expression systems are commercially available, e.g., from ThermoFisher.

In some embodiments, the proteins described herein (e.g., an Anellovirus ORF molecule, e.g., ORF1, ORF2, ORF2/2, ORF2/3, ORF1/1, or ORF1/2, or a functional fragment or splice variant thereof) may be expressed using a baculovirus expression vector (e.g., a bacmid) that comprises one or more components described herein. For example, a baculovirus expression vector may include one or more of (e.g., all of) a selectable marker (e.g., kanR), an origin of replication (e.g., one or both of a bacterial origin of replication and an insect cell origin of replication), a recombinase recognition site (e.g., an att site), and a promoter. In some embodiments, a baculovirus expression vector (e.g., a bacmid as described herein) can be produced by replacing the naturally occurring wild-type polyhedrin gene, which encodes for baculovirus occlusion bodies, with genes encoding the proteins described herein. In some embodiments, the genes encoding the proteins described herein are cloned into a baculovirus expression vector (e.g., a bacmid as described herein) containing a baculovirus promoter. In some embodiments, the baculovirual vector comprises one or more non-baculoviral promoters, e.g., a mammalian promoter or an Anellovirus promoter. In some embodiments, the genes encoding the proteins described herein are cloned into a donor vector (e.g., as described herein), which is then contacted with an empty baculovirus expression vector (e.g., an empty bacmid) such that the genes encoding the proteins described herein are transferred (e.g., by homologous recombination or transposase activity) from the donor vector into the baculovirus expression vector (e.g., bacmid). In some embodiments, the baculovirus promoter is flanked by baculovirus DNA from the nonessential polyhedrin gene locus. In some embodiments, a protein described herein is under the transcriptional control of the AcNPV polyhedrin promoter in the very late phase of viral replication. In some embodiments, a strong promoter suitable for use in baculoviral expression in insect cells include, but are not limited to, baculovirus p10 promoters, polyhedrin (polh) promoters, p6.9 promoters and capsid protein promoters. Weak promoters suitable for use in baculoviral expression in insect cells include ie1, ie2, ie0, et1, 39K (aka pp31) and gp64 promoters of baculoviruses.

In some embodiments, a recombinant baculovirus is produced by homologous recombination between a baculoviral genome (e.g., a wild-type or mutant baculoviral genome), and a transfer vector. In some embodiments, one or more genes encoding a protein described herein are cloned into the transfer vector. In some embodiments, the transfer vector further contains a baculovirus promoter flanked by DNA from a nonessential gene locus, e.g., polyhedrin gene. In some embodiments, one or more genes encoding a protein described herein are inserted into the baculoviral genome by homologous recombination between the baculoviral genome and the transfer vector. In some embodiments, the baculoviral genome is linearized at one or more unique sites. In some embodiments, the linearized sites are located near the target site for insertion of genes encoding the proteins described herein into the baculoviral genome. In some embodiments, a linearized baculoviral genome missing a fragment of the baculoviral genome downstream from a gene, e.g., polyhedrin gene, can be used for homologous recombination. In some embodiments, the baculoviral genome and transfer vector are co-transfected into insect cells. In some embodiments, the method of producing the recombinant baculovirus comprises the steps of preparing the baculoviral genome for performing homologous recombination with a transfer vector containing the genes encoding one or more protein described herein and co-transfecting the transfer vector and the baculoviral genome DNA into insect cells. In some embodiments, the baculoviral genome comprises a region homologous to a region of the transfer vector. These homologous regions may enhance the probability of recombination between the baculoviral genome and the transfer vector. In some embodiments, the homology region in the transfer vector is located upstream or downstream of the promoter. In some embodiments, to induce homologous recombination, the baculoviral genome, and transfer vector are mixed at a weight ratio of about 1:1 to 10:1.

In some embodiments, a recombinant baculovirus is generated by a method comprising site-specific transposition with Tn7, e.g., whereby the genes encoding the proteins described herein are inserted into bacmid DNA, e.g., propagated in bacteria, e.g., E. coli (e.g., DH 10Bac cells). In some embodiments, the genes encoding the proteins described herein are cloned into a pFASTBAC® vector and transformed into competent cells, e.g., DH10BAC® competent cells, containing the bacmid DNA with a mini-attTn7 target site. In some embodiments, the baculovirus expression vector. e.g., pFASTBAC® vector, may have a promoter, e.g., a dual promoter (e.g., polyhedrin promoter, p10 promoter). Commercially available pFASTBACK donor plasmids include: pFASTBAC 1, pFASTBAC HT, and pFASTBAC DUAL. In some embodiments, recombinant bacmid DNA containing-colonies are identified and bacmid DNA is isolated to transfect insect cells.

In some embodiments, a baculoviral vector is introduced into an insect cell together with a helper nucleic acid. The introduction may be concurrent or sequential. In some embodiments, the helper nucleic acid provides one or more baculoviral proteins, e.g., to promote packaging of the baculoviral vector. In some embodiments, recombinant baculovirus produced in insect cells (e.g., by homologous recombination) is expanded and used to infect insect cells (e.g., in the mid-logarithmic growth phase) for recombinant protein expression. In some embodiments, recombinant bacmid DNA produced by site-specific transposition in bacteria. e.g., E. coli, is used to transfect insect cells with a transfection agent, e.g., Cellfectin® II. Additional information on baculovirus expression systems is discussed in U.S. patent application Ser. Nos. 14/447,341, 14/277,892, and 12/278,916, which are hereby incorporated by reference.

### Insect Cell Systems

The proteins described herein may be expressed in insect cells infected or transfected with recombinant baculovirus or bacmid DNA, e.g., as described above. In some embodiments, insect cells include: the Sf9 and Sf21 cells derived from Spodoptera frugiperda and the Tn-368 and High Five™ BTI-TN-5B1-4 cells (also referred to as Hi5 cells) derived from Trichoplusia ni. In some embodiments, insect cell lines Sf21 and Sf9, derived from the ovaries of the pupal fall army worm Spodoptera frugiperda, can be used for the expression of recombinant proteins using the baculovirus expression system. In some embodiments, Sf21 and Sf9 insect cells may be cultured in commercially available serum-supplemented or serum-free media. Suitable media for culturing insect cells include: Grace's Supplemented (TNM-FH), IPL-41, TC-100, Schneider's Drosophila, SF-900 II SFM, and EXPRESS-FIVE™ SFM. In some embodiments, some serum-free media formulations utilize a phosphate buffer system to maintain a culture pH in the range of 6.0-6.4 (Licari et al. Insect cell hosts for baculovirus expression vectors contain endogenous exoglycosidase activity. Biotechnology Progress 9: 146-152 (1993) and Drugmand et al. Insect cells as factories for biomanufacturing. Biotechnology Advances 30:1140-1157 (2012)) for both cultivation and recombinant protein production. In some embodiments, a pH of 6.0-6.8 for cultivating various insect cell lines may be used. In some embodiments, insect cells are cultivated in suspension or as a monolaver at a temperature between 25° to 30° ° C. with aeration. Additional information on insect cells is discussed, for example, in U.S. patent application Ser. Nos. 14/564,512 and 14/775,154, each of which is hereby incorporated by reference.

### Mammalian Cell Systems

In some embodiments, the proteins described herein may be expressed in vitro in animal cell lines infected or transfected with a vector encoding the protein, e.g., as described herein. Animal cell lines envisaged in the context of the present disclosure include porcine cell lines, e.g., immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines suitable for expressing the proteins described herein include, but are not limited to cell lines of human or primate origin, such as human or primate kidney carcinoma cell lines.

### Genetic Element Constructs, e.g., for Assembly of Anellovectors

The genetic element of an anellovector as described herein may be produced from a genetic element construct that comprises a genetic element region and optionally other sequence such as vector backbone. Generally, the genetic element construct comprises an Anellovirus 5′ UTR (e.g., as described herein). A genetic element construct may be any nucleic acid construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, bacmid, or a minicircle, e.g., as described herein). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

In some aspects, the present disclosure provides a method for replication and propagation of the anellovector as described herein (e.g., in a cell culture system), which may comprise one or more of the following steps: (a) introducing (e.g., transfecting) a genetic element (e.g., linearized) into a cell line sensitive to anellovector infection; (b) harvesting the cells and optionally isolating cells showing the presence of the genetic element; (c) culturing the cells obtained in step (b) (e.g., for at least three days, such as at least one week or longer), depending on experimental conditions and gene expression; and (d) harvesting the cells of step (c), e.g., as described herein.

### Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp. In some embodiments, the genetic element can be excised from a plasmid (e.g., by in vitro circularization), for example, to form a minicircle, e.g., as described herein. In embodiments, excision of the genetic element separates the genetic element sequence from the plasmid backbone (e.g., separates the genetic element from a bacterial backbone).

### Small Circular Nucleic Acid Constructs

In some embodiments, the genetic element construct is a circular nucleic acid construct, e.g., lacking a backbone (e.g., lacking a bacterial origin of replication and/or selectable marker). In embodiments, the genetic element is a double-stranded circular nucleic acid construct. In embodiments, the double-stranded circular nucleic acid construct is produced by in vitro circularization (IVC), e.g., as described herein. In embodiments, the double-stranded circular nucleic acid construct can be introduced into a host cell, in which it can be converted into or used as a template for generating single-stranded circular genetic elements, e.g., as described herein. In some embodiments, the circular nucleic acid construct does not comprise a plasmid backbone or a functional fragment thereof. In some embodiments, the circular nucleic acid construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular nucleic acid construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular nucleic acid construct is between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular nucleic acid construct is a minicircle.

### In Vitro Circularization

In some instances, the genetic element to be packaged into a proteinaceous exterior is a single stranded circular DNA. The genetic element may, in some instances, be introduced into a host cell via a genetic element construct having a form other than a single stranded circular DNA. For example, the genetic element construct may be a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or TraI, e.g., as described in Wawrzyniak et al. 2017, Front. Microbiol. 8: 2353; incorporated herein by reference with respect to the listed enzymes). In some embodiments, the double-stranded circular DNA is produced by in vitro circularization (IVC), e.g., as described in Example 15.

Generally, in vitro circularized DNA constructs can be produced by digesting a genetic element construct (e.g., a plasmid comprising the sequence of a genetic element) to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an anellovector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

### Tandem Constructs

In some embodiments, a genetic element construct comprises a first copy of a genetic element sequence (e.g., the nucleic acid sequence of a genetic element, e.g., as described herein) and at least a portion of a second copy of a genetic element sequence (e.g., the nucleic acid sequence of the same genetic element, or the nucleic acid sequence of a different genetic element), arranged in tandem. Genetic element constructs having such a structure are generally referred to herein as tandem constructs. Such tandem constructs are used for producing an anellovector genetic element. The first copy of the genetic element sequence and the second copy of the genetic element sequence may, in some instances, be immediately adjacent to each other on the genetic acid construct. In other instances, the first copy of the genetic element sequence and the second copy of the genetic element sequence may be separated, e.g., by a spacer sequence. In some embodiments, the second copy of the genetic element sequence, or the portion thereof, comprises an upstream replication-facilitating sequence (uRFS), e.g., as described herein. In some embodiments, the second copy of the genetic element sequence, or the portion thereof, comprises a downstream replication-facilitating sequence (dRFS), e.g., as described herein. In some embodiments, the uRFS and/or dRFS comprises an origin of replication (e.g., a mammalian origin of replication, an insect origin of replication, or a viral origin of replication, e.g., a non-Anellovirus origin of replication, e.g., as described herein) or portion thereof. In some embodiments, the uRFS and/or dRFS does not comprise an origin of replication. In some embodiments, the uRFS and/or dRFS comprises a hairpin loop (e.g., in the 5′ UTR). In some embodiments, a tandem construct produces higher levels of a genetic element than an otherwise similar construct lacking the second copy of the genetic element or portion thereof. Without being bound by theory, a tandem construct described herein may, in some embodiments, replicate by rolling circle replication. In some embodiments, a tandem construct is a plasmid. In some embodiments, a tandem construct is circular. In some embodiments, a tandem construct is linear. In some embodiments, a tandem construct is single-stranded. In some embodiments, a tandem construct is double-stranded. In some embodiments, a tandem construct is DNA.

A tandem construct may, in some instances, include a first copy of the sequence of the genetic element and a second copy of the sequence of the genetic element, or a portion thereof. It is understood that the second copy can be an identical copy of the first copy or a portion thereof, or can comprise one or more sequence differences, e.g., substitutions, additions, or deletions. In some instances, the second copy of the genetic element sequence or portion thereof is positioned 5′ relative to the first copy of the genetic element sequence. In some instances, the second copy of the genetic element sequence or portion thereof is positioned 3′ relative to the first copy of the genetic element sequence. In some instances, the second copy of the genetic element sequence or portion thereof and the first copy of the genetic element sequence are adjacent to each other in the tandem construct. In some instances, the second copy of the genetic element sequence or portion thereof and the first copy of the genetic element sequence are separated, e.g., by a spacer sequence

In some embodiments, the tandem constructs described herein can be used to produce the genetic element of a vector (e.g., anellovector), vehicle, or particle (e.g., viral particle) comprising a capsid (e.g., a capsid comprising an Anellovirus ORF, e.g., an ORF1 molecule, e.g., as described herein) encapsulating a genetic element comprising a protein binding sequence that binds to the capsid and a heterologous (e.g., relative to the Anellovirus from which the ORF1 molecule was derived) sequence encoding a therapeutic effector. In embodiments, the vector is capable of delivering the genetic element into a mammalian, e.g., human, cell. In some embodiments, the genetic element has less than about 50% (e.g., less than 50%, 40%, 30%, 25%, 20%, 15%, 10%, 9%, 8%, 7%, 6%, 5.5%, 5%, 4.5%, 4%, 3.5%, 3%, 2.5%, 2%, 1.5%, or less) identity to a wild type Anellovirus genome sequence. In some embodiments, the genetic element has no more than 1.5%, 2%, 2.5%, 3%, 3.5%, 4%, 4.5%, 5%, 5.5%, 6%, 7%, 8%, 9%, 10%, 15%, 20%, 25%, 30%, 40%, 50%, 60%, 70%, 75%, or 80% identity to a wild type Anellovirus genome sequence. In some embodiments, the genetic element has greater than about 2000, 3000, 4000, 4500, or 5000 contiguous nucleotides of non-Anellovirus genome sequence. In some embodiments, the genetic element has greater than about 2000 to 5000, 2500 to 4500, 3000 to 4500, 2500 to 4500, 3500, or 4000, 4500 (e.g., between about 3000 to 4500) nucleotides nucleotides of non-Anellovirus genome sequence.

In some embodiments of the systems and methods herein, a vector (e.g., an anellovector) is made by introducing into a cell a first nucleic acid molecule that is a genetic element or genetic element construct, e.g., a tandem construct, and a second nucleic acid molecule encoding one or more additional proteins (e.g., a Rep molecule and/or a capsid protein), e.g., as described herein. In some embodiments, the first nucleic acid molecule and the second nucleic acid molecule are attached to each other (e.g., in a genetic element construct described herein, e.g., in cis). In some embodiments, the first nucleic acid molecule and the second nucleic acid molecule are separate (e.g, in trans). In some embodiments, the first nucleic acid molecule is a plasmid, cosmid, bacmid, minicircle, or artificial chromosome. In some embodiments, the second nucleic acid molecule is a plasmid, cosmid, bacmid, minicircle, or artificial chromosome. In some embodiments, the second nucleic acid molecule is integrated into the genome of the host cell.

In some embodiments, the method further includes introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is or comprises or is part of a helper construct, helper virus or other helper vector, e.g., as described herein.

### Cis/Trans Constructs

In some embodiments, a genetic element construct as described herein comprises one or more sequences encoding one or more Anellovirus ORFs, e.g., proteinaceous exterior components (e.g., polypeptides encoded by an Anellovirus ORF1 nucleic acid, e.g., as described herein). For example, the genetic element construct may comprise a nucleic acid sequence encoding an Anellovirus ORF1 molecule. Such genetic element constructs can be suitable for introducing the genetic element and the Anellovirus ORF(s) into a host cell in cis. In other embodiments, a genetic element construct as described herein does not comprise sequences encoding one or more Anellovirus ORFs, e.g., proteinaccous exterior components (e.g., polypeptides encoded by an Anellovirus ORF1 nucleic acid, e.g., as described herein). For example, the genetic element construct may not comprise a nucleic acid sequence encoding an Anellovirus ORF1 molecule. Such genetic element constructs can be suitable for introducing the genetic element into a host cell, with the one or more Anellovirus ORFs to be provided in trans (e.g., via introduction of a second nucleic acid construct encoding one or more of the Anellovirus ORFs, or via an Anellovirus ORF cassette integrated into the genome of the host cell). In some embodiments, an ORF1 molecule is provided in trans, e.g., as described herein. In some embodiments, an ORF2 molecule is provided in trans, e.g., as described herein. In some embodiments, an ORF1 molecule and an ORF1 molecule are both provided in trans, e.g., as described herein.

In some embodiments, the genetic element construct comprises a sequence encoding an Anellovirus ORF1 molecule, or a splice variant or functional fragment thereof (e.g., a jelly-roll region, e.g., as described herein). In embodiments, the portion of the genetic element that does not comprise the sequence of the genetic element comprises the sequence encoding the Anellovirus ORF1 molecule, or splice variant or functional fragment thereof (e.g., in a cassette comprising a promoter and the sequence encoding the Anellovirus ORF1 molecule, or splice variant or functional fragment thereof). In further embodiments, the portion of the construct comprising the sequence of the genetic element comprises a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-component anellovector (e.g., an anellovector that upon infecting a cell, enables the cell to produce additional copies of the anellovector without introducing further nucleic acid constructs, e.g., encoding one or more Anellovirus ORFs as described herein, into the cell).

In other embodiments, the genetic element does not comprise a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent anellovector (e.g., an anellovector that, upon infecting a cell, does not enable the infected cell to produce additional anellovectors, e.g., in the absence of one or more additional constructs, e.g., encoding one or more Anellovirus ORFs as described herein).

### Expression Cassettes

In some embodiments, a genetic element construct comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of an Anellovirus protein (e.g., an Anellovirus ORF1, ORF2, ORF2/2, ORF2/3, ORF1/1, or ORF1/2, or a functional fragment thereof). The expression cassettes may, in some embodiments, be located within the genetic element sequence. In embodiments, an expression cassette for an effector is located within the genetic element sequence. In embodiments, an expression cassette for an Anellovirus protein is located within the genetic element sequence. In other embodiments, the expression cassettes are located at a position within the genetic element construct outside of the sequence of the genetic element (e.g., in the backbone). In embodiments, an expression cassette for an Anellovirus protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or an Anellovirus protein (e.g., a sequence encoding an Anellovirus ORF1, ORF2, ORF2/2, ORF2/3, ORF1/1, or ORF1/2, or a functional fragment thereof). Exemplary promoters that can be included in an polypeptide expression cassette (e.g., to drive expression of the polypeptide) include, without limitation, constitutive promoters (e.g., CMV, RSV, PGK, EFla, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

### Design and Production of a Genetic Element Construct

Various methods are available for synthesizing a genetic element construct. For instance, the genetic element construct sequence may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the genetic element construct. The segments or ORFs may be assembled into the genetic element construct, e.g., by in vitro recombination or unique restriction sites at 5′ and 3′ ends to enable ligation.

The genetic element construct can be synthesized with a design algorithm that parses the construct sequence into oligo-length fragments, creating suitable design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element construct or segment of the genetic element construct may be sequence verified. In some embodiments, high-throughput sequencing of RNA or DNA can take place using Any Dot.chips (Genovoxx, Germany), which allows for the monitoring of biological processes (e.g., miRNA expression or allele variability (SNP detection). Other high-throughput sequencing systems include those disclosed in Venter, J., et al. Science 16 Feb. 2001; Adams, M. et al, Science 24 Mar. 2000; and M. J. Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. In some embodiments, shotgun sequencing is performed.

A genetic element construct can be designed such that factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, an effector is inserted into a specific site in the genome. In some embodiments, one or more viral ORFs are replaced with an effector.

In another example, when replication or packaging factors are supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

In some embodiments, the genetic element construct may be designed using computer-aided design tools.

General methods of making constructs are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

### Effectors

The compositions and methods described herein can be used to produce a genetic element of an anellovector comprising a sequence encoding an effector (e.g., an exogenous effector or an endogenous effector), e.g., as described herein. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In some embodiments, the effector-encoding sequence may be inserted into the genetic element e.g., at a non-coding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the effector-encoding sequence may be inserted into the genetic element, e.g., in a coding sequence (e.g., in a sequence encoding an Anellovirus ORF1, ORF1/1, ORF1/2, ORF2, ORF2/2, ORF2/3, and/or ORF2t/3, e.g., as described herein). In some embodiments, the effector-encoding sequence replaces all or a part of the open reading frame. In some embodiments, the genetic element comprises a regulatory sequence (e.g., a promoter or enhancer, e.g., as described herein) operably linked to the effector-encoding sequence.

### Host Cells

The anellovectors described herein can be produced, for example, in a host cell. Generally, a host cell is provided that comprises an anellovector genetic element and the components of an anellovector proteinaceous exterior (e.g., a polypeptide encoded by an Anellovirus ORF1 nucleic acid or an Anellovirus ORF1 molecule). The host cell is then incubated under conditions suitable for enclosure of the genetic element within the proteinaceous exterior (e.g., culture conditions as described herein). In some embodiments, the host cell is further incubated under conditions suitable for release of the anellovector from the host cell, e.g., into the surrounding supernatant. In some embodiments, the host cell is lysed for harvest of anellovectors from the cell lysate. In some embodiments, an anellovector may be introduced to a host cell line grown to a high cell density. In some embodiments, a host cell is an Expi-293 cell.

Introduction of Genetic Elements into Host Cells

The genetic element, or a nucleic acid construct comprising the sequence of a genetic element, may be introduced into a host cell. In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element is introduced into the host cell by contacting the host cell with an anellovector comprising the genetic element

In embodiments, the genetic element construct is capable of replication once introduced into the host cell. In embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the anellovector. To this end, cell lines that express an anellovector polymerase protein may be utilized as appropriate host cells. Host cells may be similarly engineered to provide other viral functions or additional functions.

To prepare the anellovector disclosed herein, a genetic element construct may be used to transfect cells that provide anellovector proteins and functions required for replication and production. Alternatively, cells may be transfected with a second construct (e.g., a virus) providing anellovector proteins and functions before, during, or after transfection by the genetic element or vector comprising the genetic element disclosed herein. In some embodiments, the second construct may be useful to complement production of an incomplete viral particle. The second construct (e.g., virus) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the anellovector. In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into anellovectors using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845: International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

### Methods for Providing Protein(s) in Cis or Trans

In some embodiments (e.g., cis embodiments described herein), the genetic element construct further comprises one or more expression cassettes comprising a coding sequence for an Anellovirus ORF (e.g., an Anellovirus ORF1, ORF2, ORF2/2, ORF2/3, ORF1/1, or ORF1/2, or a functional fragment thereof). In embodiments, the genetic element construct comprises an expression cassette comprising a coding sequence for an Anellovirus ORF1, or a splice variant or functional fragment thereof. Such genetic element constructs, which comprise expression cassettes for the effector as well as the one or more Anellovirus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element does not comprise an expression cassette comprising a coding sequence for one or more Anellovirus ORFs (e.g., an Anellovirus ORF1, ORF2, ORF2/2, ORF2/3, ORF1/1, or ORF1/2, or a functional fragment thereof). In embodiments, the genetic element construct does not comprise an expression cassette comprising a coding sequence for an Anellovirus ORF1, or a splice variant or functional fragment thereof. Such genetic element constructs, which comprise expression cassettes for the effector but lack expression cassettes for one or more Anellovirus ORFs (e.g., Anellovirus ORF1 or a splice variant or functional fragment thereof), may be introduced into host cells. Host cells comprising such genetic element constructs may, in some instances, require additional nucleic acid constructs or integration of expression cassettes into the host cell genome for production of one or more components of the anellovector (e.g., the proteinaceous exterior proteins). In some embodiments, host cells comprising such genetic element constructs are incapable of enclosure of the genetic elements within proteinaceous exteriors in the absence of an additional nucleic construct encoding an Anellovirus ORF1 molecule. In other words, such genetic element constructs may be used for trans anellovector production methods in host cells, e.g., as described herein.

In some embodiments (e.g., cis embodiments described herein), the genetic element construct further comprises one or more expression cassettes comprising a coding sequence for one or more non-Anellovirus ORF (e.g., a non-Anellovirus Rep molecule, e.g., an AAV Rep molecule, e.g., an AAV Rep protein, e.g., an AAV Rep2 protein). Such genetic element constructs, which comprise expression cassettes for the effector as well as the one or more non-Anellovirus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element does not comprise an expression cassette comprising a coding sequence for one or more non-Anellovirus ORFs (e.g., a non-Anellovirus Rep molecule, e.g., an AAV Rep molecule, e.g., an AAV Rep protein, e.g., an AAV Rep2 protein). Such genetic element constructs, which comprise expression cassettes for the effector but lack expression cassettes for one or more non-Anellovirus ORFs (e.g., a non-Anellovirus Rep molecule, e.g., an AAV Rep molecule, e.g., an AAV Rep protein, e.g., an AAV Rep2 protein), may be introduced into host cells. Host cells comprising such genetic element constructs may, in some instances, require additional nucleic acid constructs or integration of expression cassettes into the host cell genome for production of one or more components of the anellovector (e.g., for replication of the genetic element). In some embodiments, host cells comprising such genetic element constructs are incapable of replicating the genetic elements in the absence of an additional nucleic construct, e.g., encoding a non-Anellovirus Rep molecule, e.g., an AAV Rep molecule, e.g., an AAV Rep protein, e.g., an AAV Rep2 protein. In other words, such genetic element constructs may be used for trans anellovector production methods in host cells, e.g., as described herein.

### Exemplary Cell Types

Exemplary host cells suitable for production of anellovectors include, without limitation, mammalian cells, e.g., human cells and insect cells. In some embodiments, the host cell is a human cell or cell line. In some embodiments, the cell is an immune cell or cell line, e.g., a T cell or cell line, a cancer cell line, a hepatic cell or cell line, a neuron, a glial cell, a skin cell, an epithelial cell, a mesenchymal cell, a blood cell, an endothelial cell, an eye cell, a gastrointestinal cell, a progenitor cell, a precursor cell, a stem cell, a lung cell, a cardiac cell, or a muscle cell. In some embodiments, the host cell is an animal cell (e.g., a mouse cell, rat cell, rabbit cell, or hamster cell, or insect cell).

In some embodiments, the host cell is a lymphoid cell. In some embodiments, the host cell is a T cell or an immortalized T cell. In embodiments, the host cell is a Jurkat cell. In embodiments, the host cell is a MOLT cell (e.g., a MOLT-4 or a MOLT-3 cell). In embodiments, the host cell is a MOLT-4 cell. In embodiments, the host cell is a MOLT-3 cell. In some embodiments, the host cell is an acute lymphoblastic leukemia (ALL) cell, e.g., a MOLT cell, e.g., a MOLT-4 or MOLT-3 cell. In some embodiments, the host cell is a B cell or an immortalized B cell. In some embodiments, the host cell comprises a genetic element construct (e.g., as described herein).

In some embodiments, the host cell is a MOLT cell (e.g., a MOLT-4 or a MOLT-3 cell).

In some embodiments, the host cell is an acute lymphoblastic leukemia (ALL) cell, e.g., a MOLT cell, e.g., a MOLT-4 or MOLT-3 cell.

In some embodiments, the host cell is an Expi-293 cell. In some embodiments, the host cell is an Expi-293F cell.

In an aspect, the present disclosure provides a method of manufacturing an anellovector comprising a genetic element enclosed in a proteinaceous exterior, the method comprising providing a MOLT-4 cell comprising an anellovector genetic element, and incubating the MOLT-4 cell under conditions that allow the anellovector genetic element to become enclosed in a proteinaceous exterior in the MOLT-4 cell. In some embodiments, the MOLT-4 cell further comprises one or more Anellovirus proteins (e.g., an Anellovirus ORF1 molecule) that form part or all of the proteinaceous exterior. In some embodiments, the anellovector genetic element is produced in the MOLT-4 cell, e.g., from a genetic element construct (e.g., as described herein). In some embodiments, the method further comprises introducing the anellovector genetic element construct into the MOLT-4 cell.

In an aspect, the present disclosure provides a method of manufacturing an anellovector comprising a genetic element enclosed in a proteinaceous exterior, the method comprising providing a MOLT-3 cell comprising an anellovector genetic element, and incubating the MOLT-3 cell under conditions that allow the anellovector genetic element to become enclosed in a proteinaceous exterior in the MOLT-3 cell. In some embodiments, the MOLT-3 cell further comprises one or more Anellovirus proteins (e.g., an Anellovirus ORF1 molecule) that form part or all of the proteinaceous exterior. In some embodiments, the anellovector genetic element is produced in the MOLT-3 cell, e.g., from a genetic element construct (e.g., as described herein). In some embodiments, the method further comprises introducing the anellovector genetic element construct into the MOLT-3 cell.

In some embodiments, the host cell is a human cell. In embodiments, the host cell is a HEK293T cell, HEK293F cell, A549 cell, Jurkat cell, Raji cell, Chang cell, HeLa cell Phoenix cell, MRC-5 cell, NCI-H292 cell, or Wi38 cell. In some embodiments, the host cell is a non-human primate cell (e.g., a Vero cell, CV-1 cell, or LLCMK2 cell). In some embodiments, the host cell is a murine cell (e.g., a McCoy cell). In some embodiments, the host cell is a hamster cell (e.g., a CHO cell or BHK 21 cell). In some embodiments, the host cell is a MARC-145, MDBK, RK-13, or EEL cell. In some embodiments, the host cell is an epithelial cell (e.g., a cell line of epithelial lineage).

In some embodiments, the anellovector is cultivated in continuous animal cell line (e.g., immortalized cell lines that can be serially propagated). According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST.

### Culture Conditions

Host cells comprising a genetic element and components of a proteinaceous exterior can be incubated under conditions suitable for enclosure of the genetic element within the proteinaceous exterior, thereby producing an anellovector. Suitable culture conditions include those described, e.g., in any of Examples 4, 5, 7, 8, 9, 10, 11, or 15. In some embodiments, the host cells are incubated in liquid media (e.g., Grace's Supplemented (TNM-FH), IPL-41, TC-100, Schneider's Drosophila, SF-900 II SFM, or and EXPRESS-FIVE™ SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release anellovectors produced therein into the surrounding supernatant.

The production of anellovector-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of anellovectors.

### Harvest

Anellovectors produced by host cells can be harvested, e.g., according to methods known in the art. For example, anellovectors released into the surrounding supernatant by host cells in culture can be harvested from the supernatant (e.g., as described in Example 4). In some embodiments, the supernatant is separated from the host cells to obtain the anellovectors. In some embodiments, the host cells are lysed before or during harvest. In some embodiments, the anellovectors are harvested from the host cell lysates (e.g., as described in Example 10). In some embodiments, the anellovectors are harvested from both the host cell lysates and the supernatant. In some embodiments, the purification and isolation of anellovectors is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the anellovector may be harvested and/or purified by separation of solutes based on biophysical properties, e.g., ion exchange chromatography or tangential flow filtration, prior to formulation with a pharmaceutical excipient.

### In Vitro Assembly Methods

An anellovector may be produced, e.g., by in vitro assembly, e.g., in a cell-free suspension or in a supernatant. In some embodiments, the genetic element is contacted to an ORF1 molecule in vitro. e.g., under conditions that allow for assembly.

In some embodiments, baculovirus constructs are used to produce Anellovirus proteins. These proteins may then be used, e.g., for in vitro assembly to encapsidate a genetic element, e.g., a genetic element comprising RNA. In some embodiments, a polynucleotide encoding one or more Anellovirus protein is fused to a promoter for expression in a host cell, e.g., an insect or animal cell. In some embodiments, the polynucleotide is cloned into a baculovirus expression system. In some embodiments, a host cell, e.g., an insect cell is infected with the baculovirus expression system and incubated for a period of time. In some embodiments, an infected cell is incubated for about 1, 2, 3, 4, 5, 10, 15, or 20 days. In some embodiments, an infected cell is lysed to recover the Anellovirus protein.

In some embodiments, an isolated Anellovirus protein is purified. In some embodiments, an Anellovirus protein is purified using purification techniques including but not limited to chelating purification, heparin purification, gradient sedimentation purification, and/or SEC purification. In some embodiments, a purified Anellovirus protein is mixed with a genetic element to encapsidate the genetic element, e.g., a genetic element comprising RNA. In some embodiments, a genetic element is encapisdated using an ORF1 protein, ORF2 protein, or modified version thereof. In some embodiments two nucleic acids are encapsidated. For instance, the first nucleic acid may be an mRNA e.g., chemically modified mRNA, and the second nucleic acid may be DNA.

In some embodiments, DNA encoding Anellovirus (AV) ORF1 (e.g., wildtype ORF1 protein, ORF1 proteins harboring mutations, e.g., to improve assembly efficiency, yield or stability, chimeric ORF1 protein, or fragments thereof) are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding AV ORF1 may be untagged. In some embodiments, DNA encoding AV ORF1 may contain tags fused N-terminally and/or C-terminally. In some embodiments, DNA encoding AV ORF1 may harbor mutations, insertions or deletions within the ORF1 protein to introduce a tag, e.g., to aid in purification and/or identity determination, e.g., through immunostaining assays (including but not limited to ELISA or Western Blot). In some embodiments, DNA encoding AV ORF1 may be expressed alone or in combination with any number of helper proteins. In some embodiments, DNA encoding AV ORF1 is expressed in combination with AV ORF2 and/or ORF3 proteins.

In some embodiments, ORF1 proteins harboring mutations to improve assembly efficiency may include, but are not limited to, ORF1 proteins that harbor mutations introduced into the N-terminal Arginine Arm (ARG arm) to alter the pI of the ARG arm permitting pH sensitive nucleic acid binding to trigger particle assembly (SEQ ID 3-5). In some embodiments, ORF1 proteins harboring mutations that improve stability may include mutations to an interprotomer contacting beta strands F and G of the canonical jellyroll beta-barrel to alter hydrophobic state of the protomer surface and improve thermodynamic favorability of capsid formation.

In some embodiments, chimeric ORF1 proteins may include, but are not limited to, ORF1 proteins which have a portion or portions of their sequence replaced with comparable portions from another capsid protein, e.g., Beak and Feather Disease Virus (BFDV) capsid protein, or Hepatitis E capsid protein, e.g., ARG arm or F and G beta strands of Ring 9 ORF1 replaced with the comparable components from BFDV capsid protein. In some embodiments, chimeric ORF1 proteins may also include ORF1 proteins which have a portion or portions of their sequence replaced with comparable portions of another AV ORF1 protein (e.g., jellyroll fragments or the C-terminal portion of Ring 2 ORF1 replaced with comparable portions of Ring 9 ORF1.

In some embodiments, the present disclosure describes a method of making an anellovector, the method comprising: (a) providing a mixture comprising: (i) a genetic element comprising RNA, and (ii) an ORF1 molecule; and (b) incubating the mixture under conditions suitable for enclosing the genetic element within a proteinaceous exterior comprising the ORF1 molecule, thereby making an anellovector; optionally wherein the mixture is not comprised in a cell. In some embodiments, the method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, the method further comprises, prior to the providing of (a), purifying the ORF1 molecule expressed by the host cell. In some embodiments, the method is performed in a cell-free system. In some embodiments, the present disclosure describes a method of manufacturing an anellovector composition, comprising: (a) providing a plurality of anellovectors or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anellovectors, e.g., as a pharmaceutical composition suitable for administration to a subject, e.g., if one or more of the parameters of (b) meet a specified threshold.

### Enrichment and Purification

Harvested anellovectors can be purified and/or enriched, e.g., to produce an anellovector preparation. In some embodiments, the harvested anellovectors are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested anellovectors are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### III. Vectors

The genetic element described herein may be included in a vector. Suitable vectors as well as methods for their manufacture and their use are well known in the prior art.

In one aspect, the invention includes a vector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid.

The genetic element or any of the sequences within the genetic element can be obtained using any suitable method. Various recombinant methods are known in the art, such as, for example screening libraries from cells harboring viral sequences, deriving the sequences from a vector known to include the same, or isolating directly from cells and tissues containing the same, using standard techniques. Alternatively or in combination, part or all of the genetic element can be produced synthetically, rather than cloned.

In some embodiments, the vector includes regulatory elements, nucleic acid sequences homologous to target genes, and various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vector is substantially non-pathogenic and/or substantially non-integrating in a host cell or is substantially non-immunogenic in a host.

In some embodiments, the vector is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### IV. Compositions

The anellovector or vector described herein may also be included in pharmaceutical compositions with a pharmaceutical excipient, e.g., as described herein. In some embodiments, the pharmaceutical composition comprises at least 10⁵, 10⁶, 10⁷, 10⁸, 10⁹, 10¹⁰, 10¹¹, 10¹², 10¹³, 10¹⁴, or 10¹⁵ anellovectors. In some embodiments, the pharmaceutical composition comprises about 10⁵-10¹⁵, 10⁵-10¹⁰, or 10¹⁰-10¹⁵ anellovectors. In some embodiments, the pharmaceutical composition comprises about 10⁸ (e.g., about 10⁵, 10⁶, 10⁷, 10⁸, 10⁹, or 10¹⁰) genomic equivalents/mL of the anellovector. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, 10⁵-10⁹, 10⁵-10¹¹, 10⁵-10¹², 10⁵-10¹³, 10⁵-10¹⁴, 10⁵-10¹⁵, or 10¹⁰-10¹⁵ genomic equivalents/mL of the anellovector, e.g., as determined according to the method of Example 18. In some embodiments, the pharmaceutical composition comprises sufficient anellovectors to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the anellovectors per cell to a population of the eukaryotic cells. In some embodiments, the pharmaceutical composition comprises sufficient anellovectors to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the anellovectors per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates.

In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:
a) an anellovector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaccous exterior that is associated with, e.g., envelops or encloses, the genetic element; and
b) a pharmaceutical excipient.

### Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi: 10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi: 10.1155/2011/469679 for review).

In embodiments, lipids may be used to form lipid microparticles. Lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi: 10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention. In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means+/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

### Carriers

A composition (e.g., pharmaceutical composition) described herein may comprise, be formulated with, and/or be delivered in, a carrier. In one aspect, the invention includes a composition, e.g., a pharmaceutical composition, comprising a carrier (e.g., a vesicle, a liposome, a lipid nanoparticle, an exosome, a red blood cell, an exosome (e.g., a mammalian or plant exosome), a fusosome) comprising (e.g., encapsulating) a composition described herein (e.g., an anellovector, Anellovirus, anellovector, or genetic element described herein).

In some embodiments, the compositions and systems described herein can be formulated in liposomes or other similar vesicles. Generally, liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes generally have one or more (e.g., all) of the following characteristics: biocompatibility, nontoxicity, can deliver both hydrophilic and lipophilic drug molecules, can protect their cargo from degradation by plasma enzymes, and can transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679; and Zylberberg & Matosevic. 2016. Drug Delivery, 23:9, 3319-3329, doi: 10.1080/10717544.2016.1177136).

Vesicles can be made from several different types of lipids; however, phospholipids are most commonly used to generate liposomes as drug carriers. Methods for preparation of multilamellar vesicle lipids are known (see, for example, U.S. Pat. No. 6,693,086, the teachings of which relating to multilamellar vesicle lipid preparation are incorporated herein by reference). Although vesicle formation can be spontaneous when a lipid film is mixed with an aquecous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by, e.g., extruding through filters of decreasing size, as described in Templeton et al., Nature Biotech, 15:647-652, 1997.

Lipid nanoparticles (LNPs) are another example of a carrier that provides a biocompatible and biodegradable delivery system for the pharmaceutical compositions described herein. See, e.g., Gordillo-Galeano et al. European Journal of Pharmaceutics and Biopharmaceutics. Volume 133, December 2018, Pages 285-308. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi: 10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for a composition described herein. See, e.g., WO2015073587: WO2017123646: WO2017123644: WO2018102740; WO2016183482: WO2015153102: WO2018151829: WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a composition described herein.

### Membrane Penetrating Polypeptides

In some embodiments, the composition further comprises a membrane penetrating polypeptide (MPP) to carry the components into cells or across a membrane, e.g., cell or nuclear membrane. Membrane penetrating polypeptides that are capable of facilitating transport of substances across a membrane include, but are not limited to, cell-penetrating peptides (CPPs)(see, e.g., U.S. Pat. No. 8,603,966), fusion peptides for plant intracellular delivery (see, e.g., Ng et al., PLOS One, 2016, 11:e0154081), protein transduction domains, Trojan peptides, and membrane translocation signals (MTS) (see, e.g., Tung et al., Advanced Drug Delivery Reviews 55:281-294 (2003)). Some MPP are rich in amino acids, such as arginine, with positively charged side chains.

Membrane penetrating polypeptides have the ability of inducing membrane penetration of a component and allow macromolecular translocation within cells of multiple tissues in vivo upon systemic administration. A membrane penetrating polypeptide may also refer to a peptide which, when brought into contact with a cell under appropriate conditions, passes from the external environment in the intracellular environment, including the cytoplasm, organelles such as mitochondria, or the nucleus of the cell, in amounts significantly greater than would be reached with passive diffusion.

Components transported across a membrane may be reversibly or irreversibly linked to the membrane penetrating polypeptide. A linker may be a chemical bond, e.g., one or more covalent bonds or non-covalent bonds. In some embodiments, the linker is a peptide linker. Such a linker may be between 2-30 amino acids, or longer. The linker includes flexible, rigid or cleavable linkers.

### Combinations

In one aspect, the anellovector or composition comprising a anellovector described herein may also include one or more heterologous moiety. In one aspect, the anellovector or composition comprising a anellovector described herein may also include one or more heterologous moiety in a fusion. In some embodiments, a heterologous moiety may be linked with the genetic element. In some embodiments, a heterologous moiety may be enclosed in the proteinaceous exterior as part of the anellovector. In some embodiments, a heterologous moiety may be administered with the anellovector.

In one aspect, the invention includes a cell or tissue comprising any one of the anellovectors and heterologous moieties described herein.

In another aspect, the invention includes a pharmaceutical composition comprising a anellovector and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein. In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

### Viruses

In some embodiments, the composition may further comprise a virus as a heterologous moiety, e.g., a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the composition may further comprise a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the composition may further comprise an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus. In some embodiments, the anellovector is administered with a virus as a heterologous moiety.

In some embodiments, the heterologous moiety may comprise a non-pathogenic, e.g., symbiotic, commensal, native, virus. In some embodiments, the non-pathogenic virus is one or more anelloviruses, e.g., Alphatorquevirus (TT), Betatorquevirus (TTM), and Gammatorquevirus (TTMD). In some embodiments, the anellovirus may include a Torque Teno Virus (TT), a SEN virus, a Sentinel virus, a TTV-like mini virus, a TT virus, a TT virus genotype 6, a TT virus group, a TTV-like virus DXL1, a TTV-like virus DXL2, a Torque Teno-like Mini Virus (TTM), or a Torque Teno-like Midi Virus (TTMD). In some embodiments, the non-pathogenic virus comprises one or more sequences having at least at least about 60%, 70% 80%, 85%, 90% 95%, 96%, 97%, 98% and 99% nucleotide sequence identity to any one of the nucleotide sequences described herein, e.g., as listed in any of Tables N1-N1417.

In some embodiments, the heterologous moiety may comprise one or more viruses that are identified as lacking in the subject. For example, a subject identified as having dyvirosis may be administered a composition comprising an anellovector and one or more viral components or viruses that are imbalanced in the subject or having a ratio that differs from a reference value, e.g., a healthy subject.

In some embodiments, the heterologous moiety may comprise one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. In some embodiments, the anellovector or the virus is defective, or requires assistance in order to produce infectious particles. Such assistance can be provided, e.g., by using helper cell lines that contain a nucleic acid, e.g., plasmids or DNA integrated into the genome, encoding one or more of (e.g., all of) the structural genes of the replication defective anellovector or virus under the control of regulatory sequences within the LTR. Suitable cell lines for replicating the anellovectors described herein include cell lines known in the art, e.g., A549 cells, which can be modified as described herein.

### Targeting Moiety

In some embodiments, the composition or anellovector described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the anellovector or genetic element. For example, a targeting moiety may include a therapeutic that interacts with a specific molecule of interest to increase, decrease or otherwise modulate its function.

### Tagging or Monitoring Moiety

In some embodiments, the composition or anellovector described herein may further comprise a tag to label or monitor the anellovector or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligasc) or chemical modifications (such as reaction with FLASH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

### Nanoparticles

In some embodiments, the composition or anellovector described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

### Small Molecules

In some embodiments, the composition or anellovector described herein may further comprise a small molecule. Small molecule moieties include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids; Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, the small molecule is an epigenetic modifying agent, for example such as those described in de Groote et al. Nuc. Acids Res. (2012): 1-18. Exemplary small molecule epigenetic modifying agents are described, e.g., in Lu et al. J. Biomolecular Screening 17.5(2012):555-71, e.g., at Table 1 or 2, incorporated herein by reference. In some embodiments, an epigenetic modifying agent comprises vorinostat or romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (c.e.g, PRMTI), methylstat, 4,4-dicarboxy-2,2″-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, the epigenetic modifying agent is an inhibitor of a histone deacetylase (e.g., is vorinostat and/or trichostatin A).

In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). One or a combination of molecules from the categories and examples described herein or from (Orme-Johnson 2007, Methods Cell Biol. 2007:80:813-26) can be used. In one embodiment, the invention includes a composition comprising an antibiotic, anti-inflammatory drug, angiogenic or vasoactive agent, growth factor or chemotherapeutic agent.

### Peptides or Proteins

In some embodiments, the composition or anellovector described herein may further comprise a peptide or protein. The peptide moieties may include, but are not limited to, a peptide ligand or antibody fragment (e.g., antibody fragment that binds a receptor such as an extracellular receptor), neuropeptide, hormone peptide, peptide drug, toxic peptide, viral or microbial peptide, synthetic peptide, and agonist or antagonist peptide.

Peptides moieties may be linear or branched. The peptide has a length from about 5 to about 200 amino acids, about 15 to about 150 amino acids, about 20 to about 125 amino acids, about 25 to about 100 amino acids, or any range therebetween.

Some examples of peptides include, but are not limited to, fluorescent tags or markers, antigens, antibodies, antibody fragments such as single domain antibodies, ligands and receptors such as glucagon-like peptide-1 (GLP-1), GLP-2 receptor 2, cholecystokinin B (CCKB) and somatostatin receptor, peptide therapeutics such as those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptides useful in the invention described herein also include small antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7): 1076-113). Such small antigen binding peptides may bind a cytosolic antigen, a nuclear antigen, an intra-organellar antigen.

In some embodiments, the composition or anellovector described herein includes a polypeptide linked to a ligand that is capable of targeting a specific location, tissue, or cell.

### Oligonucleotide Aptamers

In some embodiments, the composition or anellovector described herein may further comprise an oligonucleotide aptamer. Aptamer moieties are oligonucleotide or peptide aptamers. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers may possess desirable storage properties, and elicit little or no immunogenicity in therapeutic applications.

Both DNA and RNA aptamers can show robust binding affinities for various targets. For example, DNA and RNA aptamers have been selected for t lysozyme, thrombin, human immunodeficiency virus trans-acting responsive element (HIV TAR),(see en.wikipedia.org/wiki/Aptamer-cite_note-10), hemin, interferon γ, vascular endothelial growth factor (VEGF), prostate specific antigen (PSA), dopamine, and the non-classical oncogene, heat shock factor 1 (HSF1).

### Peptide Aptamers

In some embodiments, the composition or anellovector described herein may further comprise a peptide aptamer. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene. Such experiments identify particular proteins bound by the aptamers, and protein interactions that the aptamers disrupt, to cause the phenotype. In addition, peptide aptamers derivatized with appropriate functional moieties can cause specific post-translational modification of their target proteins, or change the subcellular localization of the targets

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### V. Host Cells

The invention is further directed to a host or host cell comprising a anellovector described herein. In some embodiments, the host or host cell is a plant, insect, bacteria, fungus, vertebrate, mammal (e.g., human), or other organism or cell. In certain embodiments, as confirmed herein, provided anellovectors infect a range of different host cells. Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, the anellovector is substantially non-immunogenic in the host. The anellovector or genetic element fails to produce an undesired substantial response by the host's immune system. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., infected with) an anellovector. In some embodiments, the host is a mammal, such as a human. The amount of the anellovector in the host can be measured at any time after administration. In certain embodiments, a time course of anellovector growth in a culture is determined.

In some embodiments, the anellovector, e.g., an anellovector as described herein, is heritable. In some embodiments, the anellovector is transmitted linearly in fluids and/or cells from mother to child. In some embodiments, daughter cells from an original host cell comprise the anellovector. In some embodiments, a mother transmits the anellovector to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the anellovector in a host cell has a transmission efficiency during meiosis of at 25%, 50%, 60%, 70%, 80%, 85%, 90%, 95%, or 99%. In some embodiments, the anellovector in a host cell has a transmission efficiency during mitosis of at least 25%, 50%, 60%, 70%, 80%, 85%, 90%, 95%, or 99%. In some embodiments, the anellovector in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the anellovector, e.g., anellovector replicates within the host cell. In one embodiment, the anellovector is capable of replicating in a mammalian cell, e.g., human cell. In other embodiments, the anellovector is replication deficient or replication incompetent.

While in some embodiments the anellovector replicates in the host cell, the anellovector does not integrate into the genome of the host, e.g., with the host's chromosomes. In some embodiments, the anellovector has a negligible recombination frequency, e.g., with the host's chromosomes. In some embodiments, the anellovector has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### VI. Methods of Use

The anellovectors and compositions comprising anellovectors described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. The anellovectors may be administered alone or formulated as a pharmaceutical composition.

The anellovectors may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of a anellovector or composition comprising same, e.g., as described herein, may result in delivery of a genetic element comprised by the anellovector to a target cell, e.g., in a subject.

An anellovector or composition thereof described herein, e.g., comprising an effector (e.g., an endogenous or exogenous effector), may be used to deliver the effector to a cell, tissue, or subject. In some embodiments, the anellovector or composition thereof is used to deliver the effector to bone marrow, blood, heart, GI or skin. Delivery of an effector by administration of a anellovector composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the anellovector, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, a anellovector or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the anellovector or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with the anellovector described herein, or a composition comprising the anellovector, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the anellovector modulates (e.g., increases or decreases) an activity or function in a cell with which the anellovector is contacted. In some embodiments, the anellovector modulates (e.g., increases or decreases) the level or activity of a molecule (e.g., a nucleic acid or a protein) in a cell with which the anellovector is contacted. In some embodiments, the anellovector decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more.

### VII. Methods of Production

### Producing the Genetic Element

Methods of making the genetic element of the anellovector are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

In some embodiments, the genetic element may be designed using computer-aided design tools. The anellovector may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the anellovector. The segments or ORFs may be assembled into the anellovector, e.g., in vitro recombination or unique restriction sites at 5′ and 3′ ends to enable ligation.

The genetic element can alternatively be synthesized with a design algorithm that parses the anellovector into oligo-length fragments, creating optimal design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element or segment of the genetic element may be sequence verified. In some embodiments, high-throughput sequencing of RNA or DNA can take place using Any Dot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. Any Dot.chips and methods for using them are described in part in International Publication Application Nos. WO 02088382, WO 03020968, WO 0303 1947. WO 2005044836, PCTEP 05105657, PCMEP 05105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science 16 Feb. 2001; Adams, M. et al, Science 24 Mar. 2000; and M. J. Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. The sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In some embodiments, shotgun sequencing is performed. In shotgun sequencing, DNA is broken up randomly into numerous small segments, which are sequenced using the chain termination method to obtain reads. Multiple overlapping reads for the target DNA are obtained by performing several rounds of this fragmentation and sequencing. Computer programs then use the overlapping ends of different reads to assemble them into a continuous sequence.

In some embodiments, factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, this is done both in context of larger regions of the anellovector genome (e.g., inserting effectors into a specific site in the genome, or replacing viral ORFs with effectors).

In another example, when supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

### In Vitro Circularization

In some instances, the genetic element to be packaged into a proteinaceous exterior is a single stranded circular DNA. The genetic element may, in some instances, be introduced into a host cell in a form other than a single stranded circular DNA. For example, the genetic element may be introduced into the host cell as a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or TraI, e.g., as described in Wawrzyniak et al. 2017, Front. Microbiol. 8: 2353; incorporated herein by reference with respect to the listed enzymes). In some embodiments, the double-stranded circular DNA is produced by in vitro circularization, e.g., as described in Example 35. Generally, in vitro circularized DNA constructs can be produced by digesting a plasmid comprising the sequence of a genetic element to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an anellovector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

### Producing the Anellovector

The genetic elements and vectors comprising the genetic elements prepared as described herein can be used in a variety of ways to express the anellovector in appropriate host cells. In some embodiments, the genetic element and vectors comprising the genetic element are transfected in appropriate host cells and the resulting RNA may direct the expression of the anellovector gene products. e.g., non-pathogenic protein and protein binding sequence, at high levels. Host cell systems which provide for high levels of expression include continuous cell lines that supply viral functions, such as cell lines superinfected with APV or MPV, respectively, cell lines engineered to complement APV or MPV functions, etc.

In some embodiments, the anellovector is produced as described in any of Examples 1, 2, 5, 6, or 15-17.

In some embodiments, the anellovector is cultivated in continuous animal cell lines in vitro. According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines susceptible to infection with anellovectors include, but are not limited to cell lines of human or primate origin, such as human or primate kidney carcinoma cell lines.

In some embodiments, the genetic elements and vectors comprising the genetic elements are transfected into cell lines that express a viral polymerase protein in order to achieve expression of the anellovector. To this end, transformed cell lines that express an anellovector polymerase protein may be utilized as appropriate host cells. Host cells may be similarly engineered to provide other viral functions or additional functions.

To prepare the anellovector disclosed herein, a genetic element or vector comprising the genetic element disclosed herein may be used to transfect cells which provide anellovector proteins and functions required for replication and production. Alternatively, cells may be transfected with helper virus before, during, or after transfection by the genetic element or vector comprising the genetic element disclosed herein. In some embodiments, a helper virus may be useful to complement production of an incomplete viral particle. The helper virus may have a conditional growth defect, such as host range restriction or temperature sensitivity, which allows the subsequent selection of transfectant viruses. In some embodiments, a helper virus may provide one or more replication proteins utilized by the host cells to achieve expression of the anellovector. In some embodiments, the host cells may be transfected with vectors encoding viral proteins such as the one or more replication proteins. In some embodiments, a helper virus comprises an antiviral sensitivity.

The genetic element or vector comprising the genetic element disclosed herein can be replicated and produced into anellovector particles by any number of techniques known in the art, as described, e.g., in U.S. Pat. Nos. 4,650,764; 5,166,057; 5,854,037; European Patent Publication EP 0702085A1: U.S. patent application Ser. No. 09/152,845: International Patent Publications PCT WO97/12032: WO96/34625; European Patent Publication EP-A780475: WO 99/02657: WO 98/53078: WO 98/02530; WO 99/15672: WO 98/13501: WO 97/06270; and EPO 780 47SA1, each of which is incorporated by reference herein in its entirety.

The production of anellovector-containing cell cultures according to the present invention can be carried out in different scales, such as in flasks, roller bottles or bioreactors. The media used for the cultivation of the cells to be infected are known to the skilled person and can generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of anellovectors.

The purification and isolation of anellovectors can be performed according to methods known by the skilled person in virus production and is described for example by Rinaldi, et al., DNA Vaccines: Methods and Protocols (Methods in Molecular Biology), 3rd ed. 2014, Humana Press.

In one aspect, the present invention includes a method for the in vitro replication and propagation of the anellovector as described herein, which may comprise the following steps: (a) transfecting a linearized genetic element into a cell line sensitive to anellovector infection; (b) harvesting the cells and isolating cells showing the presence of the genetic element; (c) culturing the cells obtained in step (b) for at least three days, such as at least one week or longer, depending on experimental conditions and gene expression; and (d) harvesting the cells of step (c).

In some embodiments, an anellovector may be introduced to a host cell line grown to a high cell density. In some embodiments, the anellovector may be harvested and/or purified by separation of solutes based on biophysical properties, e.g., ion exchange chromatography or tangential flow filtration, prior to formulation with a pharmaceutical excipient.

### VIII. Administration/Delivery

The composition (e.g., a pharmaceutical composition comprising an anellovector as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering an anellovector to a subject. The method includes administering a pharmaceutical composition comprising an anellovector as described herein to the subject. In some embodiments, the administered anellovector replicates in the subject (e.g., becomes a part of the virome of the subject).

The pharmaceutical composition may include wild-type or native viral elements and/or modified viral elements. The anellovector may include one or more of the sequences (e.g., nucleic acid sequences or nucleic acid sequences encoding amino acid sequences thereof) in any of Tables N1-N1417 or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any of Tables N1-N1417. The anellovector may comprise a nucleic acid molecule comprising a nucleic acid sequence with at least about 60%, 65%, 70%, 75%, 80%, 85%, 90% 95%, 96%, 97%, 98% and 99% sequence identity to one or more of the sequences in any of Tables N1-N1417. The anellovector may comprise a nucleic acid molecule encoding an amino acid sequence with at least about 60%, 65%, 70%, 75%, 80%, 85%, 90% 95%, 96%, 97%, 98% and 99% sequence identity to any one of the amino acid sequences in any of Tables A1-A1417. The anellovector may comprise a polypeptide comprising an amino acid sequence with at least about 60%, 65%, 70%, 75%, 80%, 85%, 90% 95%, 96%, 97%, 98% and 99% sequence identity to any one of the amino acid sequences in any of Tables A1-A1417. The anellovector may include one or more of the sequences in any of Tables A1-A1417 or N1-N1417, or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any of Tables N1-N1417.

In some embodiments, the anellovector is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the anellovector is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the anellovector inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising an anellovector described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the anellovector is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the anellovector is sufficient to compete with chronic or acute viral infection. In certain embodiments, the anellovector may be administered prophylactically to protect from viral infections (e.g. a provirotic). In some embodiments, the anellovector is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more).

### Redosing

The anellovectors described herein can, in some instances, be used as a delivery vehicle that can be administered in multiple doses (e.g., doses administered separately). While not wishing to be bound by theory, in some embodiments, an anellovector (e.g., as described herein) induces a relatively low immune response (as measured, for example, as 50% GMT values, e.g., as observed in Example 12), e.g., allowing for repeated dosing of a subject with one or more anellovectors (e.g., multiple doses of the same anellovector or different anellovectors). In an aspect, the invention provides a method of delivering an effector, comprising administering to a subject a first plurality of anellovectors and then a second plurality of anellovectors. In some embodiments, the second plurality of anellovectors comprise the same proteinaceous exterior as the anellovectors of the first plurality. In another aspect, the invention provides a method of selecting a subject (e.g., a human subject) to receive an effector, wherein the subject previously received, or was identified as having received, a first plurality of anellovectors comprising a genetic element encoding an effector, in which the method involves selecting the subject to receive a second plurality of anellovectors comprising a genetic element encoding an effector (e.g., the same effector as that encoded by the genetic element of the first plurality of anellovectors, or a different effector as that encoded by the genetic element of the first plurality of anellovectors). In another aspect, the invention provides a method of identifying a subject (e.g., a human subject) as suitable to receive a second plurality of anellovectors, the method comprising identifying the subject has having previously received a first plurality of anellovectors comprising a genetic element encoding an effector, wherein the subject being identified as having received the first plurality of anellovectors is indicative that the subject is suitable to receive the second plurality of anellovectors.

In some embodiments, the second plurality of anellovectors comprises a proteinaceous exterior with at least one surface epitope in common with the anellovectors of the first plurality of anellovectors. In some embodiments, the anellovectors of the first plurality and the anellovectors of the second plurality carry genetic elements encoding the same effector. In some embodiments, the anellovectors of the first plurality and the anellovectors of the second plurality carry genetic elements encoding different effectors.

In some embodiments, the second plurality comprises about the same quantity and/or concentration of anellovectors as the first plurality (e.g., when normalized to the body mass of the subject at the time of administration), e.g., the second plurality comprises 90-110%, e.g., 95-105% of the number of anellovectors in the first plurality when normalized to body mass of the subject at the time of administration. In some embodiments, wherein the first plurality comprises a greater dosage of anellovectors than the second plurality, e.g., wherein the first plurality comprises a greater quantity and/or concentration of anellovectors relative to the second plurality. In some embodiments, wherein the first plurality comprises a lower dosage of anellovectors than the second plurality, e.g., wherein the first plurality comprises a lower quantity and/or concentration of anellovectors relative to the second plurality.

In some embodiments, the subject is evaluated between the administration of the first and second pluralities of anellovectors, e.g., for the presence (e.g., persistence) of anellovectors from the first plurality, or progeny thereof. In some embodiments, the subject is administered the second plurality of anellovectors if the presence of anellovectors from the first plurality, or the progeny thereof, are not detected.

In some embodiments, the second plurality is administered to the subject at least 1, 2, 3, or 4 weeks, or 1, 2, 3, 4, 5, 6, 7, 8, 9, 10, 11, or 12 months, or 1, 2, 3, 4, 5, 10, or 20 years after the administration of the first plurality to the subject. In some embodiments, the second plurality is administered to the subject between 1-2 weeks, 2-3 weeks, 3-4 weeks, 1-2 months, 3-4 months, 4-5 months, 5-6 months, 6-7 months, 7-8 months, 8-9 months, 9-10 months, 10-11 months, 11-12 months, 1-2 years, 2-3 years, 3-4 years, 4-5 years, 5-10 years, or 10-20 years after the administration of the first plurality to the subject. In some embodiments, the method comprises administering a repeated dose of anellovectors over the course of at least 1, 2, 3, 4, or 5 years.

In some embodiments, the method further comprises assessing, after administration of the first plurality and before administration of the second plurality, one or more of:
a) the level or activity of the effector in the subject (e.g., by detecting a protein effector, e.g., by ELISA; by detecting a nucleic acid effector, e.g., by RT-PCR, or by detecting a downstream effect of the effector, e.g., level of an endogenous gene affected by the effector);
b) the level or activity of the anellovector of the first plurality in the subject (e.g., by detecting the level of the VPI of the anellovector);
c) the presence, severity, progression, or a sign or symptom of a disease in the subject that the anellovector was administered to treat; and/or
d) the presence or level of an immune response, e.g., neutralizing antibodies, against an anellovector.

In some embodiments, the method further comprises administering to the subject a third, fourth, fifth, and/or further plurality of anellovectors, e.g., as described herein.

In some embodiments, the first plurality and the second plurality are administered via the same route of administration, e.g., intravenous administration. In some embodiments, the first plurality and the second plurality are administered via different routes of administration. In some embodiments, the first and the second pluralities are administered by the same entity (e.g., the same health care provider). In some embodiments, the first and the second pluralities are administered by different entities (e.g., different health care providers).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents

Example 1: Immunologic effects of Anellovectors (Anellovector A): in vivo effector function, e.g., expression of the miRNA, of the anellovector after administration
Example 2: Identification and use of protein binding sequences: putative protein-binding sites in the Anellovirus genome
Example 3: Replication-deficient anellovectors and helper viruses
Example 4: Manufacturing process for replication-competent anellovectors
Example 5: Manufacturing process of replication-deficient anellovectors: recovery and scaling up of production of replication-deficient anellovectors
Example 6 Production of anellovectors using suspension cells: production of anellovectors in cells in suspension.
Example 7: Quantification of anellovector genome equivalents by qPCR: development of a hydrolysis probe-based quantitative PCR assay to quantify anellovectors
Example 8: Functional effects of an anellovector expressing an exogenous microRNA sequence: use of an anellovector to express a functional nucleic acid effector
Example 9: Characterization of regions required for anellovector development
Example 10: Anellovector delivery of exogenous proteins in vivo: This example demonstrates in vivo effector function (e.g. expression of proteins) of anellovectors after administration
Example 11: Tandem copies of the Anellovirus genome
Example 12: In vitro circularized Anellovirus genomes: constructs comprising circular, double stranded Anelloviral genome DNA with minimal non-viral DNA
Example 13: Production of anellovectors containing chimeric ORF1 with hypervariable domains from different Torque Teno Virus strains
Example 14: Design of an anellovector harboring a DNA payload
Example 15: Transduction of Anellovector-encoding antibody transgene
Example 16: Anellovectors based on tth8 and LY2 each successfully transduced the EPO gene into lung cancer cells
Example 17: Anellovectors with therapeutic transgenes can be detected in vivo after intravenous (i.v.) administration
Example 18: In vitro circularized genome as input material for producing anellovectors in vitro

### Example 1: Immunologic Effects of Anellovectors (Anellovector A)

This example describes in vivo effector function, e.g., expression of the miRNA, of the anellovector after administration.

Purified anellovectors prepared as described herein are intravenously administered to healthy pigs at various doses using hundred-fold dilutions starting from 10¹⁴ genome equivalents per kilogram down to 0 genome equivalents per kilogram. In order to evaluate the effects on immune tolerance, pigs are injected daily for 3 days with anellovectors or vehicle control PBS and sacrificed after 3 days.

Spleen, bone marrow and lymph nodes are harvested. Single cell suspensions are prepared from each of the tissues and stained with extracellular markers for MHC-II, CD11c, and intracellular IFN. MHC+, CD11c+, IFN+antigen presenting cells are analyzed via flow cytometry from each tissue, e.g., wherein a cell that is positive for a given one of the above-mentioned markers is a cell that exhibits higher fluorescence than 99% of cells in a negative control population that lack expression of the marker but is otherwise similar to the the assay population of cells, under the same conditions.

In an embodiment, a decreased number of IFN+ cells in the anellovector treatment group compared to the control will indicate that the anellovectors decrease IFN production in cells after administration.

### Example 2: Identification and Use of Protein Binding Sequences

This example describes putative protein-binding sites in the Anellovirus genome, which can be used for amplifying and packaging effectors, e.g., in an anellovector as described herein. In some instances, the protein-binding sites may be capable of binding to an exterior protein, such as a capsid protein.

Two conserved domains within the Anellovirus genome are putative origins of replication: the 5′ UTR conserved domain (5CD) and the GC-rich domain (GCR) (de Villiers et al., Journal of Virology 2011; Okamoto et al., Virology 1999). In one example, in order to confirm whether these sequences act as DNA replication sites or as capsid packaging signals, deletions of each region are made in plasmids harboring an Anellovirus sequence. A539 cells are transfected with the deletion constructs. Transfected cells are incubated for four days, and then virus is isolated from supernatant and cell pellets. A549 cells are infected with virus, and after four days, virus is isolated from the supernatant and infected cell pellets. qPCR is performed to quantify viral genomes from the samples. Disruption of an origin of replication prevents viral replicase from amplifying viral DNA and results in reduced viral genomes isolated from transfected cell pellets compared to wild-type virus. A small amount of virus is still packaged and can be found in the transfected supernatant and infected cell pellets. In some embodiments, disruption of a packaging signal will prevent the viral DNA from being encapsulated by capsid proteins. Therefore, in embodiments, there will still be an amplification of viral genomes in the transfected cells, but no viral genomes are found in the supernatant or infected cell pellets.

In a further example, in order to characterize additional replication or packaging signals in the DNA, a series of deletions across the entire TTMV-LY2 genome is used. Deletions of 100 bp are made stepwise across the length of the sequence. Plasmids harboring Anellovirus genome deletions are transfected into A549 and tested as described above. In some embodiments, deletions that disrupt viral amplification or packaging will contain potential cis-regulatory domains.

Replication and packaging signals can be incorporated into effector-encoding DNA sequences (e.g., in a genetic element in an anellovector) to induce amplification and encapsulation. This is done both in context of larger regions of the anellovector genome (i.e., inserting effectors into a specific site in the genome, or replacing viral ORFs with effectors, etc.), or by incorporating minimal cis signals into the effector DNA. In cases where the anellovector lacks trans replication or packaging factors (e.g., replicase and capsid proteins, etc.), the trans factors are supplied by helper genes. The helper genes express all of the proteins and RNAs sufficient to induce amplification and packaging, but lack their own packaging signals. The anellovector DNA is co-transfected with helper genes, resulting in amplification and packaging of the effector but not of the helper genes.

### Example 3: Replication-Deficient Anellovectors and Helper Viruses

For replication and packaging of an anellovector, some elements (e.g., an ORF1, ORF1/1, ORF1/2, ORF2, ORF2/2, ORF2/3, and/or ORF2t/3 molecule, or a nucleic acid sequence encoding same) can be provided in trans. These include proteins or non-coding RNAs that direct or support DNA replication or packaging. Trans elements can, in some instances, be provided from a source alternative to the anellovector, such as a helper virus, plasmid, or from the cellular genome.

Other elements are typically provided in cis (e.g., a TATA box, cap site, initiator element, transcriptional start site, 5′ UTR conserved domain, three open-reading frame region, poly(A) signal, or GC-rich region). These elements can be, for example, sequences or structures in the anellovector DNA that act as origins of replication (e.g., to allow amplification of anellovector DNA) or packaging signals (e.g., to bind to proteins to load the genome into the capsid). Generally, a replication deficient virus or anellovector will be missing one or more of these elements, such that the DNA is unable to be packaged into an infectious virion or anellovector even if other elements are provided in trans.

Replication deficient viruses can be useful for controlling replication of an anellovector (e.g., a replication-deficient or packaging-deficient anellovector) in the same cell. In some instances, the virus will lack cis replication or packaging elements, but express trans elements such as proteins and non-coding RNAs. Generally, the therapeutic anellovector would lack some or all of these trans elements and would therefore be unable to replicate on its own, but would retain the cis elements. When co-transfected/infected into cells, the replication-deficient virus would drive the amplification and packaging of the anellovector. The packaged particles collected would thus be comprised solely of therapeutic anellovector, without contamination from the replication-deficient virus.

To develop a replication deficient anellovector, conserved elements in the non-coding regions of Anellovirus will be removed. In particular, deletions of the conserved 5′ UTR domain and the GC-rich domain will be tested, both separately and together. Both elements are contemplated to be important for viral replication or packaging. Additionally, deletion series will be performed across the entire non-coding region to identify previously unknown regions of interest.

Successful deletion of a replication element will result in reduction of anellovector DNA amplification within the cell, e.g., as measured by qPCR, but will support some infectious anellovector production, e.g., as monitored by assays on infected cells that can include any or all of qPCR, western blots, fluorescence assays, or luminescence assays. Successful deletion of a packaging element will not disrupt anellovector DNA amplification, so an increase in anellovector DNA will be observed in transfected cells by qPCR. However, the anellovector genomes will not be encapsulated, so no infectious anellovector production will be observed.

### Example 4: Manufacturing Process for Replication-Competent Anellovectors

This example describes a method for recovery and scaling up of production of replication-competent anellovectors. Anellovectors are replication competent when they encode in their genome all the required nucleic acid elements and ORFs necessary to replicate in cells. Since these anellovectors are not defective in their replication they do not need a complementing activity provided in trans. They might, however need helper activity, such as enhancers of transcriptions (e.g. sodium butyrate) or viral transcription factors (e.g. adenoviral E1, E2 E4, VA; HSV Vp16 and immediate early proteins).

In this example, double-stranded DNA encoding the full sequence of a synthetic anellovector either in its linear or circular form is introduced into 5E+05 adherent mammalian cells in a T75 flask by chemical transfection or into 5E+05 cells in suspension by electroporation. After an optimal period of time (e.g., 3-7 days post transfection), cells and supernatant are collected by scraping cells into the supernatant medium. A mild detergent, such as a biliary salt, is added to a final concentration of 0.5% and incubated at 37° C. for 30 minutes. Calcium and Magnesium Chloride is added to a final concentration of 0.5 mM and 2.5 mM, respectively. Endonuclease (e.g. DNAse I, Benzonase), is added and incubated at 25-37° C. for 0.5-4 hours. Anellovector suspension is centrifuged at 1000×g for 10 minutes at 4° C. The clarified supernatant is transferred to a new tube and diluted 1:1 with a cryoprotectant buffer (also known as stabilization buffer) and stored at −80° ° C. if desired. This produces passage 0 of the anellovector (P0). To bring the concentration of detergent below the safe limit to be used on cultured cells, this inoculum is diluted at least 100-fold or more in serum-free media (SFM) depending on the anellovector titer.

A fresh monolayer of mammalian cells in a T225 flask is overlaid with the minimum volume sufficient to cover the culture surface and incubated for 90 minutes at 37° C. and 5% carbon dioxide with gentle rocking. The mammalian cells used for this step may or may not be the same type of cells as used for the P0 recovery. After this incubation, the inoculum is replaced with 40 ml of serum-free, animal origin-free culture medium. Cells are incubated at 37° C. and 5% carbon dioxide for 3-7 days. 4 ml of a 10× solution of the same mild detergent previously utilized is added to achieve a final detergent concentration of 0.5%, and the mixture is then incubated at 37° C. for 30 minutes with gentle agitation. Endonuclease is added and incubated at 25-37° C. for 0.5-4 hours. The medium is then collected and centrifuged at 1000×g at 4° C. for 10 minutes. The clarified supernatant is mixed with 40 ml of stabilization buffer and stored at −80° C. This generates a seed stock, or passage 1 of anellovector (P1).

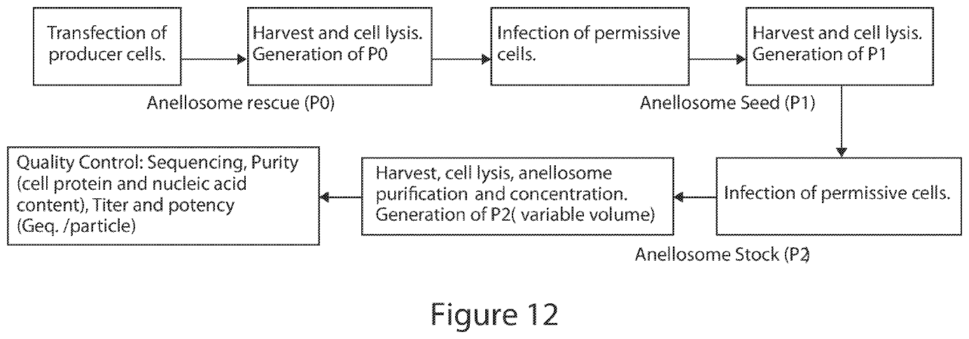

Depending on the titer of the stock, it is diluted no less than 100-fold in SFM and added to cells grown on multilayer flasks of the required size. Multiplicity of infection (MOI) and time of incubation is optimized at smaller scale to ensure maximal anellovector production. After harvest, anellovectors may then be purified and concentrated as needed. A schematic showing a workflow, e.g., as described in this example, is provided in FIG. 12.

### Example 5: Manufacturing Process of Replication-Deficient Anellovectors

This example describes a method for recovery and scaling up of production of replication-deficient anellovectors.

Anellovectors can be rendered replication-deficient by deletion of one or more ORFs (e.g., ORF1, ORF1/1, ORF1/2, ORF2, ORF2/2, ORF2/3, and/or ORF2t/3) involved in replication. Replication-deficient anellovectors can be grown in a complementing cell line. Such cell line constitutively expresses components that promote anellovector growth but that are missing or nonfunctional in the genome of the anellovector.

In one example, the sequence(s) of any ORF(s) involved in anellovector propagation are cloned into a lentiviral expression system suitable for the generation of stable cell lines that encode a selection marker, and lentiviral vector is generated as described herein. A mammalian cell line capable of supporting anellovector propagation is infected with this lentiviral vector and subjected to selective pressure by the selection marker (e.g., puromycin or any other antibiotic) to select for cell populations that have stably integrated the cloned ORFs. Once this cell line is characterized and certified to complement the defect in the engineered anellovector, and hence to support growth and propagation of such anellovectors, it is expanded and banked in cryogenic storage. During expansion and maintenance of these cells, the selection antibiotic is added to the culture medium to maintain the selective pressure. Once anellovectors are introduced into these cells, the selection antibiotic may be withheld.

Once this cell line is established, growth and production of replication-deficient anellovectors is carried out, e.g., as described in Example 15.

### Example 6: Production of Anellovectors Using Suspension Cells

This example describes the production of anellovectors in cells in suspension.

In this example, an A549 or 293T producer cell line that is adapted to grow in suspension conditions is grown in animal component-free and antibiotic-free suspension medium (Thermo Fisher Scientific) in WAVE bioreactor bags at 37 degrees and 5% carbon dioxide. These cells, seeded at 1×10⁶ viable cells/mL, are transfected using lipofectamine 2000 (Thermo Fisher Scientific) under current good manufacturing practices (cGMP), with a plasmid comprising anellovector sequences, along with any complementing plasmids suitable or required to package the anellovector (e.g., in the case of a replication-deficient anellovector, e.g., as described in Example 16). The complementing plasmids can, in some instances, encode for viral proteins that have been deleted from the anellovector genome (e.g., an anellovector genome based on a viral genoe, e.g., an Anellovirus genome, e.g., as described herein) but are useful or required for replication and packaging of the anellovectors. Transfected cells are grown in the WAVE bioreactor bags and the supernatant is harvested at the following time points: 48, 72, and 96 hours post transfection. The supernatant is separated from the cell pellets for each sample using centrifugation. The packaged anellovector particles are then purified from the harvested supernatant and the lysed cell pellets using ion exchange chromatography.

The genome equivalents in the purified prep of the anellovectors can be determined, for example, by using a small aliquot of the purified prep to harvest the anellovector genome using a viral genome extraction kit (Qiagen), followed by qPCR using primers and probes targeted towards the anellovector DNA sequence, e.g., as described in Example 18.

The infectivity of the anellovectors in the purified prep can be quantified by making serial dilutions of the purified prep to infect new A549 cells. These cells are harvested 72 hours post transfection, followed by a qPCR assay on the genomic DNA using primers and probes that are specific to the anellovector DNA sequence.

### Example 7: Quantification of Anellovector Genome Equivalents by qPCR

This example demonstrates the development of a hydrolysis probe-based quantitative PCR assay to quantify anellovectors. Sets of primers and probes are designed based on an Anellovirus genome sequence. using the software Geneious with a final user optimization. Exemplary primer sequences for TTV (Accession No. AJ620231.1) and TTMV (Accession No. JX134045.1) are shown in Table 44 below.

TABLE 44
Sequences of forward and reverse primers and hydrolysis probes used to
quantify TTMV and TTV genome equivalents by quantitative PCR.

| TTMV | | SEQ ID NO: |
|---|---|---|
| Forward Primer | 5′-GAAGCCCACCAAAAGCAATT-3′ | 5189 |
| Reverse Primer | 5′-AGTTCCCGTGTCTATAGTCGA-3′ | 5190 |
| Probe | 5′-ACTTCGTTACAGAGTCCAGGGG-3′ | 5191 |
| TTV | | |
| Forward Primer | 5′-AGCAACAGGTAATGGAGGAC-3′ | 5192 |
| Reverse Primer | 5′-TGGAAGCTGGGGTCTTTAAC-3′ | 5193 |
| Probe | 5′-TCTACCTTAGGTGCAAAGGGCC-3′ | 5194 |

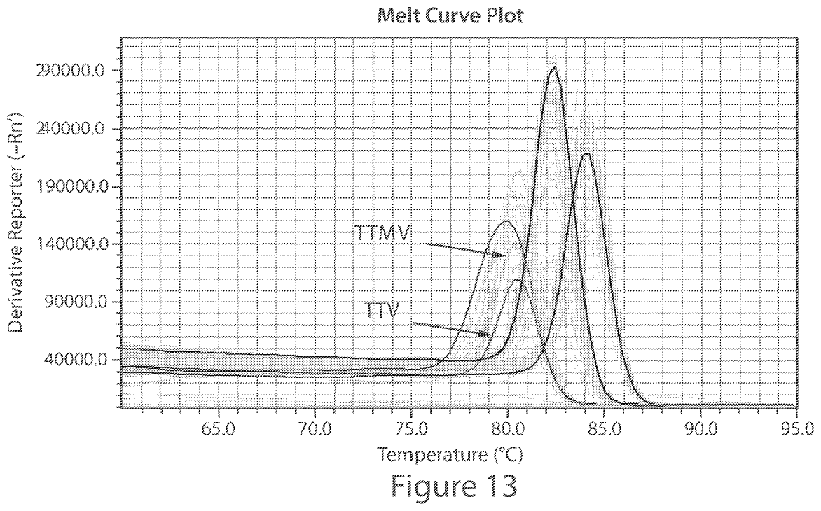

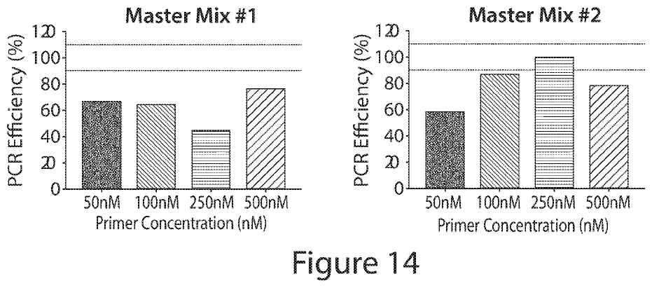

As a first step in the development process, qPCR is run using the Anellovirus primers with SYBR-green chemistry to check for primer specificity. FIG. 13 shows one distinct amplification peak for each primer pair.

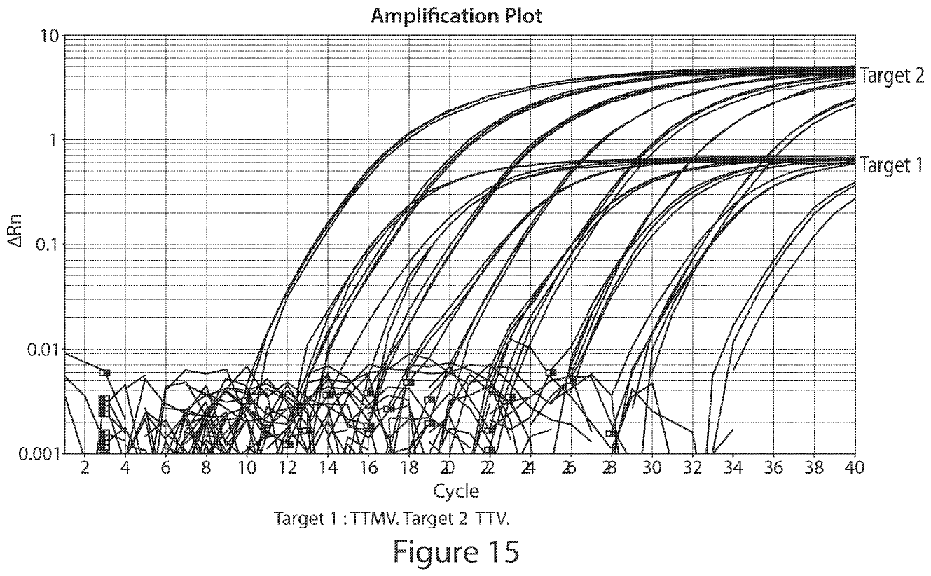

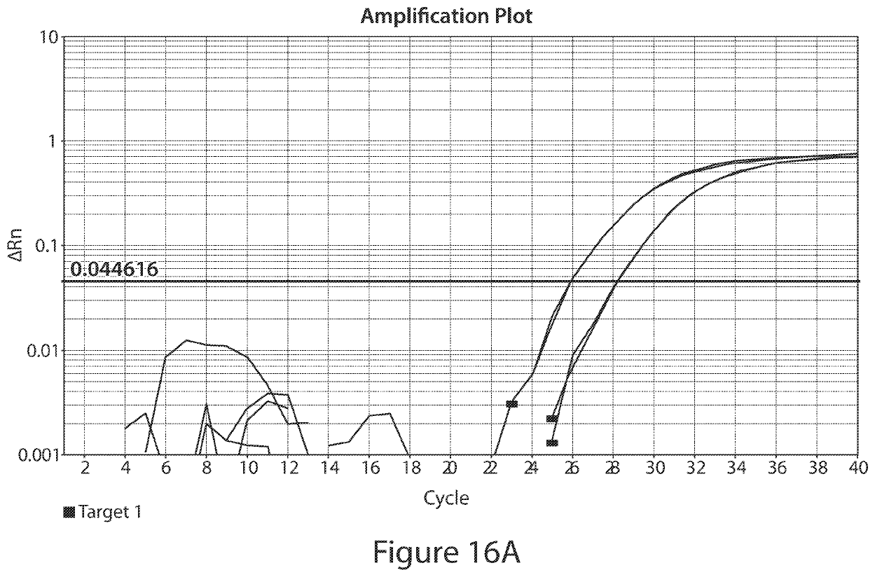

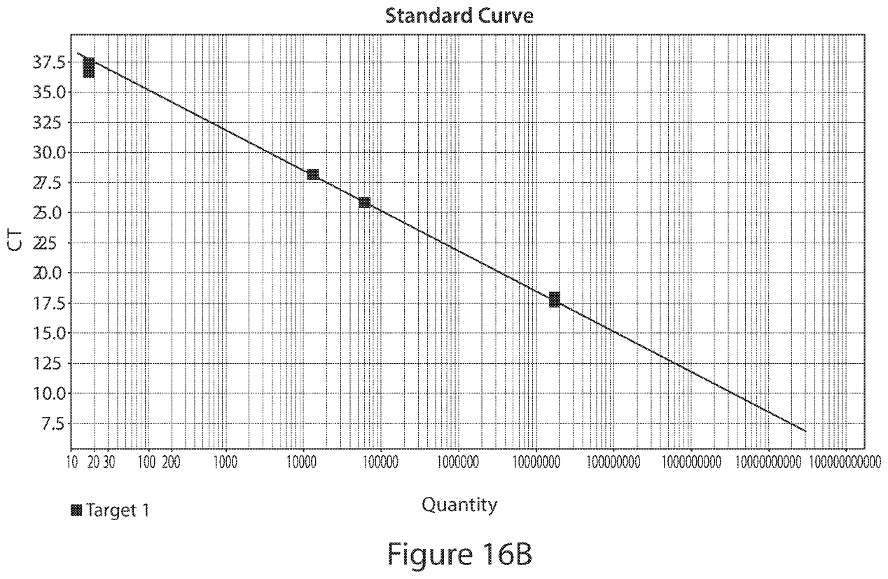

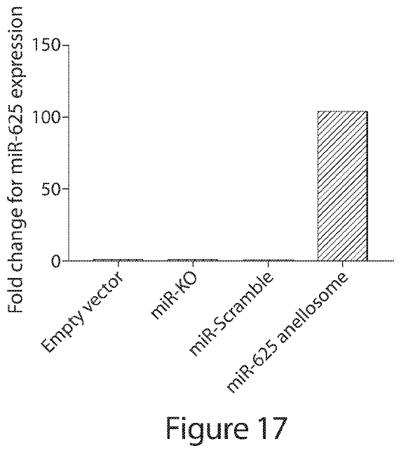

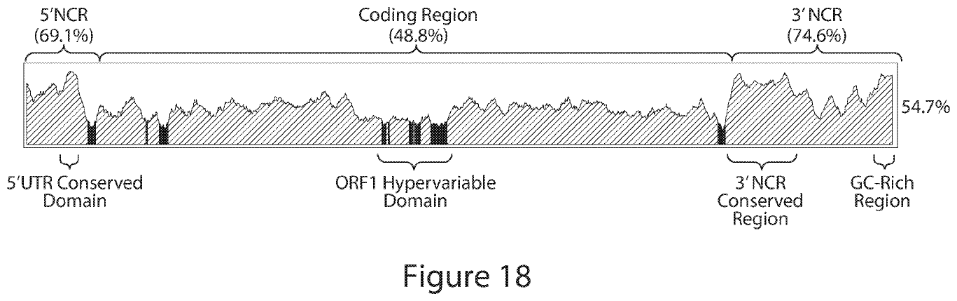

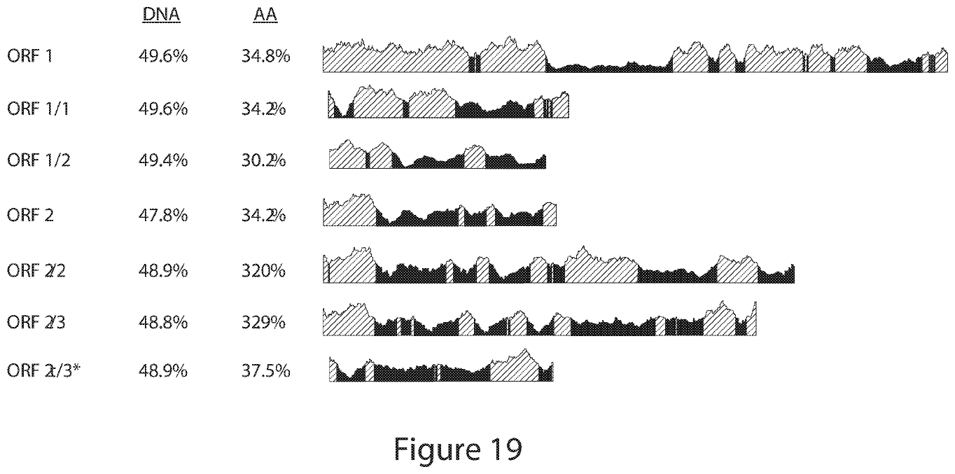

Hydrolysis probes are ordered labeled with the fluorophore 6FAM at the 5′ end and a minor groove binding, non-fluorescent quencher (MGBNFQ) at the 3′ end. The PCR efficiency of the new primers and probes was evaluated using two different commercial master mixes using purified plasmid DNA as component of a standard curve and increasing concentrations of primers. The standard curve is set up by using purified plasmids containing the target sequences for the different sets of primers-probes. Seven tenfold serial dilutions are performed to achieve a linear range over 7 logs and a lower limit of quantification of 15 copies per 20 ul reaction. All primers for qPCR are ordered from a commercial vendor such as IDT. Hydrolysis probes conjugated to the fluorophore 6FAM and a minor groove binding, non-fluorescent quencher (MGBNFQ) as well as all the qPCR master mixes are obtained from Thermo Fisher. An exemplary amplification plot is shown in FIG. 15.

Using these primer-probe sets and reagents, the genome equivalent (GEq)/ml in anellovector stocks is quantified. The linear range is then used to calculate the GEq/ml. Samples with higher concentrations than the linear range can be diluted as needed.

### Example 8: Functional Effects of an Anellovector Expressing an Exogenous microRNA Sequence

This example demonstrates the successful expression of an exogenous miRNA (miR-625) from anellovector genome using a native promoter.

500 ng of following plasmid DNAs are transfected into 60% confluent wells of HEK293T cells in a 24 well plate:
i) Empty plasmid backbone
ii) Plasmid containing an Anellovirus genome in which endogenous miRNA is knocked out (KO)
iii) Anellovirus genome in which endogenous miRNA is replaced with a non-targeting scramble miRNA
iv) Anellovirus genome in which endogenous miRNA sequence is replaced with miRNA encoding miR-625

72 hours post transfection, total miRNA is harvested from the transfected cells using the Qiagen miRNeasy kit, followed by reverse transcription using miRNA Script RT II kit. Quantitative PCR is performed on the reverse transcribed DNA using primer that should specifically detect miRNA-625 or RNU6 small RNA. RNU6 small RNA is used as a housekeeping gene and data is plotted as a fold change relative to empty vector.

### Example 9: Characterization of Regions Required for Anellovector Development

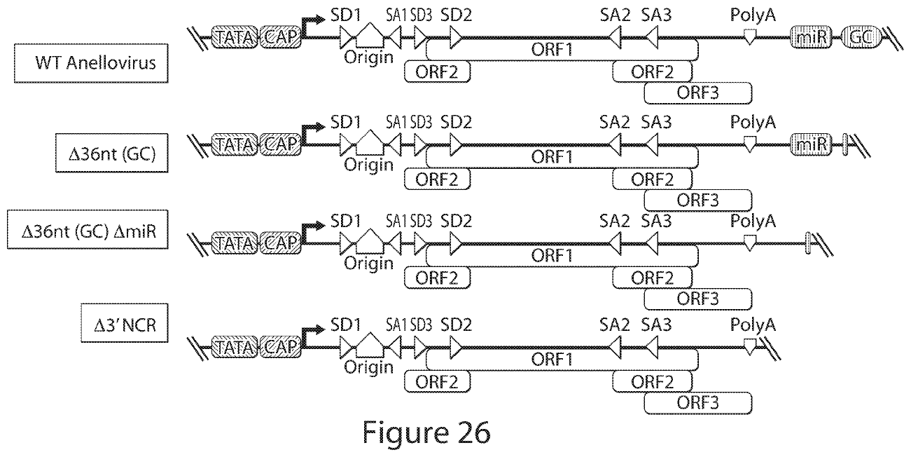

This Example describes deletions in the Anellovirus genome to help characterize the portions of the genome sufficient for replicating virus and anellovector production. A series of deletions were made in the non-coding region (NCR) of TTV-tth8 downstream of the ORFs (nts 3016 to 3753). A 36-nucleotide (nt) sequence (CGCGCTGCGCGCGCCGCCCAGTAGGGGGAGCCATGC (SEQ ID NO: 5167)) was deleted from the GC region (labeled Δ36nt (GC)). Additionally, a 78-nt pre-microRNA sequence (CCGCCATCTTAAGTAGTTGAGGCGGACGGTGGCGTGAGTTCAAAGGTCACCATCAGCCACA CCTACTCAAAATGGTGG (SEQ ID NO: 5168)) was deleted from the 3′ NCR (labeled Δ36nt (GC) AmiR). And lastly, an extra 171 nts in the 3′NCR of Δ36nt (GC) was deleted (CTTAAGTAGTTGAGGCGGACGGTGGCGTGAGTTCAAAGGTCACCATCAGCCACACCTACTC AAAATGGTGGACAATTTCTTCCGGGTCAAAGGTTACAGCCGCCATGTTAAAACACGTGACGT ATGACGTCACGGCCGCCATTTTGTGACACAAGATGGCCGACTTCCTTCC (SEQ ID NO: 5169)) and labeled Δ3′NCR (FIG. 26). 2 μg of circular pTTV-tth8 (WT), pTTV-tth8(Δ36nt (GC)), pTTV-tth8(Δ36nt (GC) ΔmiR), pTTV-tth8(Δ3′NCR) DNA plasmids harboring the altered 3′NCRs TTV-tth8 respectively described above, were transfected into HEK293 at 60% confluency in a 12-well plate using lipofectamine 2000, in triplicates. 48 hours after transfection, cell pellets were harvested and lysed to isolate mRNA transcripts (RNeasy, Qiagen cat #74104) and converted to cDNA (High-Capacity cDNA Reverse Transcription kit, ThermoFisher, cat #4368814). qPCR was performed on all samples measuring viral transcripts expression with each deletion and normalized to the internal control mRNA of GAPDH.

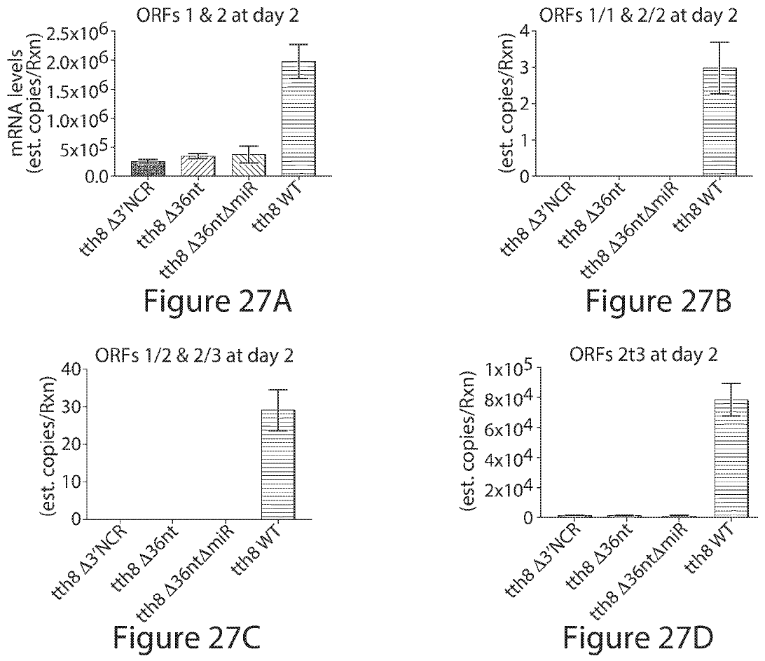

As shown in FIGS. 27A-27D, all three of the deletion mutants significantly inhibited viral transcript expression in vitro. Therefore, the 3′ NCR of TTV-tth8 is necessary for anellovector production for expression of transgene.

The TTV strain tth8, GeneBank Accession No. AJ620231.1, was deposited as a full-genome sequence. In the GC-rich region, however, there is a stretch of 36 nucleotides annotated as generic Ns. This region is highly conserved among TTV strains and therefore might be important for the biology of these viruses. The DNA sequences of several hundred TTV strains were computationally aligned and used to generate a strong consensus sequence for those 36 nucleotides (CGCGCTGCGCGCGCCGCCCAGTAGGGGGAGCCATGC (SEQ ID NO: 5167)). The TTV-tth8 genome sequence referred to herein as the “wild-type” sequence accordingly had this consensus sequence inserted in place of the stretch of 36 Ns listed in the publicly available TTV-tth8 sequence.

### Example 10: Anellovector Delivery of Exogenous Proteins In Vivo

This example demonstrates in vivo effector function (e.g. expression of proteins) of anellovectors after administration.

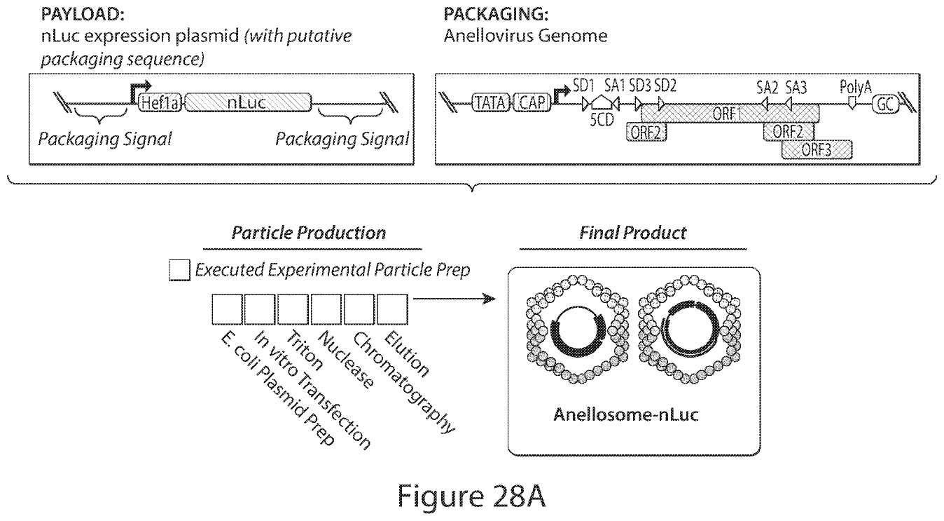

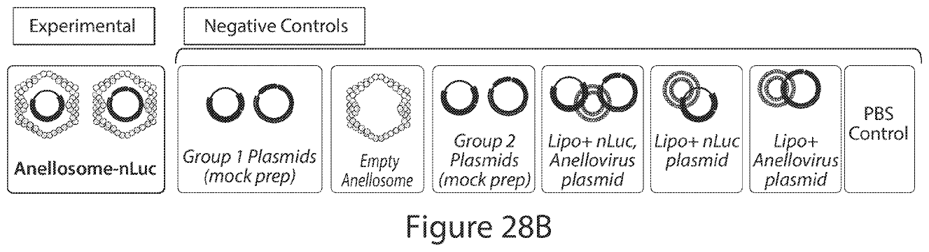

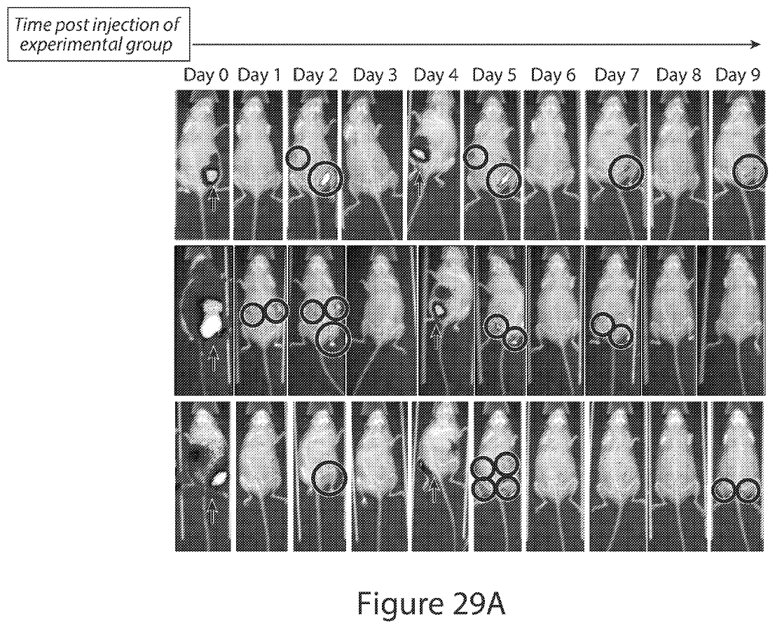

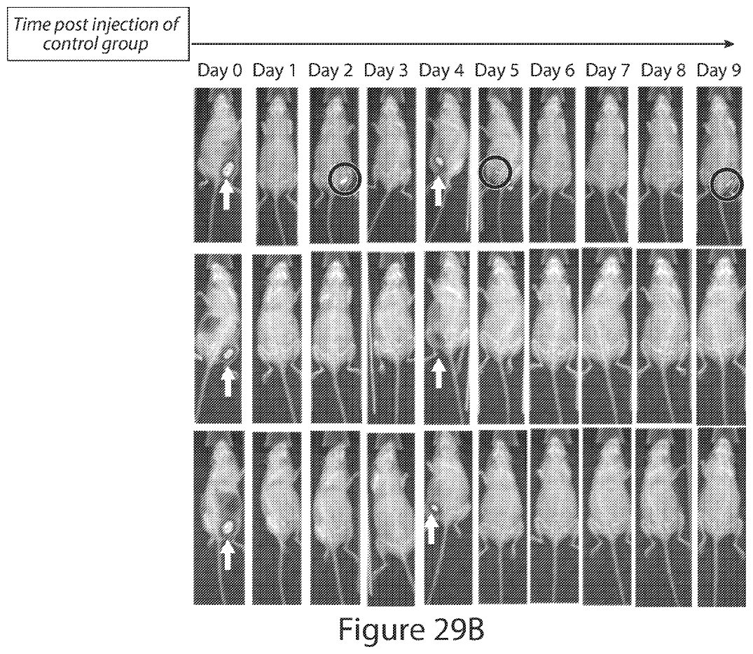

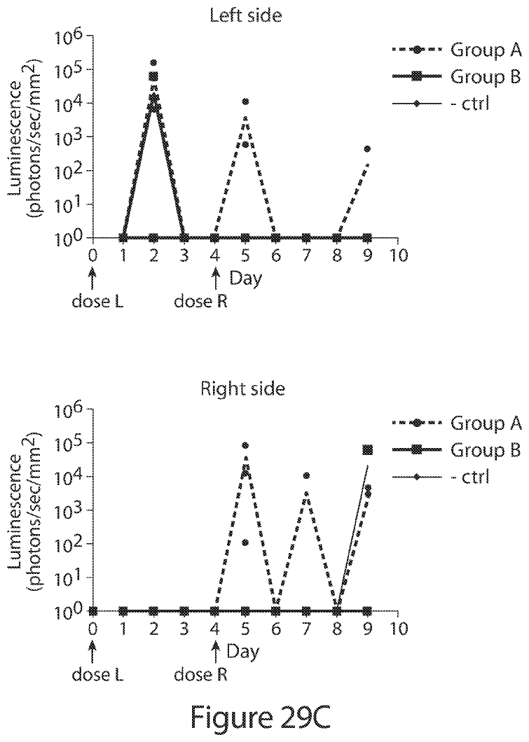

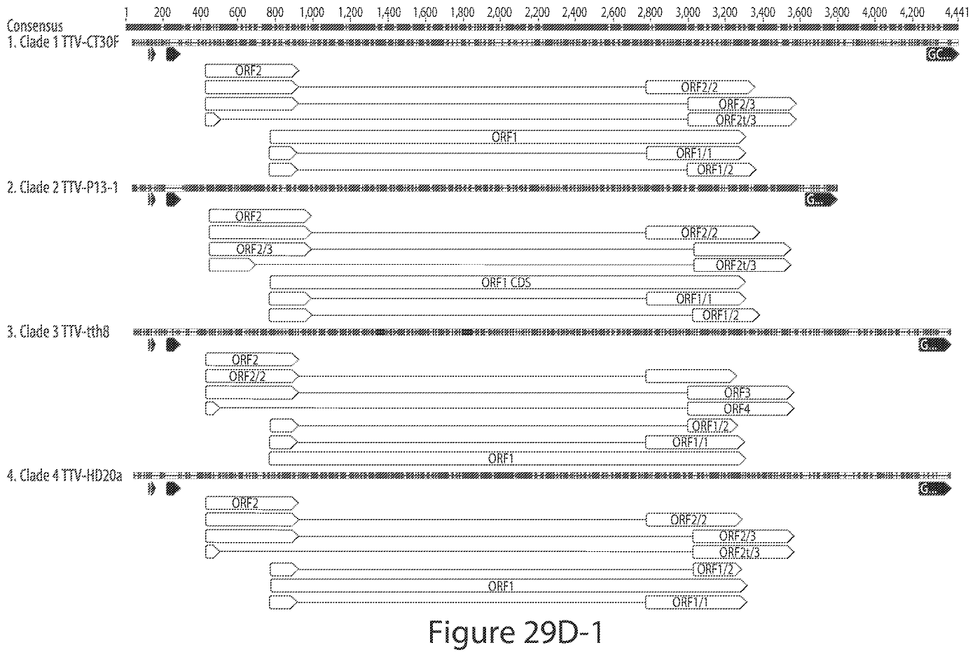

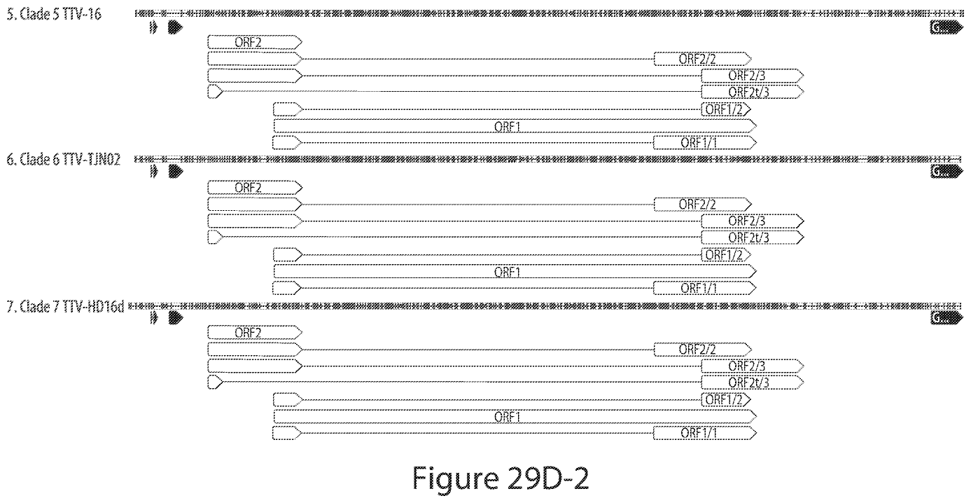

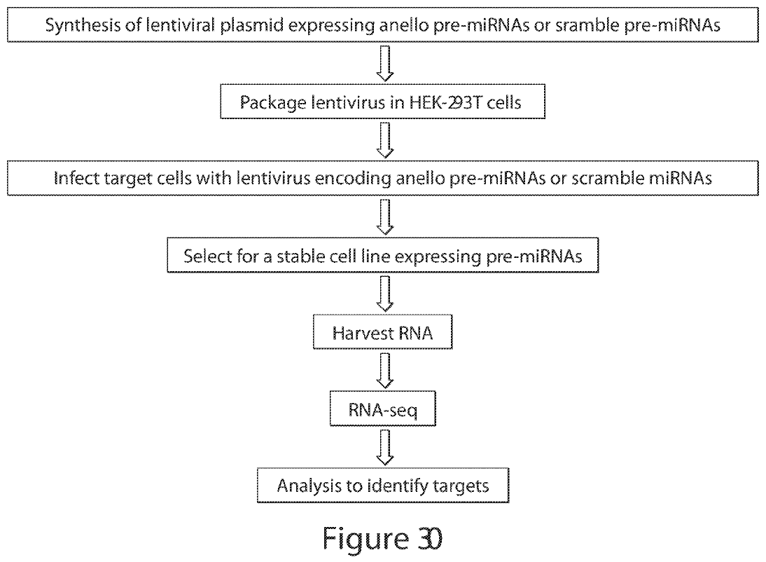

Anellovectors comprising a transgene encoding nano-luciferase (nLuc) (FIGS. 28A-28B) are prepared. Briefly, double-stranded DNA plasmids harboring the Anellovirus non-coding regions and an nLuc expression cassette are transfected into HEK293T cells along with double-stranded DNA plasmids encoding the full Anellovirus genome to act as trans replication and packaging factors. After transfection, cells are incubated to permit anellovector production and anellovector material is harvested and enriched via nuclease treatment, ultrafiltration/diafiltration, and sterile filtration. Additional HEK293T cells are transfected with non-replicating DNA plasmids harboring nLuc expression cassettes and Anellovirus ORF transfection cassettes, but lacking non-coding domains essential for replication and packaging, to act as a “non-viral” negative control. The non-viral samples are prepared following the same protocol as the anellovector material.

Anellovector preparation is administered to a cohort of three healthy mice intramuscularly, and monitored by IVIS Lumina imaging (Bruker) over the course of nine days. As a non-viral control, the non-replicating preparation is administered to three additional mice. Injections of 25 μL of anellovector or non-viral preparations are administered to the left hind leg on Day 0, and re-administered to the right hind leg on Day 4. Observation of more occurences of nLuc luminescent signal in mice injected with the anellovector preparation than the non-viral preparation would be consistent with trans gene expression after in vivo anellovector transduction.

### Example 11: Tandem Copies of the Anellovirus Genome

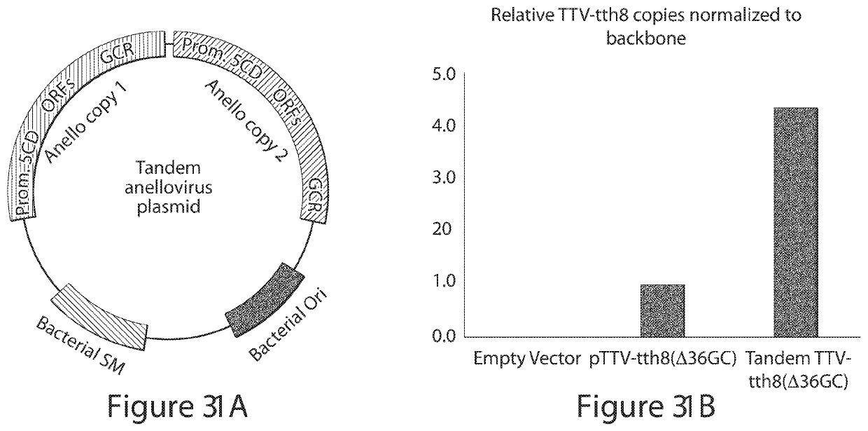

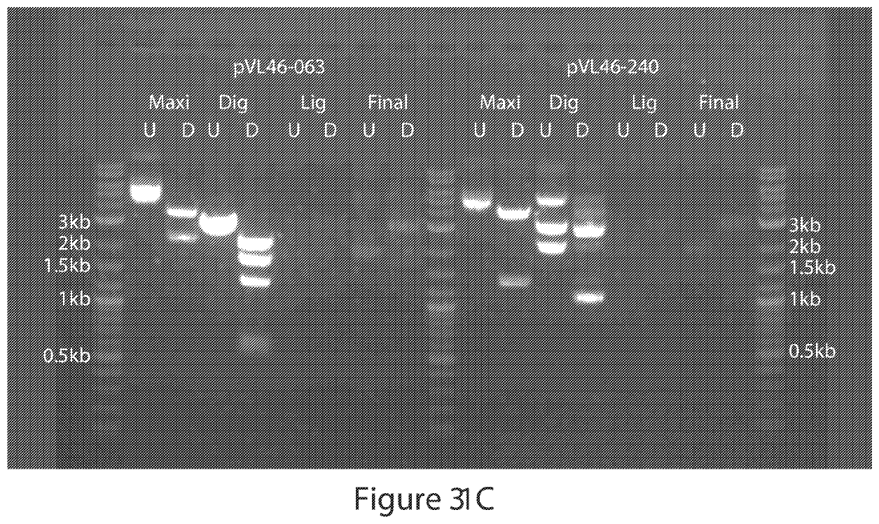

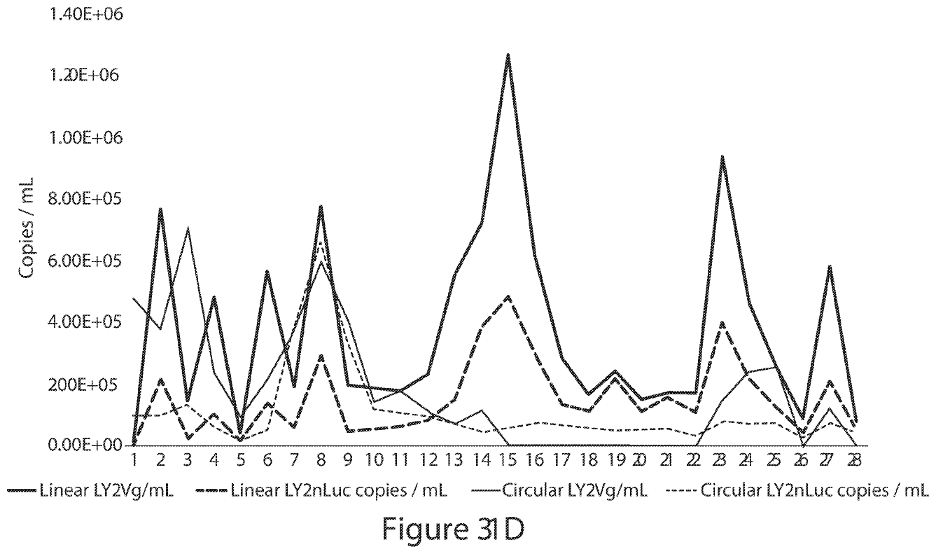

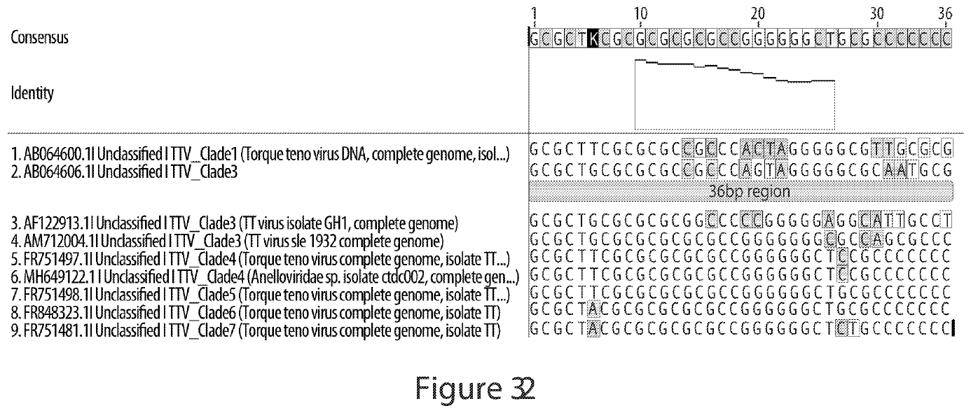

This example describes plasmid-based expression vectors harboring two copies of a single anelloviral genome, arranged in tandem such that the GC-rich region of the upstream genome is near the 5′ region of the downstream genome (FIG. 31A).

Anelloviruses replicate via rolling circle, in which a replicase (Rep) protein binds to the genome at an origin of replication and initiates DNA synthesis around the circle. For anellovirus genomes contained in plasmid backbones, this requires either replication of the full plasmid length, which is longer than the native viral genome, or recombination of the plasmid resulting in a smaller circle comprising the genome with minimal backbone. Therefore, viral replication off of a plasmid can be inefficient. To improve viral genome replication efficiency, plasmids are engineered with tandem copies of TTV-tth8 and TTMV-LY2. These plasmids present every possible circular permutation of the anelloviral genome: regardless of where the Rep protein binds, it will be able to drive replication of the viral genome from the upstream origin of replication to the downstream origin. A similar strategy has been used to produce porcine Anelloviruses (Huang et al., 2012, Journal of Virology 86 (11) 6042-6054).

Tandem anellovector can be assembled, for example, by sequentially cloning copies of the genome into a plasmid backbone, leaving 12 bp of non-viral DNA between the two sequences. Alternatively, tandem anellovector can be assembled via Golden-gate assembly, simultaneously incorporating two copies of the genome into a backbone and leaving no extra nucleotides between the genomes.

Plasmid harboring tandem copies of an anellovector genetic element sequence is transfected into HEK239T cells. Cells are incubated for five days, then lysed using 0.1% Triton X-100 and treated with nucleases to digest DNA not protected by viral capsids. qPCR is then performed using Taqman probes for the TTV-tth8 genome sequence and the plasmid backbone. TTV-tth8 genome copies are normalized to backbone copics.

### Example 12: In Vitro Circularized Anellovirus Genomes

This example describes constructs comprising circular, double stranded Anelloviral genome DNA with minimal non-viral DNA. These circular viral genomes more closely match the double-stranded DNA intermediates found during wild-type Anellovirus replication. When introduced into a cell, such circular, double stranded Anelloviral genome DNA with minimal non-viral DNA can undergo rolling circle replication to produce, for example, a genetic element as described herein.

In one example, plasmids harboring an Anellovirus genome sequence are digested with restriction endonucleases recognizing sites flanking the genomic DNA. The resulting linearized genomes are then ligated to form circular DNA. These ligation reactions are done with varying DNA concentrations to optimize the intramolecular ligations. The ligated circles are either directly transfected into mammalian cells, or further processed to remove non-circular genome DNA by digesting with restriction endonucleases to cleave the plasmid backbone and exonucleases to degrade linear DNA. To demonstrate the improvements in Anellovirus production, circularized Anellovirus genome constructs are transfected into HEK293T cells. After 7 days of incubation, cells are lysed, and qPCR is performed to compare the levels of anellovirus genome between circularized and plasmid-based anelloviral genomes. Increased levels of Anelloviral genomes show that circularization of the viral DNA is a useful strategy for increasing Anellovirus production.

Digested plasmid can be purified on 1% agarose gels prior to electroelution or Qiagen column purification and ligation with T4 DNA Ligase. Circularized DNA is concentrated on a 100 kDa UF/DF membrane before transfection. Circularization is confirmed by gel electrophoresis. T-225 flasks are seeded with HEK293T at 3×10⁴ cells/cm² one day prior to lipofection with Lipofectamine 2000. Nine micrograms of circularized Anellovirus DNA and 50 μg of circularized Anellovirus-nLuc are co-transfected one day post flask seeding. As a comparison, an additional T-225 flask is co-transfected with 50 μg of linearized Anellovirus and 50 μg of linearized Anellovirus-nLuc.

Anellovector production proceeds for eight days prior to cell harvest in Triton X-100 harvest buffer. Generally, anellovectors can be enriched, e.g., by lysis of host cells, clarification of the lysate, filtration, and chromatography. In this example, harvested cells are nuclease treated prior to sodium chloride adjustment and 1.2 μm/0.45 μm normal flow filtration. Clarified harvest is concentrated and buffer exchanged into PBS on a 750 kDa MWCO mPES hollow fiber membrane. The TFF retentate is filtered with a 0.45 μm filter before loading on a Sephacryl S-500 HR SEC column pre-equilibrated in PBS. Anellovectors are processed across the SEC column at 30 cm/hr. Individual fractions are collected and assayed by qPCR for viral genome copy number and transgene copy number. Viral genomes and transgene copies are observed beginning at the void volume, Fraction 7, of the SEC chromatogram. Agreement between copy number for Anellovirus genomes and Anellovirus-nLuc transgene for Anellovectors produced using circularized input DNA at Fraction 7-Fraction 10 indicates packaged Anellovectors containing nLuc transgene. SEC fractions are pooled and concentrated using a 100 kDa MWCO PVDF membrane and then 0.2 μm filtered prior to in vivo administration.

### Example 13: Production of Anellovectors Containing Chimeric ORF1 with Hypervariable Domains from Different Torque Teno Virus Strains

This example describes domain swapping of hypervariable regions of ORF1 to produce chimeric anellovectors containing the ORF1 arginine-rich region, jelly-roll domain, N22, and C-terminal domain of one TTV strain, and the hypervariable domain from an ORF1 protein of a different TTV strain.

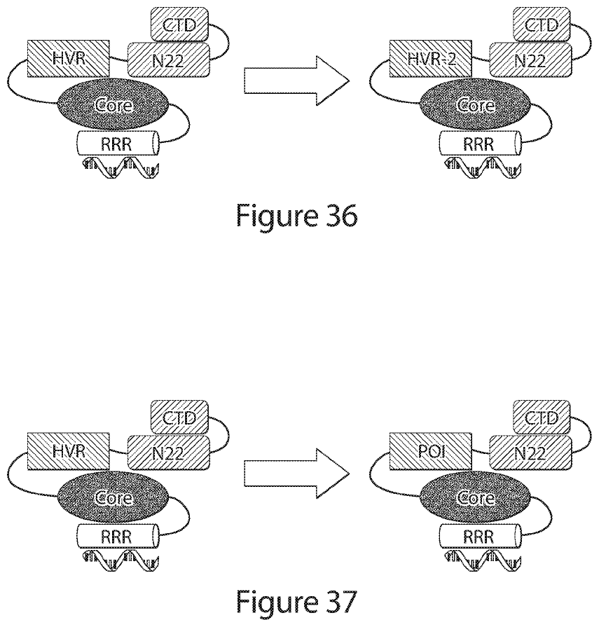

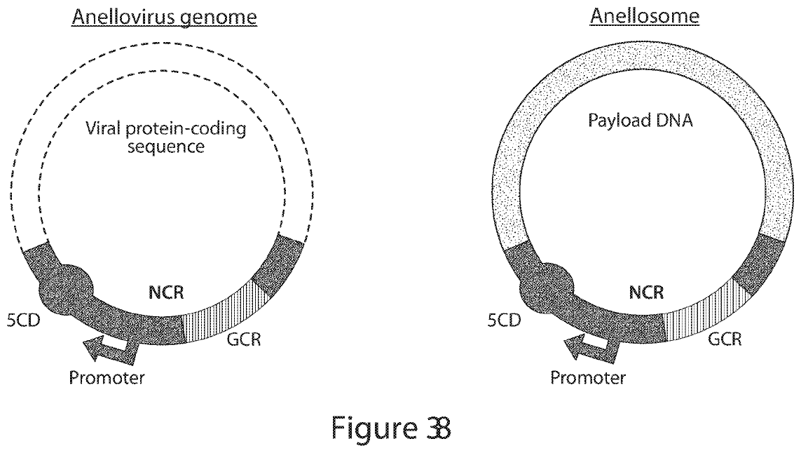

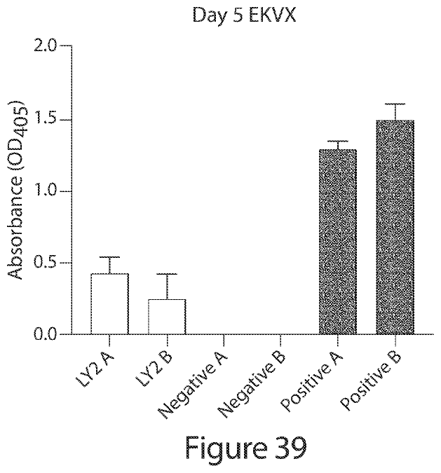

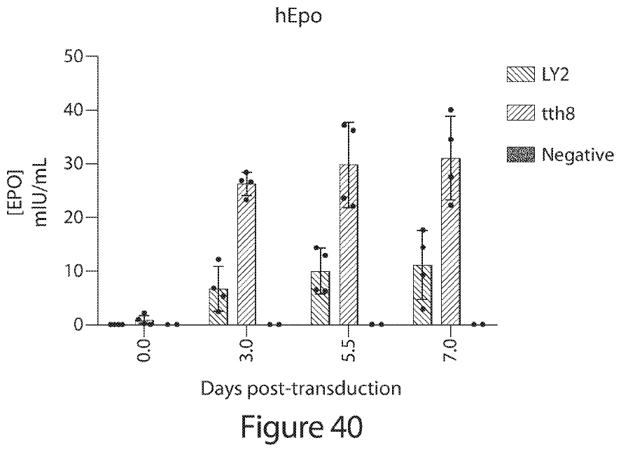

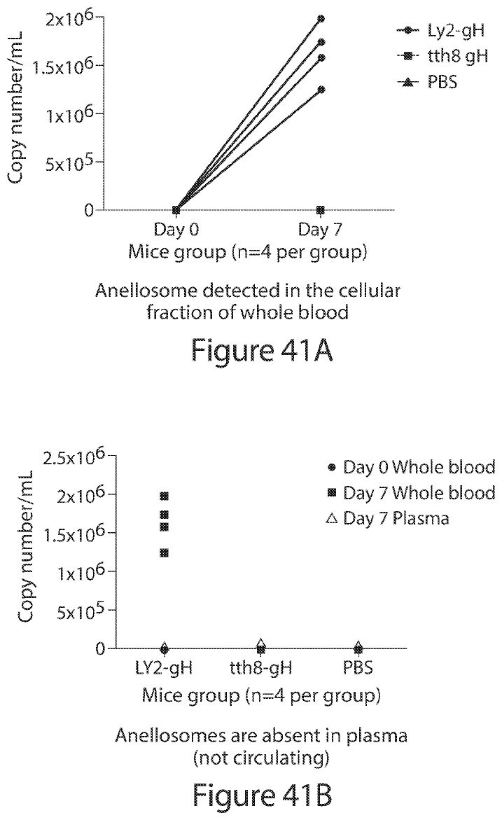

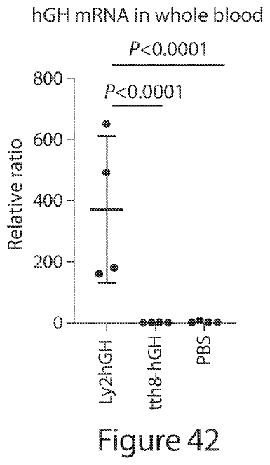

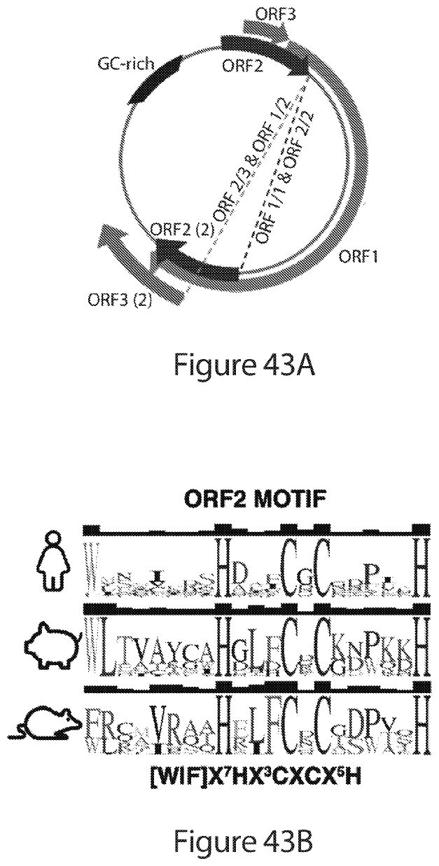

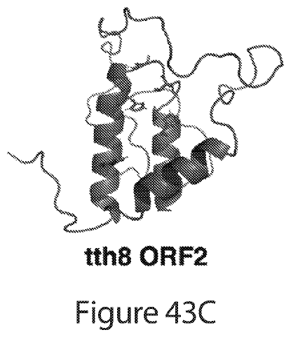

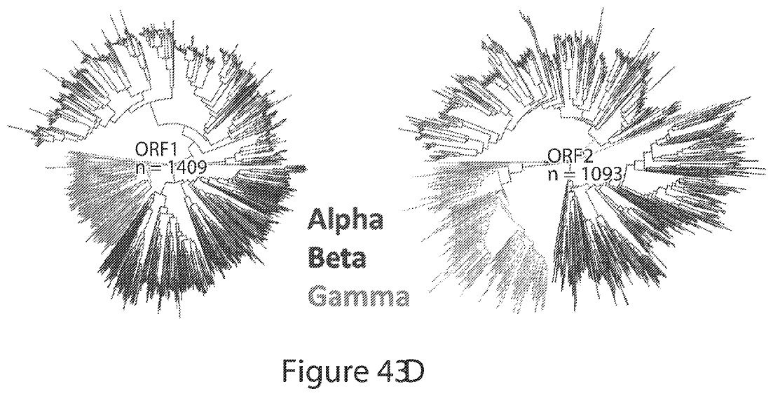

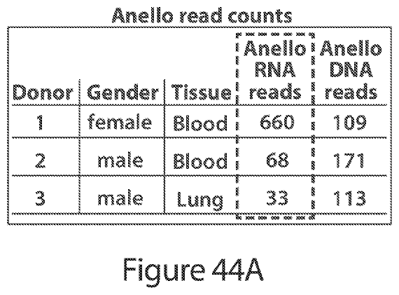

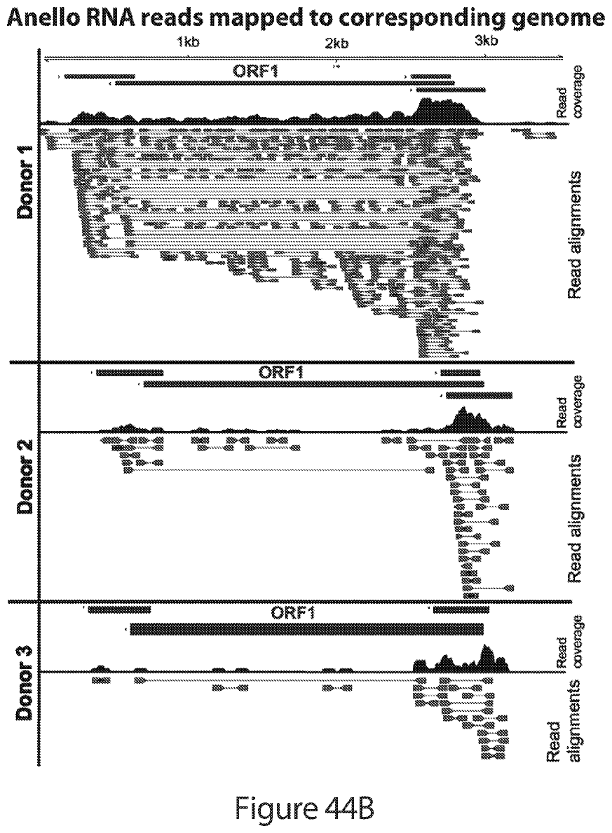

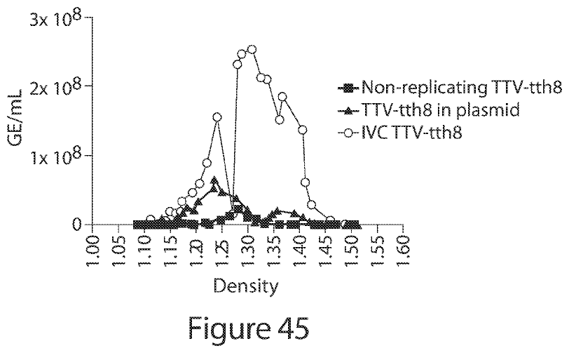

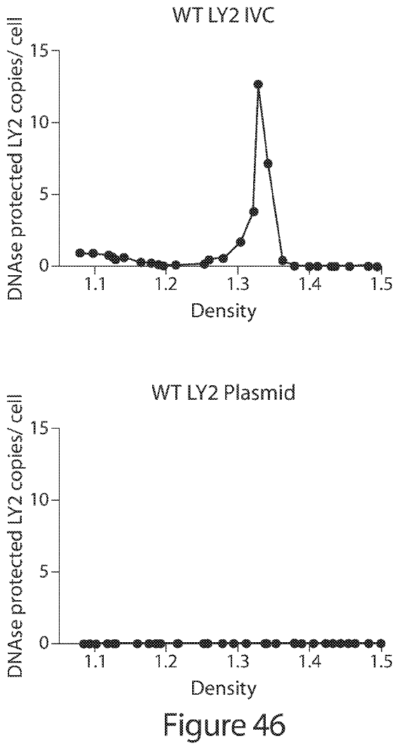

The full-length genome of a first Anellovirus is cloned into expression vectors for expression in mammalian cells. This genome is mutated to remove the hypervariable domain of the ORF1 coding sequence and replace it with the hypervariable domain of the ORF1 coding sequence from a second Anellovirus genome (FIG. 36). The plasmid containing the first Anellovirus genome with the swapped hypervariable domain is then linearized and circularized as described herein. HEK293T cells are transfected with the circularized genome and incubated for 5-7 days to allow anellovector production. After the incubation period anellovectors are purified from the supernatant and cell pellet of transfected cells by gradient ultracentrifugation.

To determine if the chimeric anellovectors are still infectious, the isolated viral particles are added to uninfected cells. The cells are incubated for 5-7 days to allow viral replication. After incubation the ability of the chimeric anellovectors to establish infection will be monitored by immunofluorescence, western blot, and qPCR. The structural integrity of the chimeric viruses is assessed by negative stain and cryo-electron microscopy. Chimeric anellovectors can further be tested for ability to infect cells in vivo. Establishment of the ability to produce functional chimeric anellovectors through hypervariable domain swapping could allow for engineering of viruses to alter tropism and potentially evade immune detection.

### Example 14: Design of an Anellovector Harboring a Payload

This example describes the design of an exemplary anellovector genetic element harboring a trans gene. The genetic element is composed of the essential cis replication and packaging domains from an Anellovirus genome (e.g., as described herein) along with a non-Anellovirus payload, which may include, e.g., protein or non-coding RNA-expressing genes. The anellovector lacks essential trans protein elements for replication and packaging, and requires proteins provided by other sources (e.g., helpers, e.g., replicating viruses, expression plasmids, or genome integrations) for rolling circle replication and encapsidation.

In one set of examples, the entire protein-coding DNA sequence is deleted, from the first start codon to the last stop codon (FIG. 38). The resulting DNA retains the viral non-coding region (NCR), including the viral promoter, the 5′ UTR conserved domain, the 3′ UTR (which encodes miRNAs in some anellovirus strains), and the GC-rich region. The anellovector NCR harbors essential cis domains, including the viral origin of replication and capsid binding domains. However, lacking the anellovirus protein-coding open reading frames, the anellovector is unable to express essential protein factors required for DNA replication and encapsidation, and therefore cannot amplify or package unless these elements are provided in trans.

Payload DNA, including but not limited to protein-encoding sequences, full trans genes (including non-anelloviral promoter sequences), and non-coding RNA genes are incorporated into the anellovector genetic element by insertion into the site of the deleted anelloviral open reading frames (FIG. 38). Expression from protein-coding sequences can be driven, for example, by either the native viral promoter or a synthetic promoter incorporated as a trans gene.

Replication-deficient or incompetent anellovector genetic elements (e.g., as described herein) may lack the protein-coding sequences for viral replication and/or capsid factors. Therefore, packaged anellovectors are produced by co-transfecting cells with the anellovector DNA described in this example and viral-protein-encoding DNA. The viral proteins are expressed off of replication-competent wild-type viral genomes, non-replicating plasmids harboring the viral proteins under control of the viral promoter, or plasmids harboring the viral proteins under control of a strong constitutive promoter.

### Example 15: Transduction of Anellovector Encoding a Transgene

In this example, an Anellovector carrying the payload immunoadhesin (IA) is made using an Anellovirus genome (e.g., as described herein), and then engineered to deliver a human immunoadhesin. A double-stranded circular IA anellovector DNA, which includes the Anellovirus non-coding regions (5′ UTR, GC-rich region) and an IA-encodnig cassette, but did not include the Anellovirus ORFs, is designed (e.g., as described herein) and then produced by in vitro circularization, as described herein. The Anellovirus ORFs are provided in trans in a separate in vitro circularized DNA. Both DNAs are co-transfected into HEK293T cells in two biological replicates. Two biological replicates each of a negative control (mock transfection) and a positive control (IA expression cassette in a plasmid) are also tested. Transduction of the anellovector preparation into the lung-derived human cell lines EKVX and A549 is expected to result in detection of secreted immunoadhesin by ELISA. Moreover, immunofluorescence analysis of the IA anellovector-transduced EKVX cells is expected to reveal cells that are positive for expression of the immunoadhesin.

### Example 16: Anellovectors Carrying the EPO Gene into Lung Cancer Cells

In this example, a non-small cell lung cancer line (EKVX) is transduced with anellovectors carrying the erythropocitin gene (EPO). The anellovectors are generated by in vitro circularization, as described herein. Each of the anellovectors included a genetic element that included the EPO-encoding cassette and non-coding regions of the Anellovirus genome (5′ UTR, GC-rich region), respectively, but did not include the Anellovirus ORFs, e.g., as described herein. Cells are inoculated with purified anellovectors or a positive control (AAV2-EPO at high dose or at the same dose as the anellovectors) and incubated for 7 days. Anellovirus ORFs are provided in trans in a separate in vitro circularized DNA. Culture supernatant is sampled 3, 5.5, and 7 days post-inoculation and assayed using a commercial ELISA kit to detect EPO. Successful transduction of cells is expected to result in significantly higher EPO titers compared to untreated (negative) control cells (P<0.013 at all time points).

### Example 17: Anellovectors with Therapeutic Transgenes can be Detected In Vivo after Intravenous (i.v.) Administration

In this example, anellovectors encoding human growth hormone (hGH) are detected in vivo after intravenous (i.v.) administration. Replication-deficient anellovectors encoding an exogenous hGH are generated by in vitro circularization as described herein. The genetic element of the hGH anellovectors includes Anellovirus non-coding regions (5′ UTR, GC-rich region) and the hGH-encoding cassette, but does not include Anellovirus ORFs. hGH anellovectors are administered to mice intravenously. The Anellovirus ORFs are provided in trans in a separate in vitro circularized DNA. Briefly, anellovectors or PBS are injected intravenously at day 0 (n=4 mice/group). Anellovectors are administered to independent animal groups at 4.66E+07 anellovector genomes per mouse.

In a first example, anellovector viral genome DNA copies are detected. At day 7, blood and plasma are collected and analyzed for the hGH DNA amplicon by qPCR. Presence of hGH anellovectors in the cellular fraction of whole blood after 7 days post infection in vivo and the absence of anellovectors in plasma would demonstrate the inability of such anellovectors to replicate in vivo.

In a second example, hGH mRNA transcripts are detected after in vivo transduction. At day 7, blood is collected and analyzed for the hGH mRNA transcript amplicon by qRT-PCR. GAPDH is used as a control housekeeping gene. hGH mRNA transcripts are measured in the cellular fraction of whole blood.

### Example 18: In Vitro Circularized Genome as Input Material for Producing Anellovectors In Vitro

This example demonstrates whether in vitro circularized (IVC) double stranded anellovirus DNA, as source material for an anellovector genetic element as described herein, is more robust than an anellovirus genome DNA in a plasmid to yield packaged anellovector genomes of the expected density.

1.2E+07 HEK293T cells (human embryonic kidney cell line) in T75 flasks are transfected with 11.25 ug of either (i) in vitro circularized double stranded Anellovirus genome (IVC Anellovirus), (ii) Anellovirus genome in a plasmid backbone, or (iii) plasmid containing just the ORF1 sequence of Anellovirus (non-replicating Anellovirus). Cells are harvested 7 days post transfection, lysed with 0.1% Triton, and treated with 100 units per ml of Benzonase. The lysates are used for cesium chloride density analysis; density is measured and TTV-tth8 copy quantification is performed for each fraction of the cesium chloride linear gradient.

1E+07 Jurkat cells (human T lymphocyte cell line) are nucleofected with either in-vitro circularized Anellovirus genome (IVC Anellovirus) or Anellovirus genome in plasmid. Cells are harvested 4 days post transfection and lysed using a buffer containing 0.5% triton and 300 mM sodium chloride, followed by two rounds of instant freeze-thaw. The lysates are treated with 100 units/ml benzonase, followed by cesium chloride density analysis. Density measurement and LY2 genome quantification is performed on each fraction of the cesium chloride linear gradient.

LENGTHY TABLES
The patent application contains a lengthy table section. A copy of the table is available in electronic form from the USPTO web site (https://seqdata.uspto.gov/?pageRequest=docDetail&DocID=US20240254512A1). An electronic copy of the table will also be available from the USPTO upon request and payment of the fee set forth in 37 CFR 1.19(b)(3).

What is claimed is:

1. An anellovector comprising:
(i) a proteinaceous exterior comprising an Anellovirus ORF1 protein comprising an amino acid sequence of SEQ ID NO: 2466, or a polypeptide comprising an amino acid sequence having at least about 70%, 75%, 80%, 85%, 90%, 91%, 92%, 93%, 94%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.

2. An anellovector comprising:
(i) a proteinaceous exterior comprising an Anellovirus ORF1 protein comprising an amino acid sequence of SEQ ID NO: 2466, or a polypeptide comprising an amino acid sequence having at least about 70%, 75%, 80%, 85%, 90%, 91%, 92%, 93%, 94%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF 1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

3. An anellovector comprising:
(i) a proteinaceous exterior comprising a polypeptide encoded by an Anellovirus ORF1 nucleic acid sequence according to nucleotides 338-2305 of SEQ ID NO: 2465, or a polypeptide encoded by a nucleic acid sequence having at least about 70%, 75%, 80%, 85%, 90%, 91%, 92%, 93%, 94%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.

4. An anellovector comprising:
(i) a proteinaceous exterior comprising a polypeptide encoded by an Anellovirus ORF1 nucleic acid sequence according to nucleotides 338-2305 of SEQ ID NO: 2465, or a polypeptide encoded by a nucleic acid sequence having at least about 70%, 75%, 80%, 85%, 90%, 91%, 92%, 93%, 94%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

5. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 91%, 92%, 93%, 94%, 95%, 96%, 97%, 98%, or 99% sequence identity thereto), and
(ii) a genetic element enclosed by the proteinaceous exterior, wherein the genetic element comprises: (a) a 5′ UTR conserved domain according to nucleotides 1-64 of SEQ ID NO: 2465, or a nucleic acid sequence having at least 70%, 75%, 80%, 85%, 90%, 91%, 92%, 93%, 94%, 95%, 96%, 97%, 98%, 99%, or 100% sequence identity thereto, and (b) a promoter element operably linked to a nucleic acid sequence (e.g., a DNA sequence) encoding an exogenous effector.

6. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%,
(ii) a genetic element enclosed by the proteinaceous exterior, wherein the genetic element comprises: (a) a 5′ UTR conserved domain according to nucleotides 1-64 of SEQ ID NO: 2465, or a nucleic acid sequence having at least 70%, 75%, 80%, 85%, 90%, 91%, 92%, 93%, 94%, 95%, 96%, 97%, 98%, 99%, or 100% sequence identity thereto, and (b) a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

7. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 91%, 92%, 93%, 94%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an exogenous effector, and wherein the genetic element has at least about 70%, 75%, 80%, 85%, 90%, 91%, 92%, 93%, 94%, 95%, 96%, 97%, 98%, 99%, or 100% sequence identity to an Anellovirus genome sequence of SEQ ID NO: 2465.

8. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 91%, 92%, 93%, 94%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector), and wherein the genetic element has at least about 70%, 75%, 80%, 85%, 90%, 91%, 92%, 93%, 94%, 95%, 96%, 97%, 98%, 99%, or 100% sequence identity to an Anellovirus genome sequence of SEQ ID NO: 2465;
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

9. An isolated ORF1 molecule comprising the amino acid sequence of SEQ ID NO: 2466, or an amino acid sequence having at least about 70%, 75%, 80%, 85%, 90%, 91%, 92%, 93%, 94%, 95%, 96%, 97%, 98%, 99%, or 100% sequence identity thereto:
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

10. An isolated ORF1 molecule comprising the amino acid sequence of the jelly-roll domain of SEQ ID NO: 2466, or an amino acid sequence having at least about 70%, 75%, 80%, 85%, 90%, 91%, 92%, 93%, 94%, 95%, 96%, 97%, 98%, 99%, or 100% sequence identity thereto:
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

11. An isolated ORF2 molecule comprising the amino acid sequence of SEQ ID NO: 2467, or an amino acid sequence having at least about 70%, 75%, 80%, 85%, 90%, 91%, 92%, 93%, 94%, 95%, 96%, 97%, 98%, 99%, or 100% sequence identity thereto:
wherein the ORF2 molecule comprises at least one difference (e.g., a mutation, chemical modification, or epigenetic alteration) relative to a wild-type ORF2 protein (e.g., as described herein), e.g., an insertion, substitution, chemical or enzymatic modification, and/or deletion, e.g., a deletion of a domain.

12. An isolated nucleic acid molecule (e.g., a genetic element construct or a genetic element) comprising the nucleic acid sequence of a 5′ UTR conserved domain according to nucleotides 1-64 of SEQ ID NO: 2465, or a nucleic acid sequence having at least 70%, 75%, 80%, 85%, 90%, 91%, 92%, 93%, 94%, 95%, 96%, 97%, 98%, or 99% sequence identity thereto.

13. An isolated nucleic acid molecule (e.g., a genetic element construct or a construct for providing an ORF1 molecule in trans. e.g., as described herein) comprising the nucleic acid sequence of an ORF1 gene according to nucleotides 338-2305 of SEQ ID NO: 2465, or a nucleic acid sequence having at least 70%, 75%, 80%, 85%, 90%, 91%, 92%, 93%, 94%, 95%, 96%, 97%, 98%, or 99% sequence identity thereto.

14. An isolated nucleic acid molecule (e.g., a genetic element construct or a construct for providing an ORF2 molecule in trans. e.g., as described herein) comprising the nucleic acid sequence of an ORF2 gene according to nucleotides 114-446 of SEQ ID NO: 2465, or a nucleic acid sequence having at least 70%, 75%, 80%, 85%, 90%, 91%, 92%, 93%, 94%, 95%, 96%, 97%, 98%, or 99% sequence identity thereto.

15. An isolated nucleic acid molecule (e.g., a genetic element construct, a genetic element, or a construct for providing an ORF1 or ORF2 molecule in trans. e.g., as described herein) comprising an Anellovirus genome sequence of SEQ ID NO: 2465, or a nucleic acid sequence having at least 50%, 60%, 70%, 75%, 80%, 85%, 90%, 91%, 92%, 93%, 94%, 95%, 96%, 97%, 98%, or 99% sequence identity thereto.

16. A genetic element comprising:
(a) a 5′ UTR conserved domain according to nucleotides 1-64 of SEQ ID NO: 2465, or a nucleic acid sequence having at least 70%, 75%, 80%, 85%, 90%, 91%, 92%, 93%, 94%, 95%, 96%, 97%, 98%, 99%, or 100% sequence identity thereto, and
(b) a promoter element operably linked to a nucleic acid sequence (e.g., a DNA sequence) encoding an exogenous effector.

17. A method of manufacturing an anellovector composition, the method comprising:
(a) providing a cell, e.g., a host cell as described herein;
(b) introducing a nucleic acid molecule encoding an ORF1 polypeptide comprising a sequence of SEQ ID NO: 2466 or ORF2 polypeptide comprising a sequence of SEQ ID NO: 2467 (or an amino acid sequence having at least about 70%, 75%, 80%, 85%, 90%, 91%, 92%, 93%, 94%, 95%, 96%, 97%, 98%, or 99% sequence identity thereto) into the cell;
(c) introducing a genetic element construct into the cell (e.g., before, after, or simultaneously with (b)),
(d) incubating the cell under conditions that allow the cell to produce anellovector; and
(e) formulating the anellovectors, e.g., as a pharmaceutical composition suitable for administration to a subject,
thereby making the anellovector composition.

18. A method of manufacturing an anellovector composition, the method comprising:
(a) providing a cell, e.g., a host cell as described herein;
(b) introducing a nucleic acid molecule encoding an ORF1 or ORF2 polypeptide into the cell;
(c) introducing a genetic element construct comprising a 5′ UTR conserved domain according to nucleotides 1-64 of SEQ ID NO: 2465 (or a nucleic acid sequence having at least about 70%, 75%, 80%, 85%, 90%, 91%, 92%, 93%, 94%, 95%, 96%, 97%, 98%,
or 99% sequence identity thereto) into the cell (e.g., before, after, or simultaneously with (b)),
(d) incubating the cell under conditions that allow the cell to produce anellovector; and
(e) formulating the anellovectors, e.g., as a pharmaceutical composition suitable for administration to a subject,
thereby making the anellovector composition.

19. A method of making an anellovector, e.g., a synthetic anellovector, comprising:
(a) providing a host cell comprising:
(i) a nucleic acid molecule, e.g., a first nucleic acid molecule, comprising the nucleic acid sequence of a Anellovirus genome comprising a sequence of SEQ ID NO: 2465 (or a nucleic acid sequence having at least 70%, 75%, 80%, 85%, 90%, 91%, 92%, 93%, 94%, 95%, 96%, 97%, 98%, or 99% sequence identity thereto), and
(ii) a nucleic acid molecule, e.g., a second nucleic acid molecule, encoding one or more of an amino acid sequence chosen from an ORF1, ORF2, ORF2/2, ORF2/3, ORF1/1, or ORF1/2 encoded by SEQ ID NO: 2465, or an amino acid sequence having at least 70% 75%, 80%, 85%, 90%, 91%, 92%, 93%, 94%, 95%, 96%, 97%, 98%, or 99% sequence identity thereto; and
(b) culturing the host cell under conditions suitable to make the anellovector.

20. An anellovector comprising:
(i) a proteinaceous exterior comprising an Anellovirus ORF1 protein comprising an amino acid sequence of SEQ ID NO: 268, or a polypeptide comprising an amino acid sequence having at least about 70%, 75%, 80%, 85%, 90%, 91%, 92%, 93%, 94%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.

21. An anellovector comprising:
(i) a proteinaceous exterior comprising an Anellovirus ORF1 protein comprising an amino acid sequence of SEQ ID NO: 268, or a polypeptide comprising an amino acid sequence having at least about 70%, 75%, 80%, 85%, 90%, 91%, 92%, 93%, 94%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

22. An anellovector comprising:
(i) a proteinaceous exterior comprising a polypeptide encoded by an Anellovirus ORF1 nucleic acid sequence according to nucleotides 407-2617 of SEQ ID NO: 267, or a polypeptide encoded by a nucleic acid sequence having at least about 70%, 75%, 80%, 85%, 90%, 91%, 92%, 93%, 94%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.

23. An anellovector comprising:
(i) a proteinaceous exterior comprising a polypeptide encoded by an Anellovirus ORF1 nucleic acid sequence according to nucleotides 407-2617 of SEQ ID NO: 267, or a polypeptide encoded by a nucleic acid sequence having at least about 70%, 75%, 80%, 85%, 90%, 91%, 92%, 93%, 94%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

24. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 91%, 92%, 93%, 94%, 95%, 96%, 97%, 98%, or 99% sequence identity thereto), and
(ii) a genetic element enclosed by the proteinaceous exterior, wherein the genetic element comprises: (a) a 5′ UTR conserved domain according to nucleotides 1-70 of SEQ ID NO: 267, or a nucleic acid sequence having at least 70%, 75%, 80%, 85%, 90%, 91%, 92%, 93%, 94%, 95%, 96%, 97%, 98%, 99%, or 100% sequence identity thereto, and (b) a promoter element operably linked to a nucleic acid sequence (e.g., a DNA sequence) encoding an exogenous effector.

25. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%,
(ii) a genetic element enclosed by the proteinaceous exterior, wherein the genetic element comprises: (a) a 5′ UTR conserved domain according to nucleotides 1-70 of SEQ ID NO: 267, or a nucleic acid sequence having at least 70%, 75%, 80%, 85%, 90%, 91%, 92%, 93%, 94%, 95%, 96%, 97%, 98%, 99%, or 100% sequence identity thereto, and (b) a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

26. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 91%, 92%, 93%, 94%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an exogenous effector, and wherein the genetic element has at least about 70%, 75%, 80%, 85%, 90%, 91%, 92%, 93%, 94%, 95%, 96%, 97%, 98%, 99%, or 100% sequence identity to an Anellovirus genome sequence of SEQ ID NO: 267.

27. An anellovector comprising:
(i) a proteinaceous exterior (e.g., comprising an Anellovirus ORF1 molecule as described herein, or a polypeptide comprising an amino acid sequence having at least about 70%, 75%, 80%, 85%, 90%, 91%, 92%, 93%, 94%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector), and wherein the genetic element has at least about 70%, 75%, 80%, 85%, 90%, 91%, 92%, 93%, 94%, 95%, 96%, 97%, 98%, 99%, or 100% sequence identity to an Anellovirus genome sequence of SEQ ID NO: 267;
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

28. An isolated ORF1 molecule comprising the amino acid sequence of SEQ ID NO: 268, or an amino acid sequence having at least about 70%, 75%, 80%, 85%, 90%, 91%, 92%, 93%, 94%, 95%, 96%, 97%, 98%, 99%, or 100% sequence identity thereto:
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

29. An isolated ORF1 molecule comprising the amino acid sequence of the jelly-roll domain of SEQ ID NO: 268, or an amino acid sequence having at least about 70%, 75%, 80%, 85%, 90%, 91%, 92%, 93%, 94%, 95%, 96%, 97%, 98%, 99%, or 100% sequence identity thereto:
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

30. An isolated ORF2 molecule comprising the amino acid sequence of SEQ ID NO: 269, or an amino acid sequence having at least about 70%, 75%, 80%, 85%, 90%, 91%, 92%, 93%, 94%, 95%, 96%, 97%, 98%, 99%, or 100% sequence identity thereto:
wherein the ORF2 molecule comprises at least one difference (e.g., a mutation, chemical modification, or epigenetic alteration) relative to a wild-type ORF2 protein (e.g., as described herein), e.g., an insertion, substitution, chemical or enzymatic modification, and/or deletion, e.g., a deletion of a domain.

31. An isolated nucleic acid molecule (e.g., a genetic element construct or a genetic element) comprising the nucleic acid sequence of a 5′ UTR conserved domain according to nucleotides 1-70 of SEQ ID NO: 267, or a nucleic acid sequence having at least 70%, 75%, 80%, 85%, 90%, 91%, 92%, 93%, 94%, 95%, 96%, 97%, 98%, or 99% sequence identity thereto.

32. An isolated nucleic acid molecule (e.g., a genetic element construct or a construct for providing an ORF1 molecule in trans. e.g., as described herein) comprising the nucleic acid sequence of an ORF1 gene according to nucleotides 407-2617 of SEQ ID NO: 267, or a nucleic acid sequence having at least 70%, 75%, 80%, 85%, 90%, 91%, 92%, 93%, 94%, 95%, 96%, 97%, 98%, or 99% sequence identity thereto.

33. An isolated nucleic acid molecule (e.g., a genetic element construct or a construct for providing an ORF2 molecule in trans. e.g., as described herein) comprising the nucleic acid sequence of an ORF2 gene according to nucleotides 87-533 of SEQ ID NO: 267, or a nucleic acid sequence having at least 70%, 75%, 80%, 85%, 90%, 91%, 92%, 93%, 94%, 95%, 96%, 97%, 98%, or 99% sequence identity thereto.

34. An isolated nucleic acid molecule (e.g., a genetic element construct, a genetic element, or a construct for providing an ORF1 or ORF2 molecule in trans. e.g., as described herein) comprising an Anellovirus genome sequence of SEQ ID NO: 267, or a nucleic acid sequence having at least 50%, 60%, 70%, 75%, 80%, 85%, 90%, 91%, 92%, 93%, 94%, 95%, 96%, 97%, 98%, or 99% sequence identity thereto.

35. A genetic element comprising:
(a) a 5′ UTR conserved domain according to nucleotides 1-70 of SEQ ID NO: 267, or a nucleic acid sequence having at least 70%, 75%, 80%, 85%, 90%, 91%, 92%, 93%, 94%, 95%, 96%, 97%, 98%, 99%, or 100% sequence identity thereto, and
(b) a promoter element operably linked to a nucleic acid sequence (e.g., a DNA sequence) encoding an exogenous effector.

36. A method of manufacturing an anellovector composition, the method comprising:
(f) providing a cell, e.g., a host cell as described herein;
(g) introducing a nucleic acid molecule encoding an ORF1 polypeptide comprising a sequence of SEQ ID NO: 268 or ORF2 polypeptide comprising a sequence of SEQ ID NO: 269 (or an amino acid sequence having at least about 70%, 75%, 80%, 85%, 90%, 91%, 92%, 93%, 94%, 95%, 96%, 97%, 98%, or 99% sequence identity thereto) into the cell;
(h) introducing a genetic element construct into the cell (e.g., before, after, or simultaneously with (b)),
(i) incubating the cell under conditions that allow the cell to produce anellovector; and
(j) formulating the anellovectors, e.g., as a pharmaceutical composition suitable for administration to a subject,
thereby making the anellovector composition.

37. A method of manufacturing an anellovector composition, the method comprising:
(f) providing a cell, e.g., a host cell as described herein;
(g) introducing a nucleic acid molecule encoding an ORF1 or ORF2 polypeptide into the cell;
(h) introducing a genetic element construct comprising a 5′ UTR conserved domain according to nucleotides 1-70 of SEQ ID NO: 267 (or a nucleic acid sequence having at least about 70%, 75%, 80%, 85%, 90%, 91%, 92%, 93%, 94%, 95%, 96%, 97%, 98%, or 99% sequence identity thereto) into the cell (e.g., before, after, or simultaneously with (b)),
(i) incubating the cell under conditions that allow the cell to produce anellovector; and
(j) formulating the anellovectors, e.g., as a pharmaceutical composition suitable for administration to a subject,
thereby making the anellovector composition.

38. A method of making an anellovector, e.g., a synthetic anellovector, comprising:
(a) providing a host cell comprising:
(i) a nucleic acid molecule, e.g., a first nucleic acid molecule, comprising the nucleic acid sequence of a Anellovirus genome comprising a sequence of SEQ ID NO: 267 (or a nucleic acid sequence having at least 70%, 75%, 80%, 85%, 90%, 91%, 92%, 93%, 94%, 95%, 96%, 97%, 98%, or 99% sequence identity thereto), and
(ii) a nucleic acid molecule, e.g., a second nucleic acid molecule, encoding one or more of an amino acid sequence chosen from an ORF1, ORF2, ORF2/2, ORF2/3, ORF1/1, or ORF1/2 encoded by SEQ ID NO: 267, or an amino acid sequence having at least 70% 75%, 80%, 85%, 90%, 91%, 92%, 93%, 94%, 95%, 96%, 97%, 98%, or 99% sequence identity thereto; and
(b) culturing the host cell under conditions suitable to make the anellovector.
